(19) United States
(12) Patent Application Publication
(10) Pub. No.: US 2025/0101032 A1
(43) Pub. Date: Mar. 27, 2025

(54) EXATECAN DERIVATIVES AND ANTIBODY-DRUG CONJUGATES THEREOF

(71) Applicant: ALX Oncology Inc., South San Francisco, CA (US)

(72) Inventors: Marija Vrljic, San Mateo, CA (US); Peter Strop, Tucson, AZ (US); Janica Cheuk-ying Wong, Foster City, CA (US); Jaume Pons, South San Francisco, CA (US)

(21) Appl. No.: 18/559,185

(22) PCT Filed: May 6, 2022
(86) PCT No.: PCT/US2022/028193

§ 371 Date: Nov. 6, 2023

Related U.S. Application Data
(60) Provisional application No. 63/321,187, filed on Mar. 18, 2022.
(60) Provisional application No. 63/248,705, filed on Sep. 27, 2021.
(60) Provisional application No. 63/185,736, filed on May 7, 2021.

(51) Int. Cl. C07D 491/22; A61K 31/4745; A61K 31/498; A61K 47/68; A61P 35/00; C07D 519/00

(52) U.S. Cl. CPC C07D 491/22; A61K 31/4745; A61K 31/498; A61K 47/68037; A61K 47/6851; A61K 47/6889; A61P 35/00; C07D 519/00

(57) ABSTRACT

Disclosed herein, in part, are novel exatecan derivatives with novel chemical linkers that include cathepsin B cleavable moieties, and conjugated to targeting antibodies.

### CROSS-REFERENCE TO RELATED APPLICATIONS

This application is a national phase filing under 35 U.S.C. § 371 of International (PCT) Application No. PCT/US2022/028193 filed May 6, 2022; which claims the benefit of, and priority to, U.S. Ser. No. 63/185,736 filed May 7, 2021; U.S. Ser. No. 63/248,705 filed Sep. 27, 2021; and U.S. Ser. No. 63/321,187 filed Mar. 18, 2022; the contents of each of which are incorporated herein by reference in their entirety.

### BACKGROUND

Antibody-drug conjugates (ADC's) provide a mechanism for selective delivery of small molecule therapeutic payloads to antigen-positive cancer cells, thereby attenuating systemic toxicity of cytotoxic drugs to antigen-negative normal cells. Three components of an ADC—the antibody, the cytotoxic payload, and the linker that joins them—are important in designing an effective therapeutic. Despite active development, challenges still exist, for example, toxicity due to the antibody binding to its target in normal tissue, and dispersion of the cytotoxic payload in normal tissue due to instability of the ADC linker. Thus, many ADCs's have not succeeded in clinical trials due to lack of safety and/or efficacy at tolerated doses.

Topoisomerase I plays a critical role in DNA replication in both normal and diseased conditions (e.g., cancer). As inhibition of topoisomerase I leads to cell death, compounds that bind to and inhibit topoisomerase I may be useful as therapeutic agents.

Camptothecin is a natural product with cytotoxic activity in a variety of cell lines. The binding of its active lactone ring to topoisomerase I inhibits DNA replication, thus causing cell apoptosis. However, its limitations for drug development include, for example, poor water solubility and an equilibrium between its active, lactone form and its inactive, ring-opened form.

Exatecan is a water-soluble camptothecin derivative. As a chemotherapeutic agent, exatecan mesylate did not gain drug approval after several clinical trials due to lack of efficacy or high toxicity at tested doses. Efforts to enable the clinical utility of exatecan have been made by converting exatecan into a prodrug form, where exatecan is covalently linked to a carboxymethyldextran polyalcohol polymer via a peptidyl spacer (a substrate for intracellular cathepsin proteases). However, this prodrug did not succeed in clinical trials.

Thus, a need exists for compounds more amenable to clinical development and success in treating human tumors. Moreover, preferential delivery of topoisomerase I inhibitors to diseased tissues through antibody-drug conjugates could lead to improved safety and efficacy, thereby providing therapeutic options for a larger number of patients and types of cancers.

### SUMMARY

The present disclosure relates to compounds useful for the treatment of cancer. The present disclosure is directed, in part, to exatecan derivatives useful as payloads in drug conjugates (e.g., antibody-drug conjugates), linker-payload constructs useful for attaching the payloads to antibodies, and exatecan-based drug conjugates. For example, provided herein are compounds representing a therapeutic payload, a linker-payload construct, or a drug conjugate.

For example, the present disclosure provides exatecan derivatives for use as therapeutic payloads. Also proved herein are linker-payload constructs and drug conjugates, each comprising a disclosed therapeutic payload. Further provided herein is the use of disclosed compounds as medicinal agents, processes for their preparation, and pharmaceutical compositions containing them as an active ingredient both alone or in combination with other agents, as well as provides for their use as medicaments and/or in the manufacture of medicaments for the treatment of cancer.

For example, disclosed herein is a therapeutic payload represented by Formula I:

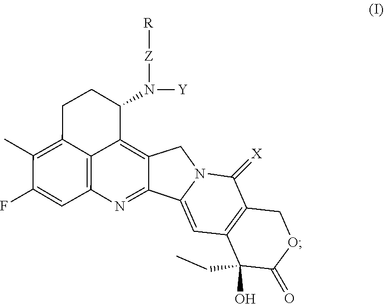

or a pharmaceutically acceptable salt or stereoisomer thereof, wherein:
X is selected from the group consisting of O and S;
Z is a bond; Y is selected from the group consisting of hydrogen, —C₁₋₃alkyl, —CHO, and —C(O)—C₁₋₃alkyl; and R is selected from the group consisting of R¹, R², R³, R⁴, R⁵ and hydrogen; or
Y and Z, together with the nitrogen to which they are attached, are joined together to form a 5-6 membered heteroaryl optionally substituted by one, two or three substituents, each independently selected from R^{Z}; R is bonded to the heteroaryl; and R is R⁶;
R¹ is selected from the group consisting of —C(O)—C₁₋₃alkyl, —C(O)—O—C₁₋₃alkyl, C₁₋₄alkyl, —C₁₋₃alkyl-O—C₁₋₃alkyl, —C(O)—C₃₋₄alkynyl, —S(O)₂—C₁₋₃alkyl, —C(S)—C₁₋₃alkyl, —C₁₋₃alkyl-S—C₁₋₃ alkyl, and —C(O)—O—[(CH₂)₂—O]₁₋₁₀—C₂alkyl; wherein R¹ is substituted by hydroxyl and optionally substituted by one or more additional substituents each independently selected from R¹¹;
R¹¹ is independently selected for each occurrence from the group consisting of halogen, hydroxyl, —C₁₋₃alkyl-OH, —C₁₋₃haloalkyl, and —C₃₋₄cycloalkyl;
R² is selected from the group consisting of —C(O)—NR^{a}—C₁₋₃alkyl, —C(O)—C₀₋₃alkyl-C(O)—NR^{a}—C₁₋₃alkyl, —C(O)—C₁₋₃alkyl-NR^{a}—C₁₋₃alkyl, —S(O)₂—C₁₋₃alkyl-NR^{a}—C(O)—C₁₋₃alkyl, and —C(O)NR^{a}—[(CH₂)₂—O]₁₋₁₀-C₂alkyl; wherein R² is substituted by hydroxyl and optionally substituted by one or more additional substituents each independently selected from R²²;
R²² is independently selected for each occurrence from the group consisting of halogen, hydroxyl, —C₁₋₃alkyl-OH, and —C₁₋₃haloalkyl;
R³ is selected from the group consisting of —C(O)—C₀₋₃alkyl-R³⁰, —C(O)—C₀₋₃alkyl-O—C₁₋₃alkyl-R³⁰, —C₀₋₃alkyl-R³⁰, and —C₁₋₃alkyl-O—C₁₋₃alkyl-R³⁰; wherein the alkyl if present may optionally be substituted by one or more substituents each independently selected from the group consisting of halogen and —C₁₋₃haloalkyl;
R³⁰ is selected from the group consisting of 5-6 membered heteroaryl and 4-10 membered heterocyclyl having one, two or three heteroatoms, each independently selected from the group consisting of N, NR³¹, and O; wherein R³⁰ is optionally substituted on one or more available carbons by one or more substituents each independently selected from R³³;
R³¹ is independently selected for each occurrence from the group consisting of hydrogen, —C₁₋₃alkyl, —C₁₋₃alkyl-OH, —CH(OH)CH₂OH, —CHO, and —C(O)—C₁₋₃alkyl;
R³³ is independently selected for each occurrence from the group consisting of —C₁₋₃alkyl-OH, halogen, hydroxyl, oxo, and —C₁₋₃haloalkyl;
R⁴ is selected from the group consisting of —C(O)—NR^{a}—C₃₋₆cycloalkyl, —C(O)—C₀₋₂alkyl-C₃₋₆cycloalkyl, —C(S)—C₀₋₂alkyl-C₃₋₆cycloalkyl, —C(O)—NR^{a}—C₃₋₆cycloalkyl, and —C₃₋₆cycloalkenyl-NR^{a}—C₁₋₃alkyl; wherein R⁴ is substituted by one or more substituents each independently selected from R⁴⁴;
R⁴⁴ is independently selected for each occurrence from the group consisting of hydroxyl, halogen, oxo, —C₁₋₃alkyl, and —C₁₋₃alkyl-OH;
R⁵ is selected from the group consisting of —S(O)₂—C₁₋₃alkyl-NR^{a}R^{b}, —C₁₋₄alkyl-NR^{a}R^{b}, —C(O)—C₁₋₃alkyl-O—NR^{a}R^{b}, —N═S(═O)(C₁₋₃alkyl)C₁₋₃alkyl, —C(O)—CH₂-phenyl-CH₂NR^{a}R^{b}, and —[(CH₂)₂—NR^{a}]₁₋₅—C₁₋₃alkyl-NR^{a}R^{b}; wherein alkyl may optionally be substituted by one or more substituents each independently selected from R⁵⁵;
R⁵⁵ is independently selected for each occurrence from the group consisting of halogen, —C₁₋₃alkyl and —C₁₋₃haloalkyl;
R⁶ is —C₁₋₃alkyl substituted by hydroxyl and optionally substituted by one or more additional substituents each independently selected from R⁶⁶
R⁶⁶ is independently selected for each occurrence from the group consisting of halogen and —C₁₋₃haloalkyl;
R^{Z} is selected from the group consisting of halogen, —C₁₋₃alkyl and —C₁₋₃alkyl-OH; and
R^{a} and R^{b} are each independently selected for each occurrence from the group consisting of the group consisting of hydrogen, —C₁₋₃alkyl-OH, and —C₁₋₃haloalkyl-OH;
wherein when X is O and Y is H, then R is not hydrogen or —C(O)CH₂OH.

Also disclosed herein is a linker-payload construct represented by Formula IIA or Formula IIB:

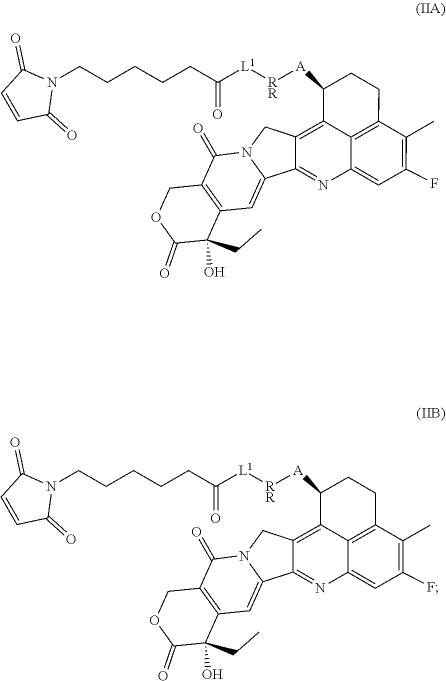

or a pharmaceutically acceptable salt or stereoisomer thereof, wherein:
A is NH or triazolyl;
L¹ is —CBP—NH—CH₂—, or —CBP—, wherein CBP is a cathepsin B cleavable peptide or a cathepsin D cleavable peptide; and
RR is an alkoxy or amino moiety formed from L¹ and a hydroxy or —NH₂ moiety of a therapeutic payload described herein.

Further disclosed herein is a linker-payload construct represented by Formula IIIA or Formula IIIB:

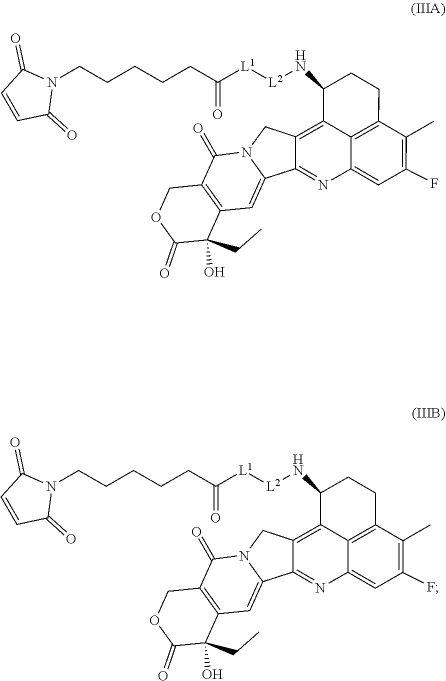

or a pharmaceutically acceptable salt or stereoisomer thereof, wherein:
L¹ is a cathepsin B cleavable peptide or a cathepsin D cleavable peptide; and
L² is a self-immolating moiety.

Additionally, disclosed herein is a drug conjugate represented by Formula IVA or Formula IVB:

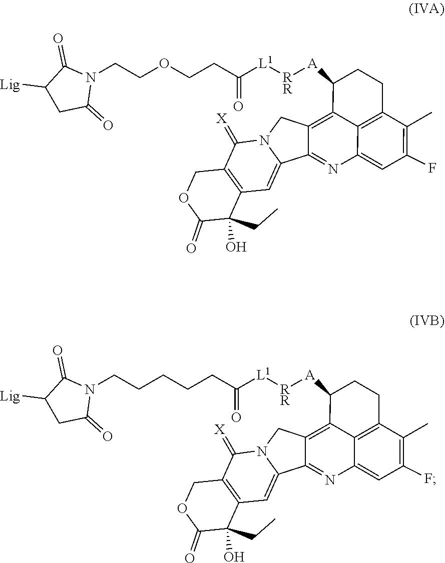

or a pharmaceutically acceptable salt or stereoisomer thereof, wherein:
X is O or S;
A is NH or triazolyl;
Lig is a targeting moiety;
L¹ is —CBP—NH—CH₂— or —CBP—, wherein CBP is a cathepsin B cleavable peptide or a cathepsin D cleavable peptide; and
RR is an alkoxy or amino moiety formed from L¹ and a hydroxy or —NH₂ moiety of R of any one of the therapeutic payloads described herein.

Methods of treating cancer are contemplated herein, comprising administering to a patient in need thereof an effective amount of a disclosed compound. For example, provided herein is a method of treating cancer in patient in need thereof, comprising administering to the patient an effective amount of a disclosed therapeutic payload, a disclosed linker-payload construct, or a disclosed drug conjugate.

Pharmaceutical compositions comprising at least one disclosed compound and a pharmaceutically acceptable carrier are additionally described herein. For example, provided herein is a pharmaceutically acceptable composition comprising a disclosed compound, e.g., a disclosed therapeutic payload, a disclosed linker-payload construct, or a disclosed drug conjugate and a pharmaceutically acceptable excipient.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein, the words “a” and “an” are meant to include one or more unless otherwise specified. For example, the term “an agent” encompasses both a single agent and a combination of two or more agents.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆ alkenyl, and C₃₋₄alkenyl, respectively. Exemplary alkenyl groups include, but are not limited to, vinyl, allyl, butenyl, pentenyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

The term “alkoxyalkyl” as used herein refers to a straight or branched alkyl group attached to oxygen, attached to a second straight or branched alkyl group (alkyl-O-alkyl-). Exemplary alkoxyalkyl groups include, but are not limited to, alkoxyalkyl groups in which each of the alkyl groups independently contains 1-6 carbon atoms, referred to herein as C₁₋₆alkoxy-C₁₋₆ alkyl. Exemplary alkoxyalkyl groups include, but are not limited to methoxymethyl, 2-methoxyethyl, 1-methoxyethyl, 2-methoxypropyl, ethoxymethyl, 2-isopropoxyethyl etc.

The term “alkyoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkenyloxy” used herein refers to a straight or branched alkenyl group attached to oxygen (alkenyl-O—). Exemplary alkenyloxy groups include, but are not limited to, groups with an alkenyl group of 3-6 carbon atoms, referred to herein as C₃₋₆alkenyloxy. Exemplary “alkenyloxy” groups include, but are not limited to allyloxy, butenyloxy, etc.

The term “alkynyloxy” used herein refers to a straight or branched alkynyl group attached to oxygen (alkynyl-O). Exemplary alkynyloxy groups include, but are not limited to, groups with an alkynyl group of 3-6 carbon atoms, referred to herein as C₃₋₆alkynyloxy. Exemplary alkynyloxy groups include, but are not limited to, propynyloxy, butynyloxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃ alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

“Alkylene” means a straight or branched, saturated aliphatic divalent radical having the number of carbons indicated. “Cycloalkylene” refers to a divalent radical of carbocyclic saturated hydrocarbon group having the number of carbons indicated.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆ alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “cyano” as used herein refers to the radical —CN.

The term “cycloalkoxy” as used herein refers to a cycloalkyl group attached to oxygen (cycloalkyl-O—). Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc.

The terms “cycloalkyl” or a “carbocyclic group” as used herein refers to a saturated or partially unsaturated hydrocarbon group of, for example, 3-6, or 4-6 carbons, referred to herein as C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

The term “heterocyclyloxy” as used herein refers to a heterocyclyl group attached to oxygen (heterocyclyl-O—).

The term “heteroaryloxy” as used herein refers to a heteroaryl group attached to oxygen (heteroaryl-O—).

The terms “hydroxy” and “hydroxyl” as used herein refers to the radical —OH.

The term “oxo” as used herein refers to the radical=O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

“Treating” includes any effect, e.g., lessening, reducing, modulating, or eliminating, that results in the improvement of the condition, disease, disorder and the like.

In the present specification, the term “therapeutically effective amount” or “effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

As will be understood by the skilled artisan, “H” is the symbol for hydrogen, “N” is the symbol for nitrogen, “S” is the symbol for sulfur, “O” is the symbol for oxygen. “Me” is an abbreviation for methyl. It will be appreciated that the present disclosure should be construed in congruity with the laws and principals of chemical bonding.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively. For example, a compound of the disclosure may have one or more H atom replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the present disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as (3-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a disclosed compound contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the present disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can be metabolically cleaved to generate a bioactive primary or secondary amine. For examples, see Simplicio, et al., Molecules 2008, 13, 519 and references therein.

Procedures for making compounds described herein are provided below in the working examples and may be supplemented or substituted by procedures known to those of skill in the art. Starting materials used in the working examples can be purchased or prepared by methods described in the chemical literature, or by adaptations thereof, using methods known by those skilled in the art. The order in which the steps are performed can vary depending on the groups introduced and the reagents used, but would be apparent to those skilled in the art. Disclosed compounds, or any of the intermediates described herein, can be further derivatized by using one or more standard synthetic methods known to those skilled in the art.

Salts of compounds disclosed herein can be prepared by the reaction of a compound disclosed herein with an appropriate acid or base in a suitable solvent, or mixture of solvents (such as an ether, for example, diethyl ether, or an alcohol, for example ethanol, or an aqueous solvent) using conventional procedures. Salts of a compound disclosed herein can be exchanged for other salts by treatment using conventional ion-exchange chromatography procedures.

### Compounds

Disclosed herein, for example, is a therapeutic payload represented by Formula I:

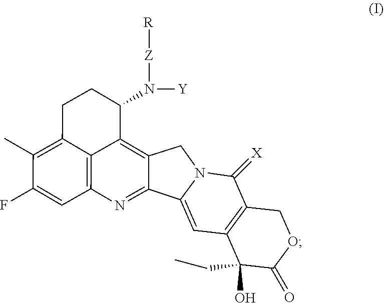

or a pharmaceutically acceptable salt or stereoisomer thereof, wherein:
X is selected from the group consisting of O and S;
Z is a bond; Y is selected from the group consisting of hydrogen, —C₁₋₃alkyl, —CHO, and —C(O)—C₁₋₃alkyl; and R is selected from the group consisting of R¹, R², R³, R⁴, R⁵ and hydrogen; or
Y and Z, together with the nitrogen to which they are attached, are joined together to form a 5-6 membered heteroaryl optionally substituted by one, two or three substituents, each independently selected from R^{Z}; R is bonded to the heteroaryl; and R is R⁶;
R¹ is selected from the group consisting of —C(O)—C₁₋₃alkyl, —C(O)—O—C₁₋₃alkyl, C₁₋₄alkyl, —C₁₋₃alkyl-O—C₁₋₃alkyl, —C(O)—C₃₋₄alkynyl, —S(O)₂—C₁₋₃alkyl, —C(S)—C₁₋₃alkyl, —C₁₋₃alkyl-S—C₁₋₃ alkyl, and —C(O)—O—[(CH₂)₂—O]₁₋₁₀-C₂alkyl; wherein R¹ is substituted by hydroxyl and optionally substituted by one or more additional substituents each independently selected from R¹¹;
R¹¹ is independently selected for each occurrence from the group consisting of halogen, hydroxyl, —C₁₋₃alkyl-OH, —C₁₋₃haloalkyl, and —C₃₋₄cycloalkyl;
R² is selected from the group consisting of —C(O)—NR^{a}—C₁₋₃alkyl, —C(O)—C₀₋₃alkyl-C(O)—NR^{a}—C₁₋₃alkyl, —C(O)—C₁₋₃alkyl-NR^{a}—C₁₋₃alkyl, —S(O)₂—C₁₋₃alkyl-NR^{a}—C(O)—C₁₋₃alkyl, and —C(O)NR^{a}—[(CH₂)₂—O]₁₋₁₀-C₂alkyl; wherein R² is substituted by hydroxyl and optionally substituted by one or more additional substituents each independently selected from R²²;
R²² is independently selected for each occurrence from the group consisting of halogen, hydroxyl, —C₁₋₃alkyl-OH, and —C₁₋₃haloalkyl;
R³ is selected from the group consisting of —C(O)—C₀₋₃alkyl-R³⁰, —C(O)—C₀₋₃alkyl-O—C₁₋₃alkyl-R³⁰, —C₀₋₃alkyl-R³⁰, and —C₁₋₃alkyl-O—C₁₋₃alkyl-R³⁰; wherein the alkyl if present may optionally be substituted by one or more substituents each independently selected from the group consisting of halogen and —C₁₋₃haloalkyl;
R³⁰ is selected from the group consisting of 5-6 membered heteroaryl and 4-10 membered heterocyclyl having one, two or three heteroatoms, each independently selected from the group consisting of N, NR³¹, and O; wherein R³⁰ is optionally substituted on one or more available carbons by one or more substituents each independently selected from R³³;
R³¹ is independently selected for each occurrence from the group consisting of hydrogen, —C₁₋₃alkyl, —C₁₋₃alkyl-OH, —CH(OH)CH₂OH, —CHO, and —C(O)—C₁₋₃alkyl;
R³³ is independently selected for each occurrence from the group consisting of —C₁₋₃alkyl-OH, halogen, hydroxyl, oxo, and —C₁₋₃haloalkyl;
R⁴ is selected from the group consisting of —C(O)—NR^{a}—C₃₋₆cycloalkyl, —C(O)—C₀₋₂alkyl-C₃₋₆cycloalkyl, —C(S)—C₀₋₂alkyl-C₃₋₆cycloalkyl, —C(O)—NR^{a}—C₃₋₆cycloalkyl, and —C₃₋₆cycloalkenyl-NR^{a}—C₁₋₃alkyl; wherein R⁴ is substituted by one or more substituents each independently selected from R⁴⁴;
R⁴⁴ is independently selected for each occurrence from the group consisting of hydroxyl, halogen, oxo, —C₁₋₃alkyl, and —C₁₋₃alkyl-OH;
R⁵ is selected from the group consisting of —S(O)₂—C₁₋₃alkyl-NR^{a}R^{b}, —C₁₋₄alkyl-NR^{a}R^{b}, —C(O)—C₁₋₃alkyl-O—NR^{a}R^{b}, —N═S(═O)(C₁₋₃alkyl)C₁₋₃alkyl, —C(O)—CH₂-phenyl-CH₂NR^{a}R^{b}, and —[(CH₂)₂—NR^{a}]₁₋₅—C₁₋₃alkyl-NR^{a}R^{b}; wherein alkyl may optionally be substituted by one or more substituents each independently selected from R⁵⁵;
R⁵⁵ is independently selected for each occurrence from the group consisting of halogen, —C₁₋₃alkyl and —C₁₋₃haloalkyl;
R⁶ is —C₁₋₃alkyl substituted by hydroxyl and optionally substituted by one or more additional substituents each independently selected from R⁶⁶
R⁶⁶ is independently selected for each occurrence from the group consisting of halogen and —C₁₋₃haloalkyl;
R^{Z} is selected from the group consisting of halogen, —C₁₋₃alkyl and —C₁₋₃alkyl-OH; and
R^{a} and R^{b} are each independently selected for each occurrence from the group consisting of the group consisting of hydrogen, —C₁₋₃alkyl-OH, and —C₁₋₃haloalkyl-OH;
   wherein when X is O and Y is H, then R is not hydrogen or —C(O)CH₂OH.

In some embodiments, X is O. In other embodiments, wherein Z is a bond. In certain embodiments, Y is selected from the group consisting of, for example, hydrogen, —CH₃, —CHO, and —COCH₃.

In some embodiments, R is R¹. For example, in some embodiments R is selected from the group consisting of —C(O)—C₁alkyl, —C(O)—C₂alkyl, —C(O)—O—C₂alkyl, —C(O)—O—C₃alkyl, —C₂alkyl, —C₃alkyl, —C₂alkyl-O—C₂alkyl, —C(S)—C₁alkyl, —S(O)₂—C₁alkyl, —S(O)₂—C₂alkyl, —S(O)₂—C₃alkyl, —C(O)—C₃alkynyl, —C₂alkyl-S—C₂alkyl, and —C(O)—O—[(CH₂)₂—O]₁₋₅—C₂alkyl; wherein R¹ is substituted by hydroxyl and optionally substituted by one or more additional substituents each independently selected from R¹¹. In certain embodiments, R¹¹ is selected from the group consisting of, for example, fluoro, hydroxyl, —CH₂—OH, —CF₃, and cyclopropyl.

For example, in some embodiments —N(Y)—Z—R may be selected from the group consisting of:

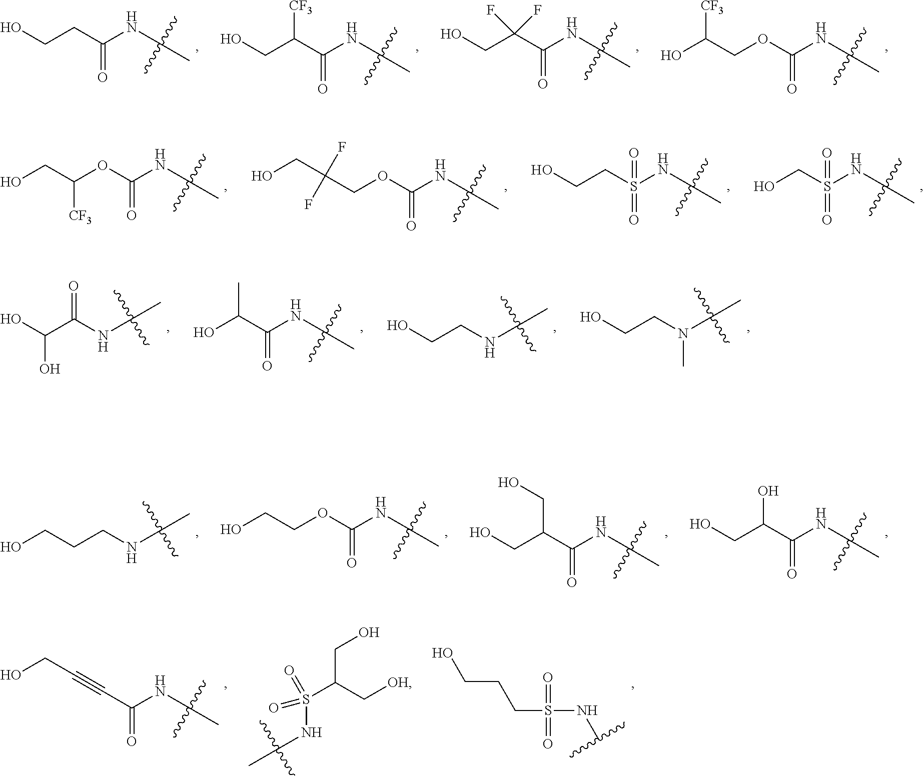

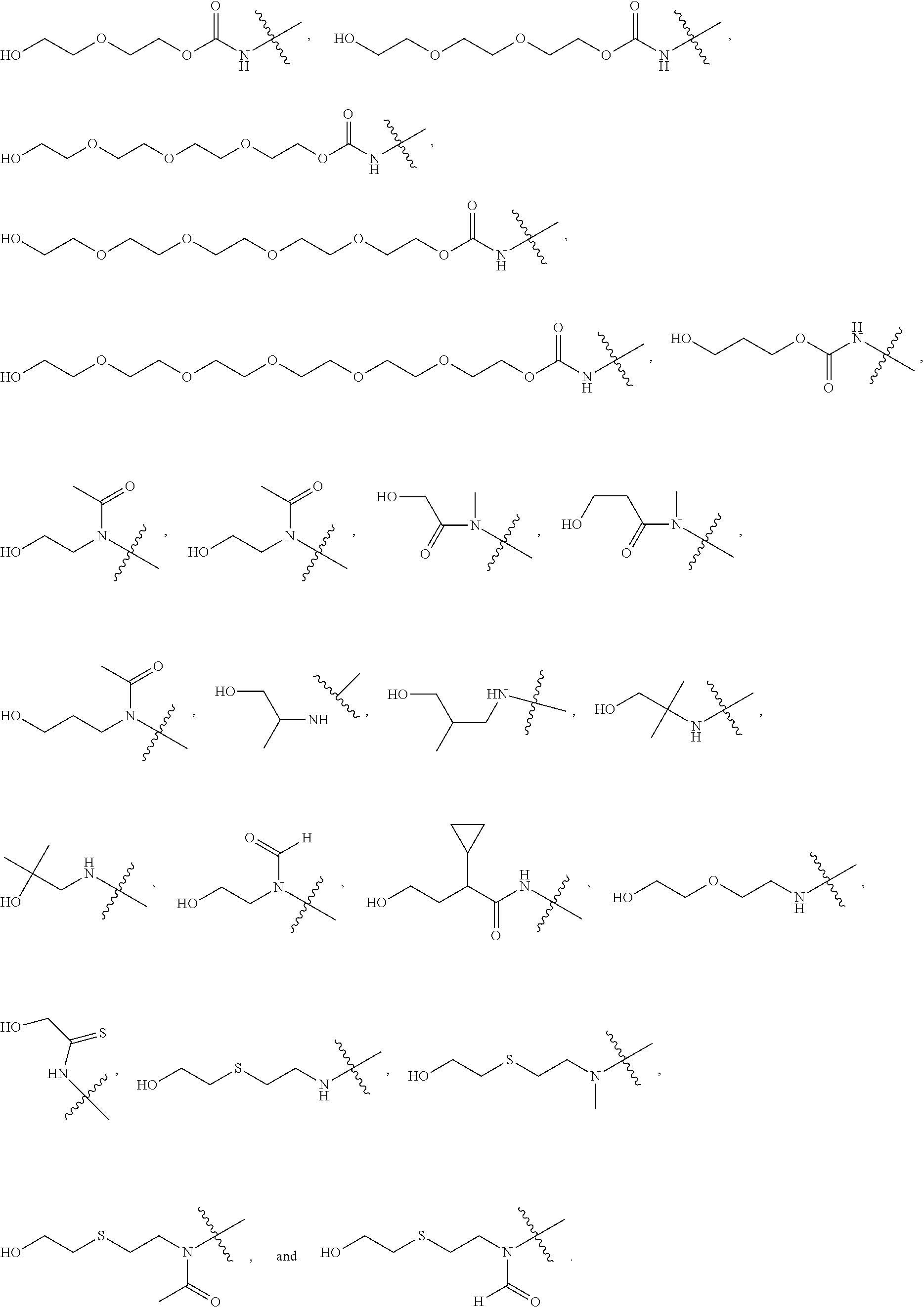

In other embodiments, R is R². In further embodiments, Y is hydrogen. In certain embodiments, R is selected from the group consisting of, for example, —C(O)—NH—C₂alkyl, —C(O)—NH—C₃alkyl, —C(O)—C(O)—NH—C₂alkyl, —C(O)—C(O)—NH—C₃alkyl, —C(O)—C₁alkyl-C(O)—NH—C₂alkyl, —C(O)—C₂alkyl-C(O)—NH—C₂alkyl, —C(O)—C₂alkyl-C(O)—NH—C₃alkyl, —S(O)₂—C₂alkyl-NH—C(O)—C₁alkyl, —S(O)₂—C₂alkyl-NH—C(O)—C₂alkyl, and —C(O)NH—[(CH₂)₂—O]¹⁻² —C₂alkyl; and wherein R² is substituted by hydroxyl and optionally substituted by one or more additional substituents one or more additional substituents each independently selected from R²² In further embodiments, R²² is selected from the group consisting of fluoro, hydroxyl, —CH₂—OH, and —CF₃.

For example, in some embodiments —N(Y)—Z—R is selected from the group consisting of:

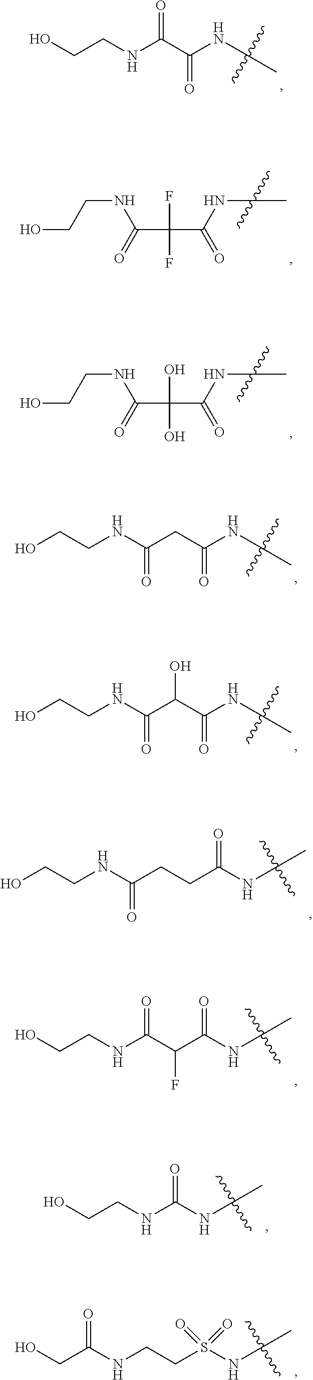

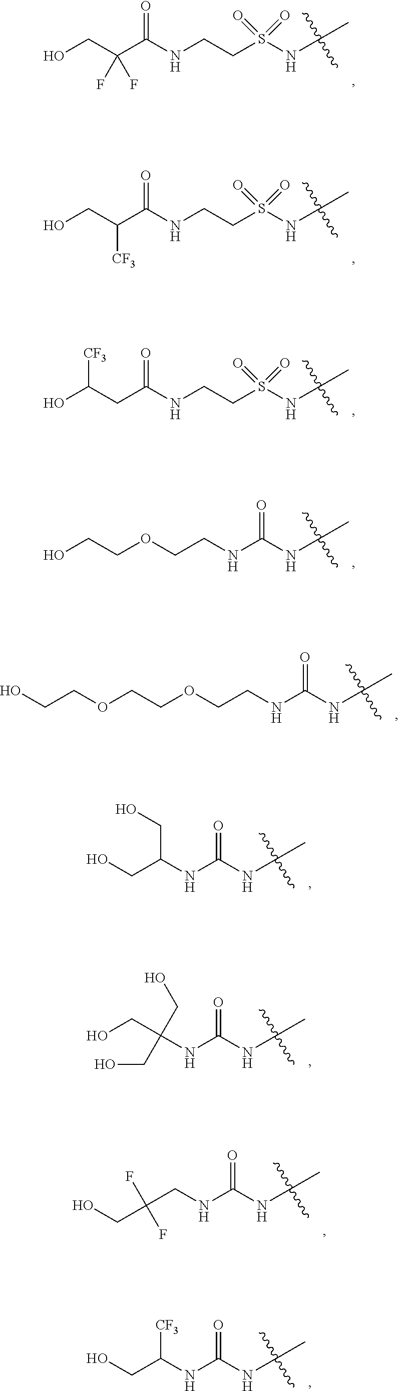

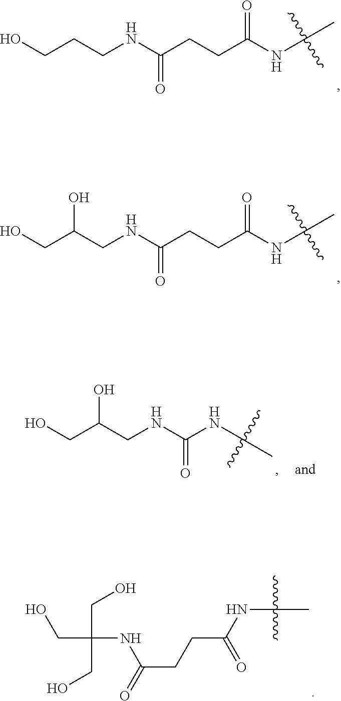 [SMILES: *NC(=O)CCC(=O)NCCCO ; *NC(=O)CCC(=O)NCC(O)CO ; *NC(=O)NCC(O)CO ; *NC(=O)CCC(=O)NC(CO)(CO)CO]

In other embodiments, R is R³. In certain embodiments, Y is hydrogen. In further embodiments, R is selected from the group consisting of, for example:
—C(O)-triazolyl, —C(O)—C₁alkyl-triazolyl, —C(O)—C₂alkyl-triazolyl, —C(O)—C₃alkyl-triazolyl, —C₁alkyl-triazolyl, —C₂alkyl-triazolyl, —C₃alkyl-triazolyl, —C(O)—O—C₁alkyl-triazolyl, —C(O)—O—C₂alkyl-triazolyl, —C(O)—C₁alkyl-O—C₂alkyl-triazolyl, —C(O)—C₂alkyl-O—C₁alkyl-triazolyl, —C(O)—C₂alkyl-O—C₂alkyl-triazolyl, —C₂alkyl-O—C₁alkyl-triazolyl, and —C₂alkyl-O—C₂alkyl-triazolyl;
   wherein:
alkyl for each occurrence may optionally be substituted by one, two or three substituents each independently selected from the group consisting of fluoro and —CF₃;
triazolyl is substituted on an available nitrogen, if present, by a substituent selected from the group consisting of hydrogen, —C₁₋₃alkyl, and C₁₋₂alkyl-OH; and
triazolyl may optionally be substituted on an available carbon by a substituent selected from the group consisting of chloro, fluoro, and C₁₋₂alkyl-OH.

For example, in some embodiments R is selected from the group consisting of:

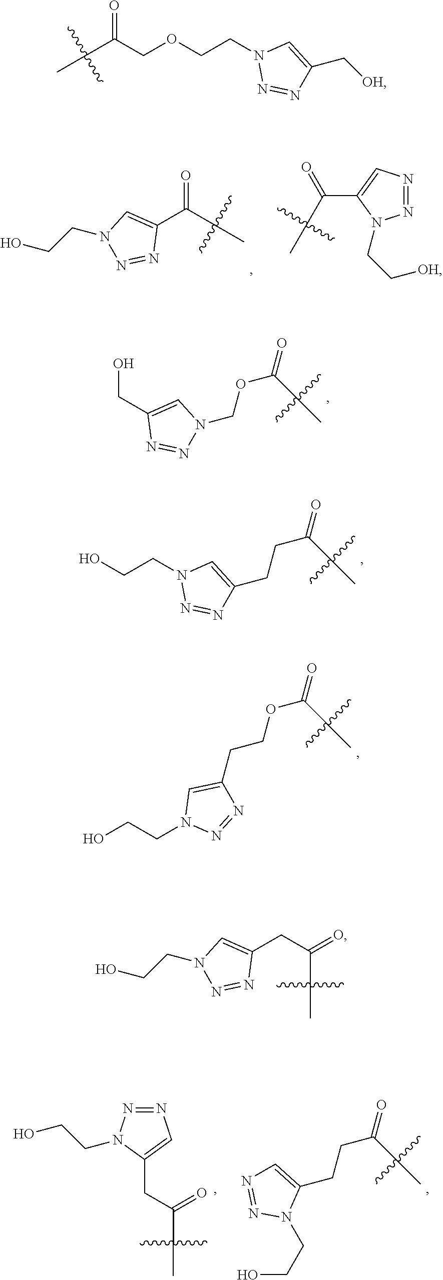

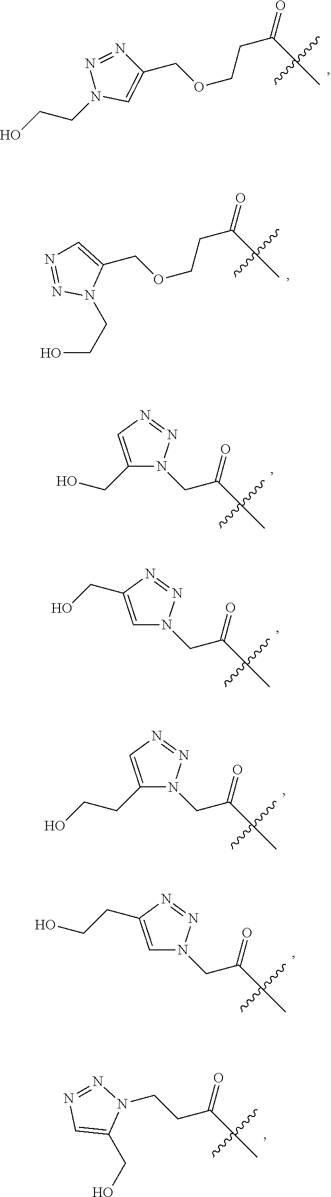

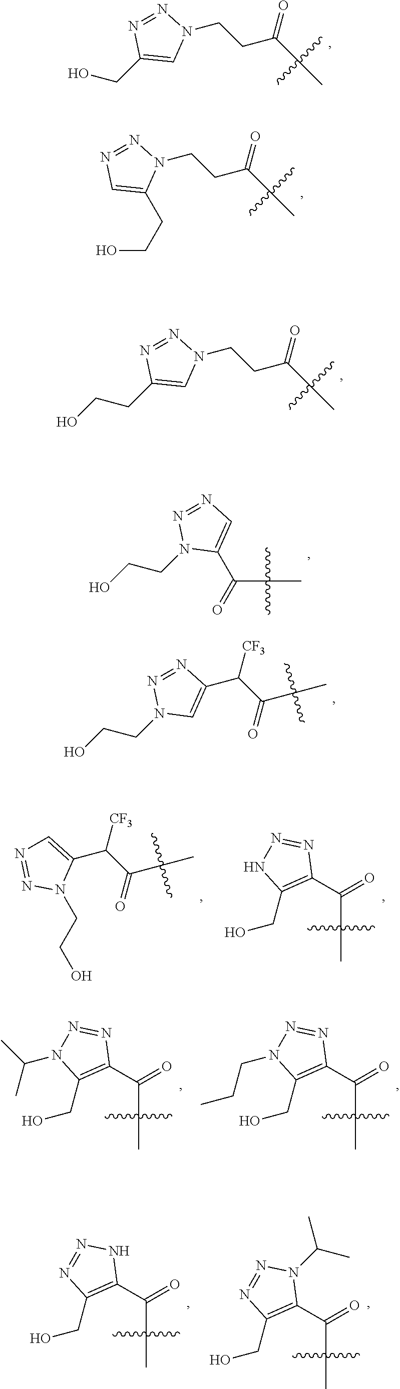

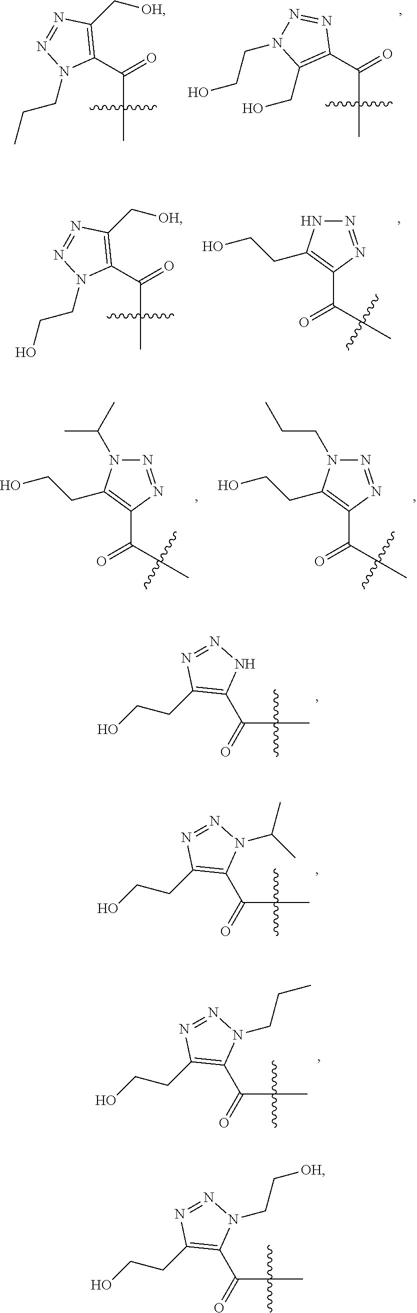

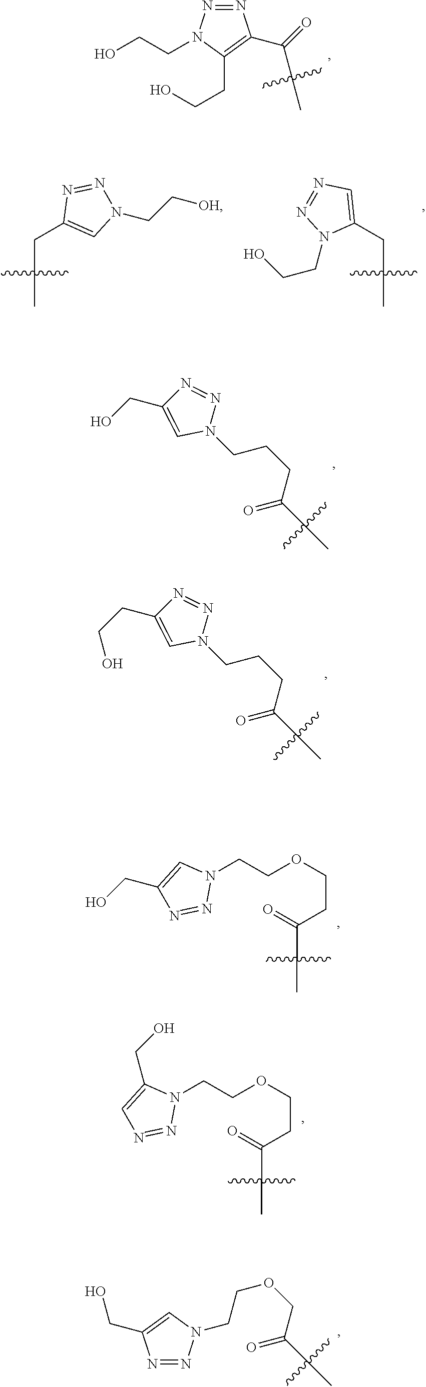

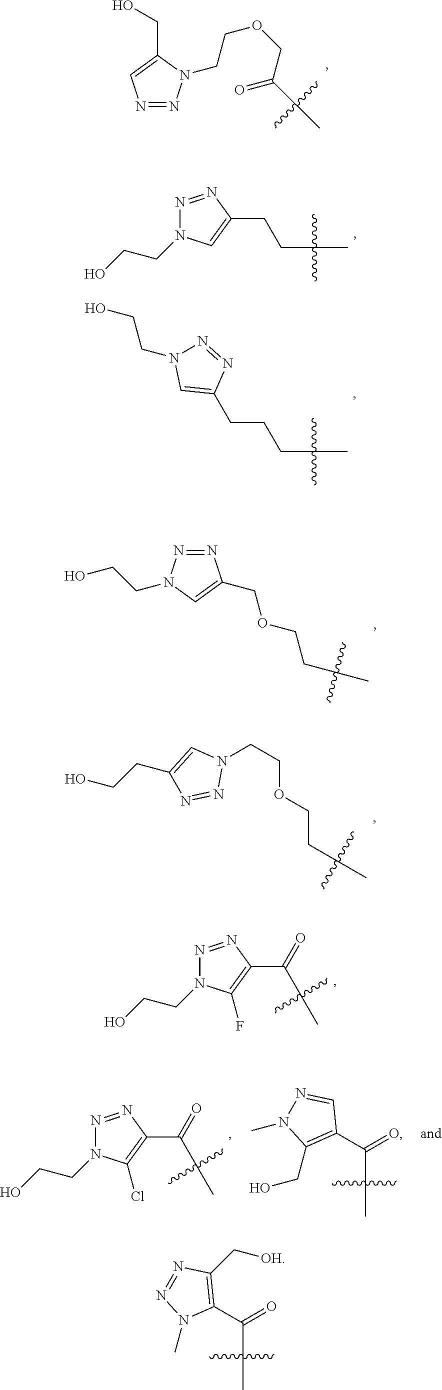

In other embodiments, R is selected from the group consisting of: —C(O)-furanyl, —C₁alkyl-furanyl, —C(O)-oxazolyl, and —C(O)-pyrrazolyl; wherein R is substituted by a substituent selected from the group consisting of hydroxyl and C₁₋₂alkyl-OH. For example, in certain embodiments R is selected from the group consisting of:

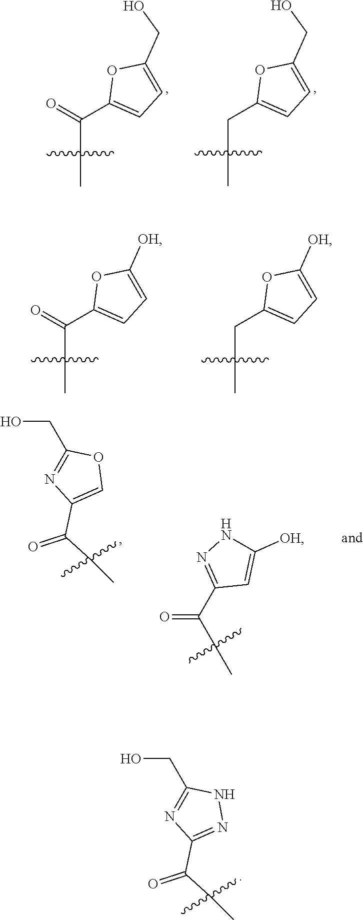

In still other embodiments, R is selected from the group consisting of:

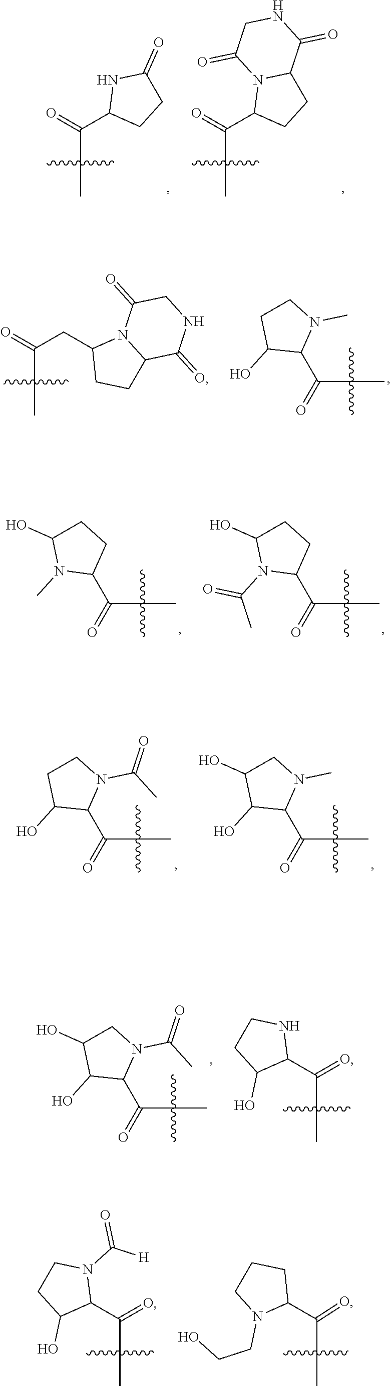

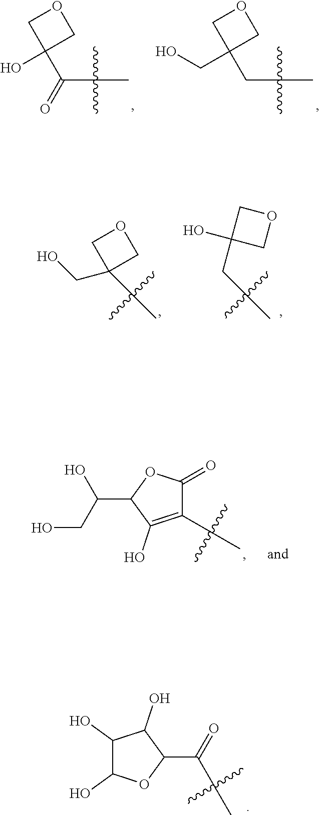 [SMILES: *C(=O)C1(O)COC1 ; *CC1(CO)COC1 ; *C1(CO)COC1 ; *CC1(O)COC1 ; *C1=C(O)C(C(O)CO)OC1=O ; *C(=O)C1OC(O)C(O)C1O]

In further embodiments, R is R⁴. In certain embodiments, Y is hydrogen. In other embodiments R is selected from the group consisting of, for example: —C(O)—C₃cycloalkyl, —C(S)—C₃cycloalkyl, —C(O)—C₄cycloalkyl, —C(O)—C₅cycloalkyl, —C(O)—C₆cycloalkyl, —C(O)—NH—C₃cycloalkyl, —C(O)—NH—C₄cycloalkyl, —C₄cycloalkenyl-NH—C₂alkyl, —C₄cycloalkenyl-NH—C₃alkyl, —C₅cycloalkenyl-NH—C₂alkyl, and —C₅cycloalkenyl-NH—C₂alkyl;

wherein:
cycloalkyl or cycloalkenyl is substituted by one or more substituents each independently selected from the group consisting of hydroxyl, oxo, —C₁₋₃alkyl, and C₁₋₂alkyl-OH; and
alkyl is substituted by one, two or three substituents each independently selected from the group consisting of hydroxyl and —CH₂OH.

For example, in some embodiments R is selected from the group consisting of:

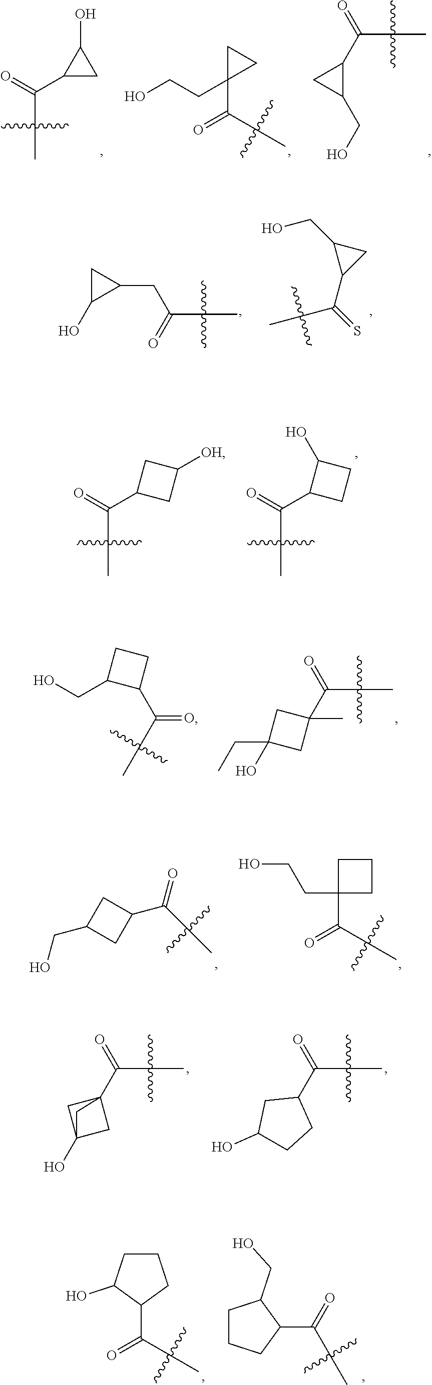

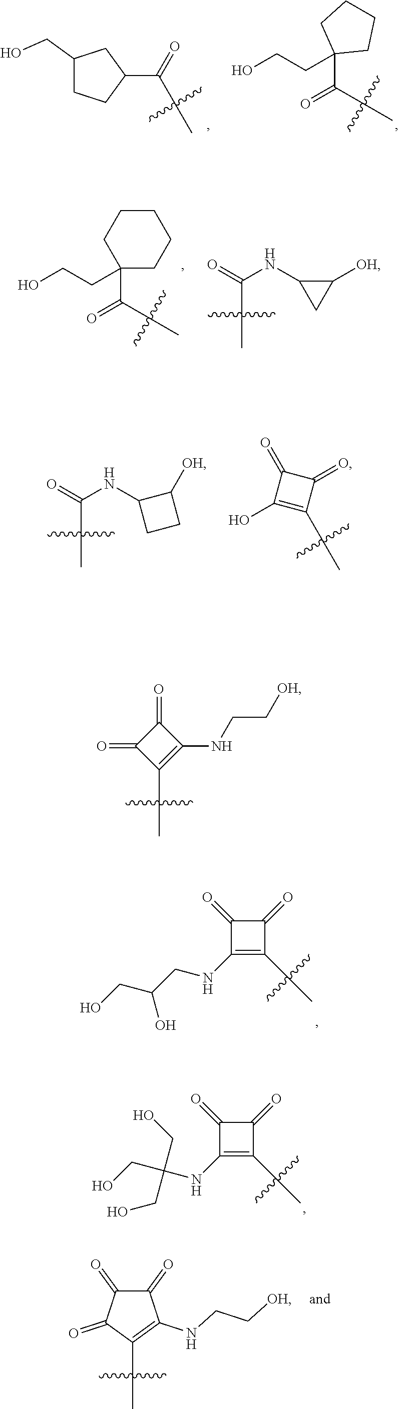

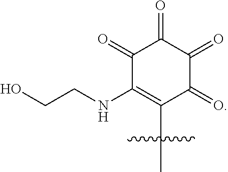 [SMILES: *c1c(NCCO)c(=O)c(=O)c(=O)c1=O]

In some embodiments, R is R⁵. In other embodiments, Y is selected from the group consisting of hydrogen, —CH₃ and —C(O)CH₃. In certain embodiments, R is selected from the group consisting of, for example, —S(O)₂—C₂alkyl-NH₂, —S(O)₂—C₃alkyl-NH₂, —C₂alkyl-NH₂, —C₃alkyl-NH₂, —C(O)—C₁alkyl-O—NH₂, —C(O)—CH₂-phenyl-CH₂NH₂, and —(CH₂)₂—NH—C₂alkyl-NH₂; wherein alkyl may optionally be substituted by one or two —CH₃ groups.

For example, in some embodiments —Z—N(Y)—R selected from the group consisting of:

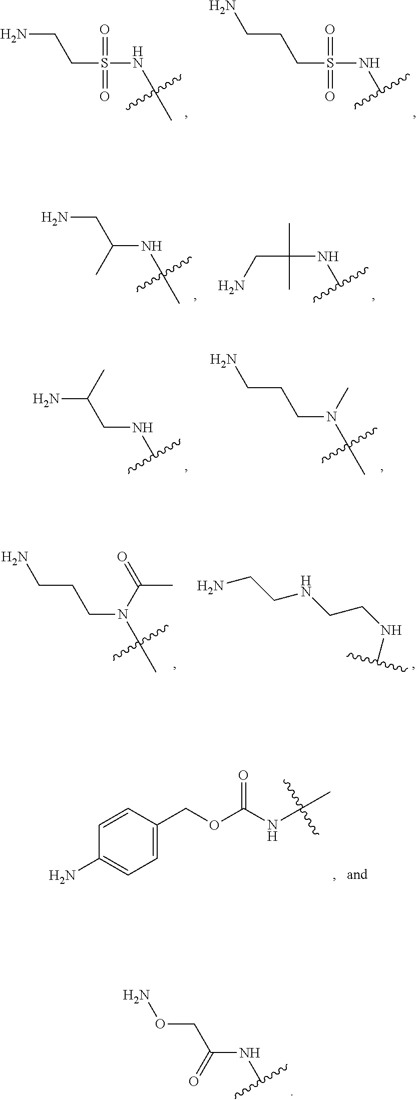

In other embodiments, Y and Z, together with the nitrogen to which they are attached, are joined together to form triazolyl substituted at a substitutable position by R. In certain embodiments, R is C₁alkyl-OH or C₂alkyl-OH, wherein R may optionally be substituted by —CF₃. In further embodiments, —Z—N(Y)—R is selected from the group consisting of, for example.

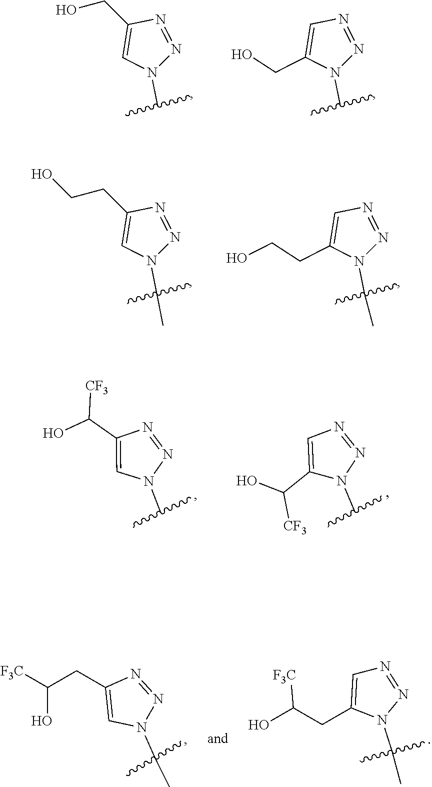

In still other embodiments, X is S. In certain embodiments, Y is hydrogen. In certain embodiments, R is selected from the group consisting of, for example, hydrogen,

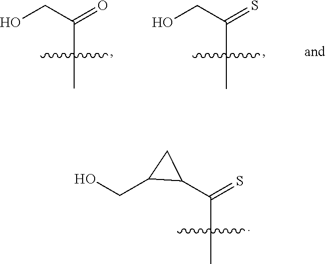 [SMILES: *C(=O)CO ; *C(=S)CO ; *C(=S)C1CC1CO]

In some embodiments, a disclosed therapeutic payload may be selected, for example, from any one of the compounds disclosed in Table 1, or a pharmaceutically acceptable salt or stereoisomer thereof.

TABLE 1

| Comp # | Structure |
|---|---|
| 1 | 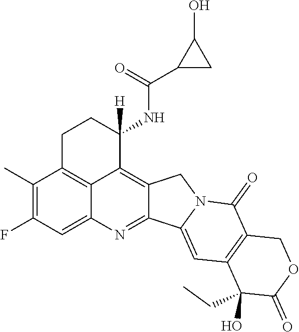 [SMILES: CC[C@@]1(O)C(=O)OCc2c1cc1n(c2=O)Cc2c-1nc1cc(F)c(C)c3c1c2[C@@H](NC(=O)C1CC1O)CC3] |
| 2 | 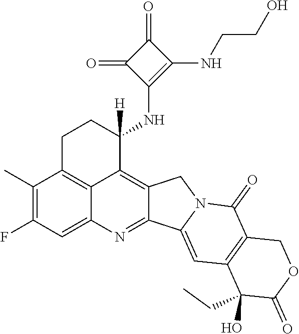 [SMILES: CC[C@@]1(O)C(=O)OCc2c1cc1n(c2=O)Cc2c-1nc1cc(F)c(C)c3c1c2[C@@H](Nc1c(NCCO)c(=O)c1=O)CC3] |
| 3 | 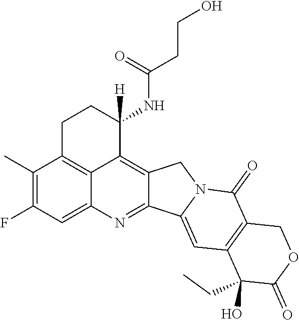 [SMILES: CC[C@@]1(O)C(=O)OCc2c1cc1n(c2=O)Cc2c-1nc1cc(F)c(C)c3c1c2[C@@H](NC(=O)CCO)CC3] |
| 4 | 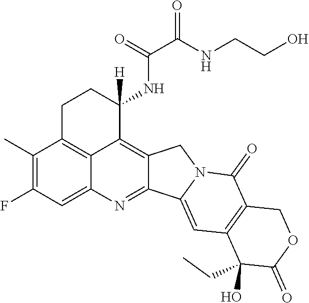 [SMILES: CC[C@@]1(O)C(=O)OCc2c1cc1n(c2=O)Cc2c-1nc1cc(F)c(C)c3c1c2[C@@H](NC(=O)C(=O)NCCO)CC3] |
| 5 | 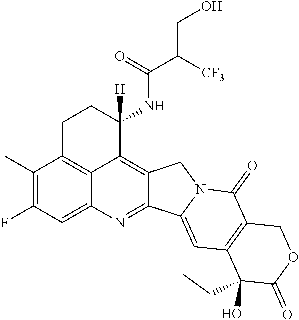 [SMILES: CC[C@@]1(O)C(=O)OCc2c1cc1n(c2=O)Cc2c-1nc1cc(F)c(C)c3c1c2[C@@H](NC(=O)C(CO)C(F)(F)F)CC3] |
| 6 | 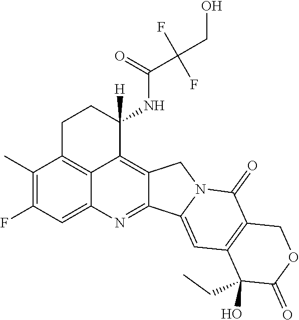 [SMILES: CC[C@@]1(O)C(=O)OCc2c1cc1n(c2=O)Cc2c-1nc1cc(F)c(C)c3c1c2[C@@H](NC(=O)C(F)(F)CO)CC3] |
| 7 | 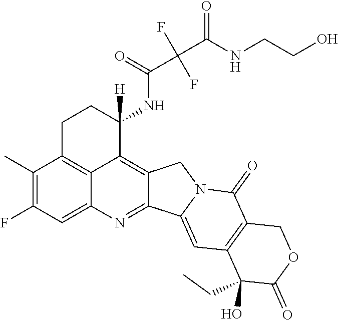 [SMILES: CC[C@@]1(O)C(=O)OCc2c1cc1n(c2=O)Cc2c-1nc1cc(F)c(C)c3c1c2[C@@H](NC(=O)C(F)(F)C(=O)NCCO)CC3] |
| 8 | 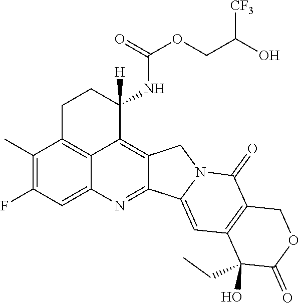 [SMILES: CC[C@@]1(O)C(=O)OCc2c1cc1n(c2=O)Cc2c-1nc1cc(F)c(C)c3c1c2[C@@H](NC(=O)OCC(O)C(F)(F)F)CC3] |
| 9 | 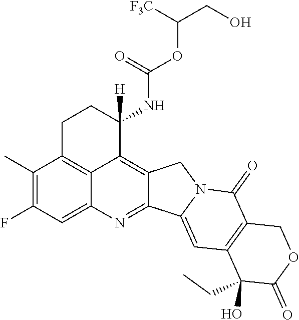 [SMILES: CC[C@@]1(O)C(=O)OCc2c1cc1n(c2=O)Cc2c-1nc1cc(F)c(C)c3c1c2[C@@H](NC(=O)OC(CO)C(F)(F)F)CC3] |
| 10 | 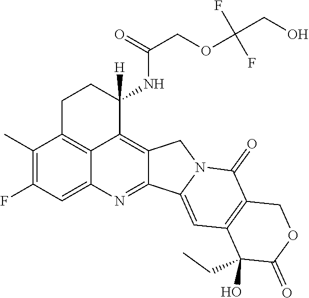 [SMILES: CC[C@@]1(O)C(=O)OCc2c1cc1n(c2=O)Cc2c-1nc1cc(F)c(C)c3c1c2[C@@H](NC(=O)COC(F)(F)CO)CC3] |
| 11 | 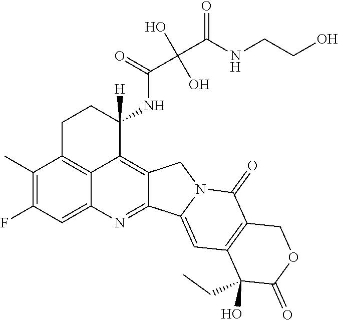 [SMILES: CC[C@@]1(O)C(=O)OCc2c1cc1n(c2=O)Cc2c-1nc1cc(F)c(C)c3c1c2[C@@H](NC(=O)C(O)(O)C(=O)NCCO)CC3] |
| 12 | 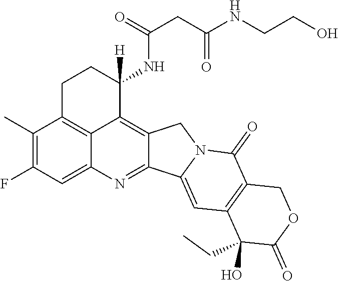 [SMILES: CC[C@@]1(O)C(=O)OCc2c1cc1n(c2=O)Cc2c-1nc1cc(F)c(C)c3c1c2[C@@H](NC(=O)CC(=O)NCCO)CC3] |
| 13 | 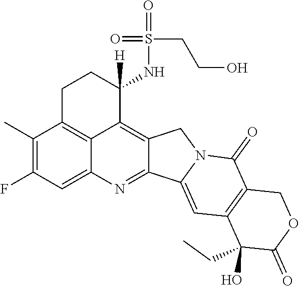 [SMILES: CC[C@@]1(O)C(=O)OCc2c1cc1n(c2=O)Cc2c-1nc1cc(F)c(C)c3c1c2[C@@H](NS(=O)(=O)CCO)CC3] |
| 14 | 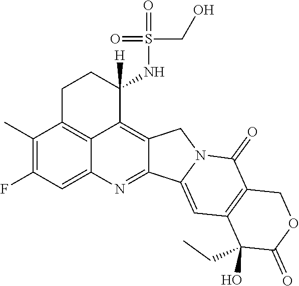 [SMILES: CC[C@@]1(O)C(=O)OCc2c1cc1n(c2=O)Cc2c-1nc1cc(F)c(C)c3c1c2[C@@H](NS(=O)(=O)CO)CC3] |
| 15 | 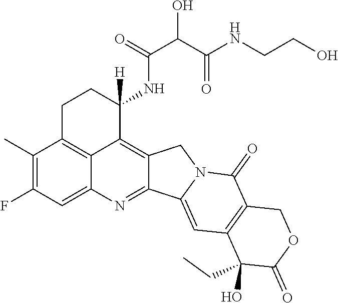 [SMILES: CC[C@@]1(O)C(=O)OCc2c1cc1n(c2=O)Cc2c-1nc1cc(F)c(C)c3c1c2[C@@H](NC(=O)C(O)C(=O)NCCO)CC3] |
| 16 | 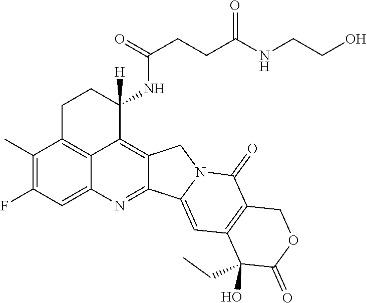 [SMILES: CC[C@@]1(O)C(=O)OCc2c1cc1n(c2=O)Cc2c-1nc1cc(F)c(C)c3c1c2[C@@H](NC(=O)CCC(=O)NCCO)CC3] |
| 17 | 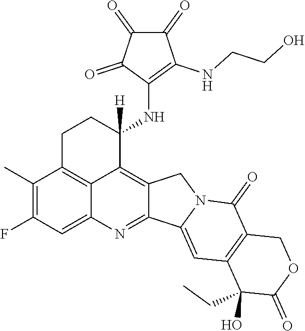 [SMILES: CC[C@@]1(O)C(=O)OCc2c1cc1n(c2=O)Cc2c-1nc1cc(F)c(C)c3c1c2[C@@H](Nc1c(NCCO)c(=O)c(=O)c1=O)CC3] |
| 18 | 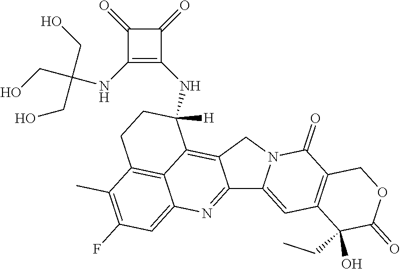 [SMILES: CC[C@@]1(O)C(=O)OCc2c1cc1n(c2=O)Cc2c-1nc1cc(F)c(C)c3c1c2[C@@H](Nc1c(NC(CO)(CO)CO)c(=O)c1=O)CC3] |
| 19 | 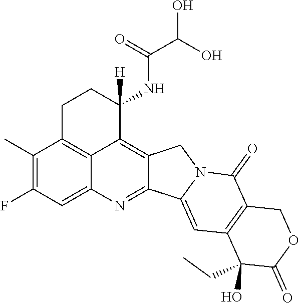 [SMILES: CC[C@@]1(O)C(=O)OCc2c1cc1n(c2=O)Cc2c-1nc1cc(F)c(C)c3c1c2[C@@H](NC(=O)C(O)O)CC3] |
| 20 | 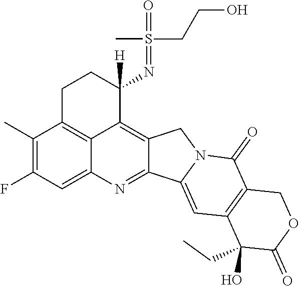 [SMILES: CC[C@@]1(O)C(=O)OCc2c1cc1n(c2=O)Cc2c-1nc1cc(F)c(C)c3c1c2[C@@H](N=S(C)(=O)CCO)CC3] |
| 21 | 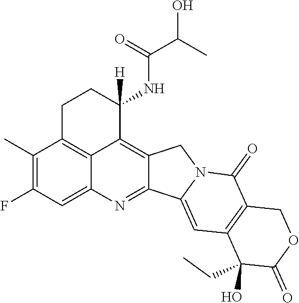 [SMILES: CC[C@@]1(O)C(=O)OCc2c1cc1n(c2=O)Cc2c-1nc1cc(F)c(C)c3c1c2[C@@H](NC(=O)C(C)O)CC3] |
| 22 | 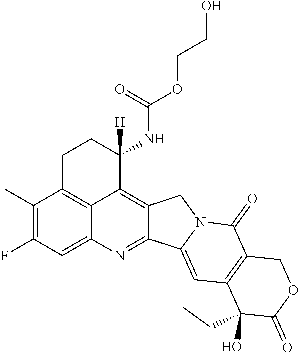 [SMILES: CC[C@@]1(O)C(=O)OCc2c1cc1n(c2=O)Cc2c-1nc1cc(F)c(C)c3c1c2[C@@H](NC(=O)OCCO)CC3] |
| 23 | 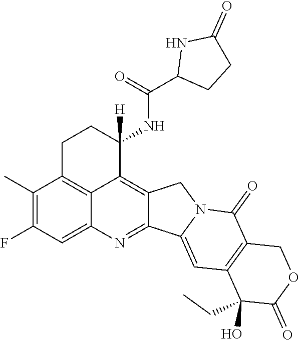 [SMILES: CC[C@@]1(O)C(=O)OCc2c1cc1n(c2=O)Cc2c-1nc1cc(F)c(C)c3c1c2[C@@H](NC(=O)C1CCC(=O)N1)CC3] |
| 24 | 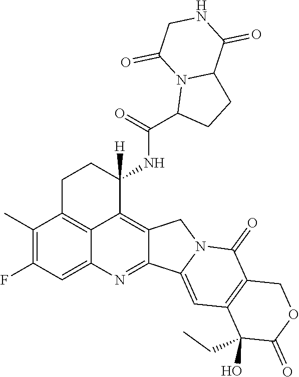 [SMILES: CC[C@@]1(O)C(=O)OCc2c1cc1n(c2=O)Cc2c-1nc1cc(F)c(C)c3c1c2[C@@H](NC(=O)C1CCC2C(=O)NCC(=O)N21)CC3] |
| 25 | 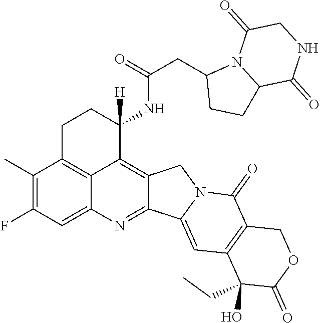 [SMILES: CC[C@@]1(O)C(=O)OCc2c1cc1n(c2=O)Cc2c-1nc1cc(F)c(C)c3c1c2[C@@H](NC(=O)CC1CCC2C(=O)NCC(=O)N12)CC3] |
| 26 | 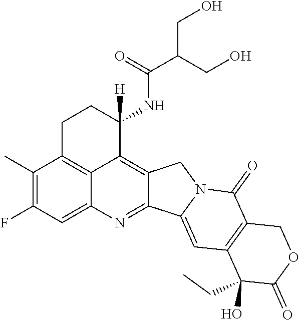 [SMILES: CC[C@@]1(O)C(=O)OCc2c1cc1n(c2=O)Cc2c-1nc1cc(F)c(C)c3c1c2[C@@H](NC(=O)C(CO)CO)CC3] |
| 27 | 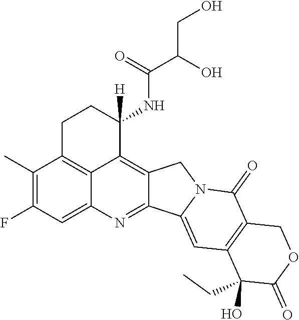 [SMILES: CC[C@@]1(O)C(=O)OCc2c1cc1n(c2=O)Cc2c-1nc1cc(F)c(C)c3c1c2[C@@H](NC(=O)C(O)CO)CC3] |
| 28 | 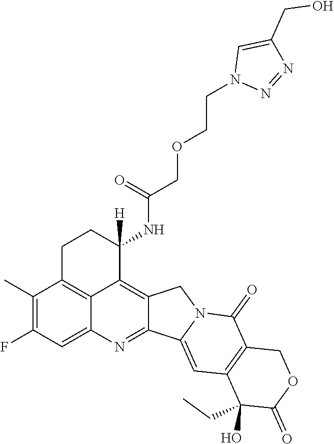 [SMILES: CC[C@@]1(O)C(=O)OCc2c1cc1n(c2=O)Cc2c-1nc1cc(F)c(C)c3c1c2[C@@H](NC(=O)COCCn1cc(CO)nn1)CC3] |
| 29 | 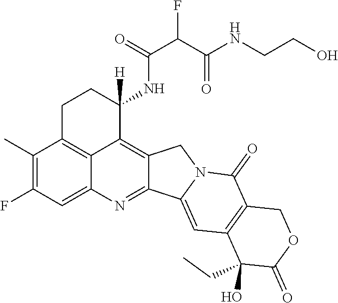 [SMILES: CC[C@@]1(O)C(=O)OCc2c1cc1n(c2=O)Cc2c-1nc1cc(F)c(C)c3c1c2[C@@H](NC(=O)C(F)C(=O)NCCO)CC3] |
| 30 | 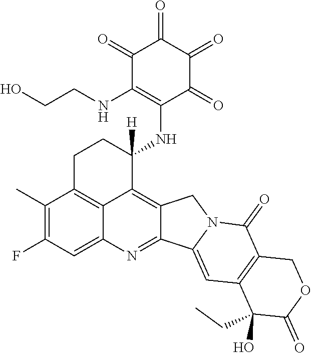 [SMILES: CC[C@@]1(O)C(=O)OCc2c1cc1n(c2=O)Cc2c-1nc1cc(F)c(C)c3c1c2[C@@H](Nc1c(NCCO)c(=O)c(=O)c(=O)c1=O)CC3] |
| 31 | 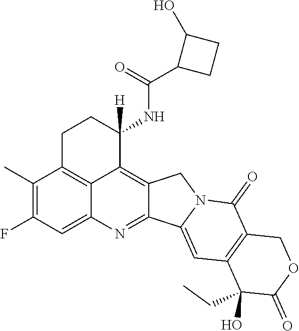 [SMILES: CC[C@@]1(O)C(=O)OCc2c1cc1n(c2=O)Cc2c-1nc1cc(F)c(C)c3c1c2[C@@H](NC(=O)C1CCC1O)CC3] |
| 32 | 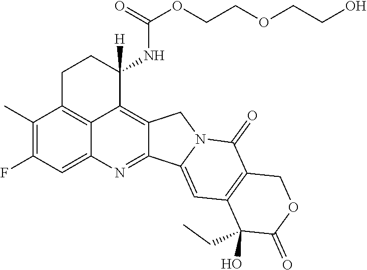 [SMILES: CC[C@@]1(O)C(=O)OCc2c1cc1n(c2=O)Cc2c-1nc1cc(F)c(C)c3c1c2[C@@H](NC(=O)OCCOCCO)CC3] |
| 33 | 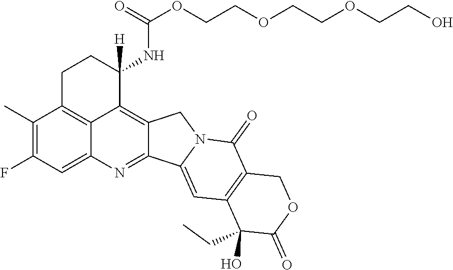 [SMILES: CC[C@@]1(O)C(=O)OCc2c1cc1n(c2=O)Cc2c-1nc1cc(F)c(C)c3c1c2[C@@H](NC(=O)OCCOCCOCCO)CC3] |
| 34 | 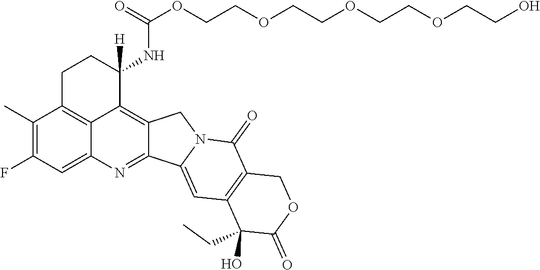 [SMILES: CC[C@@]1(O)C(=O)OCc2c1cc1n(c2=O)Cc2c-1nc1cc(F)c(C)c3c1c2[C@@H](NC(=O)OCCOCCOCCOCCO)CC3] |
| 35 | 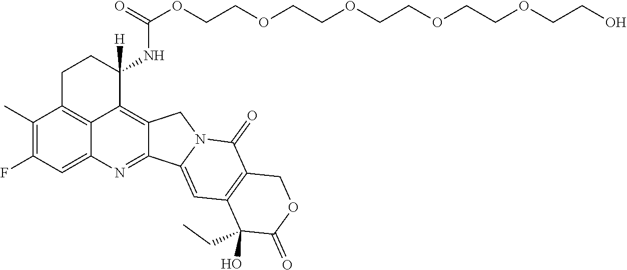 [SMILES: CC[C@@]1(O)C(=O)OCc2c1cc1n(c2=O)Cc2c-1nc1cc(F)c(C)c3c1c2[C@@H](NC(=O)OCCOCCOCCOCCOCCO)CC3] |
| 36 | 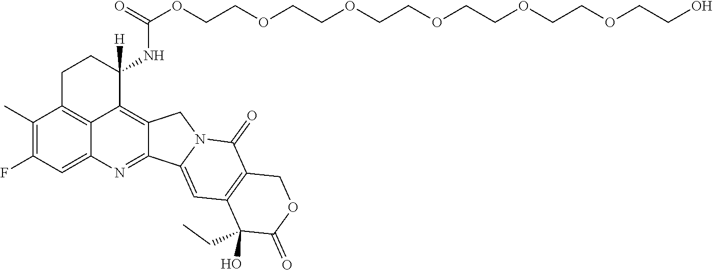 [SMILES: CC[C@@]1(O)C(=O)OCc2c1cc1n(c2=O)Cc2c-1nc1cc(F)c(C)c3c1c2[C@@H](NC(=O)OCCOCCOCCOCCOCCOCCO)CC3] |
| 37 | 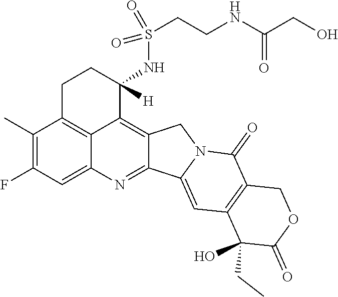 [SMILES: CC[C@@]1(O)C(=O)OCc2c1cc1n(c2=O)Cc2c-1nc1cc(F)c(C)c3c1c2[C@@H](NS(=O)(=O)CCNC(=O)CO)CC3] |
| 38 | 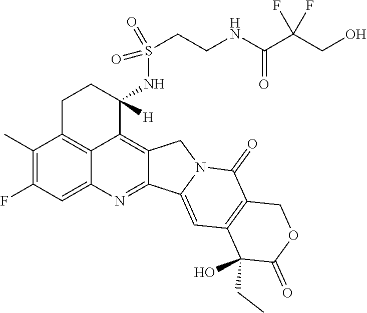 [SMILES: CC[C@@]1(O)C(=O)OCc2c1cc1n(c2=O)Cc2c-1nc1cc(F)c(C)c3c1c2[C@@H](NS(=O)(=O)CCNC(=O)C(F)(F)CO)CC3] |
| 39 | 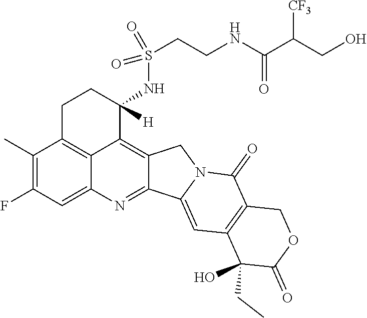 [SMILES: CC[C@@]1(O)C(=O)OCc2c1cc1n(c2=O)Cc2c-1nc1cc(F)c(C)c3c1c2[C@@H](NS(=O)(=O)CCNC(=O)C(CO)C(F)(F)F)CC3] |
| 40 | 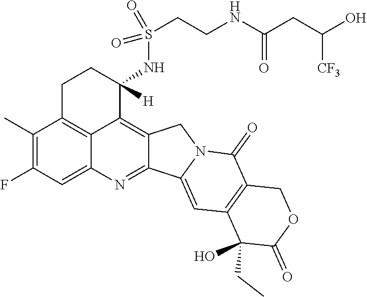 [SMILES: CC[C@@]1(O)C(=O)OCc2c1cc1n(c2=O)Cc2c-1nc1cc(F)c(C)c3c1c2[C@@H](NS(=O)(=O)CCNC(=O)CC(O)C(F)(F)F)CC3] |
| 41 | 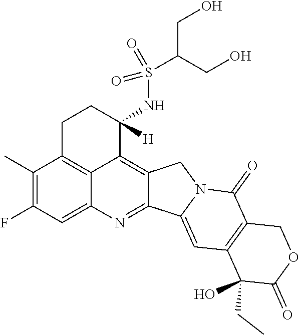 [SMILES: CC[C@@]1(O)C(=O)OCc2c1cc1n(c2=O)Cc2c-1nc1cc(F)c(C)c3c1c2[C@@H](NS(=O)(=O)C(CO)CO)CC3] |
| 42 | 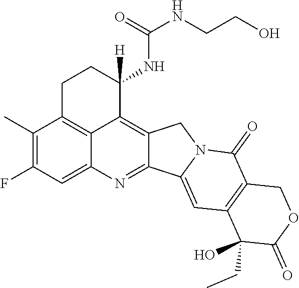 [SMILES: CC[C@@]1(O)C(=O)OCc2c1cc1n(c2=O)Cc2c-1nc1cc(F)c(C)c3c1c2[C@@H](NC(=O)NCCO)CC3] |
| 43 | 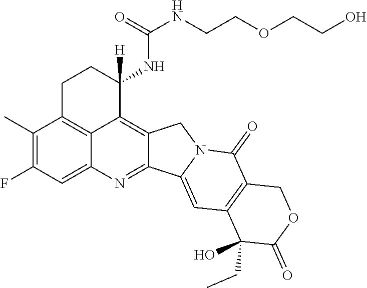 [SMILES: CC[C@@]1(O)C(=O)OCc2c1cc1n(c2=O)Cc2c-1nc1cc(F)c(C)c3c1c2[C@@H](NC(=O)NCCOCCO)CC3] |
| 44 | 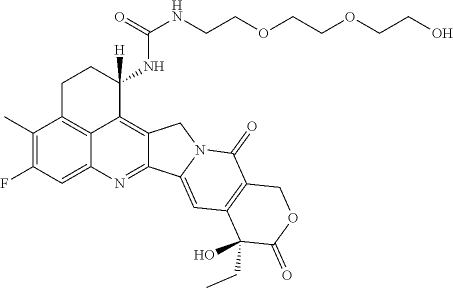 [SMILES: CC[C@@]1(O)C(=O)OCc2c1cc1n(c2=O)Cc2c-1nc1cc(F)c(C)c3c1c2[C@@H](NC(=O)NCCOCCOCCO)CC3] |
| 45 | 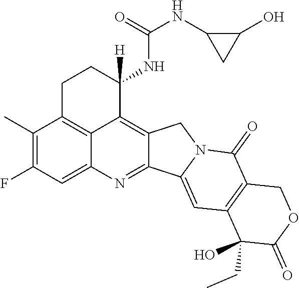 [SMILES: CC[C@@]1(O)C(=O)OCc2c1cc1n(c2=O)Cc2c-1nc1cc(F)c(C)c3c1c2[C@@H](NC(=O)NC1CC1O)CC3] |
| 46 | 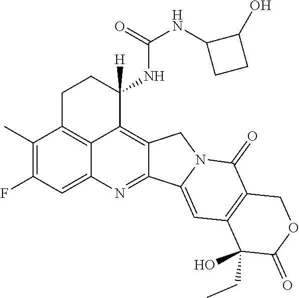 [SMILES: CC[C@@]1(O)C(=O)OCc2c1cc1n(c2=O)Cc2c-1nc1cc(F)c(C)c3c1c2[C@@H](NC(=O)NC1CCC1O)CC3] |
| 47 | 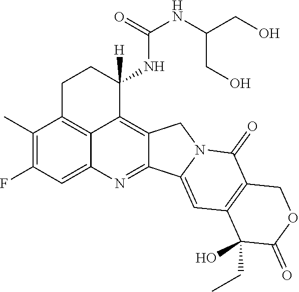 [SMILES: CC[C@@]1(O)C(=O)OCc2c1cc1n(c2=O)Cc2c-1nc1cc(F)c(C)c3c1c2[C@@H](NC(=O)NC(CO)CO)CC3] |
| 48 | 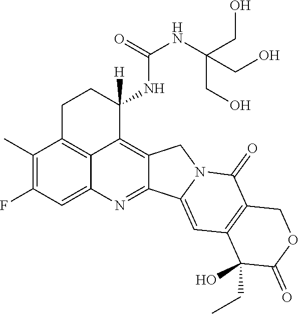 [SMILES: CC[C@@]1(O)C(=O)OCc2c1cc1n(c2=O)Cc2c-1nc1cc(F)c(C)c3c1c2[C@@H](NC(=O)NC(CO)(CO)CO)CC3] |
| 49 | 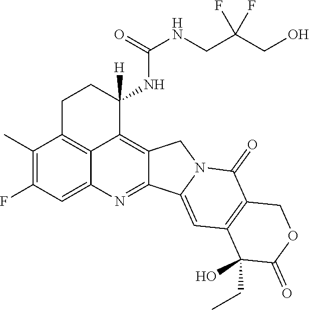 [SMILES: CC[C@@]1(O)C(=O)OCc2c1cc1n(c2=O)Cc2c-1nc1cc(F)c(C)c3c1c2[C@@H](NC(=O)NCC(F)(F)CO)CC3] |
| 50 | 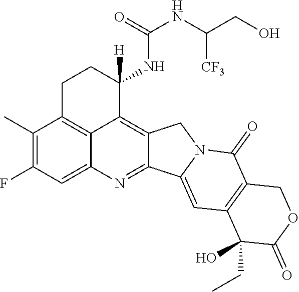 [SMILES: CC[C@@]1(O)C(=O)OCc2c1cc1n(c2=O)Cc2c-1nc1cc(F)c(C)c3c1c2[C@@H](NC(=O)NC(CO)C(F)(F)F)CC3] |
| 51 | 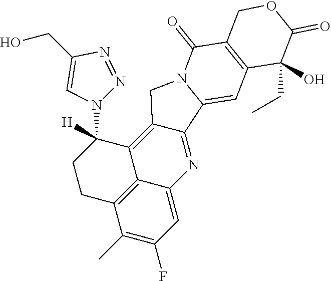 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 52 | 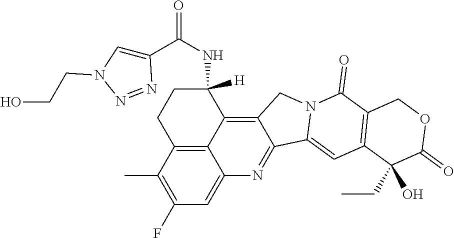 [SMILES: CC[C@@]1(O)C(=O)OCc2c1cc1n(c2=O)Cc2c-1nc1cc(F)c(C)c3c1c2[C@@H](NC(=O)c1cn(CCO)nn1)CC3] |
| 53 | 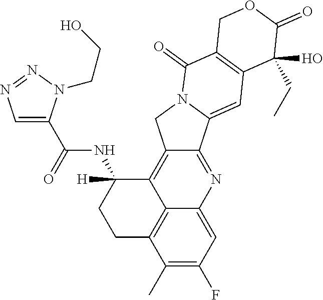 [SMILES: CC[C@@]1(O)C(=O)OCc2c1cc1n(c2=O)Cc2c-1nc1cc(F)c(C)c3c1c2[C@@H](NC(=O)c1cnnn1CCO)CC3] |
| 54 | 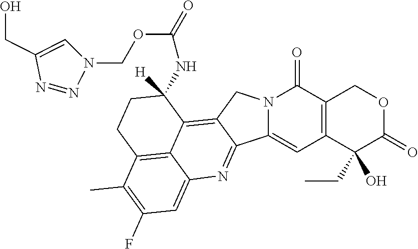 [SMILES: CC[C@@]1(O)C(=O)OCc2c1cc1n(c2=O)Cc2c-1nc1cc(F)c(C)c3c1c2[C@@H](NC(=O)OCn1cc(CO)nn1)CC3] |
| 55 | 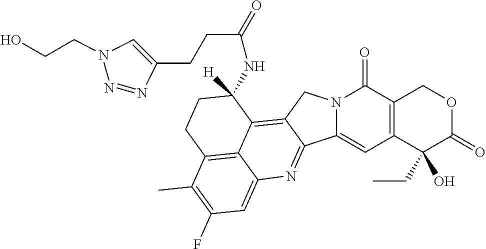 [SMILES: CC[C@@]1(O)C(=O)OCc2c1cc1n(c2=O)Cc2c-1nc1cc(F)c(C)c3c1c2[C@@H](NC(=O)CCc1cn(CCO)nn1)CC3] |
| 56 | 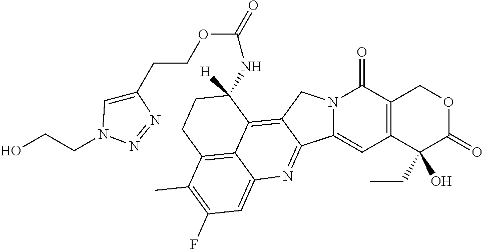 [SMILES: CC[C@@]1(O)C(=O)OCc2c1cc1n(c2=O)Cc2c-1nc1cc(F)c(C)c3c1c2[C@@H](NC(=O)OCCc1cn(CCO)nn1)CC3] |
| 57 | 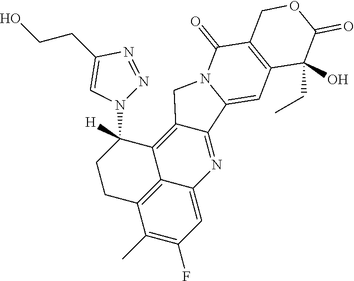 [SMILES: CC[C@@]1(O)C(=O)OCc2c1cc1n(c2=O)Cc2c-1nc1cc(F)c(C)c3c1c2[C@@H](n1cc(CCO)nn1)CC3] |
| 58 | 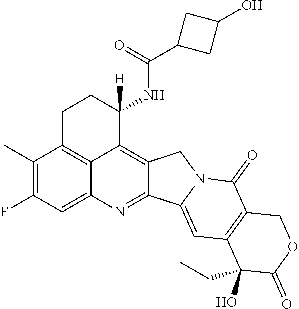 [SMILES: CC[C@@]1(O)C(=O)OCc2c1cc1n(c2=O)Cc2c-1nc1cc(F)c(C)c3c1c2[C@@H](NC(=O)C1CC(O)C1)CC3] |
| 59 | 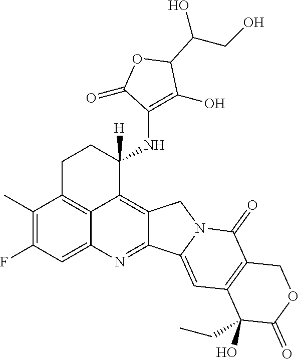 [SMILES: CC[C@@]1(O)C(=O)OCc2c1cc1n(c2=O)Cc2c-1nc1cc(F)c(C)c3c1c2[C@@H](NC1=C(O)C(C(O)CO)OC1=O)CC3] |
| 60 | 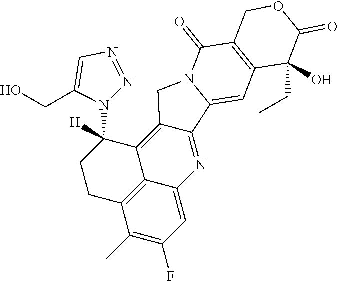 [SMILES: CC[C@@]1(O)C(=O)OCc2c1cc1n(c2=O)Cc2c-1nc1cc(F)c(C)c3c1c2[C@@H](n1nncc1CO)CC3] |
| 61 | 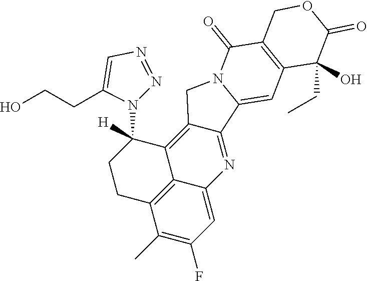 [SMILES: CC[C@@]1(O)C(=O)OCc2c1cc1n(c2=O)Cc2c-1nc1cc(F)c(C)c3c1c2[C@@H](n1nncc1CCO)CC3] |
| 62 | 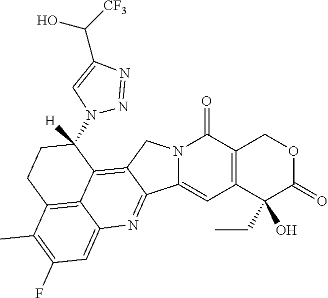 [SMILES: CC[C@@]1(O)C(=O)OCc2c1cc1n(c2=O)Cc2c-1nc1cc(F)c(C)c3c1c2[C@@H](n1cc(C(O)C(F)(F)F)nn1)CC3] |
| 63 | 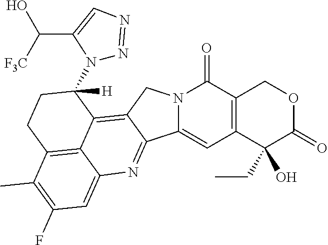 [SMILES: CC[C@@]1(O)C(=O)OCc2c1cc1n(c2=O)Cc2c-1nc1cc(F)c(C)c3c1c2[C@@H](n1nncc1C(O)C(F)(F)F)CC3] |
| 64 | 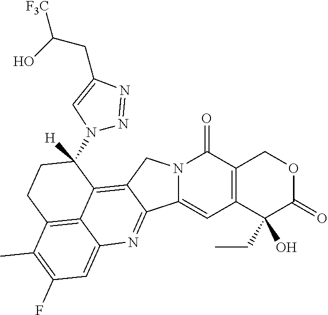 [SMILES: CC[C@@]1(O)C(=O)OCc2c1cc1n(c2=O)Cc2c-1nc1cc(F)c(C)c3c1c2[C@@H](n1cc(CC(O)C(F)(F)F)nn1)CC3] |
| 65 | 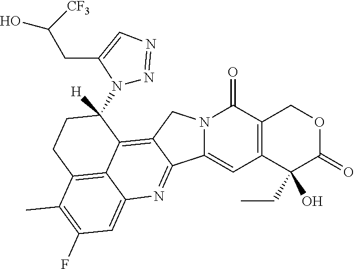 [SMILES: CC[C@@]1(O)C(=O)OCc2c1cc1n(c2=O)Cc2c-1nc1cc(F)c(C)c3c1c2[C@@H](n1nncc1CC(O)C(F)(F)F)CC3] |
| 66 | 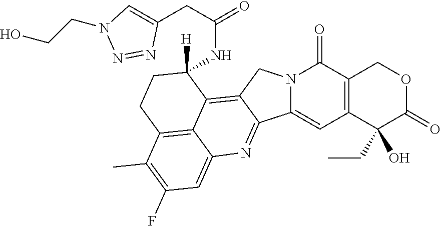 [SMILES: CC[C@@]1(O)C(=O)OCc2c1cc1n(c2=O)Cc2c-1nc1cc(F)c(C)c3c1c2[C@@H](NC(=O)Cc1cn(CCO)nn1)CC3] |
| 67 | 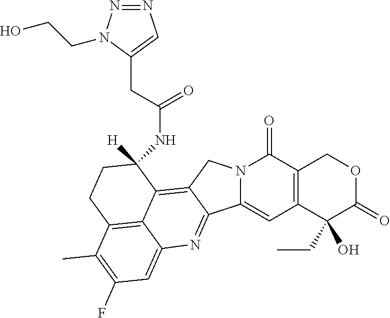 [SMILES: CC[C@@]1(O)C(=O)OCc2c1cc1n(c2=O)Cc2c-1nc1cc(F)c(C)c3c1c2[C@@H](NC(=O)Cc1cnnn1CCO)CC3] |
| 68 | 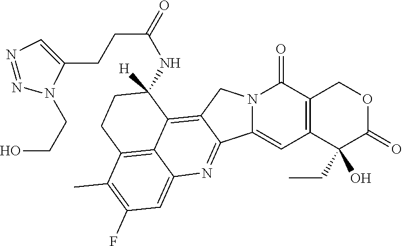 [SMILES: CC[C@@]1(O)C(=O)OCc2c1cc1n(c2=O)Cc2c-1nc1cc(F)c(C)c3c1c2[C@@H](NC(=O)CCc1cnnn1CCO)CC3] |
| 69 | 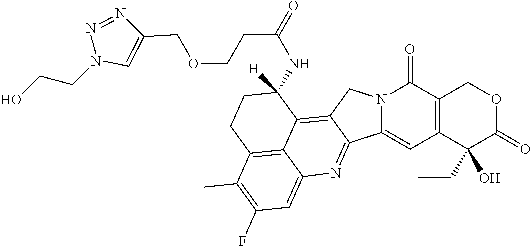 [SMILES: CC[C@@]1(O)C(=O)OCc2c1cc1n(c2=O)Cc2c-1nc1cc(F)c(C)c3c1c2[C@@H](NC(=O)CCOCc1cn(CCO)nn1)CC3] |
| 70 | 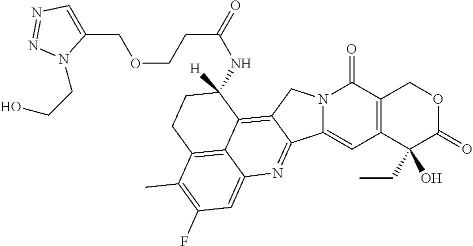 [SMILES: CC[C@@]1(O)C(=O)OCc2c1cc1n(c2=O)Cc2c-1nc1cc(F)c(C)c3c1c2[C@@H](NC(=O)CCOCc1cnnn1CCO)CC3] |
| 71 | 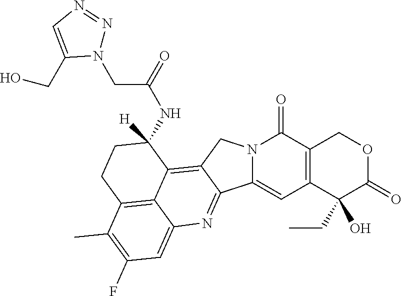 [SMILES: CC[C@@]1(O)C(=O)OCc2c1cc1n(c2=O)Cc2c-1nc1cc(F)c(C)c3c1c2[C@@H](NC(=O)Cn1nncc1CO)CC3] |
| 72 | 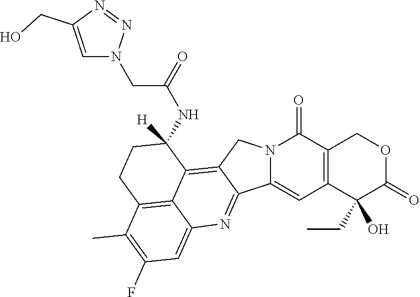 [SMILES: CC[C@@]1(O)C(=O)OCc2c1cc1n(c2=O)Cc2c-1nc1cc(F)c(C)c3c1c2[C@@H](NC(=O)Cn1cc(CO)nn1)CC3] |
| 73 | 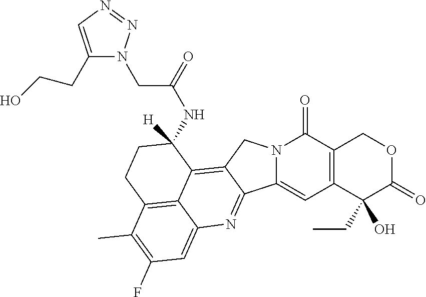 [SMILES: CC[C@@]1(O)C(=O)OCc2c1cc1n(c2=O)Cc2c-1nc1cc(F)c(C)c3c1c2[C@@H](NC(=O)Cn1nncc1CCO)CC3] |
| 74 | 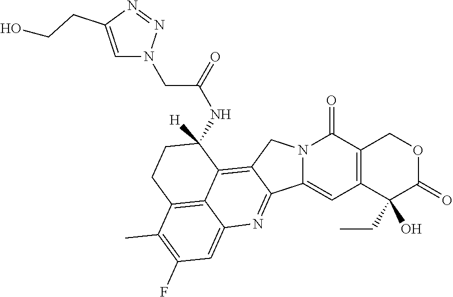 [SMILES: CC[C@@]1(O)C(=O)OCc2c1cc1n(c2=O)Cc2c-1nc1cc(F)c(C)c3c1c2[C@@H](NC(=O)Cn1cc(CCO)nn1)CC3] |
| 75 | 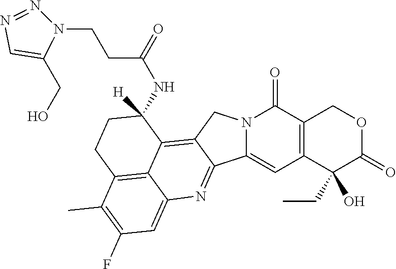 [SMILES: CC[C@@]1(O)C(=O)OCc2c1cc1n(c2=O)Cc2c-1nc1cc(F)c(C)c3c1c2[C@@H](NC(=O)CCn1nncc1CO)CC3] |
| 76 | 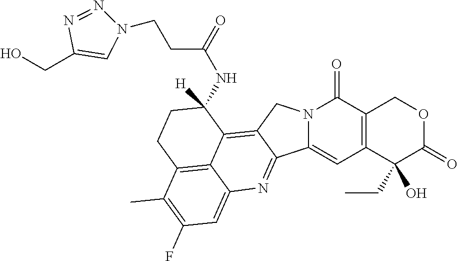 [SMILES: CC[C@@]1(O)C(=O)OCc2c1cc1n(c2=O)Cc2c-1nc1cc(F)c(C)c3c1c2[C@@H](NC(=O)CCn1cc(CO)nn1)CC3] |
| 77 | 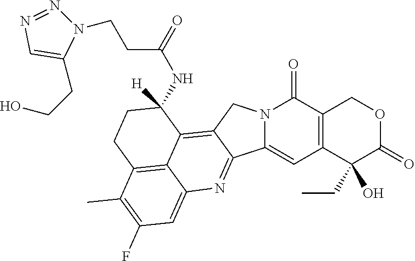 [SMILES: CC[C@@]1(O)C(=O)OCc2c1cc1n(c2=O)Cc2c-1nc1cc(F)c(C)c3c1c2[C@@H](NC(=O)CCn1nncc1CCO)CC3] |
| 78 | 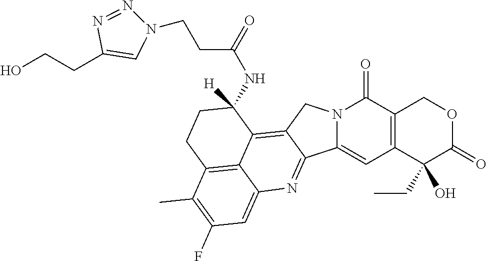 [SMILES: CC[C@@]1(O)C(=O)OCc2c1cc1n(c2=O)Cc2c-1nc1cc(F)c(C)c3c1c2[C@@H](NC(=O)CCn1cc(CCO)nn1)CC3] |
| 79 | 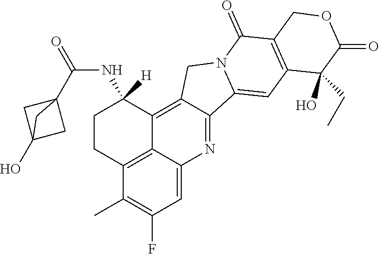 [SMILES: CC[C@@]1(O)C(=O)OCc2c1cc1n(c2=O)Cc2c-1nc1cc(F)c(C)c3c1c2[C@@H](NC(=O)C12CC(O)(C1)C2)CC3] |
| 80 | 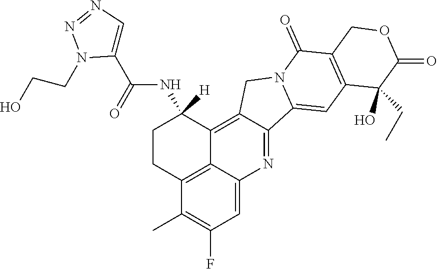 [SMILES: CC[C@@]1(O)C(=O)OCc2c1cc1n(c2=O)Cc2c-1nc1cc(F)c(C)c3c1c2[C@@H](NC(=O)c1cnnn1CCO)CC3] |
| 81 | 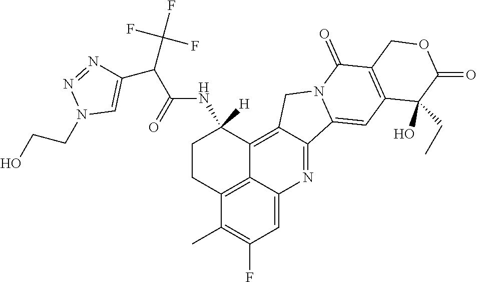 [SMILES: CC[C@@]1(O)C(=O)OCc2c1cc1n(c2=O)Cc2c-1nc1cc(F)c(C)c3c1c2[C@@H](NC(=O)C(c1cn(CCO)nn1)C(F)(F)F)CC3] |
| 82 | 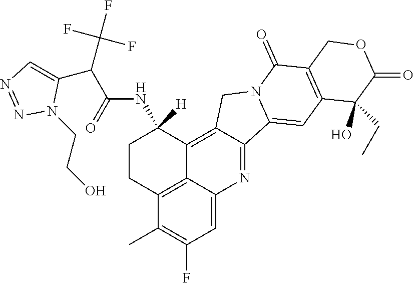 [SMILES: CC[C@@]1(O)C(=O)OCc2c1cc1n(c2=O)Cc2c-1nc1cc(F)c(C)c3c1c2[C@@H](NC(=O)C(c1cnnn1CCO)C(F)(F)F)CC3] |
| 83 | 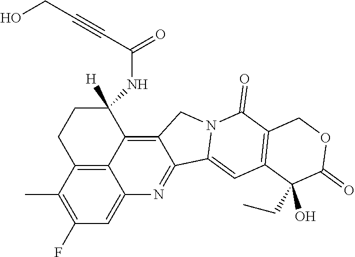 [SMILES: CC[C@@]1(O)C(=O)OCc2c1cc1n(c2=O)Cc2c-1nc1cc(F)c(C)c3c1c2[C@@H](NC(=O)C#CCO)CC3] |
| 84 | 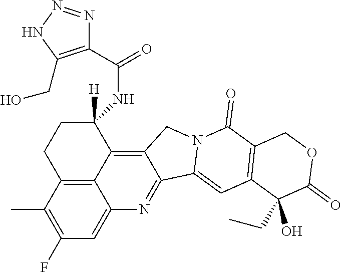 [SMILES: CC[C@@]1(O)C(=O)OCc2c1cc1n(c2=O)Cc2c-1nc1cc(F)c(C)c3c1c2[C@@H](NC(=O)c1nn[nH]c1CO)CC3] |
| 85 | 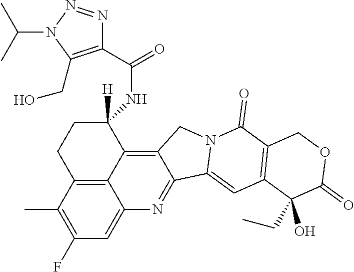 [SMILES: CC[C@@]1(O)C(=O)OCc2c1cc1n(c2=O)Cc2c-1nc1cc(F)c(C)c3c1c2[C@@H](NC(=O)c1nnn(C(C)C)c1CO)CC3] |
| 86 | 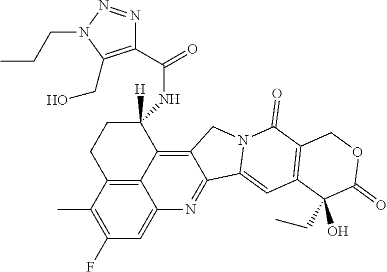 [SMILES: CCCn1nnc(C(=O)N[C@H]2CCc3c(C)c(F)cc4nc5c(c2c34)Cn2c-5cc3c(c2=O)COC(=O)[C@]3(O)CC)c1CO] |
| 87 | 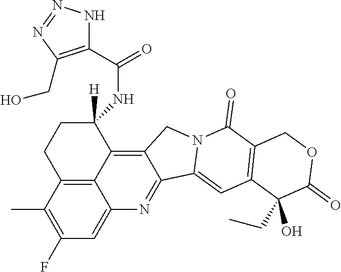 [SMILES: CC[C@@]1(O)C(=O)OCc2c1cc1n(c2=O)Cc2c-1nc1cc(F)c(C)c3c1c2[C@@H](NC(=O)c1[nH]nnc1CO)CC3] |
| 88 | 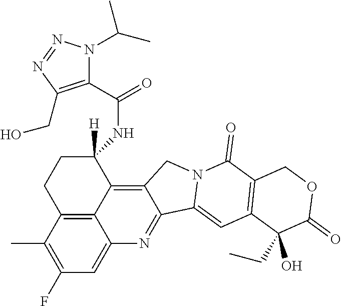 [SMILES: CC[C@@]1(O)C(=O)OCc2c1cc1n(c2=O)Cc2c-1nc1cc(F)c(C)c3c1c2[C@@H](NC(=O)c1c(CO)nnn1C(C)C)CC3] |
| 89 | 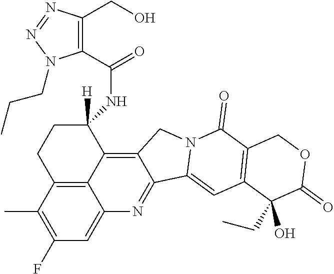 [SMILES: CCCn1nnc(CO)c1C(=O)N[C@H]1CCc2c(C)c(F)cc3nc4c(c1c23)Cn1c-4cc2c(c1=O)COC(=O)[C@]2(O)CC] |
| 90 | 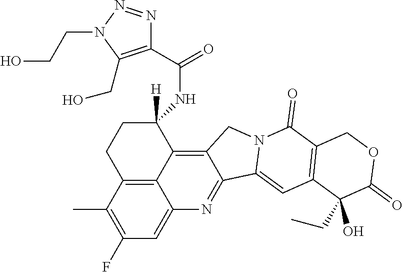 [SMILES: CC[C@@]1(O)C(=O)OCc2c1cc1n(c2=O)Cc2c-1nc1cc(F)c(C)c3c1c2[C@@H](NC(=O)c1nnn(CCO)c1CO)CC3] |
| 91 | 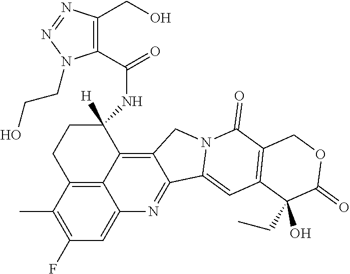 [SMILES: CC[C@@]1(O)C(=O)OCc2c1cc1n(c2=O)Cc2c-1nc1cc(F)c(C)c3c1c2[C@@H](NC(=O)c1c(CO)nnn1CCO)CC3] |
| 92 | 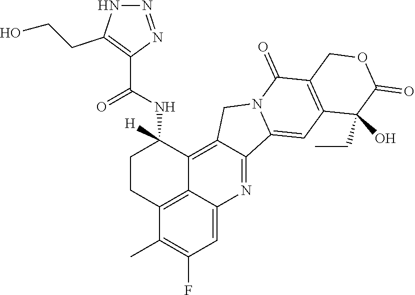 [SMILES: CC[C@@]1(O)C(=O)OCc2c1cc1n(c2=O)Cc2c-1nc1cc(F)c(C)c3c1c2[C@@H](NC(=O)c1nn[nH]c1CCO)CC3] |
| 93 | 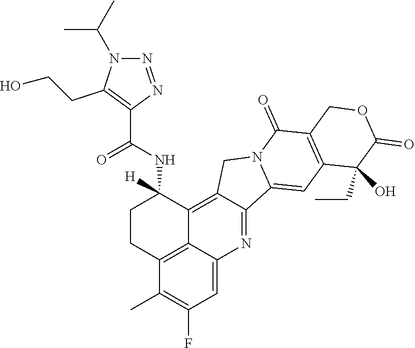 [SMILES: CC[C@@]1(O)C(=O)OCc2c1cc1n(c2=O)Cc2c-1nc1cc(F)c(C)c3c1c2[C@@H](NC(=O)c1nnn(C(C)C)c1CCO)CC3] |
| 94 | 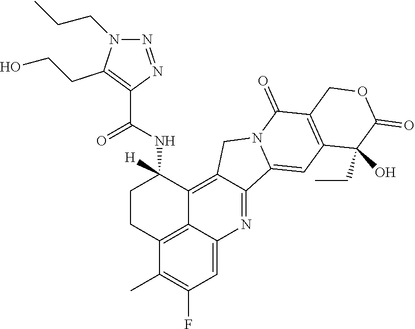 [SMILES: CCCn1nnc(C(=O)N[C@H]2CCc3c(C)c(F)cc4nc5c(c2c34)Cn2c-5cc3c(c2=O)COC(=O)[C@]3(O)CC)c1CCO] |
| 95 | 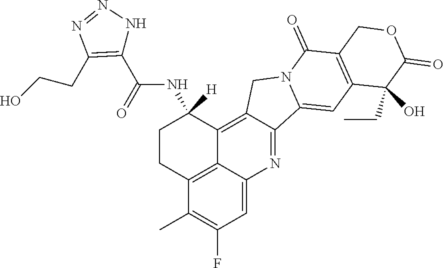 [SMILES: CC[C@@]1(O)C(=O)OCc2c1cc1n(c2=O)Cc2c-1nc1cc(F)c(C)c3c1c2[C@@H](NC(=O)c1[nH]nnc1CCO)CC3] |
| 96 | 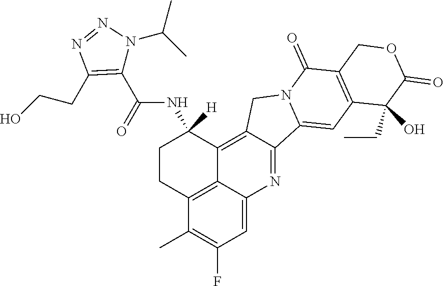 [SMILES: CC[C@@]1(O)C(=O)OCc2c1cc1n(c2=O)Cc2c-1nc1cc(F)c(C)c3c1c2[C@@H](NC(=O)c1c(CCO)nnn1C(C)C)CC3] |
| 97 | 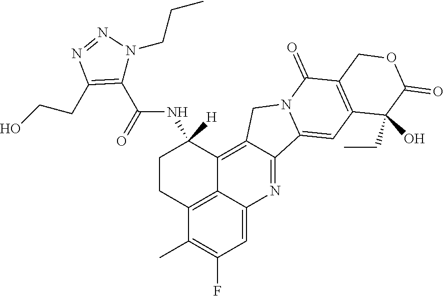 [SMILES: CCCn1nnc(CCO)c1C(=O)N[C@H]1CCc2c(C)c(F)cc3nc4c(c1c23)Cn1c-4cc2c(c1=O)COC(=O)[C@]2(O)CC] |
| 98 | 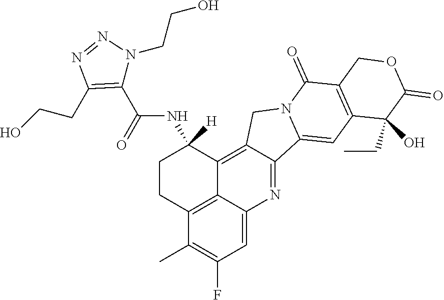 [SMILES: CC[C@@]1(O)C(=O)OCc2c1cc1n(c2=O)Cc2c-1nc1cc(F)c(C)c3c1c2[C@@H](NC(=O)c1c(CCO)nnn1CCO)CC3] |
| 99 | 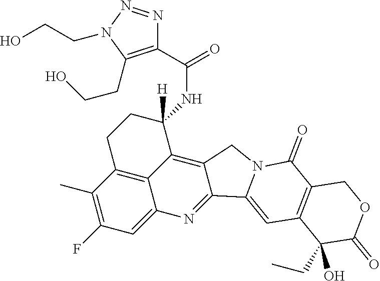 [SMILES: CC[C@@]1(O)C(=O)OCc2c1cc1n(c2=O)Cc2c-1nc1cc(F)c(C)c3c1c2[C@@H](NC(=O)c1nnn(CCO)c1CCO)CC3] |
| 100 | 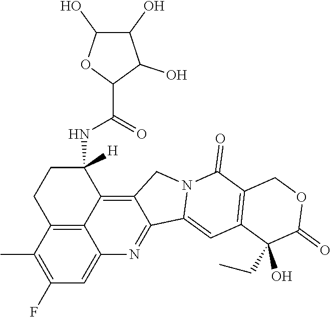 [SMILES: CC[C@@]1(O)C(=O)OCc2c1cc1n(c2=O)Cc2c-1nc1cc(F)c(C)c3c1c2[C@@H](NC(=O)C1OC(O)C(O)C1O)CC3] |
| 101 | 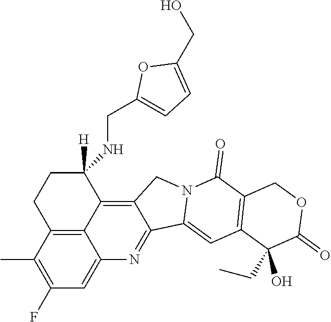 [SMILES: CC[C@@]1(O)C(=O)OCc2c1cc1n(c2=O)Cc2c-1nc1cc(F)c(C)c3c1c2[C@@H](NCc1ccc(CO)o1)CC3] |
| 102 | 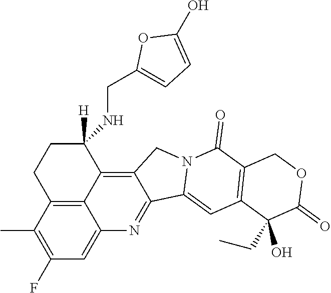 [SMILES: CC[C@@]1(O)C(=O)OCc2c1cc1n(c2=O)Cc2c-1nc1cc(F)c(C)c3c1c2[C@@H](NCc1ccc(O)o1)CC3] |
| 103 | 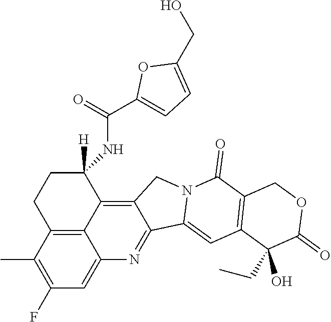 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 104 | 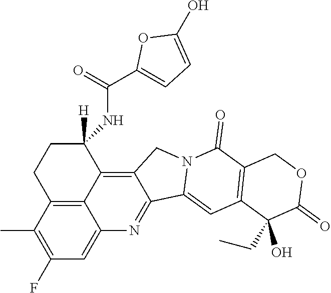 [SMILES: CC[C@@]1(O)C(=O)OCc2c1cc1n(c2=O)Cc2c-1nc1cc(F)c(C)c3c1c2[C@@H](NC(=O)c1ccc(O)o1)CC3] |
| 105 | 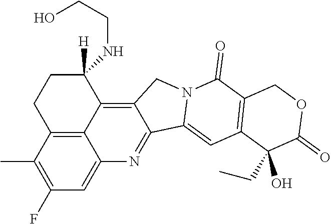 [SMILES: CC[C@@]1(O)C(=O)OCc2c1cc1n(c2=O)Cc2c-1nc1cc(F)c(C)c3c1c2[C@@H](NCCO)CC3] |
| 106 | 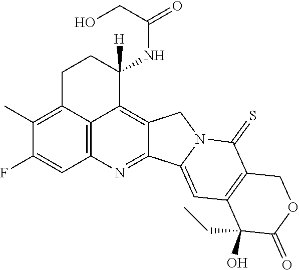 [SMILES: CC[C@@]1(O)C(=O)OCc2c1cc1n(c2=S)Cc2c-1nc1cc(F)c(C)c3c1c2[C@@H](NC(=O)CO)CC3] |
| 107 | 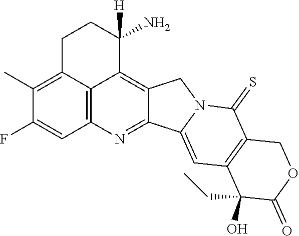 [SMILES: CC[C@@]1(O)C(=O)OCc2c1cc1n(c2=S)Cc2c-1nc1cc(F)c(C)c3c1c2[C@@H](N)CC3] |
| 108 | 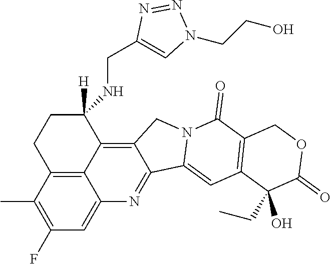 [SMILES: CC[C@@]1(O)C(=O)OCc2c1cc1n(c2=O)Cc2c-1nc1cc(F)c(C)c3c1c2[C@@H](NCc1cn(CCO)nn1)CC3] |
| 109 | 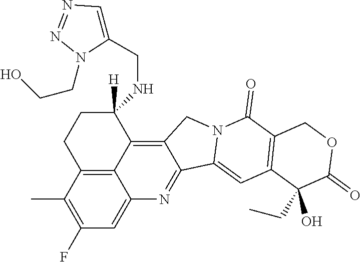 [SMILES: CC[C@@]1(O)C(=O)OCc2c1cc1n(c2=O)Cc2c-1nc1cc(F)c(C)c3c1c2[C@@H](NCc1cnnn1CCO)CC3] |
| 110 | 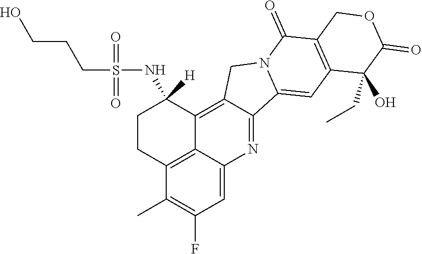 [SMILES: CC[C@@]1(O)C(=O)OCc2c1cc1n(c2=O)Cc2c-1nc1cc(F)c(C)c3c1c2[C@@H](NS(=O)(=O)CCCO)CC3] |
| 111 | 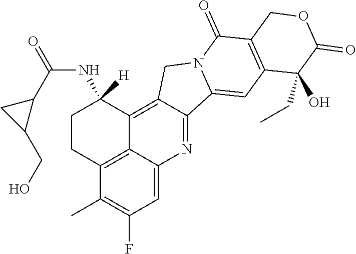 [SMILES: CC[C@@]1(O)C(=O)OCc2c1cc1n(c2=O)Cc2c-1nc1cc(F)c(C)c3c1c2[C@@H](NC(=O)C1CC1CO)CC3] |
| 111-A | 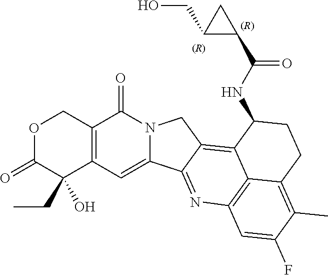 |
| 111-B | 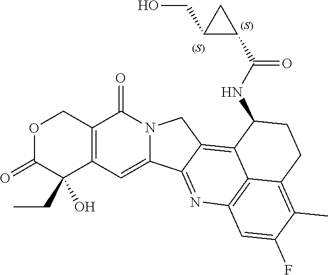 |
| 112 | 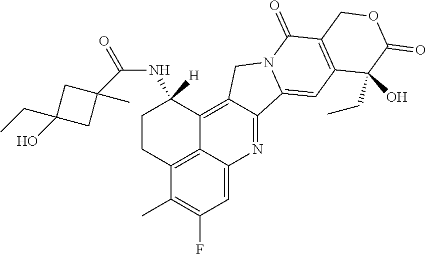 [SMILES: CCC1(O)CC(C)(C(=O)N[C@H]2CCc3c(C)c(F)cc4nc5c(c2c34)Cn2c-5cc3c(c2=O)COC(=O)[C@]3(O)CC)C1] |
| 113 | 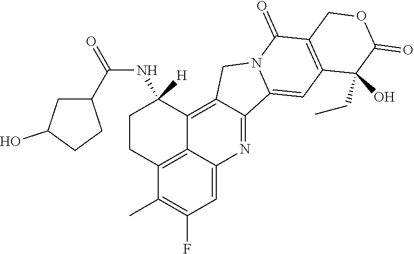 [SMILES: CC[C@@]1(O)C(=O)OCc2c1cc1n(c2=O)Cc2c-1nc1cc(F)c(C)c3c1c2[C@@H](NC(=O)C1CCC(O)C1)CC3] |
| 114 | 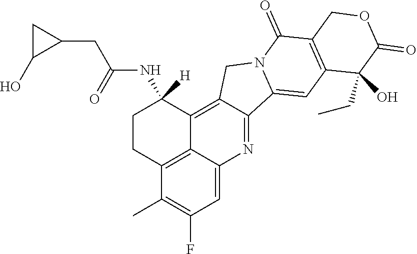 [SMILES: CC[C@@]1(O)C(=O)OCc2c1cc1n(c2=O)Cc2c-1nc1cc(F)c(C)c3c1c2[C@@H](NC(=O)CC1CC1O)CC3] |
| 115 | 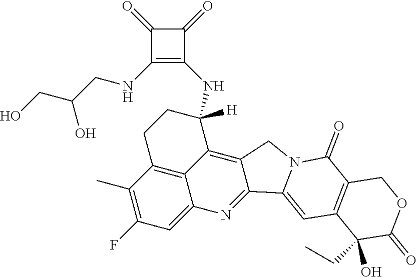 [SMILES: CC[C@@]1(O)C(=O)OCc2c1cc1n(c2=O)Cc2c-1nc1cc(F)c(C)c3c1c2[C@@H](Nc1c(NCC(O)CO)c(=O)c1=O)CC3] |
| 116 | 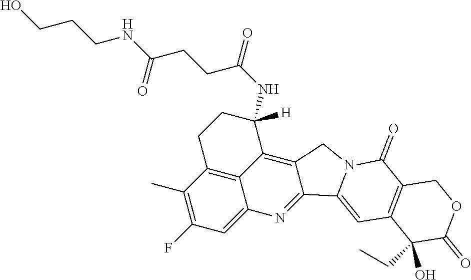 [SMILES: CC[C@@]1(O)C(=O)OCc2c1cc1n(c2=O)Cc2c-1nc1cc(F)c(C)c3c1c2[C@@H](NC(=O)CCC(=O)NCCCO)CC3] |
| 117 | 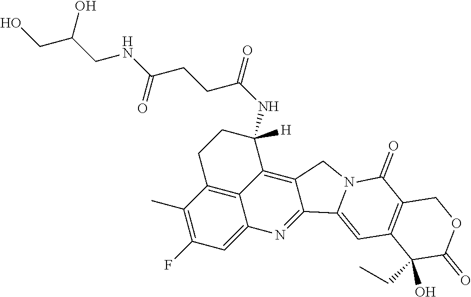 [SMILES: CC[C@@]1(O)C(=O)OCc2c1cc1n(c2=O)Cc2c-1nc1cc(F)c(C)c3c1c2[C@@H](NC(=O)CCC(=O)NCC(O)CO)CC3] |
| 118 | 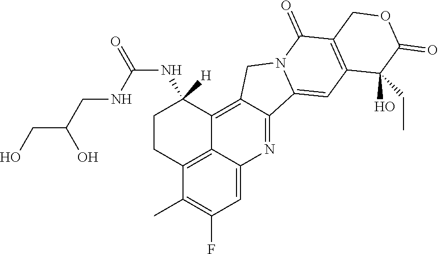 [SMILES: CC[C@@]1(O)C(=O)OCc2c1cc1n(c2=O)Cc2c-1nc1cc(F)c(C)c3c1c2[C@@H](NC(=O)NCC(O)CO)CC3] |
| 119 | 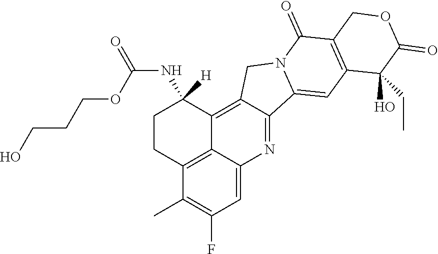 [SMILES: CC[C@@]1(O)C(=O)OCc2c1cc1n(c2=O)Cc2c-1nc1cc(F)c(C)c3c1c2[C@@H](NC(=O)OCCCO)CC3] |
| 120 | 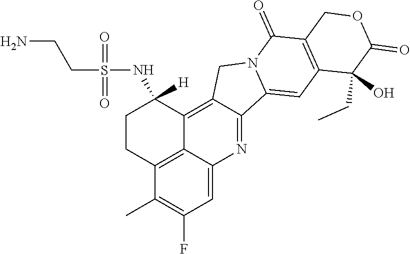 [SMILES: CC[C@@]1(O)C(=O)OCc2c1cc1n(c2=O)Cc2c-1nc1cc(F)c(C)c3c1c2[C@@H](NS(=O)(=O)CCN)CC3] |
| 121 | 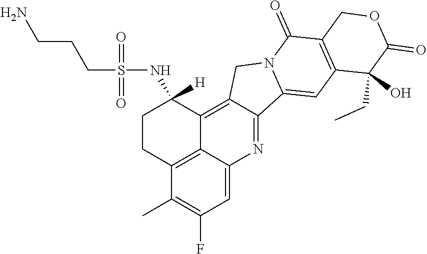 [SMILES: CC[C@@]1(O)C(=O)OCc2c1cc1n(c2=O)Cc2c-1nc1cc(F)c(C)c3c1c2[C@@H](NS(=O)(=O)CCCN)CC3] |
| 122 | 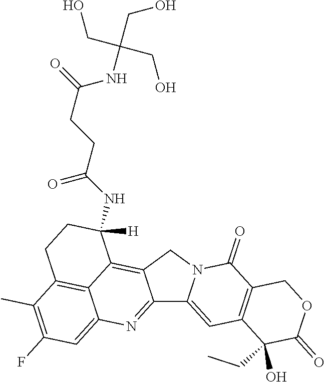 [SMILES: CC[C@@]1(O)C(=O)OCc2c1cc1n(c2=O)Cc2c-1nc1cc(F)c(C)c3c1c2[C@@H](NC(=O)CCC(=O)NC(CO)(CO)CO)CC3] |
| 123 | 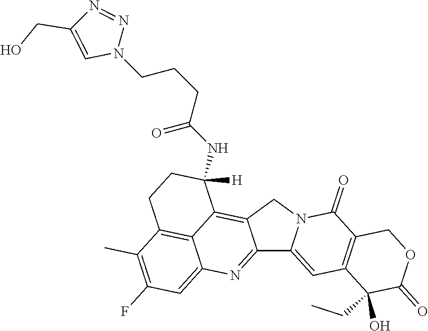 [SMILES: CC[C@@]1(O)C(=O)OCc2c1cc1n(c2=O)Cc2c-1nc1cc(F)c(C)c3c1c2[C@@H](NC(=O)CCCn1cc(CO)nn1)CC3] |
| 124 | 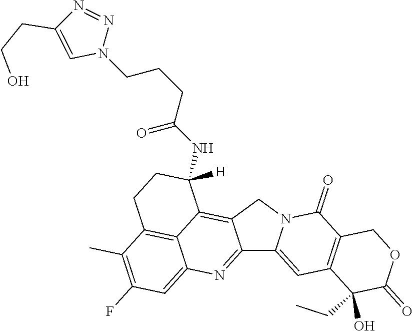 [SMILES: CC[C@@]1(O)C(=O)OCc2c1cc1n(c2=O)Cc2c-1nc1cc(F)c(C)c3c1c2[C@@H](NC(=O)CCCn1cc(CCO)nn1)CC3] |
| 125 | 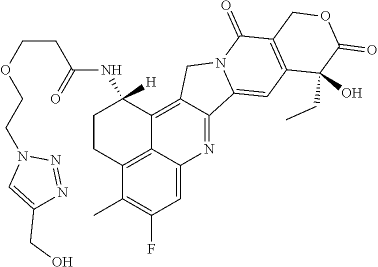 [SMILES: CC[C@@]1(O)C(=O)OCc2c1cc1n(c2=O)Cc2c-1nc1cc(F)c(C)c3c1c2[C@@H](NC(=O)CCOCCn1cc(CO)nn1)CC3] |
| 126 | 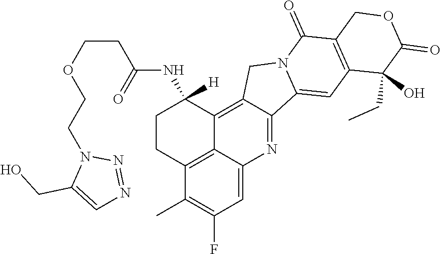 [SMILES: CC[C@@]1(O)C(=O)OCc2c1cc1n(c2=O)Cc2c-1nc1cc(F)c(C)c3c1c2[C@@H](NC(=O)CCOCCn1nncc1CO)CC3] |
| 127 | 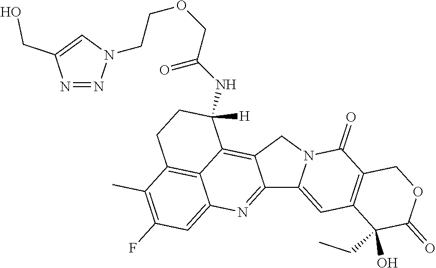 [SMILES: CC[C@@]1(O)C(=O)OCc2c1cc1n(c2=O)Cc2c-1nc1cc(F)c(C)c3c1c2[C@@H](NC(=O)COCCn1cc(CO)nn1)CC3] |
| 128 | 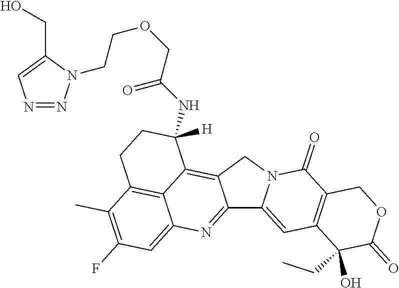 [SMILES: CC[C@@]1(O)C(=O)OCc2c1cc1n(c2=O)Cc2c-1nc1cc(F)c(C)c3c1c2[C@@H](NC(=O)COCCn1nncc1CO)CC3] |
| 129 | 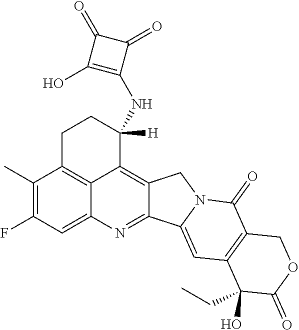 [SMILES: CC[C@@]1(O)C(=O)OCc2c1cc1n(c2=O)Cc2c-1nc1cc(F)c(C)c3c1c2[C@@H](Nc1c(O)c(=O)c1=O)CC3] |
| 130 | 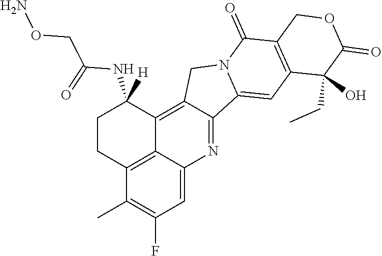 [SMILES: CC[C@@]1(O)C(=O)OCc2c1cc1n(c2=O)Cc2c-1nc1cc(F)c(C)c3c1c2[C@@H](NC(=O)CON)CC3] |
| 131 | 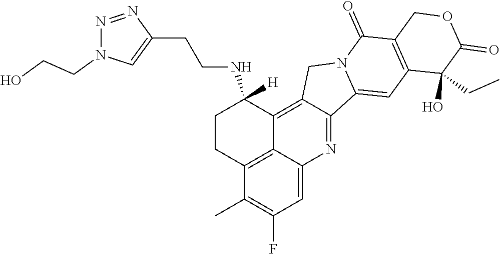 [SMILES: CC[C@@]1(O)C(=O)OCc2c1cc1n(c2=O)Cc2c-1nc1cc(F)c(C)c3c1c2[C@@H](NCCc1cn(CCO)nn1)CC3] |
| 132 | 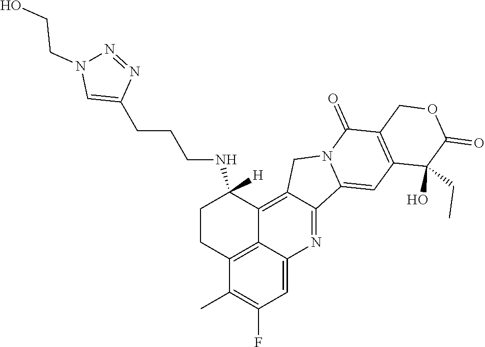 [SMILES: CC[C@@]1(O)C(=O)OCc2c1cc1n(c2=O)Cc2c-1nc1cc(F)c(C)c3c1c2[C@@H](NCCCc1cn(CCO)nn1)CC3] |
| 133 | 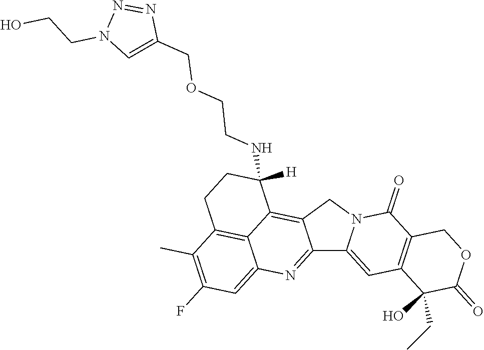 [SMILES: CC[C@@]1(O)C(=O)OCc2c1cc1n(c2=O)Cc2c-1nc1cc(F)c(C)c3c1c2[C@@H](NCCOCc1cn(CCO)nn1)CC3] |
| 134 | 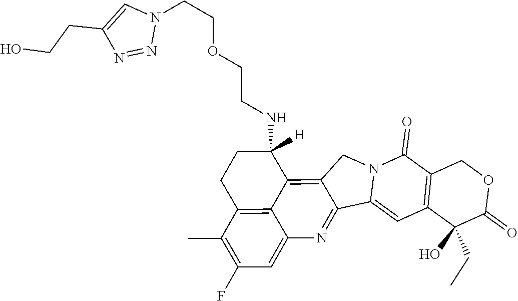 [SMILES: CC[C@@]1(O)C(=O)OCc2c1cc1n(c2=O)Cc2c-1nc1cc(F)c(C)c3c1c2[C@@H](NCCOCCn1cc(CCO)nn1)CC3] |
| 135 | 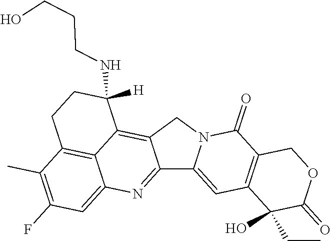 [SMILES: CC[C@@]1(O)C(=O)OCc2c1cc1n(c2=O)Cc2c-1nc1cc(F)c(C)c3c1c2[C@@H](NCCCO)CC3] |
| 136 | 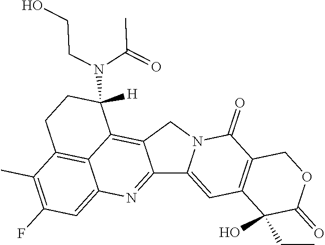 [SMILES: CC[C@@]1(O)C(=O)OCc2c1cc1n(c2=O)Cc2c-1nc1cc(F)c(C)c3c1c2[C@@H](N(CCO)C(C)=O)CC3] |
| 137 | 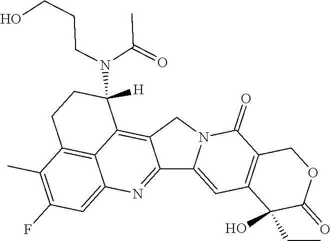 [SMILES: CC[C@@]1(O)C(=O)OCc2c1cc1n(c2=O)Cc2c-1nc1cc(F)c(C)c3c1c2[C@@H](N(CCCO)C(C)=O)CC3] |
| 138 | 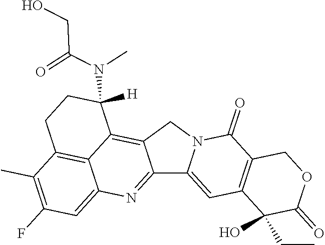 [SMILES: CC[C@@]1(O)C(=O)OCc2c1cc1n(c2=O)Cc2c-1nc1cc(F)c(C)c3c1c2[C@@H](N(C)C(=O)CO)CC3] |
| 139 | 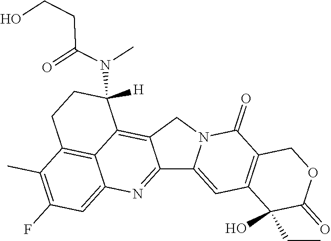 [SMILES: CC[C@@]1(O)C(=O)OCc2c1cc1n(c2=O)Cc2c-1nc1cc(F)c(C)c3c1c2[C@@H](N(C)C(=O)CCO)CC3] |
| 140 | 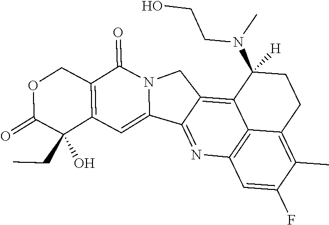 [SMILES: CC[C@@]1(O)C(=O)OCc2c1cc1n(c2=O)Cc2c-1nc1cc(F)c(C)c3c1c2[C@@H](N(C)CCO)CC3] |
| 141 | 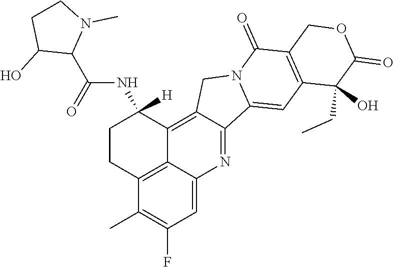 [SMILES: CC[C@@]1(O)C(=O)OCc2c1cc1n(c2=O)Cc2c-1nc1cc(F)c(C)c3c1c2[C@@H](NC(=O)C1C(O)CCN1C)CC3] |
| 142 | 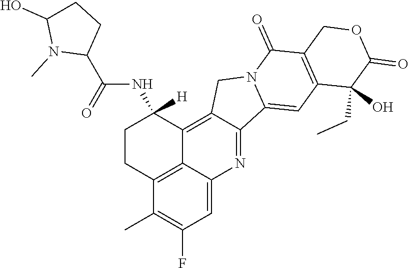 [SMILES: CC[C@@]1(O)C(=O)OCc2c1cc1n(c2=O)Cc2c-1nc1cc(F)c(C)c3c1c2[C@@H](NC(=O)C1CCC(O)N1C)CC3] |
| 143 | 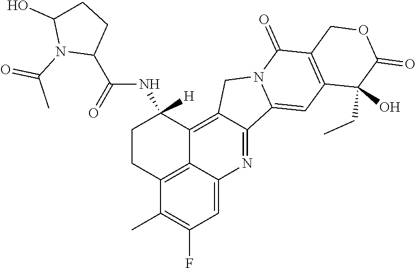 [SMILES: CC[C@@]1(O)C(=O)OCc2c1cc1n(c2=O)Cc2c-1nc1cc(F)c(C)c3c1c2[C@@H](NC(=O)C1CCC(O)N1C(C)=O)CC3] |
| 144 | 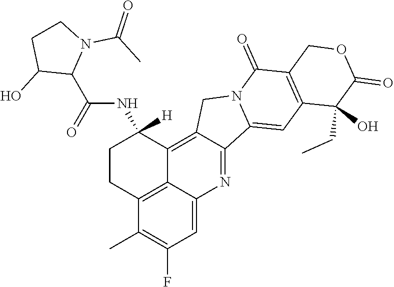 [SMILES: CC[C@@]1(O)C(=O)OCc2c1cc1n(c2=O)Cc2c-1nc1cc(F)c(C)c3c1c2[C@@H](NC(=O)C1C(O)CCN1C(C)=O)CC3] |
| 145 | 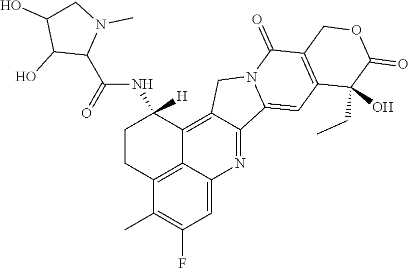 [SMILES: CC[C@@]1(O)C(=O)OCc2c1cc1n(c2=O)Cc2c-1nc1cc(F)c(C)c3c1c2[C@@H](NC(=O)C1C(O)C(O)CN1C)CC3] |
| 146 | 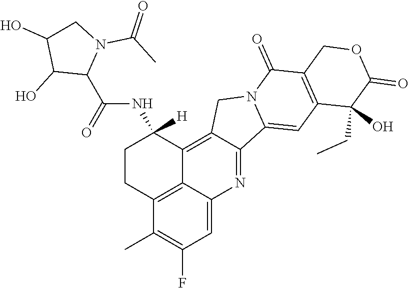 [SMILES: CC[C@@]1(O)C(=O)OCc2c1cc1n(c2=O)Cc2c-1nc1cc(F)c(C)c3c1c2[C@@H](NC(=O)C1C(O)C(O)CN1C(C)=O)CC3] |
| 147 | 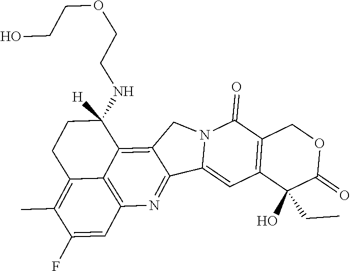 [SMILES: CC[C@@]1(O)C(=O)OCc2c1cc1n(c2=O)Cc2c-1nc1cc(F)c(C)c3c1c2[C@@H](NCCOCCO)CC3] |
| 148 | 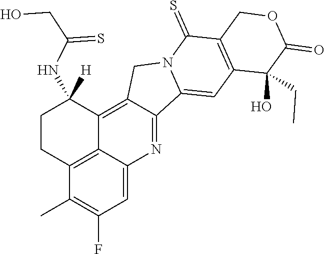 [SMILES: CC[C@@]1(O)C(=O)OCc2c1cc1n(c2=S)Cc2c-1nc1cc(F)c(C)c3c1c2[C@@H](NC(=S)CO)CC3] |
| 149 | 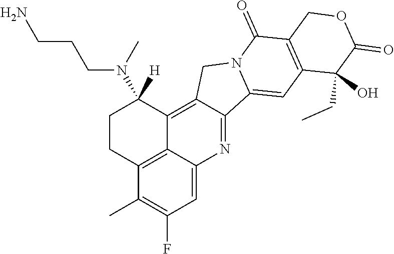 [SMILES: CC[C@@]1(O)C(=O)OCc2c1cc1n(c2=O)Cc2c-1nc1cc(F)c(C)c3c1c2[C@@H](N(C)CCCN)CC3] |
| 150 | 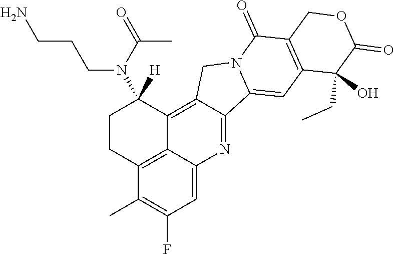 [SMILES: CC[C@@]1(O)C(=O)OCc2c1cc1n(c2=O)Cc2c-1nc1cc(F)c(C)c3c1c2[C@@H](N(CCCN)C(C)=O)CC3] |
| 151 | 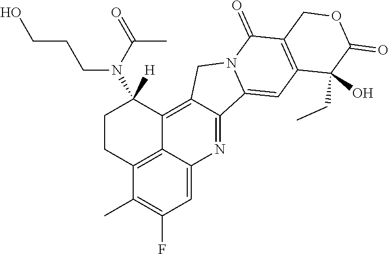 [SMILES: CC[C@@]1(O)C(=O)OCc2c1cc1n(c2=O)Cc2c-1nc1cc(F)c(C)c3c1c2[C@@H](N(CCCO)C(C)=O)CC3] |
| 152 | 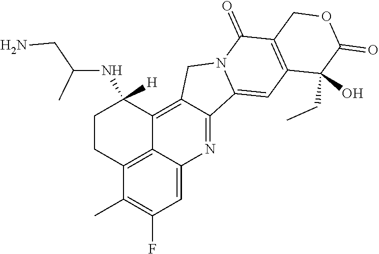 [SMILES: CC[C@@]1(O)C(=O)OCc2c1cc1n(c2=O)Cc2c-1nc1cc(F)c(C)c3c1c2[C@@H](NC(C)CN)CC3] |
| 153 | 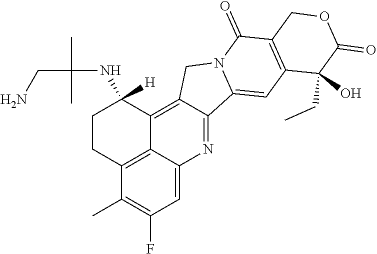 [SMILES: CC[C@@]1(O)C(=O)OCc2c1cc1n(c2=O)Cc2c-1nc1cc(F)c(C)c3c1c2[C@@H](NC(C)(C)CN)CC3] |
| 154 | 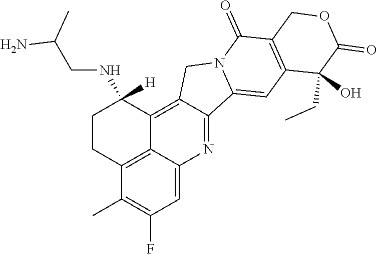 [SMILES: CC[C@@]1(O)C(=O)OCc2c1cc1n(c2=O)Cc2c-1nc1cc(F)c(C)c3c1c2[C@@H](NCC(C)N)CC3] |
| 155 | 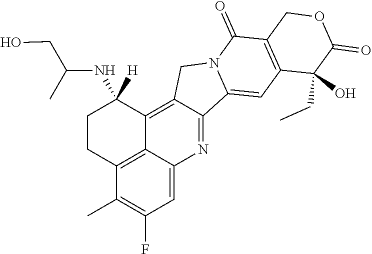 [SMILES: CC[C@@]1(O)C(=O)OCc2c1cc1n(c2=O)Cc2c-1nc1cc(F)c(C)c3c1c2[C@@H](NC(C)CO)CC3] |
| 156 | 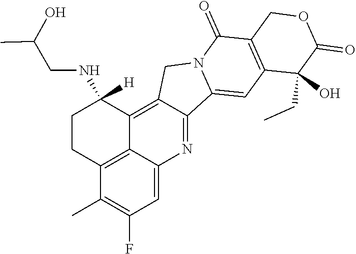 [SMILES: CC[C@@]1(O)C(=O)OCc2c1cc1n(c2=O)Cc2c-1nc1cc(F)c(C)c3c1c2[C@@H](NCC(C)O)CC3] |
| 157 | 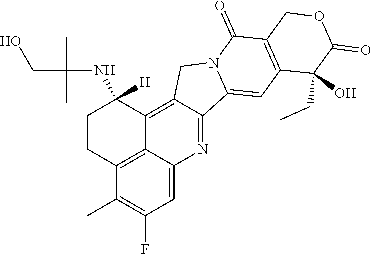 [SMILES: CC[C@@]1(O)C(=O)OCc2c1cc1n(c2=O)Cc2c-1nc1cc(F)c(C)c3c1c2[C@@H](NC(C)(C)CO)CC3] |
| 158 | 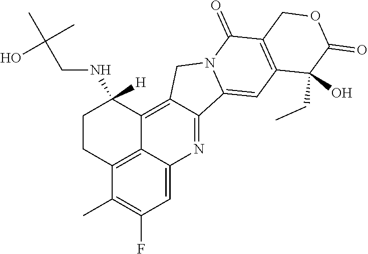 [SMILES: CC[C@@]1(O)C(=O)OCc2c1cc1n(c2=O)Cc2c-1nc1cc(F)c(C)c3c1c2[C@@H](NCC(C)(C)O)CC3] |
| 159 | 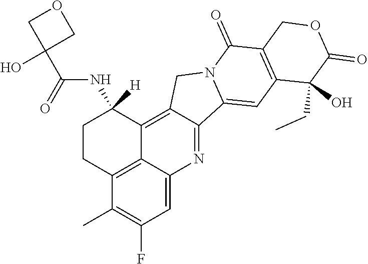 [SMILES: CC[C@@]1(O)C(=O)OCc2c1cc1n(c2=O)Cc2c-1nc1cc(F)c(C)c3c1c2[C@@H](NC(=O)C1(O)COC1)CC3] |
| 160 | 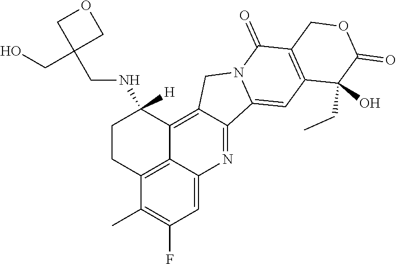 [SMILES: CC[C@@]1(O)C(=O)OCc2c1cc1n(c2=O)Cc2c-1nc1cc(F)c(C)c3c1c2[C@@H](NCC1(CO)COC1)CC3] |
| 161 | 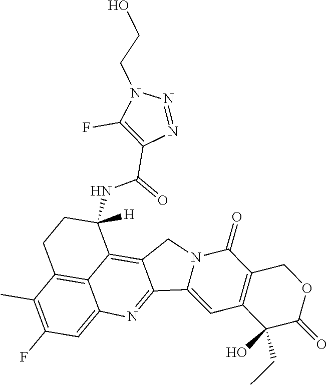 [SMILES: CC[C@@]1(O)C(=O)OCc2c1cc1n(c2=O)Cc2c-1nc1cc(F)c(C)c3c1c2[C@@H](NC(=O)c1nnn(CCO)c1F)CC3] |
| 162 | 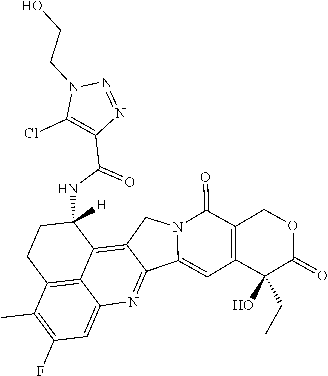 [SMILES: CC[C@@]1(O)C(=O)OCc2c1cc1n(c2=O)Cc2c-1nc1cc(F)c(C)c3c1c2[C@@H](NC(=O)c1nnn(CCO)c1Cl)CC3] |
| 163 | 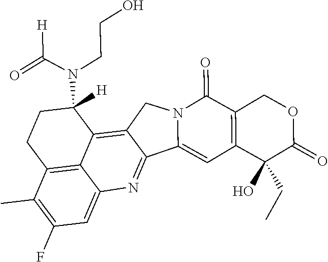 [SMILES: CC[C@@]1(O)C(=O)OCc2c1cc1n(c2=O)Cc2c-1nc1cc(F)c(C)c3c1c2[C@@H](N(C=O)CCO)CC3] |
| 164 | 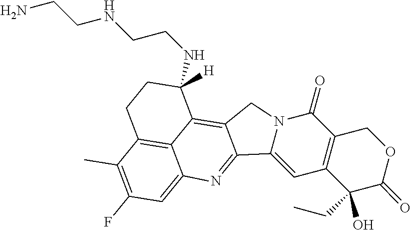 [SMILES: CC[C@@]1(O)C(=O)OCc2c1cc1n(c2=O)Cc2c-1nc1cc(F)c(C)c3c1c2[C@@H](NCCNCCN)CC3] |
| 165 | 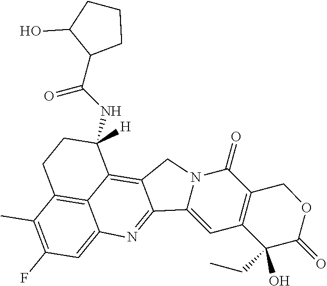 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| 166 | 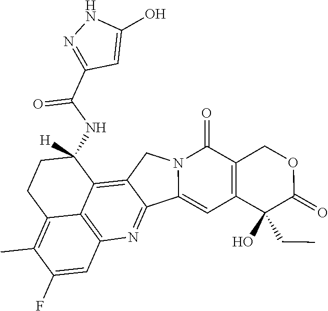 [SMILES: CC[C@@]1(O)C(=O)OCc2c1cc1n(c2=O)Cc2c-1nc1cc(F)c(C)c3c1c2[C@@H](NC(=O)c1cc(O)[nH]n1)CC3] |
| 167 | 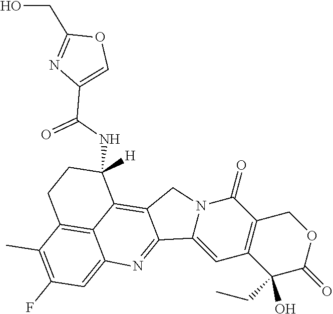 [SMILES: CC[C@@]1(O)C(=O)OCc2c1cc1n(c2=O)Cc2c-1nc1cc(F)c(C)c3c1c2[C@@H](NC(=O)c1coc(CO)n1)CC3] |
| 168 | 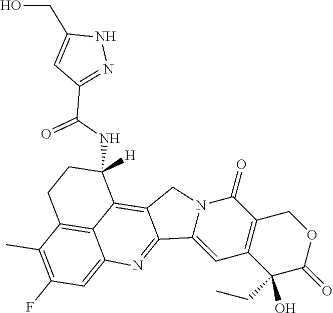 [SMILES: CC[C@@]1(O)C(=O)OCc2c1cc1n(c2=O)Cc2c-1nc1cc(F)c(C)c3c1c2[C@@H](NC(=O)c1cc(CO)[nH]n1)CC3] |
| 169 | 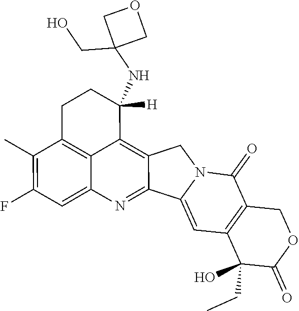 [SMILES: CC[C@@]1(O)C(=O)OCc2c1cc1n(c2=O)Cc2c-1nc1cc(F)c(C)c3c1c2[C@@H](NC1(CO)COC1)CC3] |
| 170 | 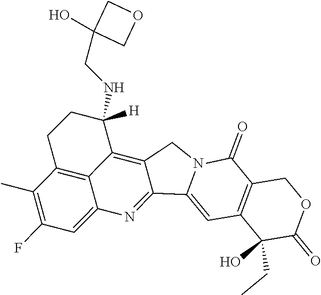 [SMILES: CC[C@@]1(O)C(=O)OCc2c1cc1n(c2=O)Cc2c-1nc1cc(F)c(C)c3c1c2[C@@H](NCC1(O)COC1)CC3] |
| 171 | 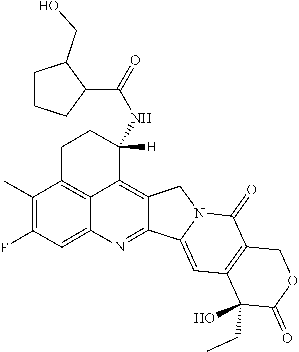 [SMILES: CC[C@@]1(O)C(=O)OCc2c1cc1n(c2=O)Cc2c-1nc1cc(F)c(C)c3c1c2[C@@H](NC(=O)C1CCCC1CO)CC3] |
| 172 | 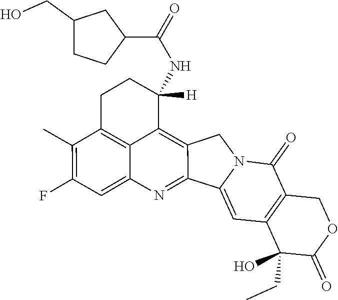 [SMILES: CC[C@@]1(O)C(=O)OCc2c1cc1n(c2=O)Cc2c-1nc1cc(F)c(C)c3c1c2[C@@H](NC(=O)C1CCC(CO)C1)CC3] |
| 173 | 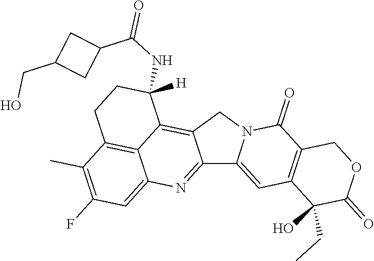 [SMILES: CC[C@@]1(O)C(=O)OCc2c1cc1n(c2=O)Cc2c-1nc1cc(F)c(C)c3c1c2[C@@H](NC(=O)C1CC(CO)C1)CC3] |
| 174 | 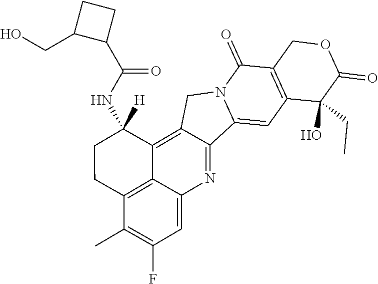 [SMILES: CC[C@@]1(O)C(=O)OCc2c1cc1n(c2=O)Cc2c-1nc1cc(F)c(C)c3c1c2[C@@H](NC(=O)C1CCC1CO)CC3] |
| 175 | 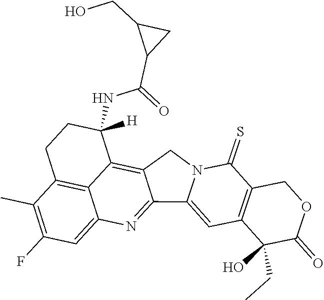 [SMILES: CC[C@@]1(O)C(=O)OCc2c1cc1n(c2=S)Cc2c-1nc1cc(F)c(C)c3c1c2[C@@H](NC(=O)C1CC1CO)CC3] |
| 175-A | 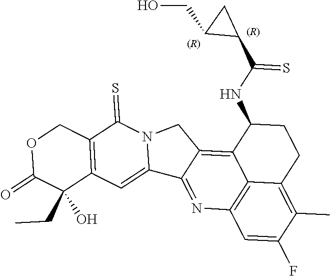 |
| 175-B | 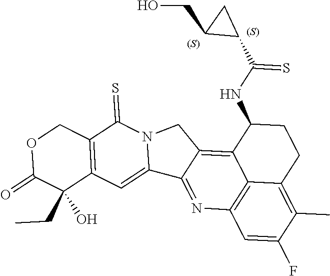 |
| 176 | 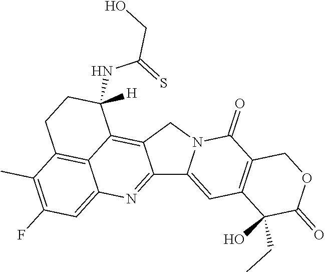 [SMILES: CC[C@@]1(O)C(=O)OCc2c1cc1n(c2=O)Cc2c-1nc1cc(F)c(C)c3c1c2[C@@H](NC(=S)CO)CC3] |
| 177 | 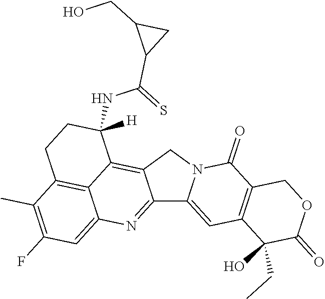 [SMILES: CC[C@@]1(O)C(=O)OCc2c1cc1n(c2=O)Cc2c-1nc1cc(F)c(C)c3c1c2[C@@H](NC(=S)C1CC1CO)CC3] |
| 178 | 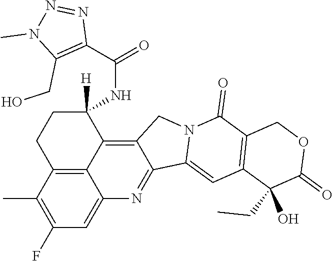 [SMILES: CC[C@@]1(O)C(=O)OCc2c1cc1n(c2=O)Cc2c-1nc1cc(F)c(C)c3c1c2[C@@H](NC(=O)c1nnn(C)c1CO)CC3] |
| 179 | 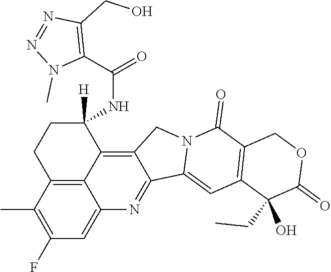 [SMILES: CC[C@@]1(O)C(=O)OCc2c1cc1n(c2=O)Cc2c-1nc1cc(F)c(C)c3c1c2[C@@H](NC(=O)c1c(CO)nnn1C)CC3] |
| 180 | 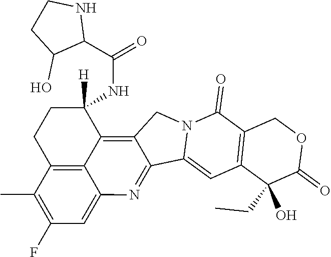 [SMILES: CC[C@@]1(O)C(=O)OCc2c1cc1n(c2=O)Cc2c-1nc1cc(F)c(C)c3c1c2[C@@H](NC(=O)C1NCCC1O)CC3] |
| 181 | 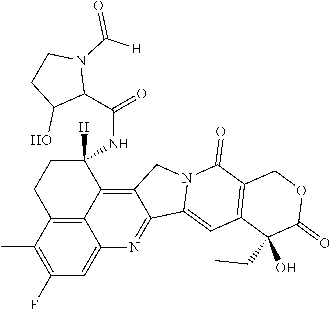 [SMILES: CC[C@@]1(O)C(=O)OCc2c1cc1n(c2=O)Cc2c-1nc1cc(F)c(C)c3c1c2[C@@H](NC(=O)C1C(O)CCN1C=O)CC3] |
| 182 | 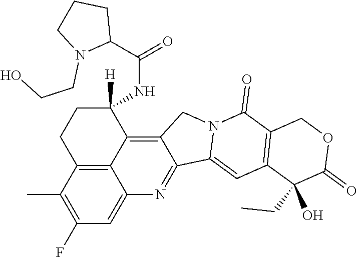 [SMILES: CC[C@@]1(O)C(=O)OCc2c1cc1n(c2=O)Cc2c-1nc1cc(F)c(C)c3c1c2[C@@H](NC(=O)C1CCCN1CCO)CC3] |
| 183 | 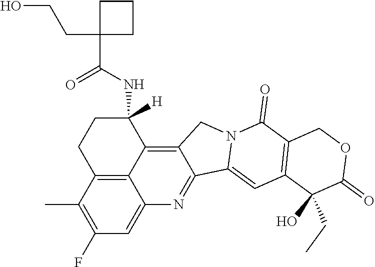 [SMILES: CC[C@@]1(O)C(=O)OCc2c1cc1n(c2=O)Cc2c-1nc1cc(F)c(C)c3c1c2[C@@H](NC(=O)C1(CCO)CCC1)CC3] |
| 184 | 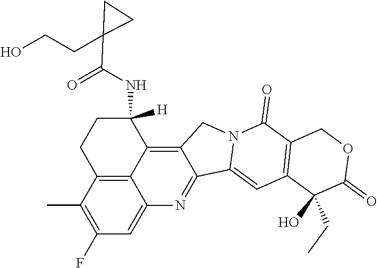 [SMILES: CC[C@@]1(O)C(=O)OCc2c1cc1n(c2=O)Cc2c-1nc1cc(F)c(C)c3c1c2[C@@H](NC(=O)C1(CCO)CC1)CC3] |
| 185 | 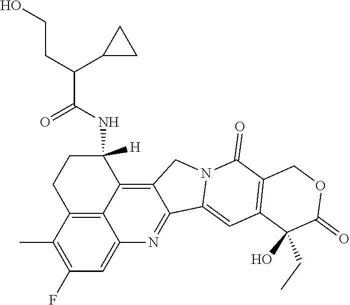 [SMILES: CC[C@@]1(O)C(=O)OCc2c1cc1n(c2=O)Cc2c-1nc1cc(F)c(C)c3c1c2[C@@H](NC(=O)C(CCO)C1CC1)CC3] |
| 186 | 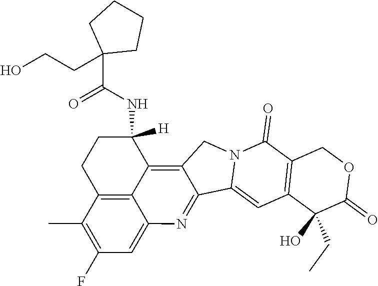 [SMILES: CC[C@@]1(O)C(=O)OCc2c1cc1n(c2=O)Cc2c-1nc1cc(F)c(C)c3c1c2[C@@H](NC(=O)C1(CCO)CCCC1)CC3] |
| 187 | 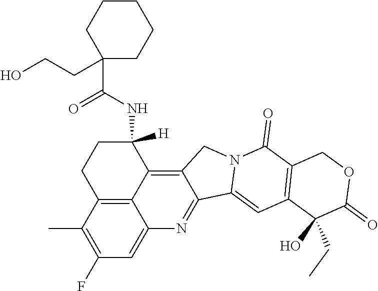 [SMILES: CC[C@@]1(O)C(=O)OCc2c1cc1n(c2=O)Cc2c-1nc1cc(F)c(C)c3c1c2[C@@H](NC(=O)C1(CCO)CCCCC1)CC3] |
| 188 | 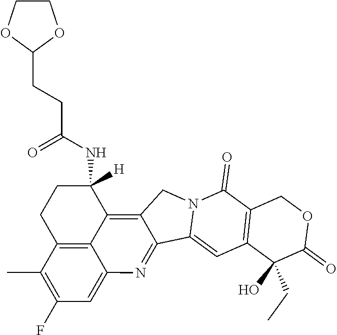 [SMILES: CC[C@@]1(O)C(=O)OCc2c1cc1n(c2=O)Cc2c-1nc1cc(F)c(C)c3c1c2[C@@H](NC(=O)CCC1OCCO1)CC3] |
| 189 | 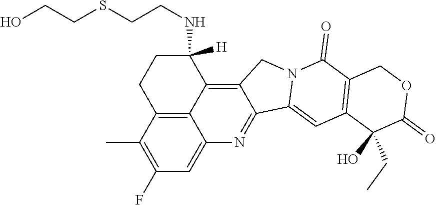 [SMILES: CC[C@@]1(O)C(=O)OCc2c1cc1n(c2=O)Cc2c-1nc1cc(F)c(C)c3c1c2[C@@H](NCCSCCO)CC3] |
| 190 | 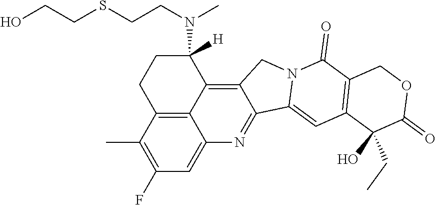 [SMILES: CC[C@@]1(O)C(=O)OCc2c1cc1n(c2=O)Cc2c-1nc1cc(F)c(C)c3c1c2[C@@H](N(C)CCSCCO)CC3] |
| 191 | 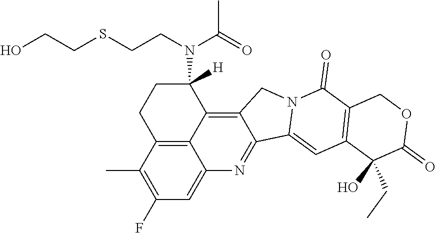 [SMILES: CC[C@@]1(O)C(=O)OCc2c1cc1n(c2=O)Cc2c-1nc1cc(F)c(C)c3c1c2[C@@H](N(CCSCCO)C(C)=O)CC3] |
| 192 | 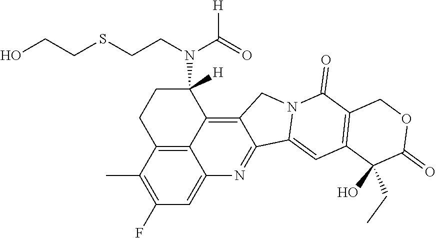 [SMILES: CC[C@@]1(O)C(=O)OCc2c1cc1n(c2=O)Cc2c-1nc1cc(F)c(C)c3c1c2[C@@H](N(C=O)CCSCCO)CC3] |

In some embodiments, a therapeutic payload contemplated herein may be formed, for example, by contacting a cell or tissue at a pH of about 5 to about 7.7 at 37° C. with a drug conjugate represented by Formula IA:

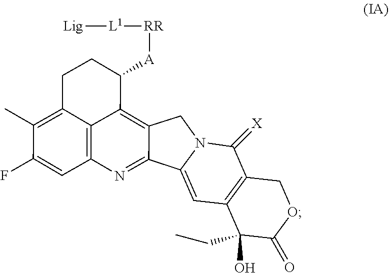

or a pharmaceutically acceptable salt or stereoisomer thereof, wherein:
X is O or S;
A is NH or triazolyl;
Lig is a targeting moiety;
L¹ is a linker moiety; and
RR is an alkoxy or amino moiety formed from L¹ and a hydroxy or —NH₂ moiety of R of any one of the therapeutic payloads described herein.

Also disclosed herein is a method of delivering a therapeutically effective amount of a therapeutic payload moiety to a patient in need thereof, comprising administering to the patient a drug conjugate represented by Formula IA:

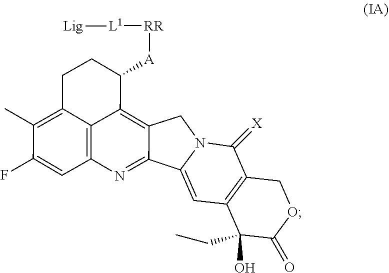

or a pharmaceutically acceptable salt or stereoisomer thereof, wherein:
X is O or S;
A is NH or triazolyl;
Lig is a targeting moiety;
L¹ is a linker moiety; and
RR is an alkoxy or amino moiety formed from L¹ and a hydroxy or —NH₂ moiety of R of any one of the therapeutic payloads described herein.

Also contemplated herein is the drug conjugate represented by:

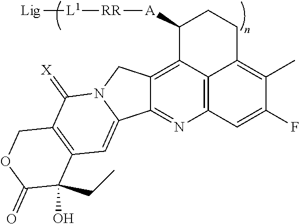

wherein n is 1 to about 10, e.g., about 6.5 to 8.5.

In some embodiments, Lig is a monoclonal antibody. For example, in some embodiments Lig is an antibody selected, for example, from the group consisting of: an anti-TROP2 antibody, an anti-EGRF antibody, an anti-HER2 antibody, an anti-B7-H3 antibody, an anti-CD30 antibody, an anti-CD33 antibody, and an anti-CD70 antibody. In an embodiment, Lig is, for example, an anti-TROP2 antibody.

In other embodiments, L¹ is represented by:
Succinimidyl-(CH₂)₂—O—(CH₂)₂—C(O)—CBP—NH—CH₂—;
Succinimidyl-(CH₂)₂—O—(CH₂)₂—C(O)—CBP—;
Succinimidyl-(CH₂)₅—C(O)—CBP—NH—CH₂—; or
Succinimidyl-(CH₂)₅—C(O)—CBP—;
wherein CBP is a cathepsin B cleavable moiety or a cathepsin D cleavable moiety.

In further embodiments, CBP is, for example, a cathepsin B cleavable peptide or a cathepsin D cleavable peptide. In an embodiment, CBP is -Gly-Gly-Phe-Gly- or -Val-Cit-.

In some embodiments, L¹ is selected for example, from the group consisting of:

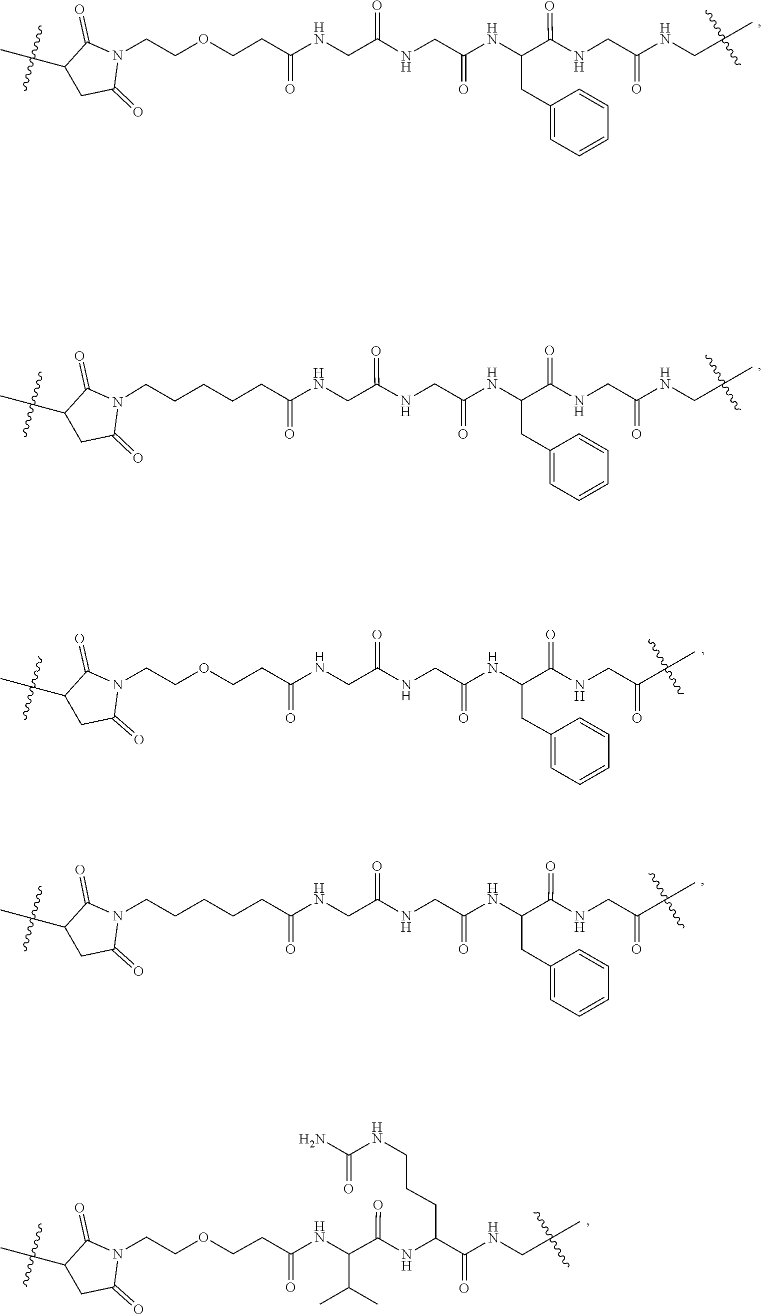 [SMILES: *CNC(=O)CNC(=O)C(Cc1ccccc1)NC(=O)CNC(=O)CNC(=O)CCOCCN1C(=O)CC(*)C1=O ; *CNC(=O)CNC(=O)C(Cc1ccccc1)NC(=O)CNC(=O)CNC(=O)CCCCCN1C(=O)CC(*)C1=O ; *C(=O)CNC(=O)C(Cc1ccccc1)NC(=O)CNC(=O)CNC(=O)CCOCCN1C(=O)CC(*)C1=O ; *C(=O)CNC(=O)C(Cc1ccccc1)NC(=O)CNC(=O)CNC(=O)CCCCCN1C(=O)CC(*)C1=O ; *CNC(=O)C(CCCNC(N)=O)NC(=O)C(NC(=O)CCOCCN1C(=O)CC(*)C1=O)C(C)C]

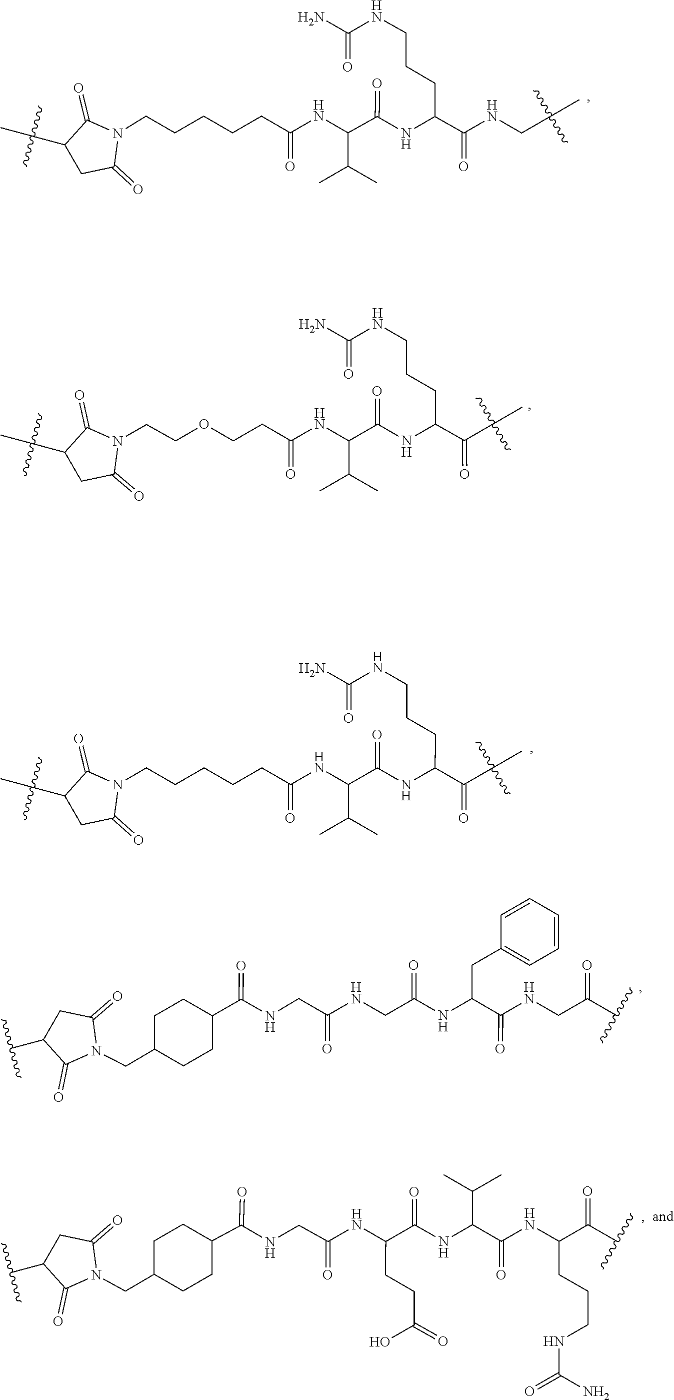 [SMILES: *CNC(=O)C(CCCNC(N)=O)NC(=O)C(NC(=O)CCCCCN1C(=O)CC(*)C1=O)C(C)C ; *C(=O)C(CCCNC(N)=O)NC(=O)C(NC(=O)CCOCCN1C(=O)CC(*)C1=O)C(C)C ; *C(=O)C(CCCNC(N)=O)NC(=O)C(NC(=O)CCCCCN1C(=O)CC(*)C1=O)C(C)C ; *C(=O)CNC(=O)C(Cc1ccccc1)NC(=O)CNC(=O)CNC(=O)C1CCC(CN2C(=O)CC(*)C2=O)CC1 ; *C(=O)C(CCCNC(N)=O)NC(=O)C(NC(=O)C(CCC(=O)O)NC(=O)CNC(=O)C1CCC(CN2C(=O)CC(*)C2=O)CC1)C(C)C]

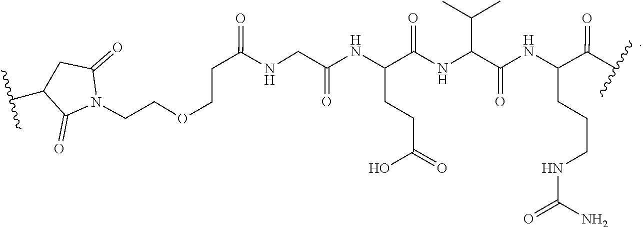 [SMILES: *C(=O)C(CCCNC(N)=O)NC(=O)C(NC(=O)C(CCC(=O)O)NC(=O)CNC(=O)CCOCCN1C(=O)CC(*)C1=O)C(C)C]

Further disclosed herein is a method if delivering a therapeutically effective amount of a therapeutic payload moiety to a patient in need thereof, comprising administering to the patient a drug conjugate represented by Formula IB:

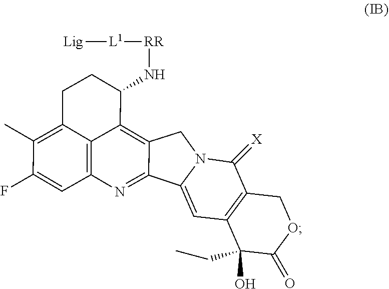

or a pharmaceutically acceptable salt or stereoisomer thereof, wherein:
X is O or S;
Lig is a targeting moiety;
L¹ is a linking moiety; and
L² is a self-immolating moiety.

In some embodiments, Lig is a monoclonal antibody. For example, in some embodiments Lig is an antibody selected, for example, from the group consisting of: an anti-TROP2 antibody, an anti-EGRF antibody, an anti-HER2 antibody, an anti-B7-H3 antibody, an anti-CD30 antibody, an anti-CD33 antibody, and an anti-CD70 antibody. In an embodiment, Lig is, for example, an anti-TROP2 antibody.

In other embodiments, L¹ is represented by:
Succinimidyl-(CH₂)₂—O—(CH₂)₂—C(O)—CBP— or -Succinimidyl-(CH₂)₅—C(O)—CBP—; wherein CBP is a cathepsin B cleavable moiety or a cathepsin D cleavable moiety.

In some embodiments, CBP is, for example, a cathepsin B cleavable peptide or a cathepsin D cleavable peptide. In an embodiment, CBP is -Gly-Gly-Phe-Gly- or -Val-Cit-.

In further embodiments, L¹ is, for example, selected from the group consisting of:

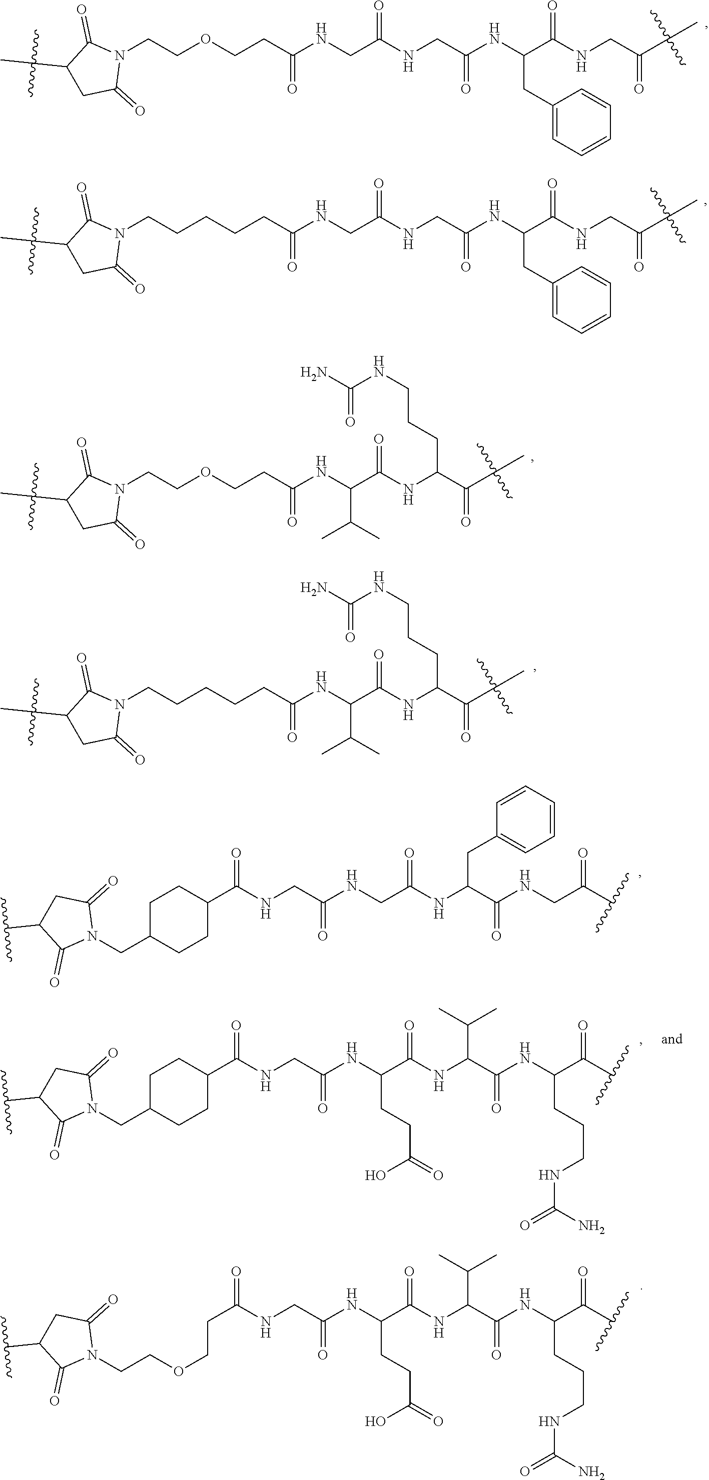

In still further embodiments, L² is, e.g., selected from the group consisting of:

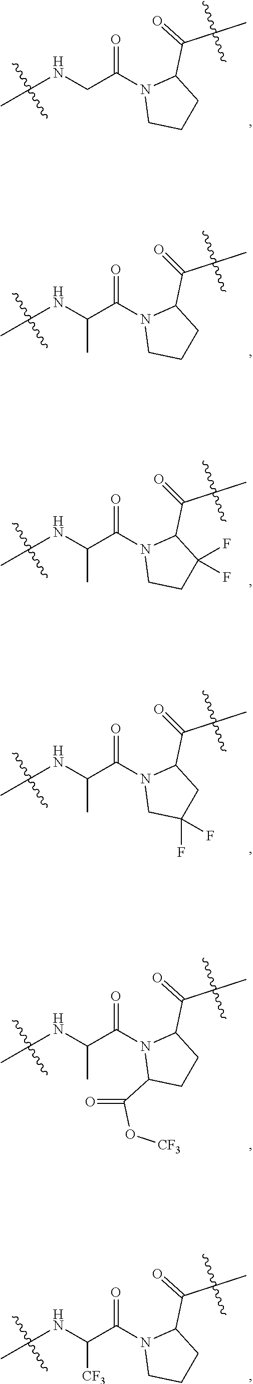 [SMILES: *NCC(=O)N1CCCC1C(*)=O ; *NC(C)C(=O)N1CCCC1C(*)=O ; *NC(C)C(=O)N1CCC(F)(F)C1C(*)=O ; *NC(C)C(=O)N1CC(F)(F)CC1C(*)=O ; *NC(C)C(=O)N1C(C(*)=O)CCC1C(=O)OC(F)(F)F ; *NC(C(=O)N1CCCC1C(*)=O)C(F)(F)F]

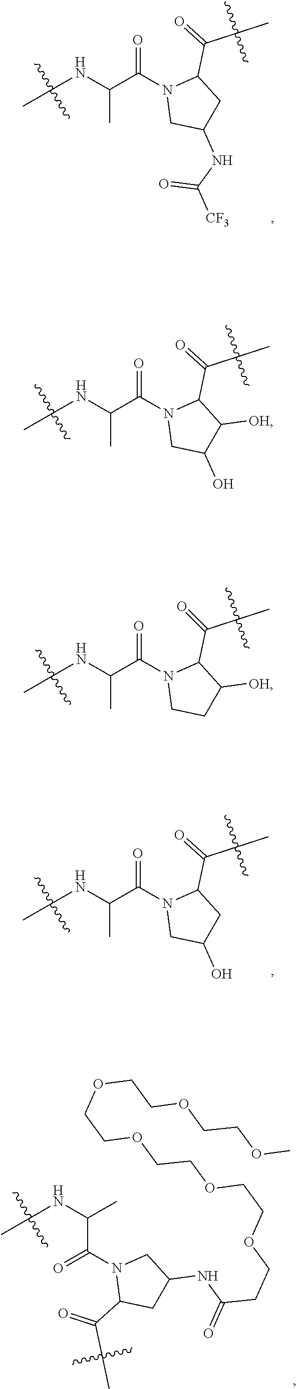 [SMILES: *NC(C)C(=O)N1CC(NC(=O)C(F)(F)F)CC1C(*)=O ; *NC(C)C(=O)N1CC(O)C(O)C1C(*)=O ; *NC(C)C(=O)N1CCC(O)C1C(*)=O ; *NC(C)C(=O)N1CC(O)CC1C(*)=O ; *NC(C)C(=O)N1CC(NC(=O)CCOCCOCCOCCOCCOCCOC)CC1C(*)=O]

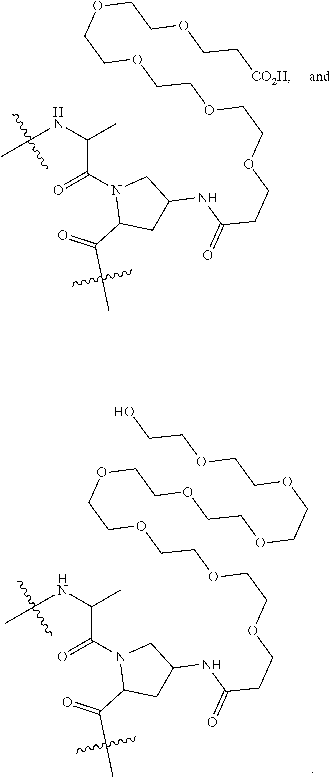 [SMILES: *NC(C)C(=O)N1CC(NC(=O)CCOCCOCCOCCOCCOCCC(=O)O)CC1C(*)=O ; *NC(C)C(=O)N1CC(NC(=O)CCOCCOCCOCCOCCOCCOCCOCCOCCO)CC1C(*)=O]

Disclosed herein, for example, is a linker-payload construct Formula IIA or Formula IIB:

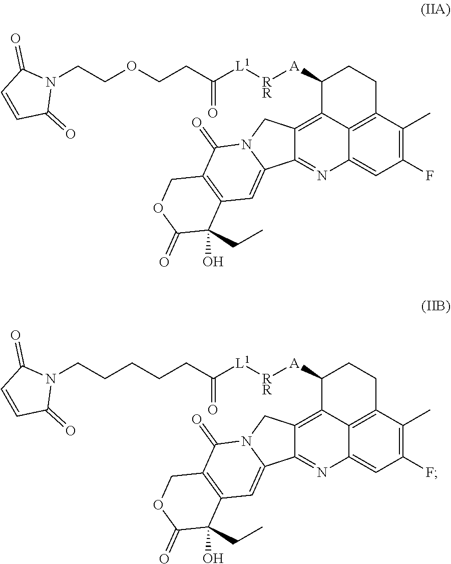

or a pharmaceutically acceptable salt or stereoisomer thereof, wherein:
A is NH or triazolyl;
L¹ is —CBP—NH—CH₂—, or —CBP—, wherein CBP is a cathepsin B cleavable peptide or a cathepsin D cleavable peptide; and
RR is an alkoxy or amino moiety formed from L¹ and a hydroxy or —NH₂ moiety of R of any one of the therapeutic payloads described herein.

In some embodiments, L¹ is selected from the group consisting of:

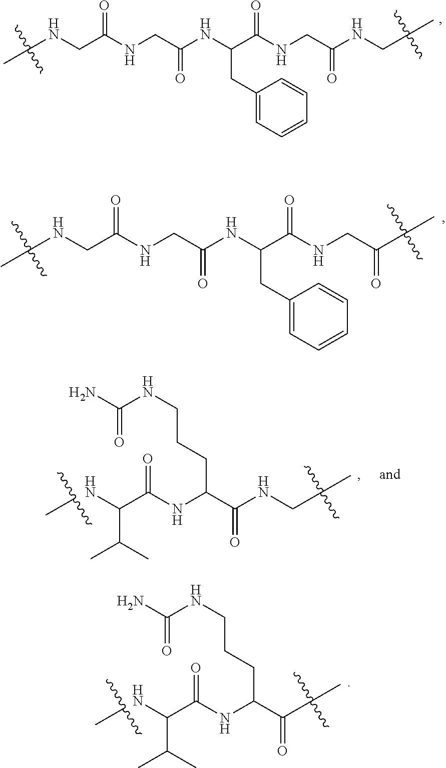

In other embodiments, the linker-payload construct is selected from the group consisting of:

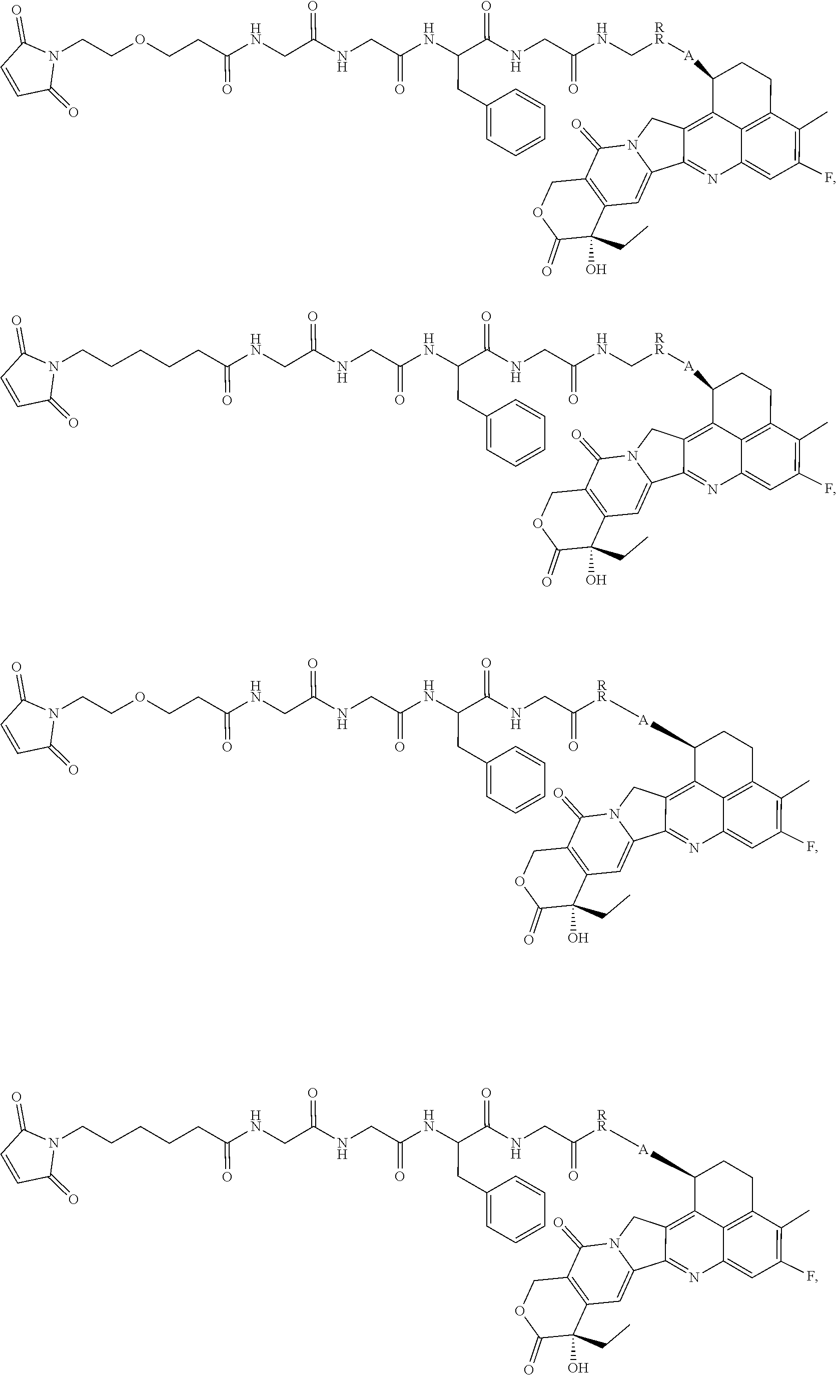

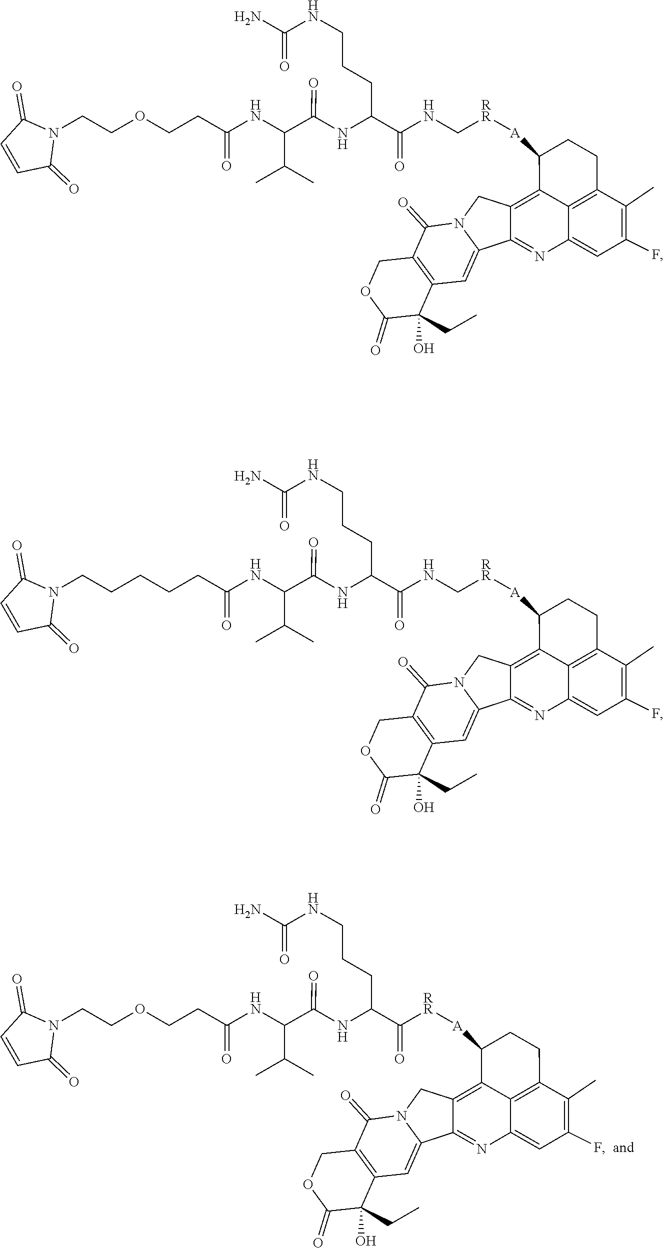

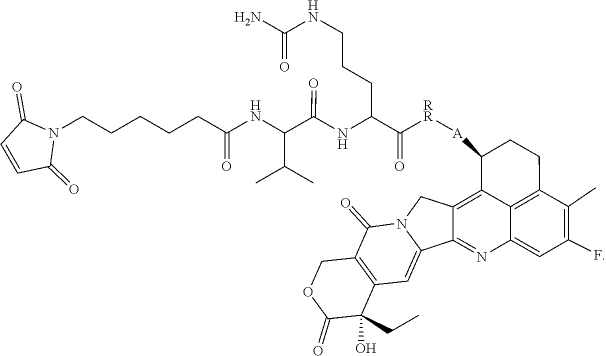

In some embodiments, a disclosed linker-payload construct may be selected, for example, from any one of the compounds disclosed in Table 2, or a pharmaceutically acceptable salt or stereoisomer thereof.

TABLE 2

| Comp | |
|---|---|
| # | Structure |
| 1001 | 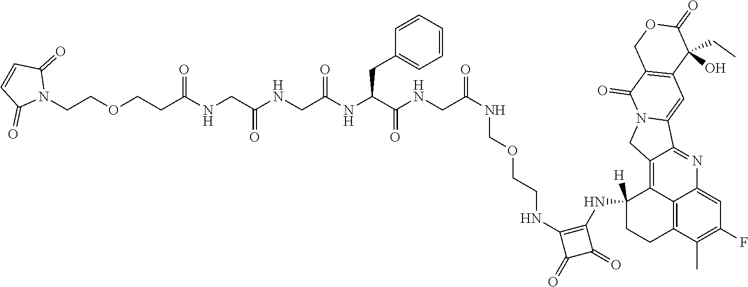 [SMILES: CC[C@@]1(O)C(=O)OCc2c1cc1n(c2=O)Cc2c-1nc1cc(F)c(C)c3c1c2[C@@H](Nc1c(NCCOCNC(=O)CNC(=O)[C@H](Cc2ccccc2)NC(=O)CNC(=O)CNC(=O)CCOCCN2C(=O)C=CC2=O)c(=O)c1=O)CC3] |
| 1002 | 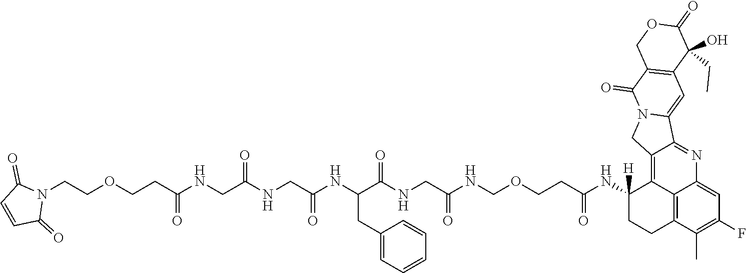 [SMILES: CC[C@@]1(O)C(=O)OCc2c1cc1n(c2=O)Cc2c-1nc1cc(F)c(C)c3c1c2[C@@H](NC(=O)CCOCNC(=O)CNC(=O)C(Cc1ccccc1)NC(=O)CNC(=O)CNC(=O)CCOCCN1C(=O)C=CC1=O)CC3] |
| 1003 | 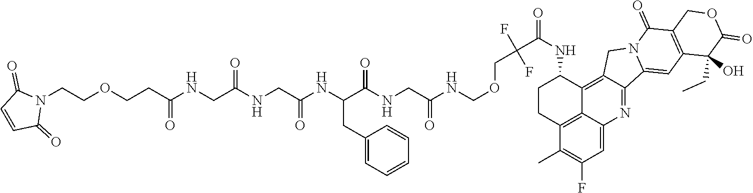 [SMILES: CC[C@@]1(O)C(=O)OCc2c1cc1n(c2=O)Cc2c-1nc1cc(F)c(C)c3c1c2[C@@H](NC(=O)C(F)(F)COCNC(=O)CNC(=O)C(Cc1ccccc1)NC(=O)CNC(=O)CNC(=O)CCOCCN1C(=O)C=CC1=O)CC3] |
| 1004 | 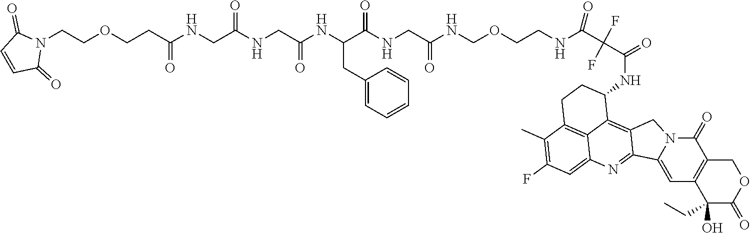 [SMILES: CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CNC(=O)CCOCCN1C(=O)C=CC1=O)CC3] |
| 1005 | 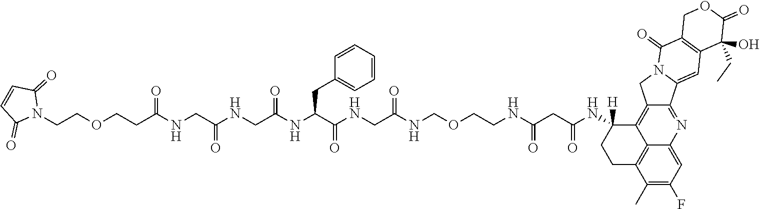 [SMILES: CC[C@@]1(O)C(=O)OCc2c1cc1n(c2=O)Cc2c-1nc1cc(F)c(C)c3c1c2[C@@H](NC(=O)CC(=O)NCCOCNC(=O)CNC(=O)[C@H](Cc1ccccc1)NC(=O)CNC(=O)CNC(=O)CCOCCN1C(=O)C=CC1=O)CC3] |
| 1006 | 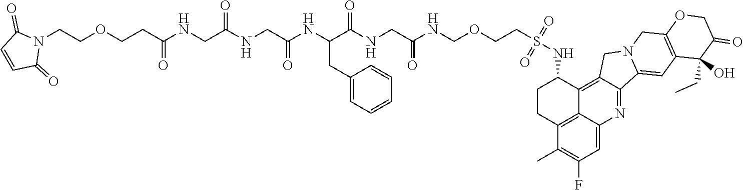 [SMILES: CC[C@@]1(O)C(=O)COC2=C1C=C1c3nc4cc(F)c(C)c5c4c(c3CN1C2)[C@@H](NS(=O)(=O)CCOCNC(=O)CNC(=O)C(Cc1ccccc1)NC(=O)CNC(=O)CNC(=O)CCOCCN1C(=O)C=CC1=O)CC5] |
| 1007 | 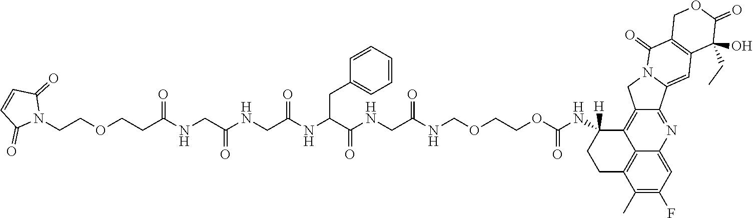 [SMILES: CC[C@@]1(O)C(=O)OCc2c1cc1n(c2=O)Cc2c-1nc1cc(F)c(C)c3c1c2[C@@H](NC(=O)OCCOCNC(=O)CNC(=O)C(Cc1ccccc1)NC(=O)CNC(=O)CNC(=O)CCOCCN1C(=O)C=CC1=O)CC3] |
| 1008 | 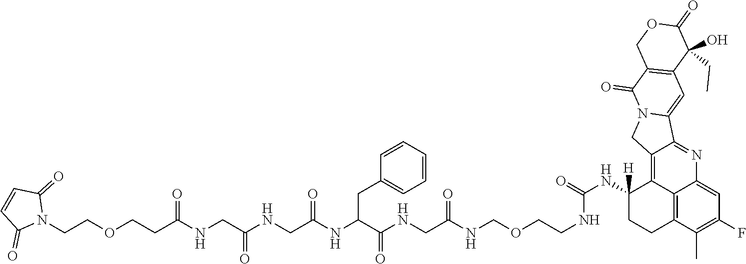 [SMILES: CC[C@@]1(O)C(=O)OCc2c1cc1n(c2=O)Cc2c-1nc1cc(F)c(C)c3c1c2[C@@H](NC(=O)NCCOCNC(=O)CNC(=O)C(Cc1ccccc1)NC(=O)CNC(=O)CNC(=O)CCOCCN1C(=O)C=CC1=O)CC3] |
| 1009 | 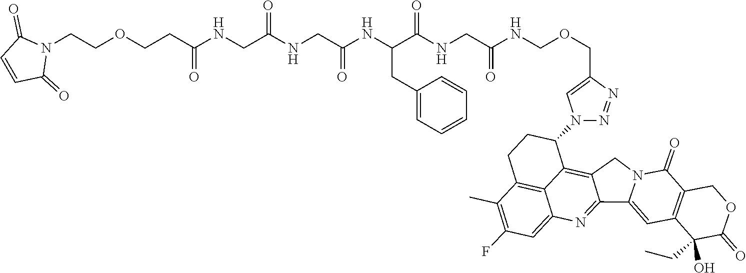 [SMILES: CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CNC(=O)CCOCCN2C(=O)C=CC2=O)nn1)CC3] |
| 1010 | 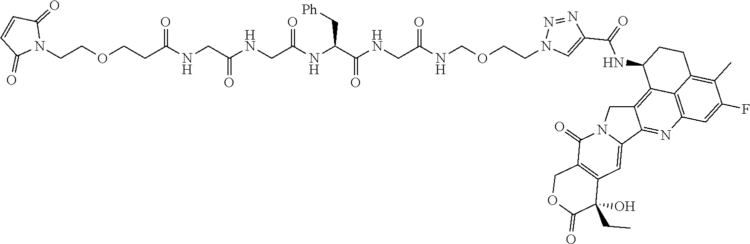 [SMILES: CC[C@@]1(O)C(=O)OCc2c1cc1n(c2=O)Cc2c-1nc1cc(F)c(C)c3c1c2[C@@H](NC(=O)c1cn(CCOCNC(=O)CNC(=O)[C@H](Cc2ccccc2)NC(=O)CNC(=O)CNC(=O)CCOCCN2C(=O)C=CC2=O)nn1)CC3] |
| 1011 | 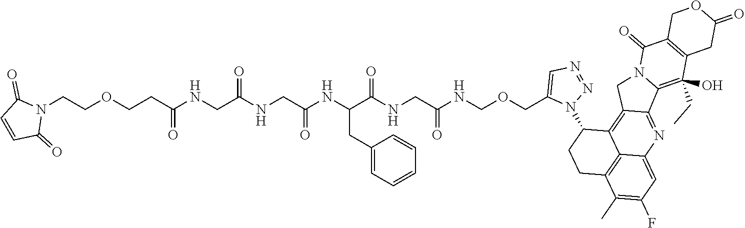 |
| 1012 | 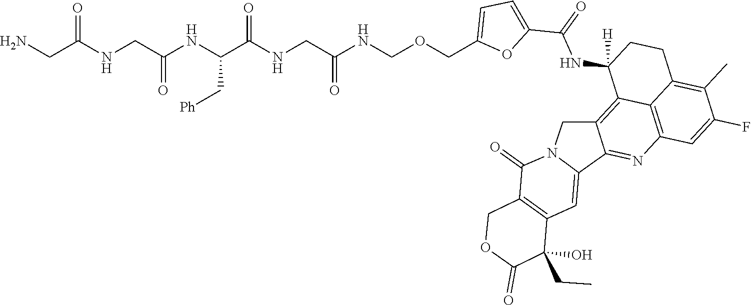 [SMILES: CC[C@@]1(O)C(=O)OCc2c1cc1n(c2=O)Cc2c-1nc1cc(F)c(C)c3c1c2[C@@H](NC(=O)c1ccc(COCNC(=O)CNC(=O)[C@H](Cc2ccccc2)NC(=O)CNC(=O)CN)o1)CC3] |
| 1013 | 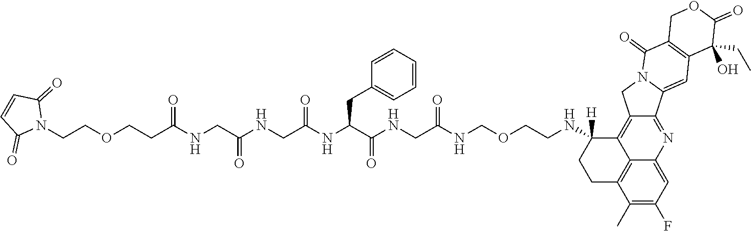 [SMILES: CC[C@@]1(O)C(=O)OCc2c1cc1n(c2=O)Cc2c-1nc1cc(F)c(C)c3c1c2[C@@H](NCCOCNC(=O)CNC(=O)[C@H](Cc1ccccc1)NC(=O)CNC(=O)CNC(=O)CCOCCN1C(=O)C=CC1=O)CC3] |
| 1014 | 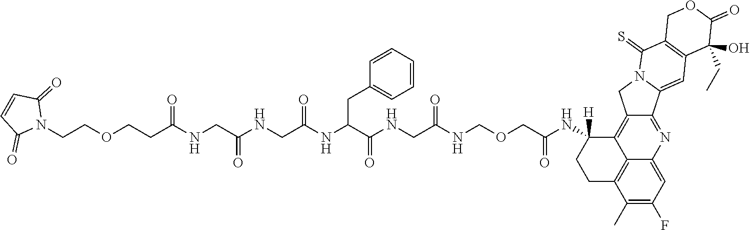 [SMILES: CC[C@@]1(O)C(=O)OCc2c1cc1n(c2=S)Cc2c-1nc1cc(F)c(C)c3c1c2[C@@H](NC(=O)COCNC(=O)CNC(=O)C(Cc1ccccc1)NC(=O)CNC(=O)CNC(=O)CCOCCN1C(=O)C=CC1=O)CC3] |
| 1015 | 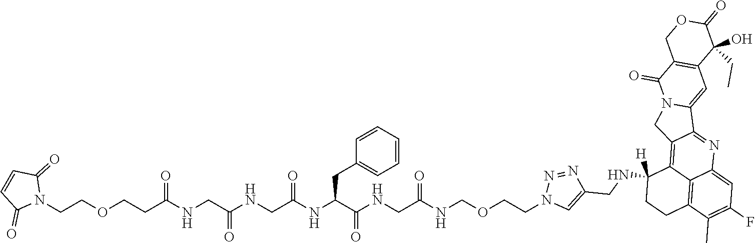 [SMILES: CC[C@@]1(O)C(=O)OCc2c1cc1n(c2=O)Cc2c-1nc1cc(F)c(C)c3c1c2[C@@H](NCc1cn(CCOCNC(=O)CNC(=O)[C@H](Cc2ccccc2)NC(=O)CNC(=O)CNC(=O)CCOCCN2C(=O)C=CC2=O)nn1)CC3] |
| 1016 | 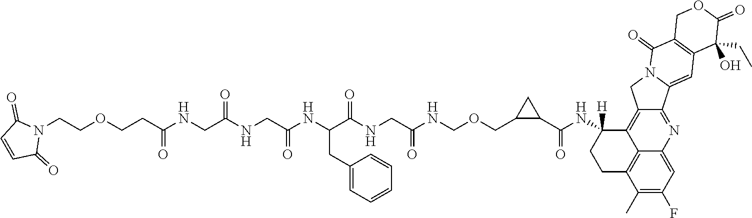 [SMILES: CC[C@@]1(O)C(=O)OCc2c1cc1n(c2=O)Cc2c-1nc1cc(F)c(C)c3c1c2[C@@H](NC(=O)C1CC1COCNC(=O)CNC(=O)C(Cc1ccccc1)NC(=O)CNC(=O)CNC(=O)CCOCCN1C(=O)C=CC1=O)CC3] |
| 1017 | 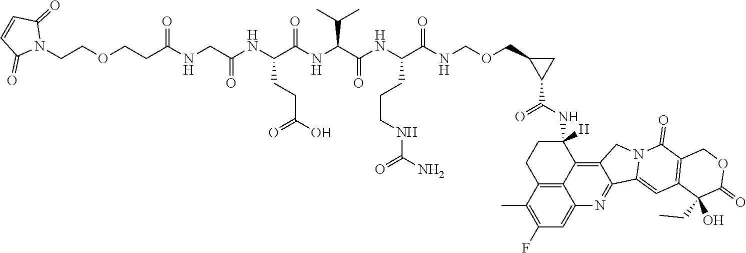 [SMILES: CC[C@@]1(O)C(=O)OCc2c1cc1n(c2=O)Cc2c-1nc1cc(F)c(C)c3c1c2[C@@H](NC(=O)[C@H]1C[C@@H]1COCNC(=O)[C@H](CCCNC(N)=O)NC(=O)[C@@H](NC(=O)[C@H](CCC(=O)O)NC(=O)CNC(=O)CCOCCN1C(=O)C=CC1=O)C(C)C)CC3] |
| 1018 | 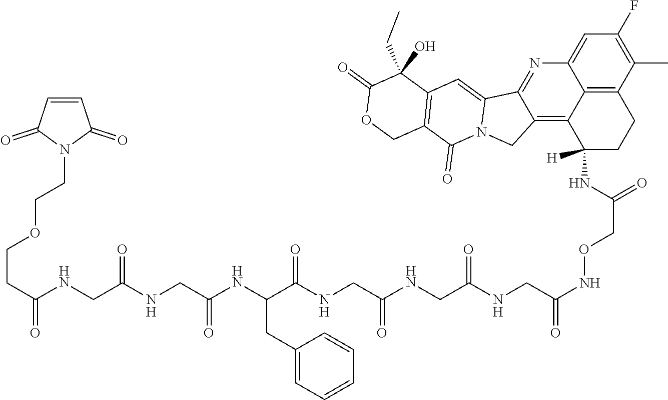 [SMILES: CC[C@@]1(O)C(=O)OCc2c1cc1n(c2=O)Cc2c-1nc1cc(F)c(C)c3c1c2[C@@H](NC(=O)CONC(=O)CNC(=O)CNC(=O)CNC(=O)C(Cc1ccccc1)NC(=O)CNC(=O)CNC(=O)CCOCCN1C(=O)C=CC1=O)CC3] |
| 1019 | 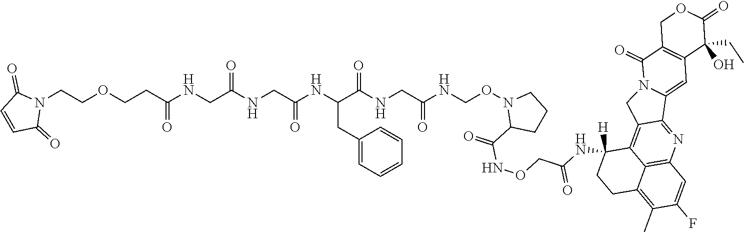 [SMILES: CC[C@@]1(O)C(=O)OCc2c1cc1n(c2=O)Cc2c-1nc1cc(F)c(C)c3c1c2[C@@H](NC(=O)CONC(=O)C1CCCN1OCNC(=O)CNC(=O)C(Cc1ccccc1)NC(=O)CNC(=O)CNC(=O)CCOCCN1C(=O)C=CC1=O)CC3] |
| 1020 | 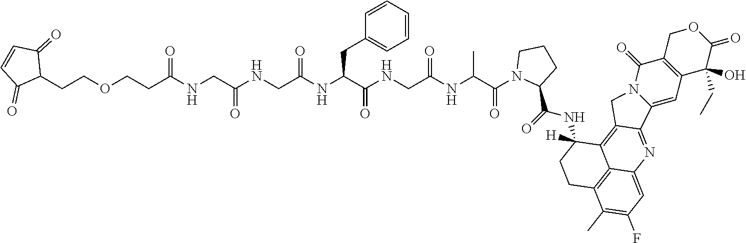 [SMILES: CC[C@@]1(O)C(=O)OCc2c1cc1n(c2=O)Cc2c-1nc1cc(F)c(C)c3c1c2[C@@H](NC(=O)[C@@H]1CCCN1C(=O)C(C)NC(=O)CNC(=O)[C@H](Cc1ccccc1)NC(=O)CNC(=O)CNC(=O)CCOCCC1C(=O)C=CC1=O)CC3] |
| 1021 | 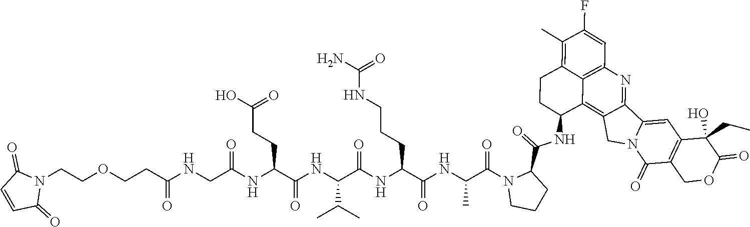 [SMILES: CC[C@@]1(O)C(=O)OCc2c1cc1n(c2=O)Cc2c-1nc1cc(F)c(C)c3c1c2[C@@H](NC(=O)[C@H]1CCCN1C(=O)[C@H](C)NC(=O)[C@H](CCCNC(N)=O)NC(=O)[C@@H](NC(=O)[C@H](CCC(=O)O)NC(=O)CNC(=O)CCOCCN1C(=O)C=CC1=O)C(C)C)CC3] |
| 1022 | 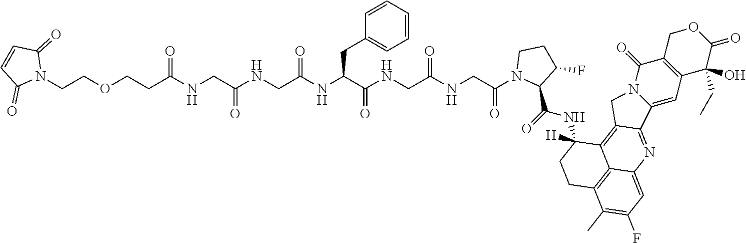 [SMILES: CC[C@@]1(O)C(=O)OCc2c1cc1n(c2=O)Cc2c-1nc1cc(F)c(C)c3c1c2[C@@H](NC(=O)[C@@H]1[C@@H](F)CCN1C(=O)CNC(=O)CNC(=O)[C@H](Cc1ccccc1)NC(=O)CNC(=O)CNC(=O)CCOCCN1C(=O)C=CC1=O)CC3] |
| 1023 | 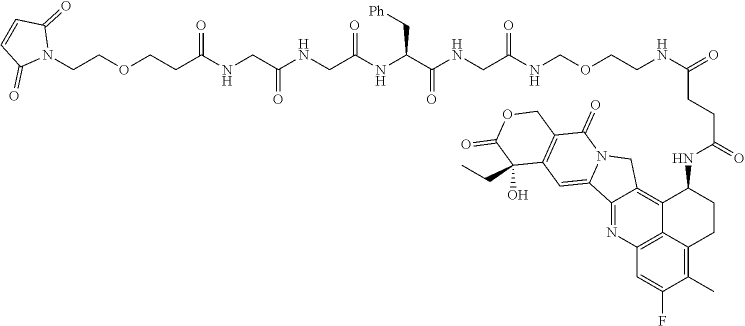 [SMILES: CC[C@@]1(O)C(=O)OCc2c1cc1n(c2=O)Cc2c-1nc1cc(F)c(C)c3c1c2[C@@H](NC(=O)CCC(=O)NCCOCNC(=O)CNC(=O)[C@H](Cc1ccccc1)NC(=O)CNC(=O)CNC(=O)CCOCCN1C(=O)C=CC1=O)CC3] |
| 1024 | 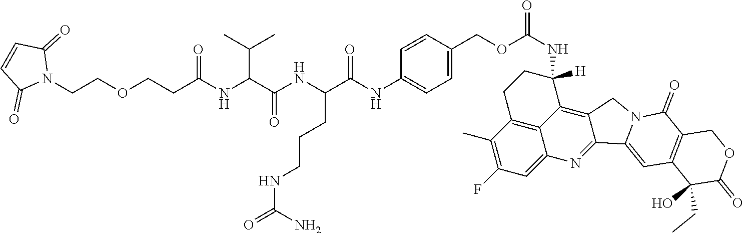 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)C(CCCNC(N)=O)NC(=O)C(NC(=O)CCOCCN2C(=O)C=CC2=O)C(C)C)cc1)CC3] |

Further disclosed herein is a linker-payload construct by Formula IIIA or Formula IIIB:

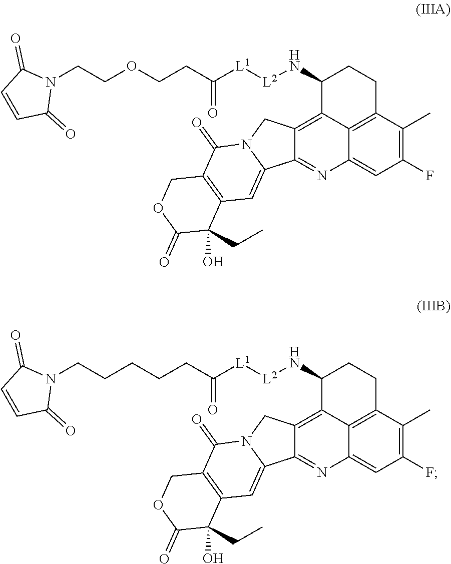

or a pharmaceutically acceptable salt or stereoisomer thereof, wherein:
L¹ is a cathepsin B cleavable peptide or a cathepsin D cleavable peptide; and
L² is a self-immolating moiety.

In some embodiments, L¹ is selected from the group consisting of:

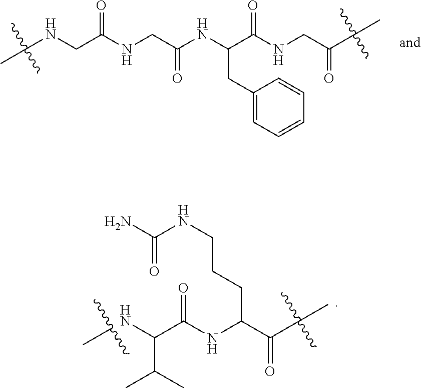

In other embodiments, the linker-payload construct is selected for example, from the group consisting of:

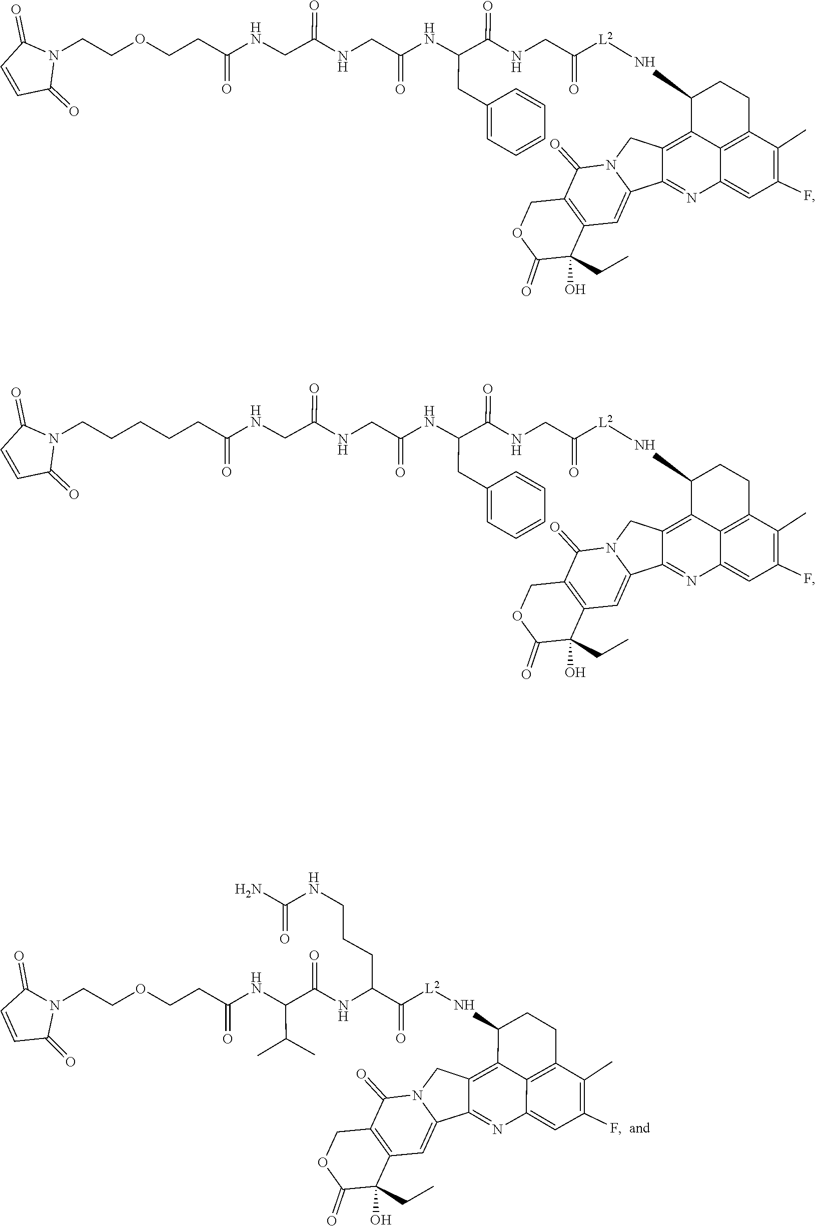

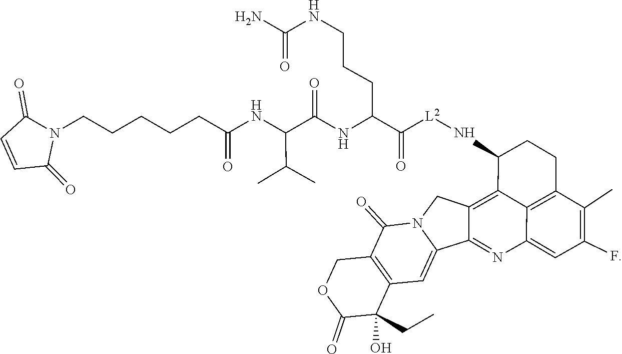

In some embodiments, L² is selected, for example, from the group consisting of:

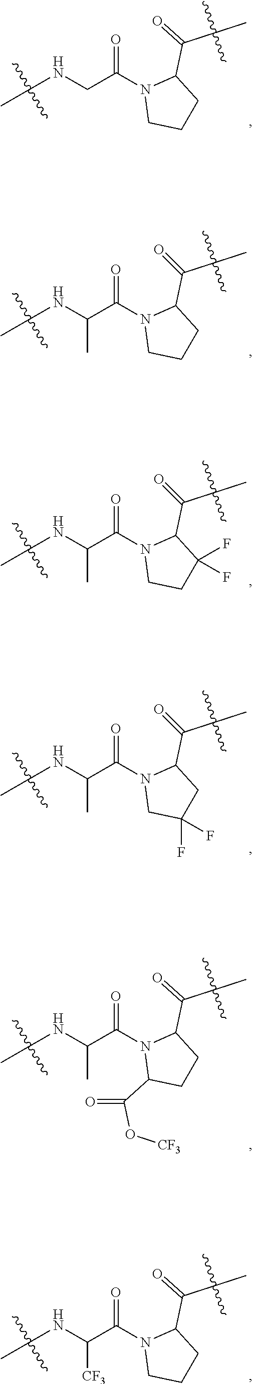 [SMILES: *NCC(=O)N1CCCC1C(*)=O ; *NC(C)C(=O)N1CCCC1C(*)=O ; *NC(C)C(=O)N1CCC(F)(F)C1C(*)=O ; *NC(C)C(=O)N1CC(F)(F)CC1C(*)=O ; *NC(C)C(=O)N1C(C(*)=O)CCC1C(=O)OC(F)(F)F ; *NC(C(=O)N1CCCC1C(*)=O)C(F)(F)F]

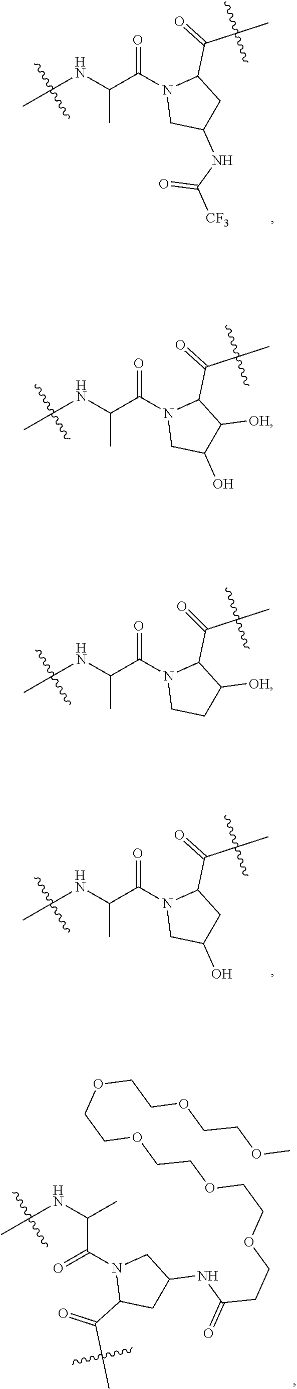 [SMILES: *NC(C)C(=O)N1CC(NC(=O)C(F)(F)F)CC1C(*)=O ; *NC(C)C(=O)N1CC(O)C(O)C1C(*)=O ; *NC(C)C(=O)N1CCC(O)C1C(*)=O ; *NC(C)C(=O)N1CC(O)CC1C(*)=O ; *NC(C)C(=O)N1CC(NC(=O)CCOCCOCCOCCOCCOCCOC)CC1C(*)=O]

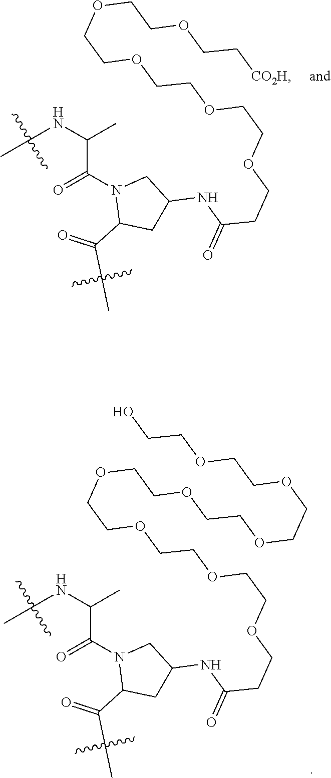 [SMILES: *NC(C)C(=O)N1CC(NC(=O)CCOCCOCCOCCOCCOCCC(=O)O)CC1C(*)=O ; *NC(C)C(=O)N1CC(NC(=O)CCOCCOCCOCCOCCOCCOCCOCCOCCO)CC1C(*)=O]

Also disclosed herein, for example, is a drug conjugate represented by Formula IVA or Formula IVB:

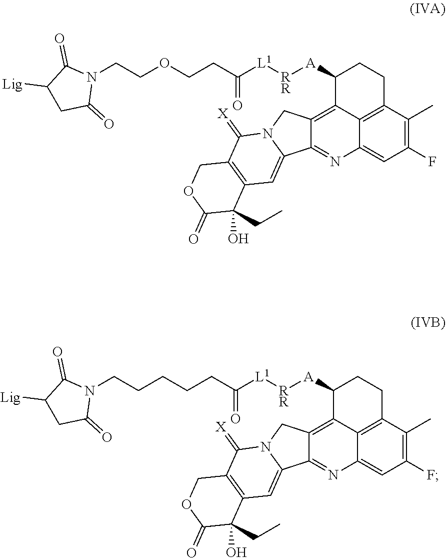

or a pharmaceutically acceptable salt or stereoisomer thereof, wherein:
X is O or S;
A is NH or triazolyl;
Lig is a targeting moiety;
L¹ is —CBP—NH—CH₂— or —CBP—, wherein CBP is a cathepsin B cleavable peptide or a cathepsin D cleavable peptide; and
RR is an alkoxy or amino moiety formed from L¹ and a hydroxy or —NH₂ moiety of R of any one of the therapeutic payloads described herein.

In some embodiments, Lig is a monoclonal antibody. For example, in some embodiments Lig is an antibody selected, for example, from the group consisting of: an anti-TROP2 antibody, an anti-EGRF antibody, an anti-HER2 antibody, an anti-B7-H3 antibody, an anti-CD30 antibody, an anti-CD33 antibody, and an anti-CD70 antibody. In an embodiment, Lig is, for example, an anti-TROP2 antibody.

In other embodiments, CBP is, for example, -Gly-Gly-Phe-Gly- or -Val-Cit-.

In further embodiments, L¹ is, for example, selected from the group consisting of:

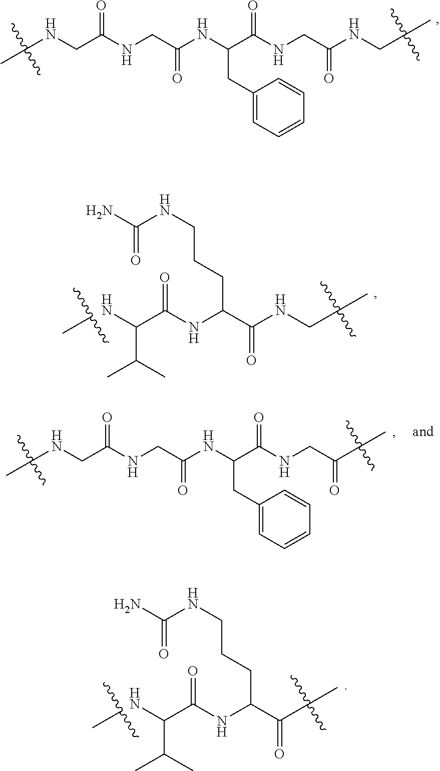

In still further embodiments, the drug conjugate is selected, for example, from the group consisting of:

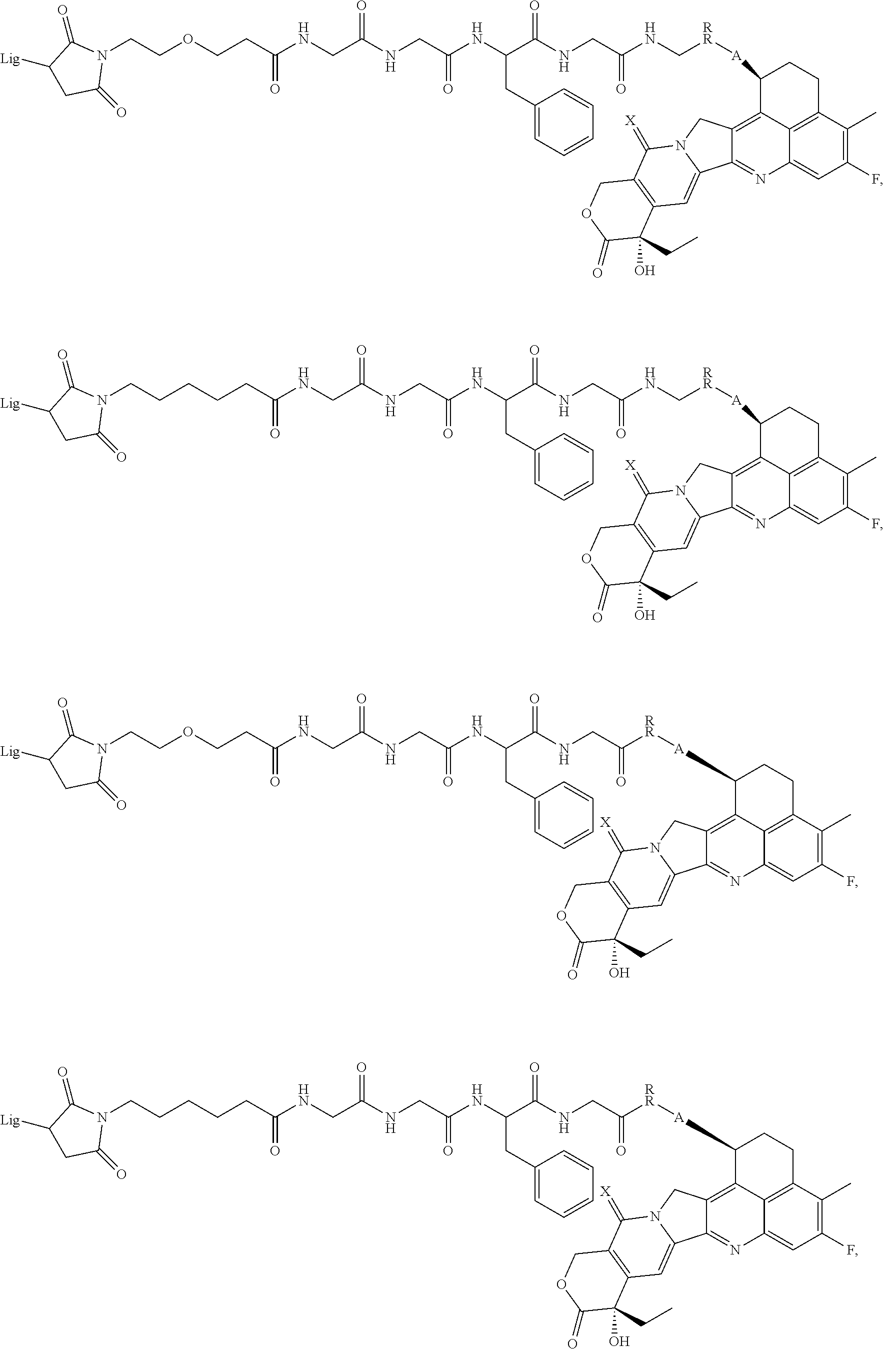

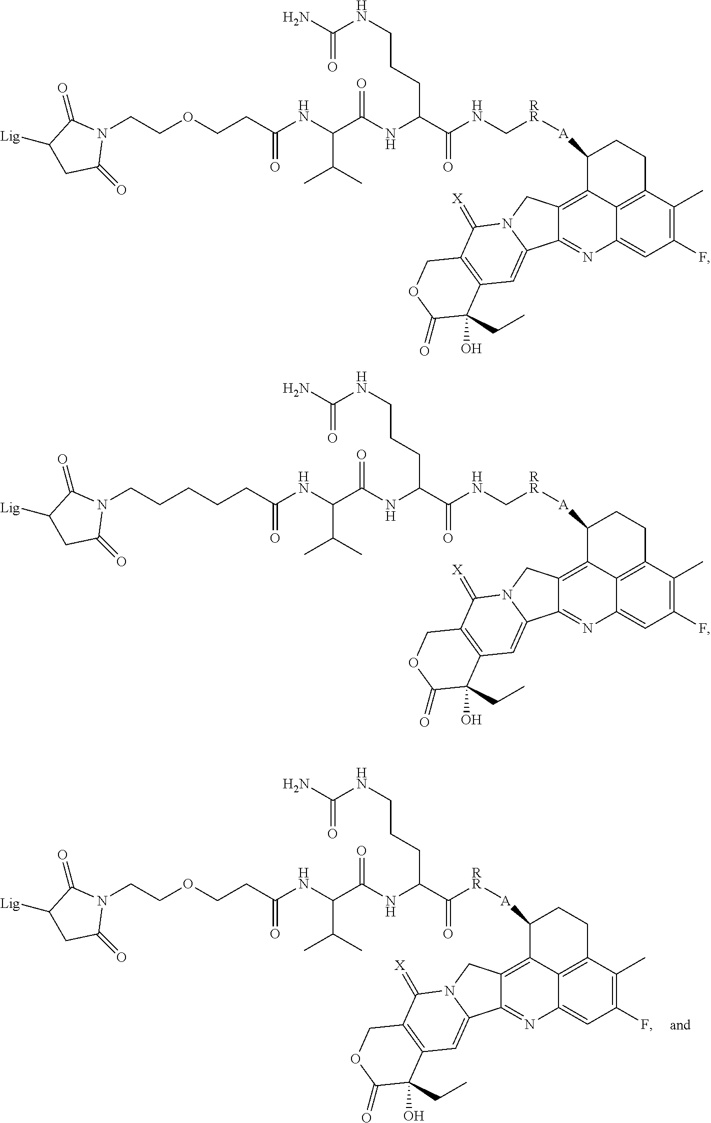

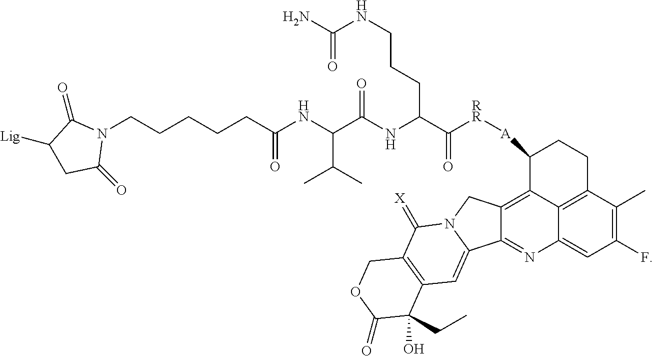

Also disclosed herein is a drug conjugate represented by Formula VA or Formula VB:

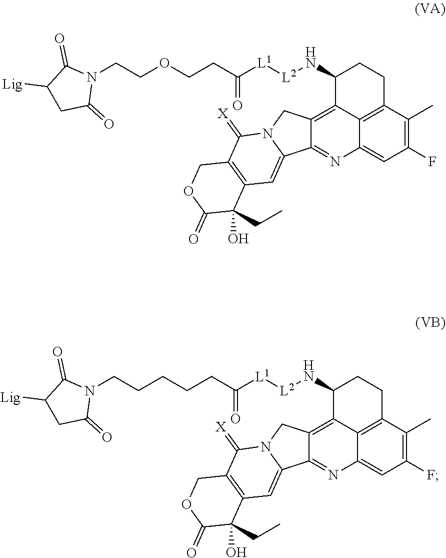

or a pharmaceutically acceptable salt or stereoisomer thereof, wherein:
X is G or S;
Lig is a targeting moiety;
L¹ is a cathepsin B cleavable peptide or a cathepsin D cleavable peptide; and
L² is a self-immolating moiety.

In some embodiments, Lig is a monoclonal antibody. For example, in some embodiments Lig is an antibody selected, for example, from the group consisting of: an anti-TROP2 antibody, an anti-EGRF antibody, an anti-HER2 antibody, an anti-B7-H3 antibody, an anti-CD30 antibody, an anti-CD33 antibody, and an anti-CD70 antibody. In an embodiment, Lig is, for example, an anti-TROP2 antibody.

In other embodiments, L¹ is selected, for example, from the group consisting of:

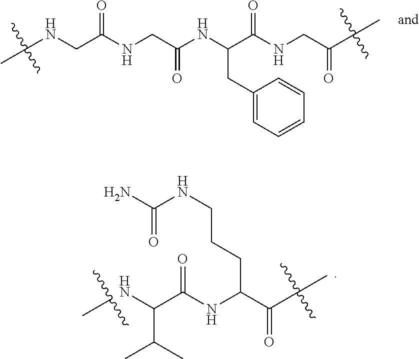

In further embodiments, the drug conjugate is selected, for example, from the group consisting of:

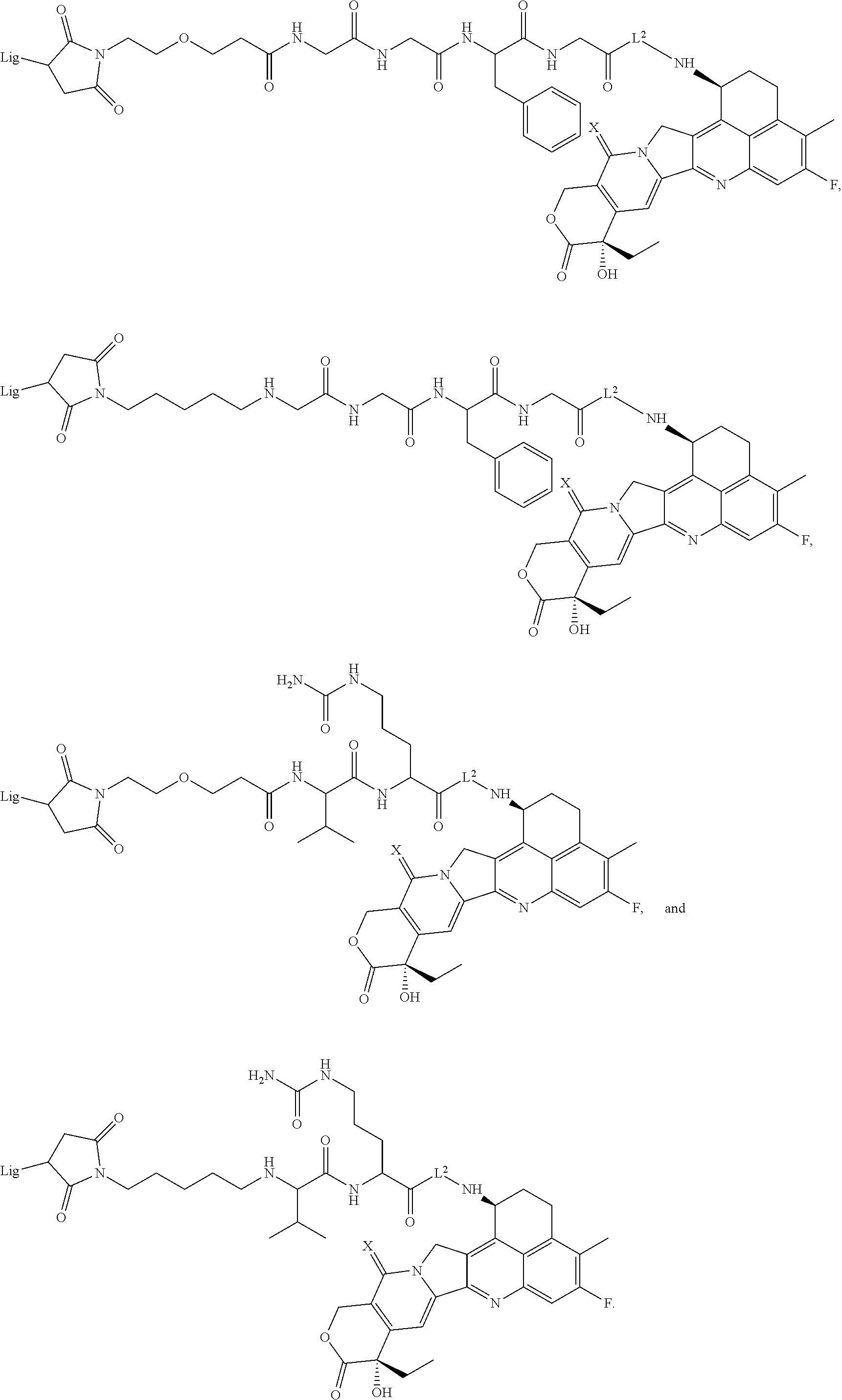

In still further embodiments, L² is selected from the group consisting of:

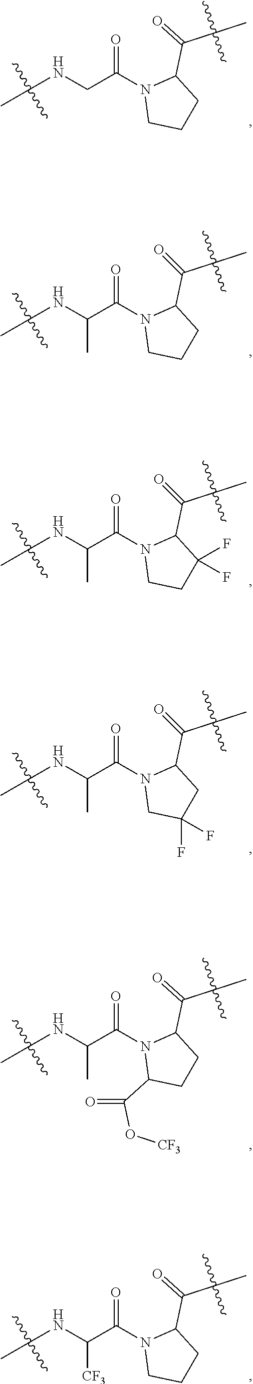 [SMILES: *NCC(=O)N1CCCC1C(*)=O ; *NC(C)C(=O)N1CCCC1C(*)=O ; *NC(C)C(=O)N1CCC(F)(F)C1C(*)=O ; *NC(C)C(=O)N1CC(F)(F)CC1C(*)=O ; *NC(C)C(=O)N1C(C(*)=O)CCC1C(=O)OC(F)(F)F ; *NC(C(=O)N1CCCC1C(*)=O)C(F)(F)F]

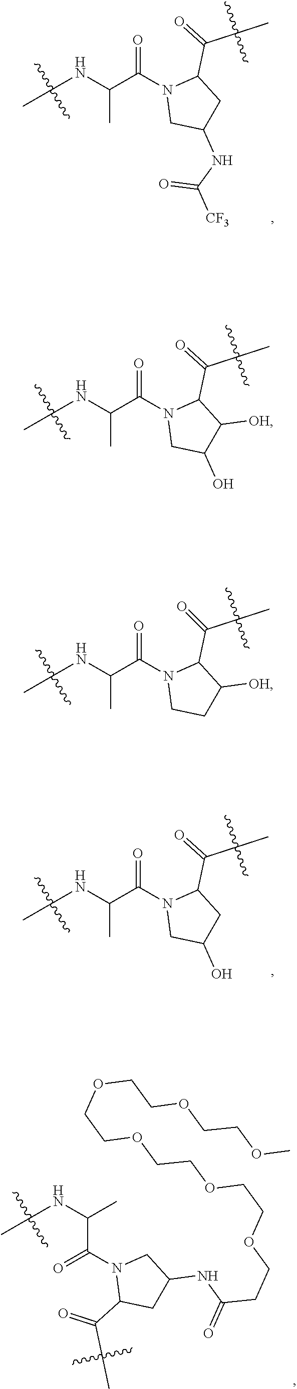 [SMILES: *NC(C)C(=O)N1CC(NC(=O)C(F)(F)F)CC1C(*)=O ; *NC(C)C(=O)N1CC(O)C(O)C1C(*)=O ; *NC(C)C(=O)N1CCC(O)C1C(*)=O ; *NC(C)C(=O)N1CC(O)CC1C(*)=O ; *NC(C)C(=O)N1CC(NC(=O)CCOCCOCCOCCOCCOCCOC)CC1C(*)=O]

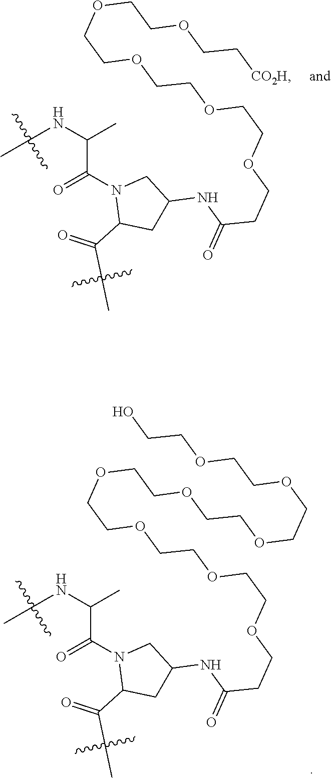 [SMILES: *NC(C)C(=O)N1CC(NC(=O)CCOCCOCCOCCOCCOCCC(=O)O)CC1C(*)=O ; *NC(C)C(=O)N1CC(NC(=O)CCOCCOCCOCCOCCOCCOCCOCCOCCO)CC1C(*)=O]

Also disclosed herein is a drug conjugate selected from the group consisting of:

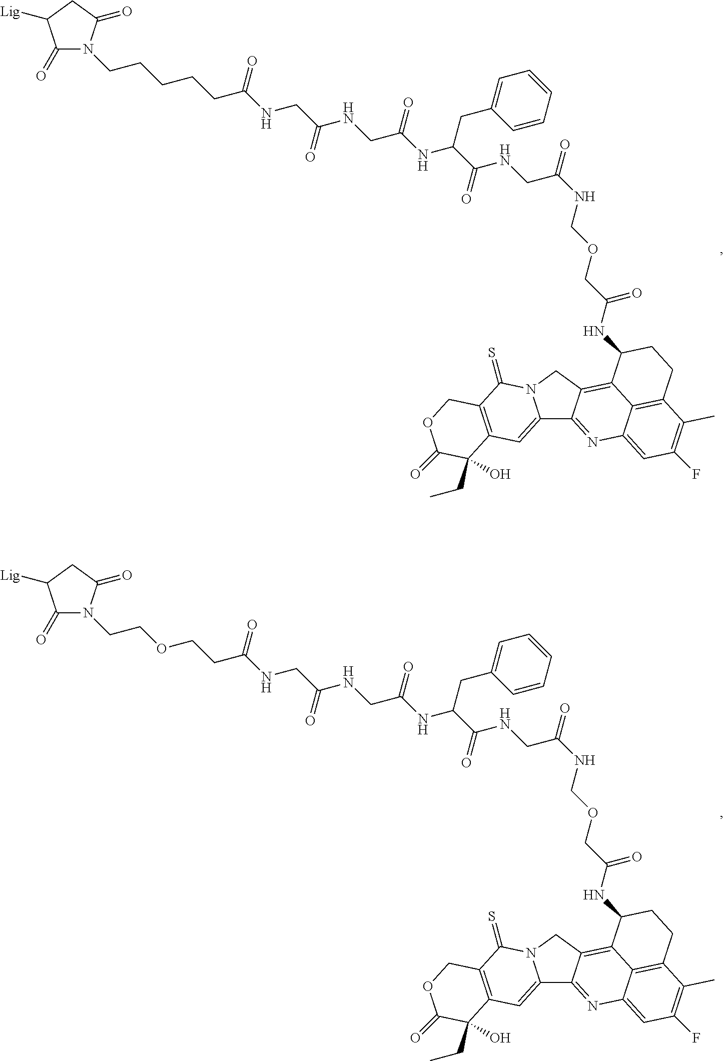

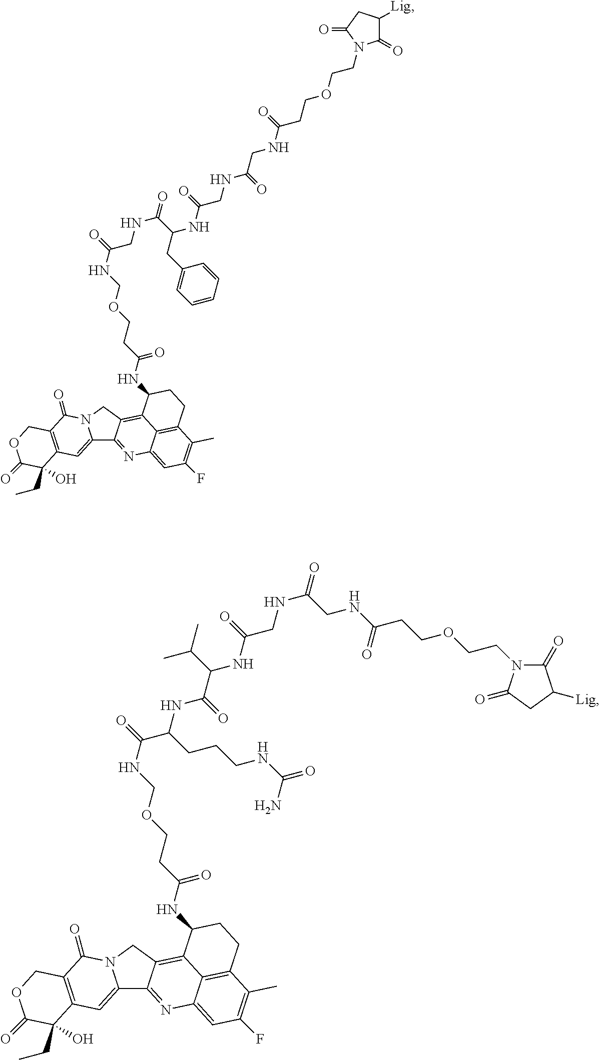

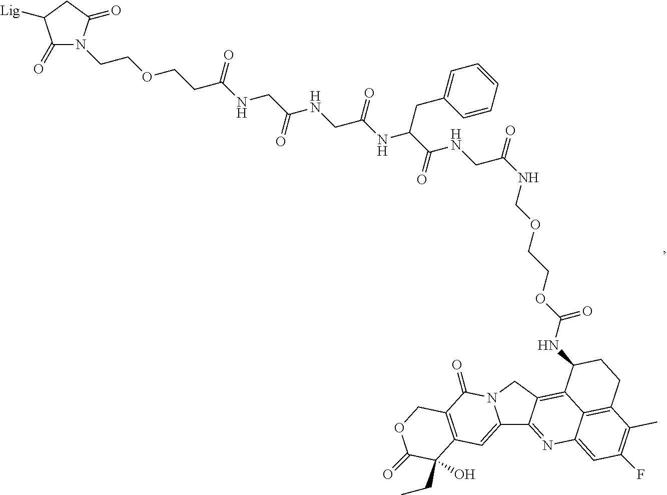

and a pharmaceutically acceptable salt or stereoisomer thereof, wherein Lig is a targeting moiety.

In some embodiments, Lig is a monoclonal antibody. For example, in some embodiments Lig is an antibody selected, for example, from the group consisting of: an anti-TROP2 antibody, an anti-EGRF antibody, an anti-HER2 antibody, an anti-B7-H3 antibody, an anti-CD30 antibody, an anti-CD33 antibody, and an anti-CD70 antibody. In an embodiment, Lig is, for example, an anti-TROP2 antibody.

Contemplated targets and corresponding antibodies of the present disclosure are provided in Table 3.

TABLE 3

| Target | Antibody name |
|---|---|
| EGFR | Cetuximab |
| EGFR | Panitumumab |
| EGFR | RN765C |
| EGFR | Laprituximab |
| EGFR | Serclutamab |
| EGFRviii | Depatuxizumab |
| HER2 | Trastuzumab |
| HER2 | Disitumab |
| TROP2 | RS7 |
| TROP2 | Sacituzumab |
| TROP2 | Datopotomab |
| TROP2 | RN927C |
| HER3 | Patritumab |
| NECTIN-4 | Enfortumab |
| CD-33 | Gemtuzumab |
| CD-33 | Vadastuximab |
| CD-30 | Brentuximab |
| CD-22 | Inotuzumab |
| CD-22 | Pinatuzumab |
| CD-22 | Moxetumomab |
| CD-22 | Epratuzumab |
| anti-CD79b | Polatuzumab |
| CD-19 | Tafasitamab |
| Anti-Angiopoietin-2; | Vanucizumab |
| Anti-VEGF-A | |
| Anti-AFP | Tacatuzumab |
| FRα | Mirvetuximab |
| CD-166 | Praluzatamab |
| Mesothelin | Anetumab |
| CD74 | Milatuzumab |
| CD56 | Lorvotuzumab |
| NaPi2b | Lifastuzumab |
| STEAP1 | Vandortuzumab |
| LIV-1 | Ladiratuzumab |
| CD74 | Milatuzumab |
| CD56 | Lorvotuzumab |
| GPNMB | Glembatumomab |
| CD66e (CAECAM5) | Labetuzumab; |
| | SAR408701 |
| TF | Tisotumab |
| DLL3 | SC16LD6.5 |
| Mesotherin | Anetumab |
| CD37 | Naratuximab |
| BCMA | Belantamab |
| AXL | Enapotamab |
| CD-20 | Ibritumomab |
| CD-25 | Camidanlumab |
| PTK7 | Cofetuzumab |
| c-Met | Telisotuzumab |
| CD138 | Indatuximab |
| BH-73 (CD126) | Omburtamab |
| CD19 | SAR3419 |
| CD138 | BT-062 |
| PSMA | |
| SC-16 | SC16LD6.5 |
| EGFR | ABT-414 |
| CAIX | BAY 79-4620 |
| ETBR | DEDN6526A (RG7636) |
| Nectin-4 | ASG-22ME (ASG- |
| | 22M6E, AGS-22CE) |
| SLITRK6 | ASG15E-13-1, ASG- |
| | 15ME |
| CA6 | SAR566658 |
| CD19 | SGN-CD19A |
| CD70 | SGN-CD70A |
| ENPP3 | AGS-16M8F |
| GCC | MLN0264 |
| EGFR | IMGN289, J2898A |
| EGFRvIII | AMG 595 |
| CD70 | AMG 172 |
| 5T4 | PF-06263507 |
| CD98 | IGN523 |
| CD-33 | IMGN-633; AVE9633 |
| HER3 | Patritumab |
| Integrin-beta-6 | SGN-B6A |
| STn (Sialyl-Thomsen | L2A5; SGN-STVN; |
| nouveau antigen) | 8C2-2D6 |
| (CD175s) | |

### Methods

Disclosed herein, for example, is a method of treating cancer in patient in need thereof, comprising administering to the patient an effective amount of a therapeutic payload disclosed herein, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

Also disclosed herein is a method of treating cancer in patient in need thereof, comprising administering to the patient an effective amount of a linker-payload construct disclosed herein, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

Further disclosed herein is method of treating cancer in patient in need thereof, comprising administering to the patient an effective amount of a drug conjugate comprising any of the payloads as disclosed herein, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

In certain embodiments, the patient is a human.

In certain embodiments, administering a disclosed compound may comprise subcutaneous administration. In certain embodiments, administering a disclosed compound may comprise intravenous administration. In certain embodiments, administering a disclosed compound may comprise oral administration.

Provided methods of treatment may include administering a disclosed compound once, twice, or three times daily; about every other day (e.g. every 2 days); twice weekly (e.g. every 3 days, every 4 days, every 5 days, every 6 days, or e.g. administered with an interval of about 2 to about 3 days between doses); once weekly; three times weekly; every other week; twice monthly; once a month; every other month; or even less often.

In particular, in certain embodiments, the present disclosure provides a method of treating one or more of the above medical indications comprising administering to a subject in need thereof a therapeutically effective amount of a compound described herein.

In certain embodiments, the compound utilized by one or more of the methods disclosed herein is one of the generic, subgeneric, or specific compounds described herein.

The compounds of the present disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted herein, a compound of the present disclosure may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. A suitable treatment period can be, for example, at least about one week, at least about two weeks, at least about one month, at least about six months, at least about 1 year, or indefinitely. A treatment period can terminate when a desired result is achieved.

### Pharmaceutical Compositions and Kits

Another aspect of the present disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous), vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

For example, disclosed herein is a pharmaceutical composition comprising a therapeutic payload disclosed herein, and a pharmaceutically acceptable excipient. Also disclosed herein is a pharmaceutical composition comprising a linker-payload construct disclosed herein, and a pharmaceutically acceptable excipient. Further disclosed herein is a pharmaceutical composition comprising a drug conjugate disclosed herein, and a pharmaceutically acceptable excipient.

Exemplary pharmaceutical compositions of this disclosure may be used in the form of a pharmaceutical preparation, for example, in solid, semisolid or liquid form, which contains one or more disclosed compounds, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the present disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present invention.

Advantageously, the present disclosure also provides kits for use by e.g. a consumer in need of treatment of cancer. Such kits include a suitable dosage form such as those described herein and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . “etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent. Contemplated herein are disclosed compounds in combination with at least one other agent previously been shown to treat cancer.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as “Intermediates” herein are contemplated as compounds of the present disclosure.

Unless stated otherwise, all reactions were performed in a heat gun dried glassware under argon atmosphere, using standard septa techniques. All commercially available starting building blocks were purchased from commercial vendors. Reactions were monitored by HPLC-MS analyses using a Shimadzu UFLC-MS-2020 system with ESI, and/or by thin-layer chromatography (TLC) using silica gel 60 F254 plates (Merck) and visualized by UV at 254 nm. Purifications were performed using automated flash chromatography system (ECOM), using prepacked column containing C₁₈ or modified C18 silica gel (Interchim, PT-15C18AQ, 15 μm Puriflash 200, 5 g, 12 g, or 25 g). Semipreparative HPLC were performed on ECOM HPLC system, using a modified C18 semipreparative column (YMC-Actus, Triart Prep C18, 250×20 mm, S-10 μm, 12 nm). HPLC-MS analyses were performed on Shimadzu UFLC-MS-2020 system with ESI. Column: Acquity UPLC BEH C18 1.7 μm, 2.1×50 mm. Solvent A: H₂O 0.1% HCOOH; Solvent B: MeCN+0.1% HCOOH. Total flow 0.6 ml/min. Total time of the method 10 min. Mass spectrum was recorded in range 100-3000 m/z both in positive and negative mode with event time 0.2 s. UV-Vis spectra were recorded with a Shimadzu SPD-M20A Prominence diode array detector, in the range 200-800 nm. NMR spectra were recorded using >99% deuterated solvents, on a 400 MHz Bruker AVANCE III spectrometer (1H at 400 MHz) and/or on a Bruker AVANCE 500 (1H at 500.0 MHz). Chemical shifts (in ppm, 6 scale) were solvent signal in 1H spectra. Intermediates and final products were freeze-dried using a Gregory instruments lyophilizer (model L4-110), from water or water mixtures of dioxane or acetonitrile.

### Abbreviations

| | |
|---|---|
| DCM | Dichloromethane |
| DMF | N,N-Dimethyl formamide |
| DMSO | Dimethylsulfoxide |
| DIPEA | N,N-Diisopropylethylamine |
| EDTA | Ethylenediaminetetraacetic acid |
| DMTMM | (4-(4,6-dimethoxy-1,3,5-triazin-2-yl)- |
| | 4-methyl-morpholinium chloride |
| EPPS | 4-(2-Hydroxyethyl)-1- |
| | piperazinepropanesulfonic acid |
| equiv | equivalent(s) |
| h, hr | hour(s) |
| Mal-PEG1-NHS | Maleimide-polyethylene glycol |
| | 1-N-hydroxysuccinimide |
| Mg | milligram(s) |
| Min | Minute(s) |
| mL, ml | milliliter(s) |
| RP | Reverse phase |
| NCS | N-Chlorosuccinimide |
| NHS | N-Hydroxysuccinimide |
| rt, RT | Room temperature |
| TBAI | Tetrabutylammonium iodide |
| TCEP | Tris(2-carboxyethyl)phosphine |
| TEA | Triethylamine |
| TFA | Trifluoroacetic acid |
| THF | Tetrahydrofuran |

### Example 1: Synthesis of Compound 2

### Step 1:

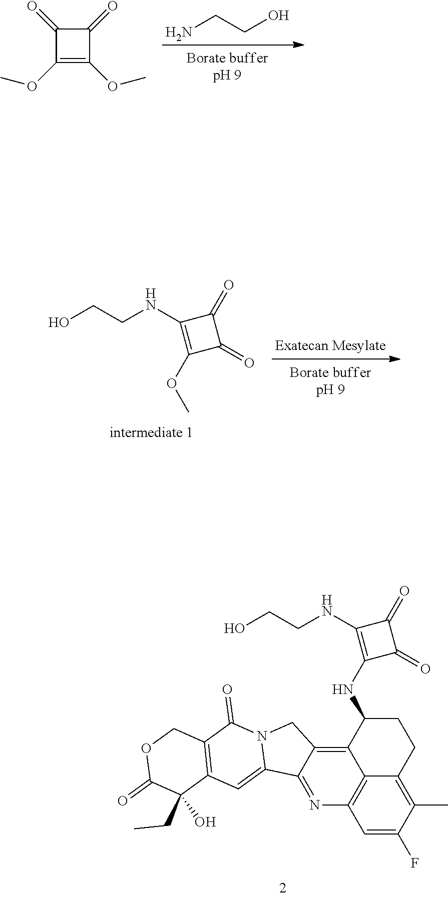

Intermediate 1. A mixture of ethanolamine (23 mg, 0.4142 mmol) and dimethoxysquarate (3 equiv., 177 mg, 1.242 mmol) were suspended in 10 mL of 1M borate buffer (pH=9), and the mixture was stirred at 55° C. for 16 hours. Two mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (46 mg, 65%). MS calc. for C₇H₁₀NO₄: 172.06, found: 172.25, [M+H]⁺.

Compound 2. Exatecan mesylate (20 mg, 0.0377 mmol) and the previously synthesized intermediate 1 (1,5 equiv., 9.7 mg, 0.0564) were suspended in 5 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (12 mg, 57%). MS calc. for C₃₀H₂₈FN₄O₇: 575.19, found: 575.45, [M+H]⁺. ¹H NMR (400 MHz, DMSO-d₆) δ 8.45 (s, 1H), 7.83 (d, J=10.9 Hz, 1H), 7.78 (s, 1H), 7.32 (s, 1H), 5.79 (s, 1H), 5.42 (s, 2H), 5.29 (d, J=7.5 Hz, 2H), 3.68-3.43 (m, 4H), 3.23 (d, J=7.7 Hz, 2H), 2.42 (d, J=1.9 Hz, 3H), 2.33 (td, J=5.7, 4.8, 2.9 Hz, 1H), 1.96-1.78 (m, 2H), 1.76 (s, 1H), 1.26-1.15 (m, 2H), 0.87 (t, J=7.3 Hz, 3H).

### Example 2: Synthesis of Compound 1001

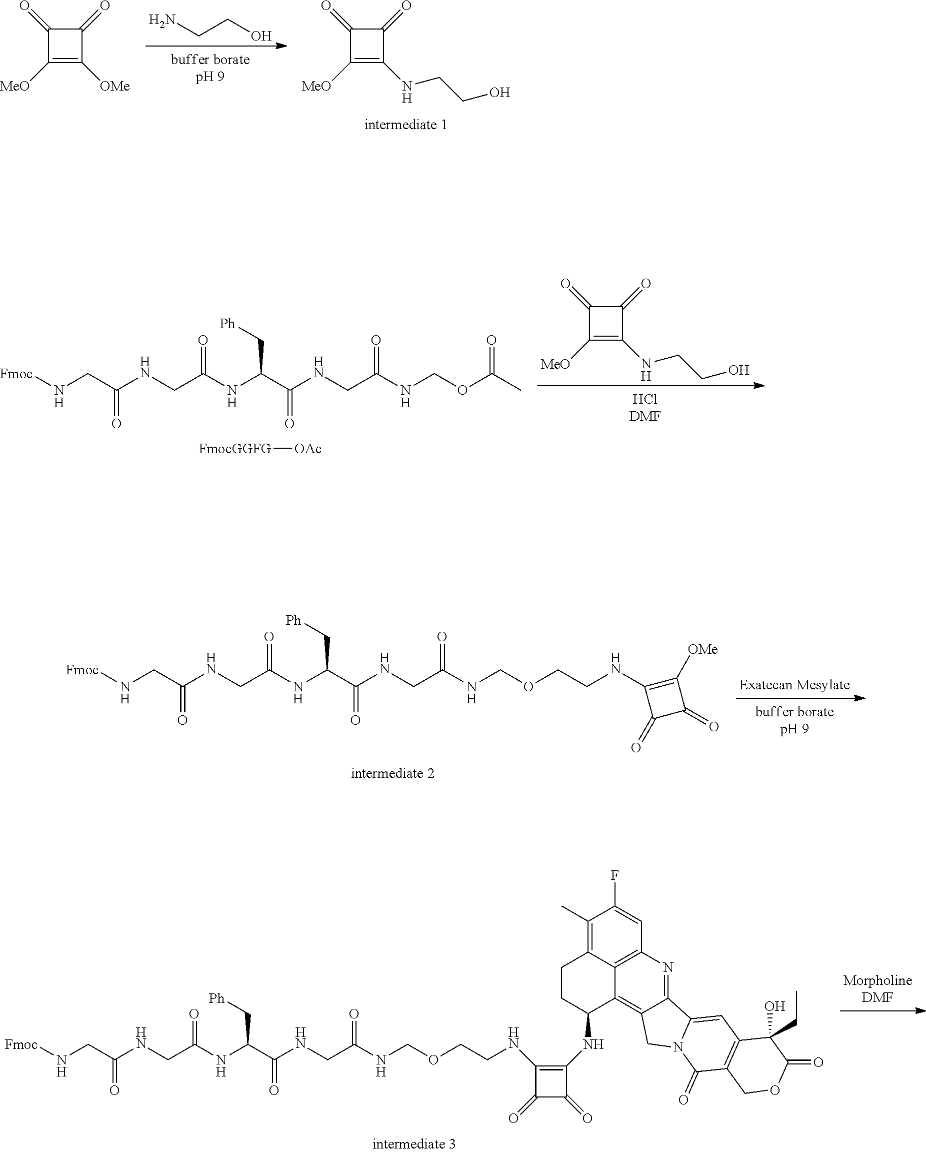

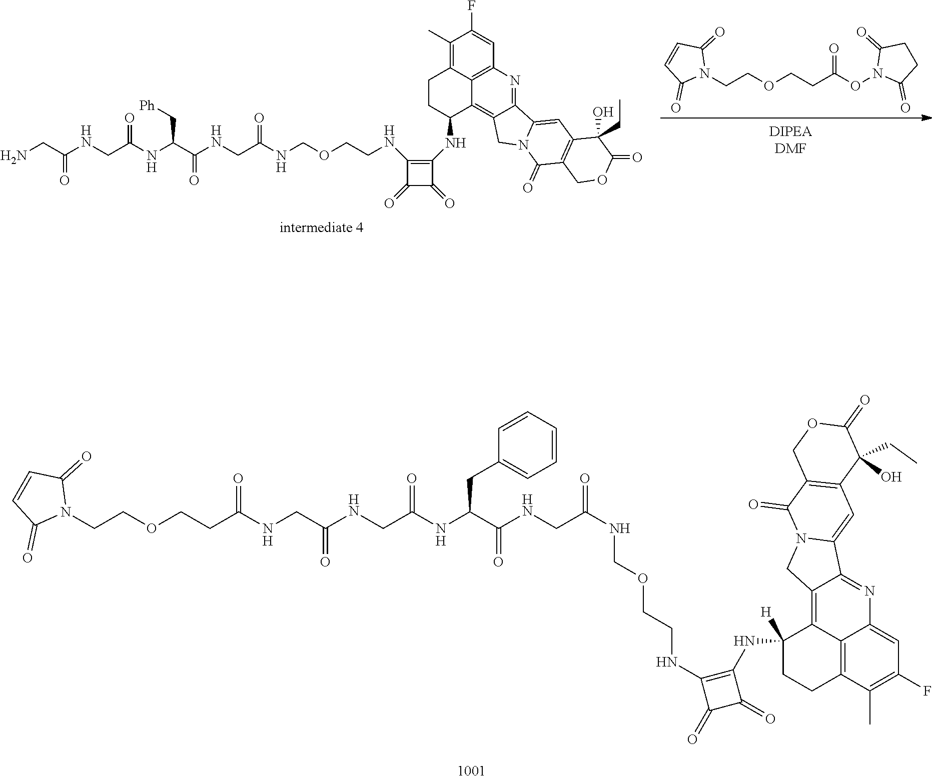 [SMILES: CC[C@@]1(O)C(=O)OCc2c1cc1n(c2=O)Cc2c-1nc1cc(F)c(C)c3c1c2[C@@H](Nc1c(NCCOCNC(=O)CNC(=O)[C@H](Cc2ccccc2)NC(=O)CNC(=O)CN)c(=O)c1=O)CC3 ; O=C(CCOCCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](Nc1c(NCCOCNC(=O)CNC(=O)[C@H](Cc2ccccc2)NC(=O)CNC(=O)CNC(=O)CCOCCN2C(=O)C=CC2=O)c(=O)c1=O)CC3]

Intermediate 1. Ethanolamine (100 mg, 1.637 mmol) and dimethoxysquarate (1.2 equiv., 1.964 mmol, 279 mg) were dissolved in 10 mL of 1 M borate buffer (pH 9). The reaction mixture was stirred at room temperature for 16 h. The solvent was evaporated under reduced pressure, the resulting solid was re-dissolved in DMF and directly loaded on column. The product was purified by reverse-phase flash chromatography, using a column containing 40 g of C18, and using a gradient of ACN in water (0→20% ACN in water). The desired product was recovered as a white solid, after lyophilization from water (205 mg, 73%). MS calc. for C₇H₁₀NO₄: 172.06, found: 172.17, [M+H]⁺.

Intermediate 2. Intermediate 1 (10 mg, 0.058 mmol) and the starting peptide FmocGGFG-OAc (1 equiv., 0.058 mmol, 37 mg) were dissolved in 2 mL of anhydrous DMF under an argon atmosphere, and 100 μL of HCl (2M in Et₂O) were added. The reaction mixture was stirred for 1 h at room temperature, to be then directly loaded on column. The product was purified by reverse-phase flash HPLC, using a semipreparative column containing 25 g of diol-modified C18, and using a gradient of ACN in water (0→80% ACN in water). The desired product was recovered as a white solid, after lyophilization from water (25 mg, 58%). MS calc. for C₃₈H₄₀N₆NaO₁₀: 763.27, found: 763.80, [M+Na]⁺.

Intermediate 3. Intermediate 2 (25 mg, 0.0338 mmol) and exatecan mesylate (1.5 equiv., 0.508 mmol, 27 mg) were suspended in 4 mL of 1M borate buffer (pH 9), and the reaction mixture was stirred at 55° C. for 4 hours. DMF (2 mL) was added and the solvents were evaporated under reduced pressure until a final volume of approx. 2 mL. The product was purified by reverse-phase flash HPLC, using a semipreparative column containing diol-modified C18, and using a gradient of ACN in water (0→100% ACN in water). The desired product was recovered as a white solid, after lyophilization from water-dioxane (11 mg, 28%). MS calc. for C₆₁H₅₉FN₉O₁₃: 1144.42: 1144.42, found: 1144.01, [M+H]⁺.

Intermediate 4. Intermediate 3 (11 mg, 0.0096 mmol) was dissolved in 1 mL of DMF, and morpholine (20 μL) was added. The reaction mixture was stirred at room temperature for 30 min. The mixture was filtered through a 0.2 μm syringe filter and directly loaded on column. The product was purified by reverse-phase flash HPLC, using a semipreparative column containing diol-modified C18, and using a gradient of ACN in water (0→100% ACN in water). The desired product was recovered as a yellowish solid, after lyophilization from water-dioxane (7.5 mg, 88%). MS calc. for C₄₆H₅₀FN₉O₁₁: 923.36, found: 923.75, [M+H]⁺.

Compound 1001. Intermediate 4 (7.5 mg, 0.0081 mmol) was dissolved in 1 mL of DMF. 2,5-Dioxopyrrolidin-1-yl 3-(2-(2,5-dioxo-2,5-dihydro-1H-pyrrol-1-yl)ethoxy)propanoate (2 equiv., 0.0163 mmol, 5 mg) and DIPEA (20 μL) were added. The reaction mixture was stirred at room temperature for 30 minutes. The mixture was then filtered through a 0.2 μm syringe filter and directly loaded on column. The product was purified by reverse-phase flash HPLC, using a semipreparative column containing diol-modified C18, and using a gradient of ACN in water (0→80% ACN in water). The desired product was recovered as a white solid, after lyophilization from water (7 mg, 77%). MS calc. for C₅₅H₅₈FN₁₀O₁₅: 1117.41, found: 1117.44, [M+H]⁺. ¹H NMR (500 MHz, DMSO-d₆) δ 8.80 (s, 1H), 8.62 (d, J=10.9 Hz, 1H), 8.52 (s, 1H), 8.27 (s, 1H), 8.15 (d, J=33.5 Hz, 1H), 8.02 (d, J=16.0 Hz, 1H), 7.82 (d, J=11.5 Hz, 1H), 7.67 (s, 1H), 7.36-7.20 (m, 4H), 7.19-7.12 (m, 2H), 6.68 (s, 1H), 6.05 (t, J=20.7 Hz, 1H), 5.86 (s, 1H), 5.75 (s, 1H), 5.30 (d, J=23.9 Hz, 1H), 5.18 (m, 2H), 4.85 (d, J=11.1 Hz, 2H), 4.66-4.54 (m, 1H), 4.54 (s, 2H), 4.44 (m, 1H), 3.85-3.79 (m, 1H), 3.70-3.65 (m, 3H), 3.56-3.47 (m, 2H), 3.47-3.42 (m, 1H), 3.41-3.36 (m, 1H), 3.17 (s, 1H), 3.07-2.97 (m, 2H), 2.85-2.69 (m, 4H), 2.67-2.52 (m, 2H), 2.44-2.36 (m, 3H), 2.33-2.23 (m, 1H), 1.97 (m, 2H), 1.82 (d, J=7.9 Hz, 2H), 1.23 (s, 2H), 0.87-0.81 (m, 3H).

### Example 3: Synthesis of Compound 12

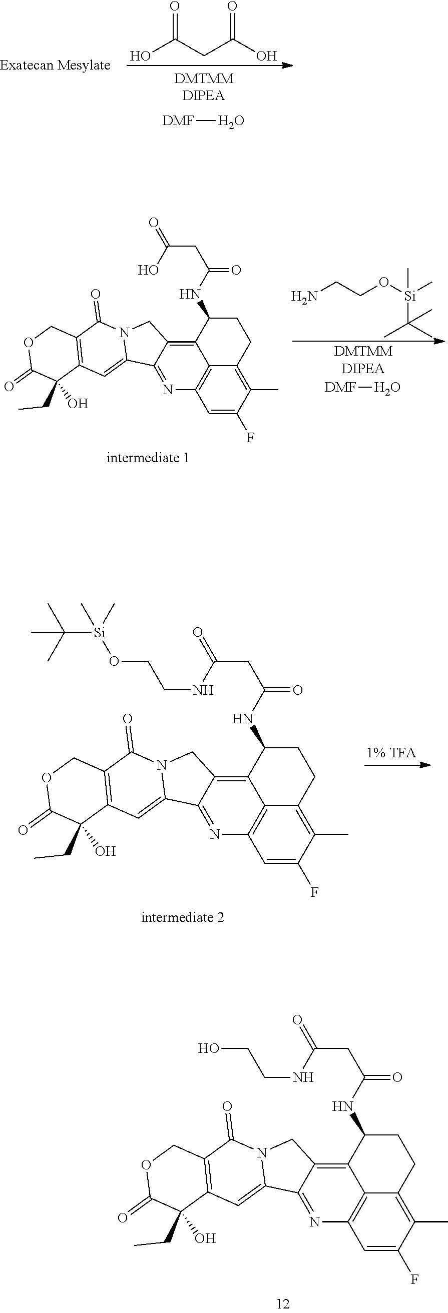

Intermediate 1. Exatecan mesylate (39 mg, 0.0737 mmol), malonic acid (5 equiv., 0.3687 mmol, 38 mg) and 4-(4,6-dimethoxy-1,3,5-triazin-2-yl)-4-methyl-morpholinium chloride (DMTMM, 5 equiv., 0.3687 mmol, 102 mg) were dissolved in a 5:1 mixture of DMF and water (6 mL). Triethylamine (50 equiv., 3.6873 mmol, 514 μL) was added and the reaction mixture was stirred for 3 hours at room temperature. Solvents were evaporated under reduced pressure, and the crude reaction mixture was purified by reverse-phase flash chromatography, using a column containing 25 g of diol-modified C18, and using a gradient of ACN in 1% TFA (0→40% ACN in 1% TFA). The desired product was recovered as a white powder, after lyophilization from water (34 mg, 88%). MS calc. for C₂₇H₂₃FN₃O₇: 520.15, found: 520.49 [M−H]⁻.

Intermediate 2. The previously synthesized intermediate 1 (24 mg, 0.0461 mmol), 2-((tert-butyldimethylsilyl)oxy)ethan-1-amine (5 equiv., 0.2303 mmol, 48 μL) and 4-(4,6-dimethoxy-1,3,5-triazin-2-yl)-4-methyl-morpholinium chloride (DMTMM, 5 equiv., 0.2303 mmol, 64 mg) were dissolved in a 5:1 mixture of DMF and water (6 mL). Triethylamine (50 equiv., 2.303 mmol, 321 μL) was added and the reaction mixture was stirred for 3 hours at room temperature. Solvents were evaporated under reduced pressure, and the crude reaction mixture was purified by reverse-phase flash chromatography, using a column containing 25 g of diol-modified C18, and using a gradient of ACN in water (0→70% ACN in H₂O). The desired product was recovered as a yellowish foam, after lyophilization from water-DMF (7 mg, 22%). MS calc. for C₃₅H₄₄FN₄O₇Si: 679.30, found: 679.00, [M+H]⁺.

Compound 12. The previously synthesized intermediate 2 (7 mg, 0.0103 mmol) was suspended in 1% TFA (2 mL) and the mixture was stirred at room temperature for 1 h. The crude reaction mixture was directly loaded on column and purified by reverse-phase flash chromatography, using a column containing 25 g of diol-modified C18, and using a gradient of ACN in 1% TFA (0→40% ACN in 1% TFA). The desired product was recovered as a yellowish powder, after lyophilization from water (3 mg, 53%). MS calc. for C₂₉H₃₀FN₄O₇: 565.21, found: 565.70, [M+H]⁺. ¹H NMR (400 MHz, DMSO-d₆) δ 8.62 (d, J=8.6 Hz, 1H), 8.05 (t, J=5.6 Hz, 1H), 7.80 (d, J=10.9 Hz, 1H), 7.31 (s, 1H), 5.58-5.54 (m, 1H), 5.43 (s, 2H), 5.26 (d, J=5.2 Hz, 2H), 3.38 (t, J=6.3 Hz, 2H), 3.18 (s, 2H), 3.15-3.04 (m, 2H), 2.58-2.52 (m, 2H), 2.46 (m, 3H), 2.25-2.16 (m, 1H), 2.11 (s, 1H), 1.87 (m, 2H), 1.76 (s, 2H), 0.88 (t, J=7.4 Hz, 3H).

### Example 4: Synthesis of Compound 1005

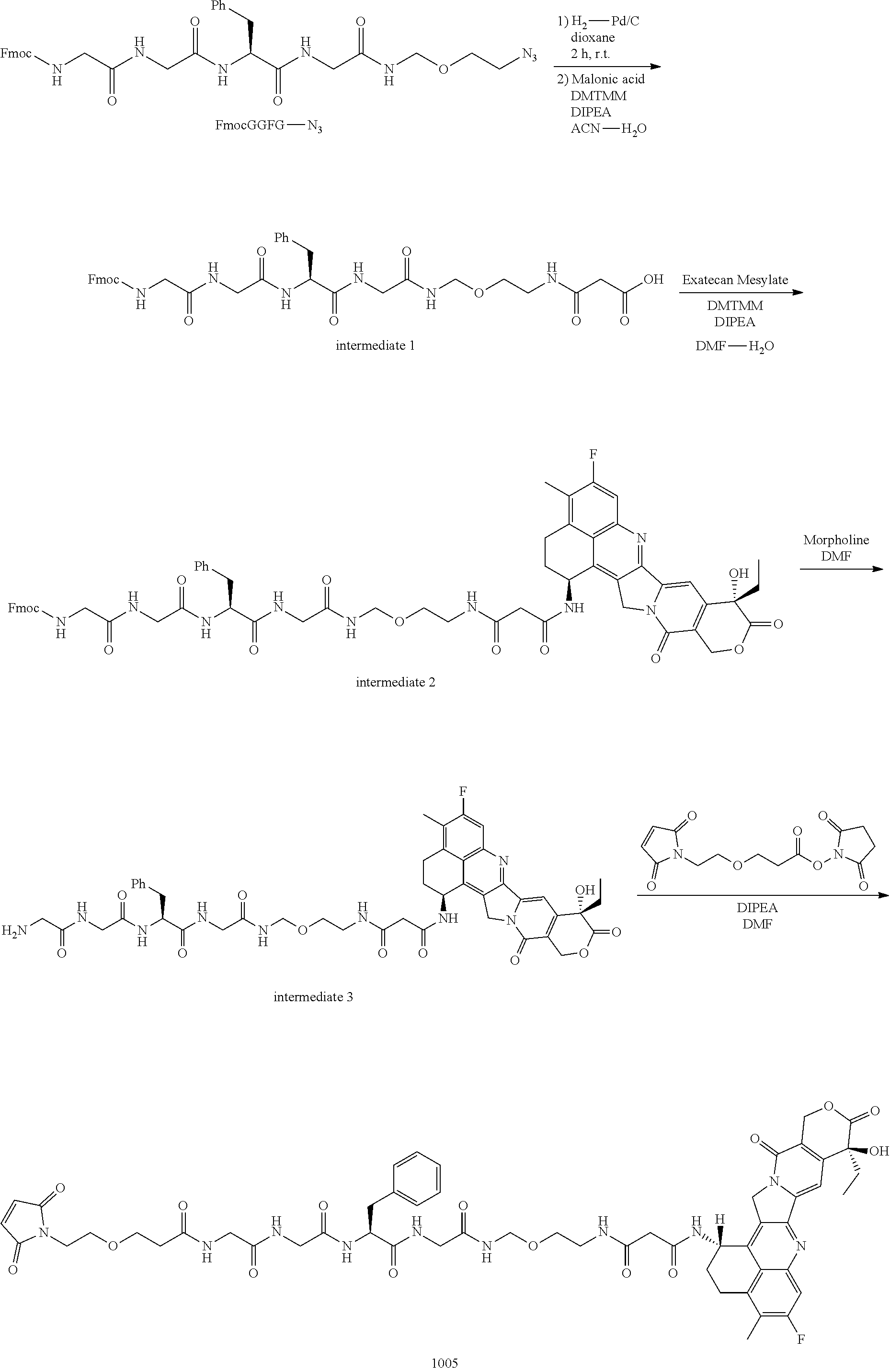

Intermediate 1. FmocGGFG-N3 (23 mg, 0.0350 mmol) was dissolved in 2 mL of dioxane. Pd/C (10% w/w, 5 mg) was suspended in the mixture, and H₂ was bubbled using a balloon into the suspension while stirring at room temperature for 2 h. The suspension was taken with a syringe and filtrate through 0.2 μm syringe filter directly into a flask containing a previously prepared solution of malonic acid (5 equiv., 0.1750 mmol, 18 mg), DMTMM (5 equiv., 0,1750 mmol, 48 mg) and DIPEA (100 μL) in ACN (2 mL) and water (0.5 mL). The reaction mixture was stirred at room temperature for 2 h. The solvents were evaporated under reduced pressure, the resulting solid was re-dissolved in DMF and directly loaded on column. The product was purified by reverse-phase flash chromatography, using a column containing 25 g of diol-modified C18, and using a gradient of ACN in water (0→40% ACN in water). The desired product was recovered as a white solid, after lyophilization from water (15 mg, 60%). MS calc. for C₃₆H₄₁N₆O₁₀: 715.27, found: 715.55 [M−H]⁻.

Intermediate 2. Intermediate 1 (14 mg, 0.0195 mmol) was dissolved in a mixture of DMF (2 mL) and water (0.5 mL). To the solution were added exatecan mesylate (1.5 equiv., 0.0293 mmol, 16 mg), DMTMM (3 equiv., 0.0587 mmol, 16 mg) and DIPEA (20 μL), and the reaction mixture was stirred for 4 h at room temperature. The solvents were evaporated under reduced pressure, the resulting solid was re-dissolved in DMF and directly loaded on column. The product was purified by reverse-phase flash HPLC, using a semipreparative column containing diol-modified C18, and using a gradient of ACN in water (0→100% ACN in water). The desired product was recovered as a yellow solid, after lyophilization from water (15 mg, 66%). MS calc. for C₆₀H₆₁FN₉O₁₃: 1134.44, found: 1134.40 [M+H]⁺.

Intermediate 3. Intermediate 2 (15 mg, 0.0132 mmol) was dissolved in 1 mL of DMF, and morpholine (20 μL) was added. The reaction mixture was stirred at room temperature for 30 min. The mixture was filtered through a 0.2 μm syringe filter and directly loaded on column. The product was purified by reverse-phase flash HPLC, using a semipreparative column containing diol-modified C18, and using a gradient of ACN in water (0→100% ACN in water). The desired product was recovered as a yellow solid, after lyophilization from water-dioxane (10 mg, 83%). MS calc. for C₄₅H₅₁FN₉O₁₁: 912.37, found: 912.91 [M+H]⁺.

Compound 1005. Intermediate 3 (10 mg, 0.0110 mmol) was dissolved in 1 mL of DMF. 2,5-Dioxopyrrolidin-1-yl 3-(2-(2,5-dioxo-2,5-dihydro-1H-pyrrol-1-yl)ethoxy)propanoate (2 equiv., 0.0219 mmol, 7 mg) and DIPEA (20 μL) were added. The reaction mixture was stirred at room temperature for 30 minutes. The mixture was then filtered through a 0.2 μm syringe filter and directly loaded on column. The product was purified by reverse-phase flash HPLC, using a semipreparative column containing diol-modified C18, and using a gradient of ACN in water (0→80% ACN in water). The desired product was recovered as a yellow solid, after lyophilization from water-dioxane (11 mg, 90%). MS calc. for C₅₄H₆₀FN₁₀O₁₅: 1107.42, found: 1107.50 [M+H]⁺. ¹H NMR (500 MHz, DMSO-d₆) δ 8.67 (m, 1H), 8.61 (m, 1H), 8.52 (m, 1H), 8.38 (s, 1H), 8.24 (s, 1H), 8.12 (t, J=5.6 Hz, 1H), 7.81 (d, J=11.0 Hz, 1H), 7.67 (s, 1H), 7.28 (m, 4H), 7.25-7.24 (m, 2H), 7.212 (s, 1H), 7.15 (m, 1H), 6.68 (s, 1H), 6.09-6.00 (m, 1H), 5.58-5.47 (m, 1H), 5.19-5.08 (m, 2H), 4.88 (d, J=11.8 Hz, 1H), 4.65 (d, J=11.7 Hz, 1H), 4.50 (dd, J=11.7, 6.6 Hz, 1H), 4.47 (m, 2H), 3.78-3.63 (m, 3H), 3.63-3.52 (m, 4H), 3.19 (m, 3H), 3.11-3.01 (m, 4H), 2.63 (p, J=1.9 Hz, 5H), 2.43-2.36 (m, 6H), 2.24-2.14 (m, 1H), 1.75 (m, 2H), 1.23 (s, 2H), 0.85 (dd, J=7.9, 6.3 Hz, 3H).

### Example 5: Synthesis of Compound 16

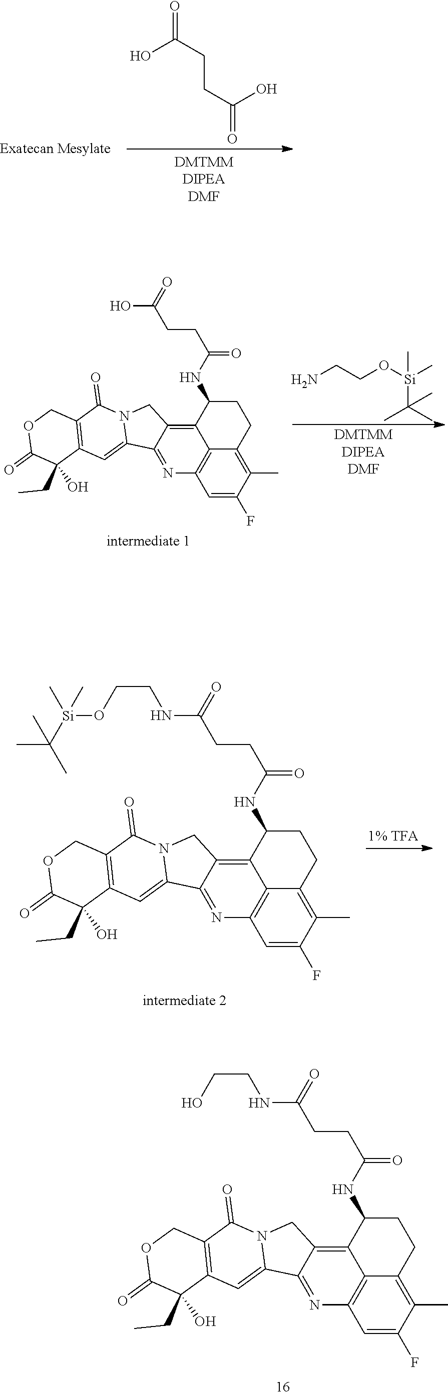

Intermediate 1. Exatecan mesylate (20 mg, 0.0376 mmol), succinic acid (5 equiv., 0.1881 mmol, 22 mg) and 4-(4,6-dimethoxy-1,3,5-triazin-2-yl)-4-methyl-morpholinium chloride (DMTMM, 5 equiv., 0.1881 mmol, 53 mg) were dissolved in a 5:1 mixture of DMF and water (4 mL). Triethylamine (200 μL) was added and the reaction mixture was stirred for 3 hours at room temperature. Solvents were evaporated under reduced pressure, and the crude reaction mixture was purified by reverse-phase flash chromatography, using a column containing 25 g of diol-modified C18, and using a gradient of ACN in 1% TFA (0→40% ACN in 1% TFA). The desired product was recovered as a brown powder, after lyophilization from water-dioxane (18 mg, 89%). MS calc. for C₂₈H₂₅FN₃O₇: 534.17, found: 534.80 [M−H]⁻.

Intermediate 2. The previously synthesized intermediate 1 (15 mg, 0.0424 mmol), 2-((tert-butyldimethylsilyl)oxy)ethan-1-amine (3 equiv., 0.1272 mmol, 22 mg) and hexafluorophosphate azabenzotriazole tetramethyl uronium (HATU, 3 equiv., 0.1272 mmol, 48 mg) were dissolved in DMF (1 mL). Diisopropylethylamine (50 μL) was added and the reaction mixture was stirred for 1 hour at room temperature. Solvents were evaporated under reduced pressure, and the crude reaction mixture was purified by reverse-phase flash chromatography, using a column containing 25 g of diol-modified C18, and using a gradient of ACN in water (0→80% ACN in H₂O). The desired product was recovered as a yellow foam, after lyophilization from water-dioxane (22 mg, 74%). MS calc. for C₃₆H₄₆FN₄O₇Si: 693.31, found: 693.55, [M+H]⁺.

Compound 16. The previously synthesized intermediate 2 (22 mg, 0.0317 mmol) was suspended in 1% TFA (2 mL) and the mixture was stirred at room temperature for 1 h. The crude reaction mixture was directly loaded on column and purified by reverse-phase flash chromatography, using a column containing 25 g of diol-modified C18, and using a gradient of ACN in 1% TFA (0→40% ACN in 1% TFA). The desired product was recovered as a white powder, after lyophilization from water (11 mg, 60%). MS calc. for C₃₀H₃₂FN₄O₇: 579.23, found: 579.25, [M+H]⁺. ¹H NMR (400 MHz, DMSO-d₆) δ 8.46 (d, J=8.7 Hz, 1H), 7.82 (m, 1H), 7.79 (d, J=11.0 Hz, 1H), 7.31 (s, 1H), 5.56 (t, J=4.8 Hz, 1H), 5.42 (s, 2H), 5.30-5.10 (m, 2H), 3.47 (m, 2H), 3.35 (t, J=6.2 Hz, 2H), 3.18 (m, 2H), 3.07 (td, J=6.1, 4.4 Hz, 2H), 2.56-2.52 (m, 2H), 2.42-2.32 (m, 5H), 2.12 (q, J=5.3 Hz, 2H), 1.95-1.79 (m, 2H), 0.88 (t, J=7.3 Hz, 3H).

### Example 6: Synthesis of Compound 18

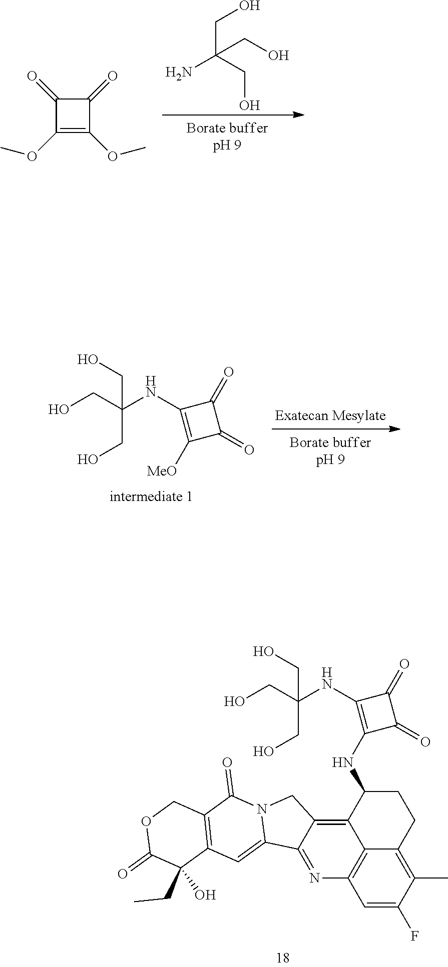

Intermediate 1. Tris(hydroxymethyl)aminomethane (25 mg, 0.206 mmol) and dimethoxysquarate (3 equiv., 0.619 mmol, 88 mg,) were suspended in 10 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (22 m, 48%). MS calc. for C₉H₁₄NO₆: 232.08, found: 232.19, [M+H]⁺.

Compound 18. Exatecan mesylate (20 mg, 0.0377 mmol) and the previously synthesized intermediate 1 (1,5 equiv., 0.0564 mmol, 13 mg) were suspended in 5 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (9 mg, 38%). MS calc. for C₃₂H₃₂FN₄O₉: 635.22, found: 635.63, [M+H]⁺. ¹H NMR (400 MHz, DMSO-d₆) δ 8.31 (d, J=8.9 Hz, 1H), 7.86 (d, J=10.9 Hz, 1H), 7.32 (s, 1H), 7.14 (s, 1H), 6.54 (s, 1H), 5.86 (dd, J=8.8, 4.3 Hz, 1H), 5.42 (s, 1H), 5.39 (d, J=19.0 Hz, 2H), 5.15 (d, J=19.0 Hz, 2H), 4.70 (t, J=5.6 Hz, 3H), 3.61 (d, J=5.7 Hz, 6H), 3.27 (m, 1H), 2.44 (m, 3H), 2.41 (m, 1H), 2.26 (m, 1H), 1.85 (m, 2H), 0.87 (t, J=7.3 Hz, 3H).

### Example 7: Synthesis of Compound 22

### Step 1:

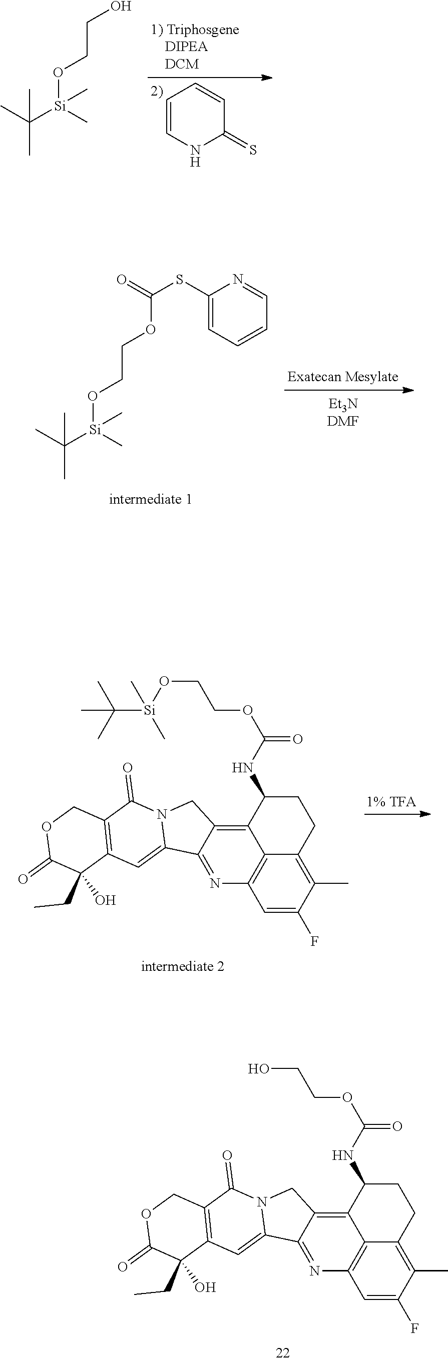 [SMILES: CC(C)(C)[Si](C)(C)OCCO ; S=c1cccc[nH]1 ; CC(C)(C)[Si](C)(C)OCCOC(=O)Sc1ccccn1 ; CC[C@@]1(O)C(=O)OCc2c1cc1n(c2=O)Cc2c-1nc1cc(F)c(C)c3c1c2[C@@H](NC(=O)OCCO[Si](C)(C)C(C)(C)C)CC3 ; CC[C@@]1(O)C(=O)OCc2c1cc1n(c2=O)Cc2c-1nc1cc(F)c(C)c3c1c2[C@@H](NC(=O)OCCO)CC3]

Intermediate 1. 2-[[Tert-Butyl(dimethyl)silyl]oxy]ethanol (1.14 mmol, 200 mg) was dissolved in 4 mL of anhydrous dichloromethane under an argon atmosphere. The reaction mixture was cooled at 0° C., and diisopropylethylamine (1.1 equiv., 1.25 mmol, 218 μL) was added, followed by triphosgene (1.2 equiv., 0.45 mmol, 135 mg). The reaction mixture was stirred at 0° C. for 2 h. Further diisopropylethylamine (1.2 equiv., 1.25 mmol, 218 μL) was added, followed by 2-mercaptopyridine (1.1 equiv., 1.25 mmol, 155 mg). After stirring for 2 more hours at 0° C., the reaction mixture was diluted with 20 mL of dichloromethane and saturated NH₄Cl (10 mL) was added. The organic phase was washed with saturated NH₄Cl (2×100 mL) and brine (100 mL). Silica gel flash chromatography afforded, using a gradient of EtOAc in cyclohexane (0→50% EtOAc in cyclohexane) afforded the desired product (100 mg, 28%). MS calc. for C₁₄H₂₄NO₃SSi: 314.12, found: 314.10, [M+H]⁺.

Compound 22. Exatecan mesylate (0.0376 mmol, 20 mg) and triethylamine (2 equiv., 0.0752 mmol, 11 μL) were dissolved in 2 mL of anhydrous DMF under an argon atmosphere. The intermediate 1 (1.5 equiv., 0.0752 mmol, 18 mg) was added, and the reaction mixture was stirred at room temperature for 48 h. Solvents were evaporated under reduced pressure, and the crude reaction mixture was purified by reverse-phase flash chromatography, using a column containing 25 g of diol-modified C18, and using a gradient of ACN in water (0→40% ACN in H₂O). Fractions containing the desired reaction product (intermediate 2) were evaporated, the solid was re-suspended in 1% TFA and stirred at room temperature for 1 h. The desired product was re-purified by reverse-phase flash chromatography, using a column containing 25 g of diol-modified C18, and using a gradient of ACN in 1% TFA (0→40% ACN in 1% TFA), and recovered as a white powder, after lyophilization from water (16 mg, 81% over 2 steps, calculated on Exatecan). MS calc. for C₂₇H₂₇FN₃O₇: 524.18, found: 524.20, [M+H]⁺. ¹H NMR (400 MHz, DMSO-d₆) δ 7.97 (d, J=9.0 Hz, 1H), 7.77 (d, J=11.0 Hz, 1H), 7.32 (s, 1H), 5.43 (s, 2H), 5.32-5.16 (m, 2H), 4.11 (m, 2H), 3.33 (s, 1H), 3.25 (d, J=6.0 Hz, 1H), 3.11 (d, J=18.7 Hz, 1H), 2.68 (m, 1H), 2.55 (m, 2H), 2.39-2.29 (m, 4H), 2.24-2.12 (m, 2H), 1.87 (m, 2H), 0.88 (t, J=7.3 Hz, 3H).

### Example 8: Synthesis of Compound 1007

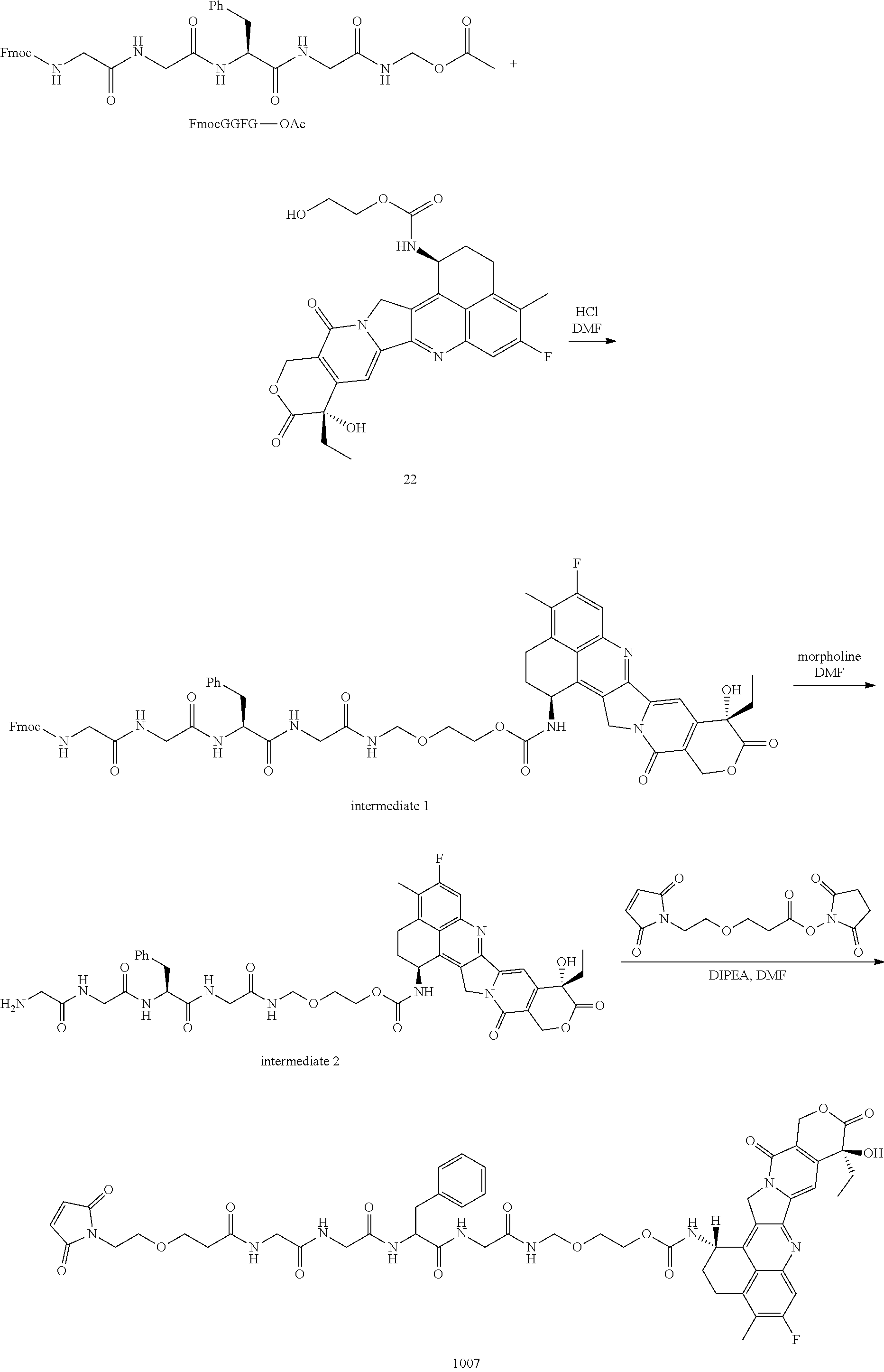

Intermediate 1. Compound 22 (28 mg, 0.0535 mmol) and FmocGGFG-OAc (2 equiv., 0.107 mmol, 67 mg) were dissolved in 1 mL of anhydrous DMF. HCl (100 μL, 2 M in Et₂O) was added and the reaction mixture was stirred at room temperature for 2 h. The mixture was directly loaded on column. The product was purified by reverse-phase flash chromatography, using a column containing 25 g of C18, and using a gradient of ACN in water (0→70% ACN in water). The desired product was recovered as a white solid, after lyophilization from water (23 mg, 39%). MS calc. for C₅₈H₅₈FN₈O₁₃: 1093.41, found: 1093.63, [M+H]⁺.

Intermediate 2. Intermediate 1 (23 mg, 0.0211 mmol) was dissolved in 1 mL of anhydrous DMF, and morpholine (100 μL) was added. The reaction mixture was stirred for 1 h at room temperature, to be then directly loaded on column. The product was purified by reverse-phase flash HPLC, using a semipreparative column containing 25 g of diol-modified C18, and using a gradient of ACN in water (0→100% ACN in water). The desired product was recovered as a white solid, after lyophilization from water (16 mg, 85%). MS calc. for C₄₃H₄₈FN₈₀O₁₁: 871.34, found: 871.44, [M+H]⁺.

Compound 1007. Intermediate 2 (16 mg, 0.0179 mmol) was dissolved in 1 mL of DMF. 2,5-Dioxopyrrolidin-1-yl 3-(2-(2,5-dioxo-2,5-dihydro-1H-pyrrol-1-yl)ethoxy)propanoate (2 equiv., 0.0358 mmol, 11 mg) and DIPEA (20 μL) were added. The reaction mixture was stirred at room temperature for 30 minutes. The mixture was then filtered through a 0.2 μm syringe filter and directly loaded on column. The product was purified by reverse-phase flash HPLC, using a semipreparative column containing diol-modified C18, and using a gradient of ACN in water (0→100% ACN in water). The desired product was recovered as a white solid, after lyophilization from water (7 mg, 37%). MS calc. for C₅₂H₅₇FN₉O₁₅: 1066.40, found: 1065.98, [M+H]⁺. ¹H NMR (500 MHz, DMSO-d₆) δ 8.79 (s, 1H), 8.65 (d, J=6.9 Hz, 1H), 8.58-8.49 (m, 1H), 8.45 (m, 1H), 8.37 (s, 1H), 8.31-8.22 (m, 1H), 8.15-8.08 (m, 2H), 8.07-7.96 (m, 1H), 7.91 (s, 1H), 7.79 (t, J=11.5 Hz, 2H), 7.34-7.28 (m, 1H), 7.25-7.17 (m, 3H), 7.01 (d, J=10.9 Hz, 2H), 6.67 (s, 1H), 6.61-6.46 (m, 1H), 5.42 (s, 1H), 5.39-5.28 (m, 1H), 5.26-5.23 (m, 2H), 5.15 (d, J=7.3 Hz, 1H), 4.57 (m, 2H), 4.49 (d, J=11.1 Hz, 2H), 4.18 (d, J=4.8 Hz, 2H), 3.77-3.63 (m, 3H), 3.61 (m, 2H), 3.58-3.42 (m, 3H), 3.25-3.17 (m, 1H), 3.04 (s, 2H), 2.77 (dd, J=8.5, 5.5 Hz, 1H), 2.47-2.29 (m, 4H), 2.20-2.11 (m, 1H), 2.00 (m, 1H), 1.86 (m, 2H), 1.23 (s, 2H), 0.97-0.82 (m, 3H).

### Example 9: Synthesis of Compound 42

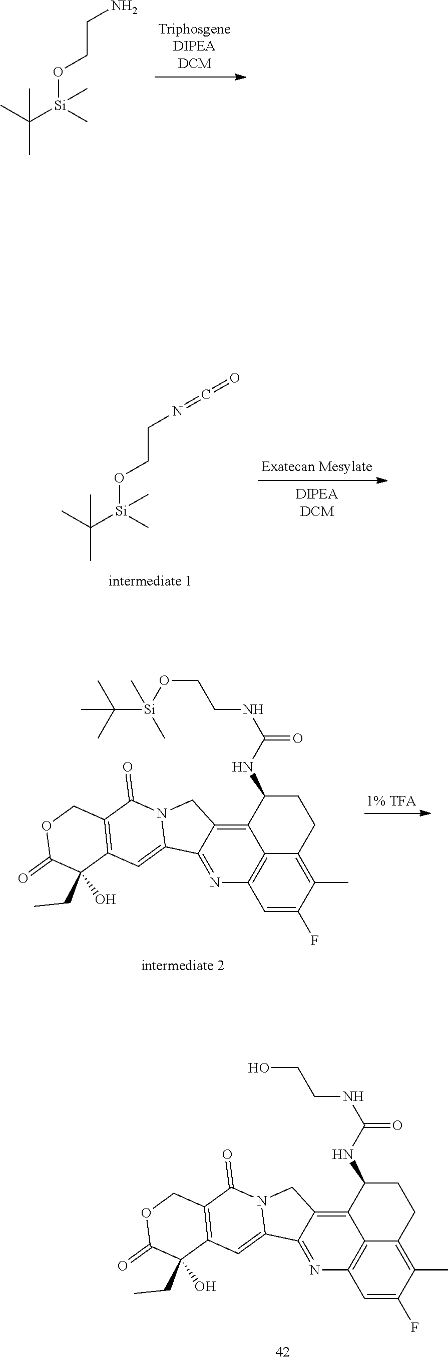 [SMILES: CC(C)(C)[Si](C)(C)OCCN ; CC(C)(C)[Si](C)(C)OCCN=C=O ; CC[C@@]1(O)C(=O)OCc2c1cc1n(c2=O)Cc2c-1nc1cc(F)c(C)c3c1c2[C@@H](NC(=O)NCCO[Si](C)(C)C(C)(C)C)CC3 ; CC[C@@]1(O)C(=O)OCc2c1cc1n(c2=O)Cc2c-1nc1cc(F)c(C)c3c1c2[C@@H](NC(=O)NCCO)CC3]

Intermediate 1. 2-((Tert-butyldimethylsilyl)oxy)ethan-1-amine (21 mg, 0.121 mmol), triphosgene (0.95 equiv., 0.0383 mmol, 11 mg) and diisopropylethylamine (5 equiv., 0.605 mmol, 105 μL) were dissolved in dichloromethane (2 mL) under an argon atmosphere. The reaction mixture was stirred at room temperature for 1 h. The full reaction conversion to afford the intermediate 1 was confirmed by LCMS analysis. The reaction product was used in the next step without any further purification.

Compound 42. Exatecan mesylate (0.0602 mmol, 32 mg) and diisopropylethylamine (2 equiv., 0.120 mmol, 21 μL) were dissolved in 1 mL of anhydrous DMF, and the solution was cooled at 0° C. A dichloromethane solution of the previously prepared isocyanate intermediate 1 (2 mL, 0.1150 mmol) was added at 0° C., and the reaction mixture was allowed to reach room temperature and stirred for 1 h. Solvents were evaporated under reduced pressure, and the crude reaction mixture was purified by reverse-phase flash chromatography, using a column containing 25 g of diol-modified C18, and using a gradient of ACN in water (0→60% ACN in H₂O). Fractions containing the desired reaction product (intermediate 2) were evaporated, the solid was re-suspended in 1% TFA and stirred at room temperature for 1 h. The desired product was re-purified by reverse-phase flash chromatography, using a column containing 25 g of diol-modified C18, and using a gradient of ACN in 1% TFA (0→40% ACN in 1% TFA), and recovered as a white powder, after lyophilization from water (15 mg, 48% over 3 steps, calculated from exatecan). MS calc. for C₂₇H₂₈FN₄O₆: 523.20, found: 523.25, [M+H]⁺. ¹H NMR (400 MHz, DMSO-d₆) δ 7.76 (dd, J=11.0, 2.0 Hz, 1H), 7.31 (s, 1H), 6.82 (d, J=8.9 Hz, 1H), 6.61 (m, 1H), 5.43 (d, J=2.6 Hz, 2H), 5.40-5.30 (m, 2H), 5.22 (s, 1H), 5.17 (s, 1H), 3.48-3.40 (m, 2H), 3.21-3.01 (m, 3H), 2.38 (d, J=1.9 Hz, 3H), 2.23-2.06 (m, 2H), 1.96-1.80 (m, 2H), 1.76 (s, 2H), 0.90 (t, J=7.3 Hz, 3H).

### Example 10: Synthesis of Compound 1008

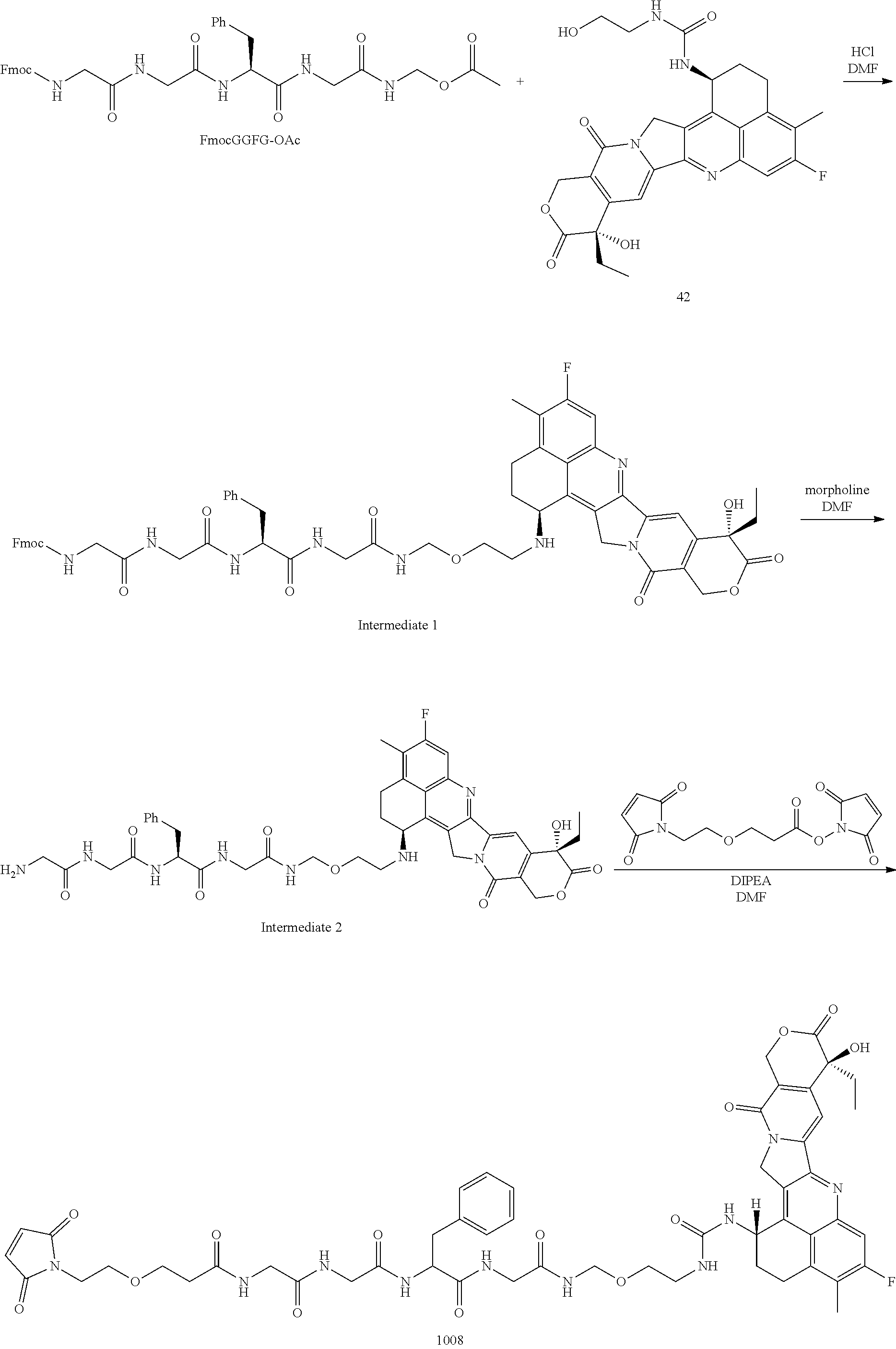

Intermediate 1. Compound 48 (17 mg, 0.0325 mmol) and FmocGGFG-OAc (3 equiv., 0.0976 mmol, 61 mg) were dissolved in 1 mL of anhydrous DMF. HCl (100 μL, 2 M in Et₂O) was added and the reaction mixture was stirred at room temperature for 2 h. The mixture was directly loaded on column. The product was purified by reverse-phase flash chromatography, using a column containing 25 g of C18, and using a gradient of ACN in water (0→80% ACN in water). The desired product was recovered as a white solid, after lyophilization from water (15 mg, 44%). MS calc. for C₅₈H₅₉FN₉O₁₂: 1092.43, found: 1093.03, [M+H]⁺.

Intermediate 2. Intermediate 1 (15 mg, 0.0135 mmol) was dissolved in 1 mL of anhydrous DMF, and morpholine (100 μL) was added. The reaction mixture was stirred for 1 h at room temperature, to be then directly loaded on column. The product was purified by reverse-phase flash HPLC, using a semipreparative column containing 25 g of diol-modified C18, and using a gradient of ACN in water (0→100% ACN in water). The desired product was recovered as a white solid, after lyophilization from water (10 mg, 87%). MS calc. for C₄₃H₄₉FN₉O₁₀: 870.36, found: 870.88, [M+H]⁺.

Compound 1008. Intermediate 2 (10 mg, 0.0118 mmol) was dissolved in 1 mL of DMF. 2,5-Dioxopyrrolidin-1-yl 3-(2-(2,5-dioxo-2,5-dihydro-1H-pyrrol-1-yl)ethoxy)propanoate (2 equiv., 0.0237 mmol, 7 mg) and DIPEA (20 μL) were added. The reaction mixture was stirred at room temperature for 30 minutes. The mixture was then filtered through a 0.2 μm syringe filter and directly loaded on column. The product was purified by reverse-phase flash HPLC, using a semipreparative column containing diol-modified C18, and using a gradient of ACN in water (0→100% ACN in water). The desired product was recovered as a white solid, after lyophilization from water (4 mg, 32%). MS calc. for C₅₂H₅₈FN₁₀O₁₄: 1065.41, found: 1065.79, [M+H]⁺.

### Example 11: Synthesis of Compound 48

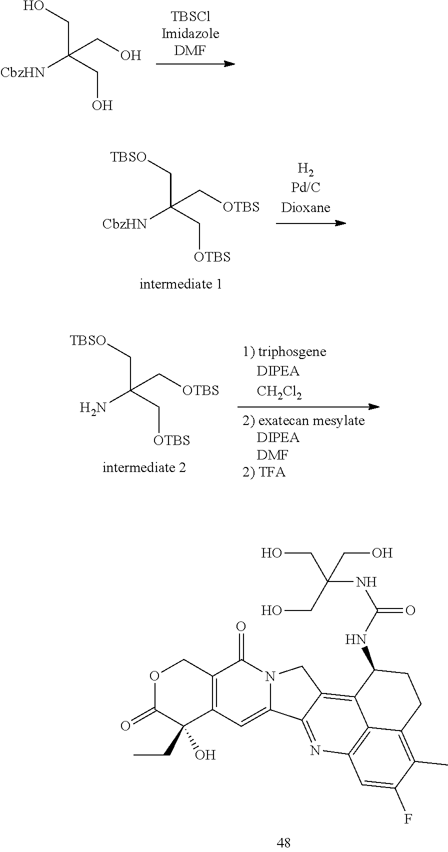 [SMILES: O=C(NC(CO)(CO)CO)OCc1ccccc1 ; CC(C)(C)[Si](C)(C)OCC(CO[Si](C)(C)C(C)(C)C)(CO[Si](C)(C)C(C)(C)C)NC(=O)OCc1ccccc1 ; CC(C)(C)[Si](C)(C)OCC(N)(CO[Si](C)(C)C(C)(C)C)CO[Si](C)(C)C(C)(C)C ; CC[C@@]1(O)C(=O)OCc2c1cc1n(c2=O)Cc2c-1nc1cc(F)c(C)c3c1c2[C@@H](NC(=O)NC(CO)(CO)CO)CC3]

Intermediate 1. Benzyl (1,3-dihydroxy-2-(hydroxymethyl)propan-2-yl)carbamate (100 mg, 0.392 mmol), tertbutyldimethylsilyl chloride (1.5 equiv., 1.764 mmol, 266 mg) and imidazole ((1.5 equiv., 1.764 mmol, 120 mg) were dissolved in 5 mL of anhydrous DMF. The reaction mixture was stirred at room temperature overnight. Solvents were evaporated and purification by flash chromatography on silica (0→50% EtOAc in hexane) offered the intermediate 1 (188 mg, 80%). MS calc. for C₃₀H₆₀NO₅Si₃: 598.38, found: 598.25, [M+H]⁺.

Intermediate 2. The previously prepared intermediate 1 (188 mg, 0.314 mmol) was dissolved into 5 mL of anhydrous dioxane. Pd/C was added and the hydrogen was bubbled into the suspension through a balloon, while the reaction mixture was stirred at room temperature for 3 h. Solvents were evaporated and purification by flash chromatography on silica (0→50% EtOAc in hexane) offered the desired intermediate 2 (101 mg, 69%). MS calc. for C₂₂H₅₄NO₃Si₃: 464.34, found: 463.98, [M+H]⁺.

Compound 48. The previously prepared intermediate 2 (56 mg, 0.121 mmol), triphosgene (1 equiv., 11.4 mg), and diidopropylethylamine (100 μL) were dissolved into 2 mL of anhydrous dichlorometane. The reaction mixture was stirred for 1 h at room temperature, to be then added to a pre-mixed solution of exatecan (30 mg, 0.0564 mmol) and diisopropylethylamine (30 μL) in anhydrous DMF. The reaction mixture was stirred at room temperature for 24 hours. Solvents were evaporated and the crude solid was re-dissolved into 2 mL of DMF. Water (2 mL) and TFA (1 mL) were added and the mixture was stirred at room temperature for 1 h. The solvents were evaporated under reduced pressure, and co-evaporated with water 3 times. The crude solid was re-dissolved in DMF (2 mL) and purification by reverse-phase flash chromatography on semipreparative column (diol-modified C18, 0→70% ACN/1% TFA in H₂O) offered the product (15 mg, 70%) as a white powder after lyophilization. MS calc. for C₂₉H₃₂FN₄O₈: 383.22, found: 383.54, [M+H]⁺. ¹H NMR (400 MHz, DMSO-d₆) δ 7.69 (d, J=10.9 Hz, 1H), 7.30 (s, 1H), 7.13 (d, J=8.9 Hz, 1H), 5.87 (s, 1H), 5.43 (d, J=1.6 Hz, 2H), 5.40-5.33 (m, 1H), 5.29 (d, J=19.5 Hz, 1H), 5.12 (d, J=19.2 Hz, 1H), 3.55 (s, 6H), 3.16 (dt, J=10.6, 4.3 Hz, 2H), 2.35 (d, J=1.9 Hz, 3H), 2.26-2.15 (m, 1H), 2.14-2.03 (m, 1H), 1.86 (hept, J=7.1 Hz, 2H), 0.88 (t, J=7.3 Hz, 3H).

### Example 12: Synthesis of Compound 52

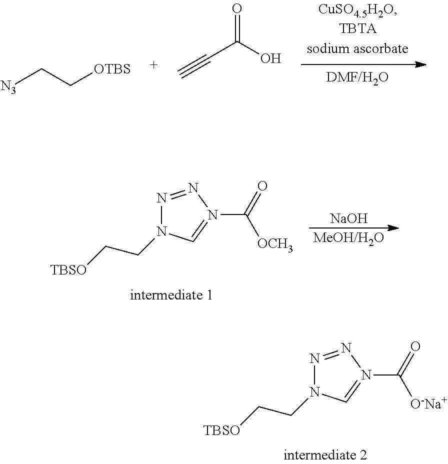

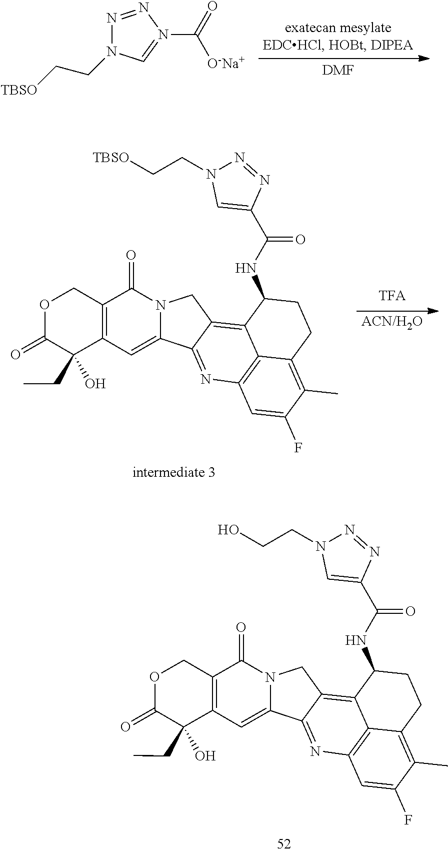

Intermediate 1. To a solution of methyl propiolate (1.0 equiv., 55 mg, 0.65 mmol), (2-azidoethoxy)(tert-butyl)dimethylsilane (1.15 equiv., 150 mg, 0.74 mmol) and tris[(1-benzyltriazol-4-yl)methyl]amine (0.15 equiv., 50 mg, 0.094 mmol) in DMF (3 ml) was added 1M aqueous CuSO₄·5H₂O (0.1 equiv., 0.06 mmol, 60 μL) and 2M aqueous sodium ascorbate (0.2 equiv., 0.12 mmol, 60 μL) and the resulting mixture was stirred at room temperature for 2 hours. DMF was evaporated and the residue was taken into EtOAc and the organic phase was washed with water, 0.2M aqueous HCl, saturated aqueous NH₄Cl and brine, and dried by Na₂SO₄. Purification by flash chromatography (silica, 0% to 30% EtOAc/cyclohexane) offered the triazole intermediate 1 (158 mg, 85%) as a white solid.

Intermediate 2. To a solution of triazole intermediate 1 (1.0 equiv., 158 mg, 0.55 mmol) in MeOH (2 mL) was added 2M aqueous NaOH (1.0 equiv., 0.55 mL) and the resulting mixture was stirred at room temperature overnight. Solvents were then evaporated and the residue was re-evaporated two times with toluene, suspended in EtOAc and filtered. Solid material was washed with Et₂O and dried on vacuum, giving the triazole intermediate 2 (135 mg, 84%) as a white solid.

Intermediate 3. To a suspension of exatecan mesylate (15 mg, 0.028 mmol), triazole intermediate 2 (2.1 equiv., 0.06 mmol, 17 mg), N-ethyl-N′-(3-dimethylaminopropyl)carbodiimide hydrochloride (2.0 equiv., 0.056 mmol, 11 mg) and 1-hydroxybenzotriazole (2.0 equiv., 0.056 mmol, 8 mg) in DMF (1 mL) was added diisopropylethylamine (5.0 equiv., 0.14 mmol, 18 mg, 25 μL) under an argon atmosphere and the mixture was stirred at room temperature for 5 hours. LC-MS indicated the full consumption of the starting material. Purification of the mixture by reverse-phase flash chromatography (25 g, diol-modified C18, 0% to 75% ACN/H₂O) offered triazole intermediate 3 (14 mg, 73%) as a white powder after lyophilisation.

Compound 52. The triazole intermediate 3 (14 mg, 0.02 mmol) was dissolved in ACN/0.1% aq. TFA mixture (1:1, 2 ml), followed by the addition of 2 drops of TFA and the resulting mixture was stirred at room temperature for 2 hours. LC-MS indicated the full consumption of the intermediate 1 and solvents were evaporated under reduced pressure. The residue was purified by reverse-phase flash chromatography using semipreparative column (diol-modified C18, 0→50% ACN/H₂O), giving compound 52 (10 mg, 85%) as a white powder after lyophilisation. MS calc. for C₂₉H₂₈FN₆O₆: 575.20, found: 575.25, [M+H]⁺. ¹H NMR (400 MHz, DMSO-d₆) δ: 8.63 (s, 1H), 7.77 (d, J=10.9 Hz, 1H), 7.29 (s, 1H), 5.73 (dd, J=7.7, 5.3 Hz, 1H), 5.36 (s, 2H), 5.14 (d, J=3.7 Hz, 2H), 4.48 (t, J=5.3 Hz, 2H), 3.81 (t, J=5.4 Hz, 2H), 3.33 (s, 2H), 3.32-3.21 (m, 1H), 3.19-3.07 (m, 1H), 2.38 (d, J=1.9 Hz, 3H), 2.34-2.20 (m, 2H), 1.93-1.76 (m, 2H), 0.85 (t, J=7.3 Hz, 3H).

### Example 13: Synthesis of Compound 1010

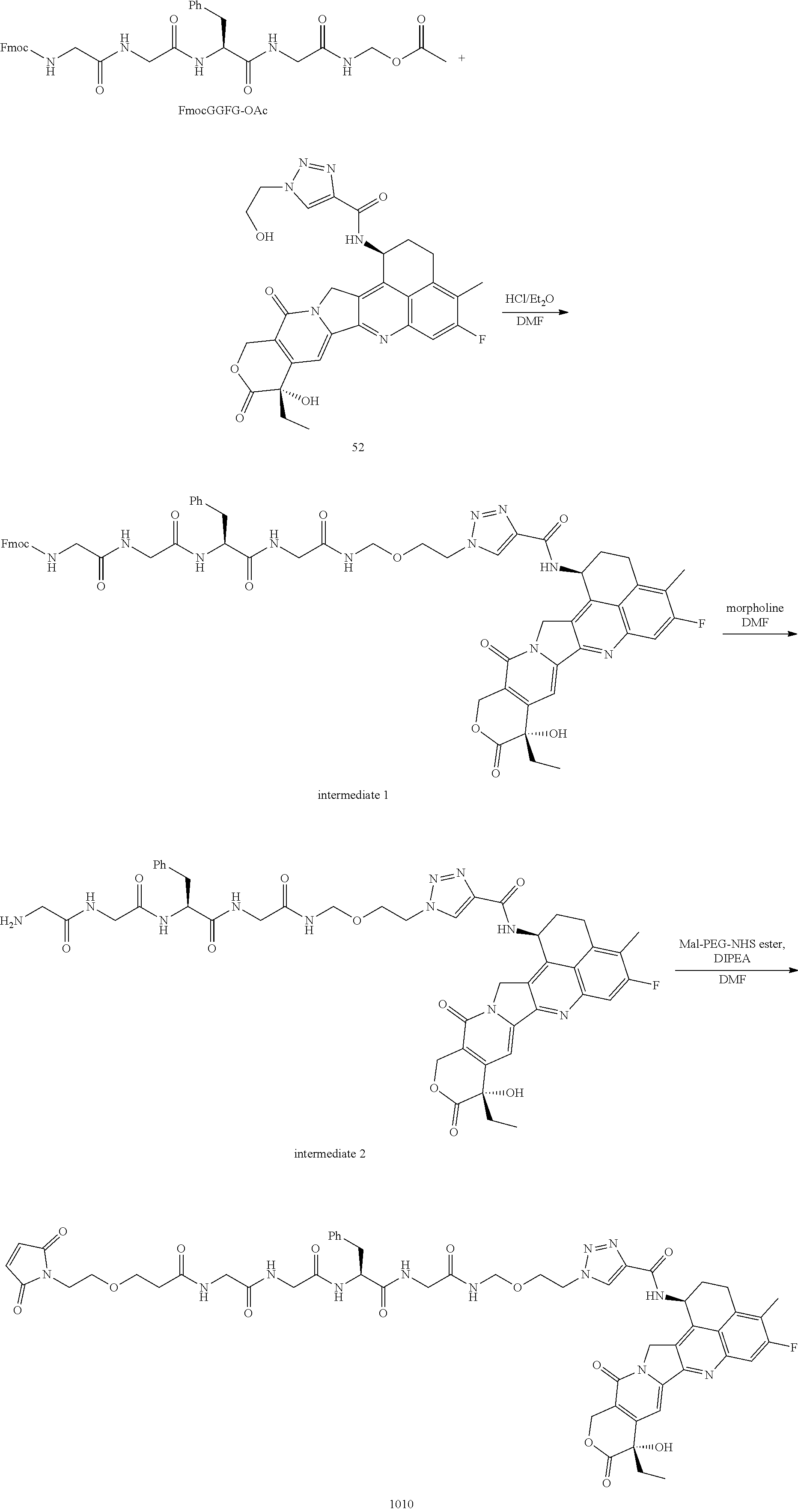

Intermediate 1. The mixture of compound 52 (1.0 equiv., 30 mg, 0.052 mmol) and FmocGGFG-OAc (2.0 equiv., 0.104 mmol, 66 mg) was dissolved in anhydrous DMF (1.5 mL), followed by the addition of 2M HCl/Et₂O (150 μL). The reaction mixture was stirred at room temperature for 4 h and during that time, several portions (ca. 0.5 equiv. each) of FmocGGFG-OAc were added to the reaction mixture. Then the reaction mixture was purified by reverse-phase flash chromatography (25 g, diol-modified C18, 0% to 75% ACN/H₂O). Fractions containing the product were lyophilised and the residue (product+co-eluting impurities) was used directly into next step. MS calc. for C₆₀H₅₉FN₁₁O₁₂: 1144.43, found: 1144.40, [M+H]⁺.

Intermediate 2. Morpholine (140 μL) was added to the solution of intermediate 1 (as obtained in previous step) in anhydrous DMF (2 ml) and the reaction mixture was stirred for 1 h at room temperature. LC-MS indicated the full consumption of starting material. The mixture was directly purified by reverse-phase flash chromatography (25 g, diol-modified C18, 0% to 60% ACN/H₂O), giving the product as a white solid after lyophilisation (20 mg, 42%, 2 steps). MS calc. for C₄₅H₄₉FN₁₁O₁₀: 922.36, found: 922.35, [M+H]⁺.

Compound 1010. Mal-PEG-NHS ester (1.0 equiv., 0.022 mmol, 6.7 mg) and DIPEA (2.4 equiv., 0.053 mmol, 7 mg, 9 μL) were added to the solution of intermediate 2 (1.0 equiv., 20 mg, 0.022 mmol) in anhydrous DMF (1 ml). The reaction mixture was stirred at room temperature for 40 minutes, as LC-MS indicated the full consumption of starting material. Purification by reverse-phase flash HPLC using a semipreparative column (diol-modified C18, 0% to 60% ACN/H₂O) offered the desired product as a white solid after lyophilisation (7.5 mg, 31%). MS calc. for C₅₄H₅₈FN₁₂O₁₄: 1117.42, found: 1117.35, [M+H]⁺. ¹H NMR (500 MHz, DMSO-d₆) δ: 9.28 (t, J=9.8 Hz, 1H), 8.65 (d, J=2.1 Hz, 1H), 8.56 (t, J=6.7 Hz, 1H), 8.32 (t, J=5.9 Hz, 1H), 8.15-8.08 (m, 2H), 8.00 (t, J=5.8 Hz, 1H), 7.80 (d, J=10.8 Hz, 1H), 7.31 (d, J=3.4 Hz, 1H), 7.28-7.21 (m, 5H), 7.20-7.15 (m, 1H), 7.00 (s, 1H), 6.51 (s, 1H), 5.78-5.71 (m, 1H), 5.41-5.33 (m, 2H), 5.25-5.11 (m, 2H), 4.63-4.60 (m, 2H), 4.58 (d, J=6.7 Hz, 2H), 4.53-4.47 (m, 1H), 3.86-3.83 (m, 2H), 3.74 (td, J=17.3, 5.8 Hz, 2H), 3.67 (d, J=5.7 Hz, 2H), 3.64-3.50 (m, 5H), 3.46 (t, J=5.7 Hz, 2H), 3.18-3.11 (m, 1H), 3.05 (dd, J=14.0, 4.6 Hz, 1H), 2.84-2.75 (m, 1H), 2.40 (s, 3H), 2.33 (t, J=6.5 Hz, 2H), 2.30-2.23 (m, 3H), 1.93-1.78 (m, 2H), 0.90-0.83 (m, 3H).

### Example 14: Synthesis of Compound 58

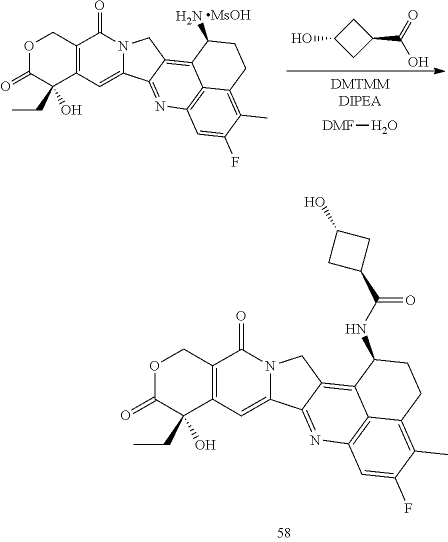

To a 4:1 DMF/water mixture (4 mL) were added exatecan mesylate (20 mg, 0.038 mmol), trans-3-hydroxycyclobutane-1-carboxylic acid (1.25 equiv., 6 mg, 0.048 mmol), DMTMM (2.0 equiv., 21 mg, 0.076 mmol) and diisopropylethylamine (20 μL). The resulting solution was stirred for 1 hour at room temperature, as LC-MS indicated the full consumption of the starting material. The mixture was directly purified by reverse-phase HPLC chromatography using a semipreparative column (diol-modified C18, 0→100% ACN/H₂O). The desired product was obtained as a white powder after lyophilisation (13 mg, 64%). MS calc. for C₂₉H₂₉FN₃O₆: 534.20, found: 534.21, [M+H]⁺. ¹H NMR (401 MHz, DMSO-d₆) δ 8.38 (d, J=8.7 Hz, 1H), 7.76 (d, J=11.0 Hz, 1H), 7.29 (s, 1H), 6.52 (s, 1H), 5.56 (m, 1H), 5.42 (s, 2H), 5.11 (d, J=6.5 Hz, 2H), 5.07 (d, J=6.2 Hz, 1H), 4.37 (m, 1H), 3.15 (m, 2H), 2.91 (m, 1H), 2.50-2.40 (m, 4H), 2.40 (s, 3H), 2.17-2.08 (m, 1H), 2.05-1.99 (m, 1H), 1.86 (p, J=7.0 Hz, 2H), 0.87 (t, J=7.3 Hz, 3H).

### Example 15: Synthesis of Compound 66

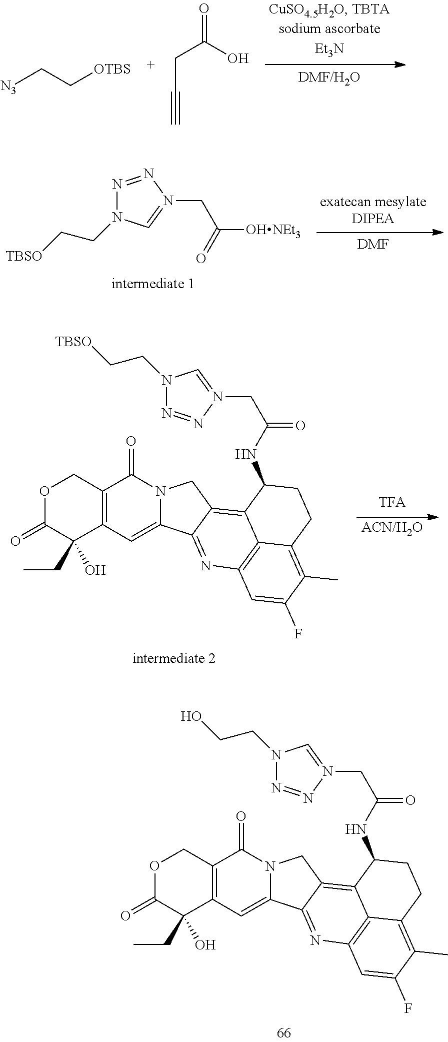

Intermediate 1. To the solution of 3-butynoic acid (1.0 equiv., 17 mg, 0.2 mmol), (2-azidoethoxy)(tert-butyl)dimethylsilane (1.25 equiv., 50 mg, 0.25 mmol) and tris[(1-benzyltriazol-4-yl)methyl]amine (0.15 equiv., 16 mg, 0.03 mmol) in DMF (1 ml) was added triethylamine (1.0 equiv., 0.2 mmol, 20 mg, 28 μL), 1M aqueous CuSO₄·5H₂O (0.1 equiv., 0.02 mmol, 20 μL) and 2M aqueous sodium ascorbate (0.2 equiv., 0.04 mmol, 20 μL) and the resulting mixture was stirred at room temperature for overnight. Purification of the mixture by reverse-phase flash chromatography (25 g, diol-modified C18, 0% to 75% ACN/H₂O) offered the triazole intermediate 1 (47 mg, 61%).

Triazole intermediate 2. To the suspension of exatecan mesylate (10 mg, 0.019 mmol), triazole intermediate 1 (2.0 equiv., 0.038 mmol, 15 mg), N-ethyl-N-(3-dimethylaminopropyl)carbodiimide hydrochloride (2.0 equiv., 0.038 mmol, 8 mg) and 1-hydroxybenzotriazole (2.0 equiv., 0.038 mmol, 5.5 mg) in DMF (0.5 mL) was added diisopropylethylamine (5.0 equiv., 0.095 mmol, 12 mg, 17 μL) under an argon atmosphere and the mixture was stirred at room temperature for 5 hours. LC-MS indicated the full consumption of the starting material. Purification of the mixture by reverse-phase flash chromatography (12 g, diol-modified C18, 0% to 75% ACN/H₂O) offered the triazole intermediate 2 (8 mg, 60%) as a white powder after lyophilisation.

Compound 66. The triazole intermediate 2 (8 mg, 0.011 mmol) was dissolved in ACN/0.1% aq. TFA mixture (1:1, 1 ml), followed by the addition of 2 drops of TFA and the resulting mixture was stirred at room temperature for 2 hours. LC-MS indicated the full consumption of the intermediate 1 and solvents were evaporated under reduced pressure. The residue was purified by reverse-phase flash chromatography using semipreparative column (diol-modified C18, 0% to 50% ACN/H₂O), giving compound 76 (5 mg, 77%) as a white powder after lyophilisation. MS calc. for C₃₀H₃₀FN₆O₆: 589.22, found: 589.30, [M+H]⁺. ¹H NMR (400 MHz, DMSO-d₆) δ: 7.99 (s, 1H), 7.81 (d, J=11.0 Hz, 1H), 7.31 (s, 1H), 5.56 (q, J=4.6 Hz, 1H), 5.43 (s, 2H), 5.26 (d, J=18.9 Hz, 1H), 5.16 (d, J=18.9 Hz, 1H), 4.41-4.34 (m, 2H), 3.80-3.73 (m, 2H), 3.61 (s, 2H), 3.34 (s, 2H), 3.24-3.13 (m, 2H), 2.41 (s, 3H), 2.24-2.07 (m, 2H), 1.95-1.78 (m, 2H), 0.88 (t, J=7.3 Hz, 3H).

### Example 16: Synthesis of Compound 79

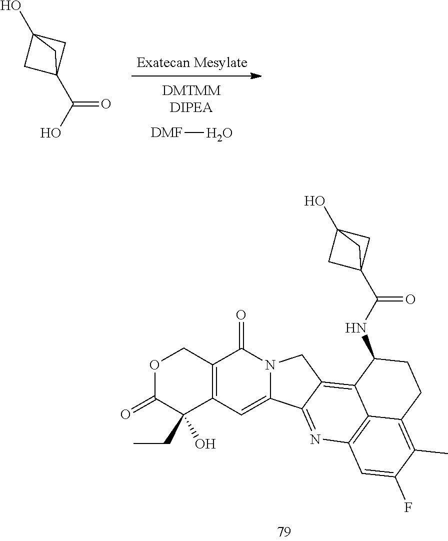

3-Hydroxybicyclo[1.1.1]pentane-1-carboxylic acid (5.8 mg, 0.0451 mmol) and exatecan mesylate (0.8 equiv., 20 mg, 0.0376 mmol) were dissolved in 5 mL of a 1:4 H2O/DMF mixture containing 38 μL of 1M NaOH solution (0.8 equiv. NaOH). The mixture was stirred at room temperature for 1 hour. Solvents were evaporated under reduced pressure to a final volume of approx. 2 mL. The crude reaction mixture was purified by reverse-phase flash chromatography, using a column containing 25 g of diol-modified C₁₈, and using a gradient of ACN in water (0→50% ACN in H₂O). A second purification was performed using a semipreparative column loaded with diol-modified C18, and using a gradient of ACN in water (0→80% ACN in H₂O). The desired product was recovered as a white powder, after lyophilization from water (14 mg, 68%). MS calc. for C₃₀H₂₉FN₃O₆: 546.20, found: 546.22, [M+H]⁺. ¹H NMR (400 MHz, DMSO-d₆) δ 8.42 (d, J=8.9 Hz, 1H), 7.72 (d, J=10.9 Hz, 1H), 7.29 (s, 1H), 6.50 (s, 1H), 6.35 (s, 1H), 5.53 (td, J=8.9, 4.6 Hz, 1H), 5.42 (s, 2H), 5.19-5.08 (m, 1H), 4.96 (d, J=18.8 Hz, 1H), 3.98 (s, 1H), 3.15-3.03 (m, 1H), 2.36 (d, J=1.8 Hz, 3H), 2.22-2.14 (m, 1H), 2.14-2.04 (m, 7H), 1.94-1.79 (m, J=7.1 Hz, 2H), 0.88 (t, J=7.3 Hz, 3H).

### Example 17: Synthesis of Compound 83

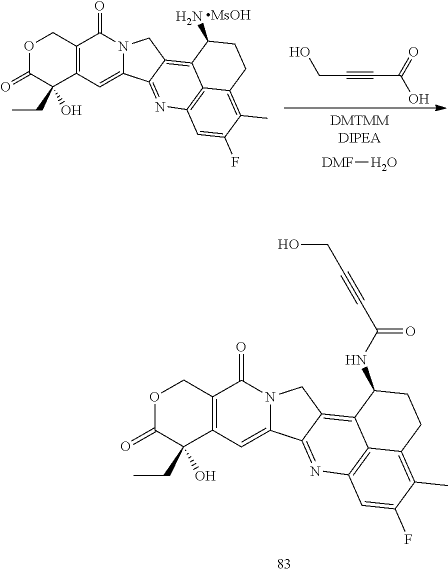

To a 4:1 DMF/water mixture (4 mL) were added exatecan mesylate (20 mg, 0.0376 mmol), 4-hydroxybut-2-ynoic acid (2 eqiuv., 0.0753 mmol, 8 mg), DMTMM (1.5 equiv., 0.0564 mmol, 16 mg) and diisopropylethylamine (20 μL). The resulting solution was stirred for 1 hour at room temperature, as LC-MS indicated the full consumption of the starting material. The mixture was directly purified by reverse-phase HPLC chromatography using a semipreparative column (diol-modified C18, 0→100% ACN/H₂O). The desired product was obtained as a white powder after lyophilisation (15 mg, 76%). MS calc. for C₂₈H₂₅FN₃O₆: 518.17, found: 518.24, [M+H]⁺. ¹H NMR (400 MHz, DMSO-d₆) δ 9.33 (d, J=8.7 Hz, 1H), 7.78 (d, J=10.9 Hz, 1H), 7.30 (s, 1H), 6.52 (s, 1H), 5.58 (dt, J=8.8, 5.4 Hz, 1H), 5.48 (t, J=6.0 Hz, 1H), 5.42 (s, 2H), 5.18 (q, J=18.9 Hz, 2H), 4.23 (d, J=6.0 Hz, 1H), 3.98 (s, 1H), 3.28-3.07 (m, 2H), 2.38 (d, J=1.8 Hz, 3H), 2.18 (q, J=6.2 Hz, 2H), 1.97-1.77 (m, 2H), 0.88 (t, J=7.3 Hz, 3H).

### Example 18: Synthesis of Compound 100

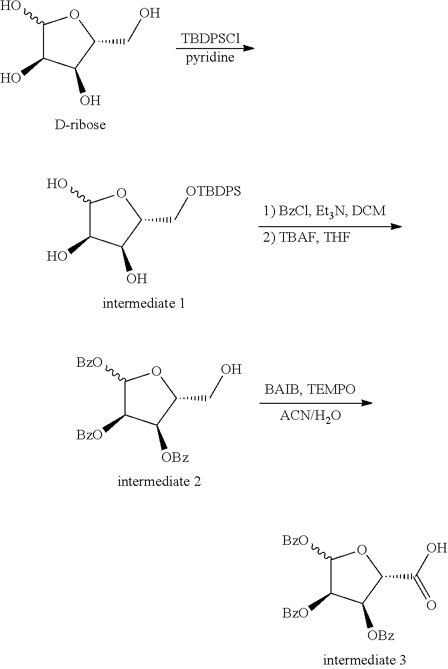

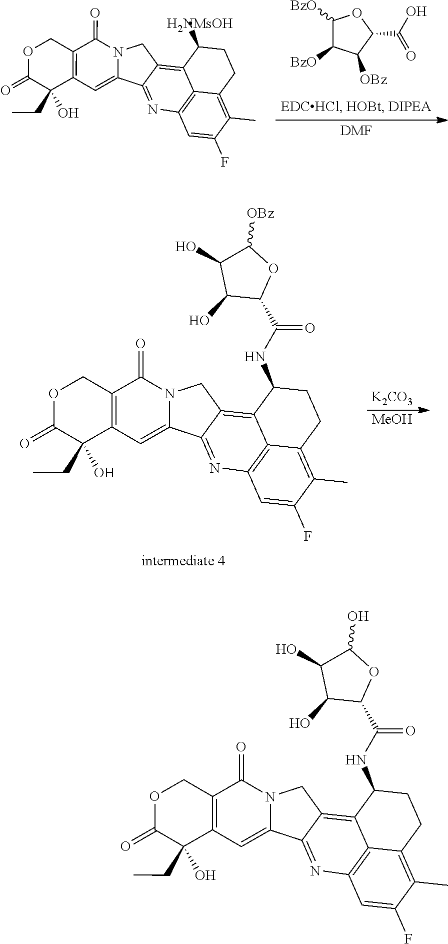

Intermediate 1. The solution of d-ribose (1.00 g, 6.66 mmol) in pyridine was cooled to 0° C. and tert-butyl(chloro)diphenylsilane (1.2 equiv., 2.20 g, 2.08 mL) was added dropwise. The resulting solution was stirred for 2 hours at 0° C. and then at room temperature overnight. Purification by flash chromatography (silica, 0→30% EtOAc/cyclohexane) gave the intermediate 2 (1.73 g, 67%).

Intermediate 2. To the solution of intermediate 1 (1.73 g, 4.45 mmol) in DCM (40 ml) was added triethylamine (6.0 equiv., 26.7 mmol, 2.7 g, 3.72 mL) and the resulting solution was cooled to 0° C. Then, the solution of benzoyl chloride (4.0 equiv., 17.8 mmol, 2.5 g, 2.07 mL) in DCM (10 mL) was added dropwise over 15 minutes and the reaction mixture was allowed to warm to room temperature and stirred overnight. The resulting mixture was then washed with 1M aqueous HCl, saturated NaHCO₃ solution and brine, and the organic phase was dried by Na₂SO₄. Solvents were removed using rotary evaporator and the crude product was dissolved THE (100 ml). To the solution, TBAF (2.2 equiv., 9.6 mmol, 3.0 g) was added in one portion and the resulting solution was stirred for 2 hours at room temperature. Solvents were evaporated and the residue was portioned between EtOAc and saturated NH₄Cl aqueous solution. The organic phase was washed with brine and dried by Na₂SO₄. The crude product was purified by reverse-phase flash chromatography (40 g, diol-modified C18, 0→75% ACN/H₂O) to obtain intermediate 2 (1.27 g, 62%, 2 steps).

Intermediate 3. To the solution of intermediate 2 (1.27 g, 2.75 mmol) in 30% water/CAN (30 ml) were added successively TEMPO (0.1 equiv., 0.275 mmol, 43 mg) and bis(acetoxy)iodobenzene (2.0 equiv., 5.5 mmol, 1.78 g) and the resulting mixture was stirred at room temperature overnight. Solvents were evaporated and the residue was co-evaporated with water 3 times. Then, it was taken up to EtOAc and the organic phase was washed by water, brine, and dried by Na₂SO₄. Removal of solvents offered intermediate 3 (1.27 g, 97%).

Intermediate 4. To the suspension of exatecan mesylate (30 mg, 0.056 mmol), intermediate 3 (3.0 equiv., 0.168 mmol, 80 mg), N-ethyl-N′-(3-dimethylaminopropyl)carbodiimide hydrochloride (2.5 equiv., 0.14 mmol, 27 mg) and 1-hydroxybenzotriazole (2.5 equiv., 0.14 mmol, 19 mg) in DMF (2 mL) was added diisopropylethylamine (6.0 equiv., 0.34 mmol, 44 mg, 59 μL) under an argon atmosphere and the mixture was stirred at room temperature for 1 hour. LC-MS indicated the full consumption of the starting material. Purification of the mixture by reverse-phase flash chromatography (25 g, diol-modified C18, 0→100% ACN/H₂O) offered intermediate 4 (33 mg, 66%) as an off-white powder after lyophilisation.

Compound 100. To the solution of intermediate 4 (33 mg, 0.037 mmol) in methanol (2 ml) was added dry K₂CO₃ (2.0 equiv., 0.074 mmol, 10 mg) and the resulting mixture was stirred at room temperature for 30 minutes. LC-MS indicated the full consumption of the starting material. The mixture was acidified by 0.1% TFA/water and solvents were evaporated under reduced pressure. The residue was purified by reverse-phase flash chromatography using semipreparative column (diol-modified C18, 0→75% ACN/0.1% TFA in H₂O), giving the product (14 mg, 65%, mixture of stereoisomers) as a white powder after lyophilisation. MS calc. for C₂₉H₂₉FN₃O₉: 582.19, found: 582.20, [M+H]⁺. ¹H NMR (400 MHz, DMSO-d₆, major isomer) δ: 7.96 (d, J=9.8 Hz, 1H), 7.79 (d, J=10.9 Hz, 1H), 7.30 (s, 1H), 6.58 (d, J=4.6 Hz, 1H), 6.52 (s, 1H), 5.66-5.52 (m, 1H), 5.42 (s, 2H), 5.25 (d, J=6.8 Hz, 1H), 5.23-5.18 (m, 1H), 5.09 (d, J=4.6 Hz, 1H), 5.06 (dd, J=4.5, 1.7 Hz, 1H), 4.27-4.20 (m, 1H), 4.15 (d, J=5.7 Hz, 1H), 3.69 (td, J=4.6, 1.7 Hz, 1H), 3.17 (t, J=6.5 Hz, 2H), 2.38 (s, 3H), 2.20-2.11 (m, 2H), 1.95-1.77 (m, 2H), 0.87 (t, J=7.3 Hz, 3H).

### Example 19: Synthesis of Compound 103

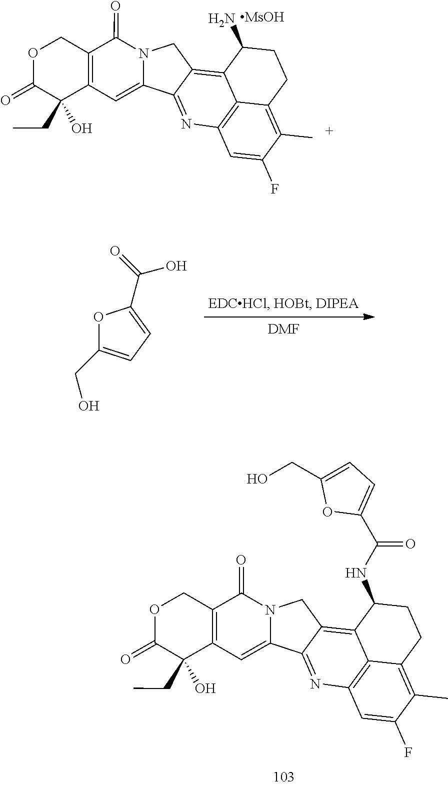

Step 1: Compound 103. To the suspension of exatecan mesylate (10 mg, 0.019 mmol), 5-(hydroxymethyl)furan-2-carboxlic acid (3 equiv., 8 mg, 0.057 mmol), N-ethyl-N-(3-dimethylaminopropyl)carbodiimide hydrochloride (2.5 equiv., 10 mg, 0.048 mmol) and 1-hydroxybenzotriazole (2.5 equiv., 7 mg, 0.048 mmol) in DMF (1 mL) was added diisopropylethylamine (5 equiv., 17 μL, 0.095 mmol) under an argon atmosphere and the mixture was stirred at room temperature for 40 minutes. LC-MS indicated the full consumption of the starting material. Purification by reverse-phase flash chromatography (25 g, diol-modified C18, 0→70% ACN/H₂O) followed by another purification on semipreparative column (diol-modified C18, 0→70% ACN/H₂O) offered the product (7 mg, 66%) as a yellowish powder after lyophilization. MS calc. for C₃₀H₂₇FN₃O₇: 560.18, found: 560.15, [M+H]⁺. ¹H NMR (400 MHz, DMSO-d₆) δ: 8.90 (d, J=8.6 Hz, 1H), 7.79 (d, J=11.0 Hz, 1H), 7.30 (s, 1H), 7.17 (d, J=3.4 Hz, 1H), 6.50 (s, 1H), 6.45 (d, J=3.4 Hz, 1H), 5.77-5.67 (m, 1H), 5.40-5.33 (m, 3H), 5.18 (d, J=18.9 Hz, 1H), 5.10 (d, J=19.0 Hz, 1H), 4.44 (d, J=5.7 Hz, 2H), 3.29-3.09 (m, 1H), 2.40 (d, J=1.9 Hz, 3H), 2.24 (q, J=6.2 Hz, 2H), 0.94-1.75 (m, 2H), 0.86 (t, J=7.3 Hz, 3H).

### Example 20: Synthesis of Compound 1012

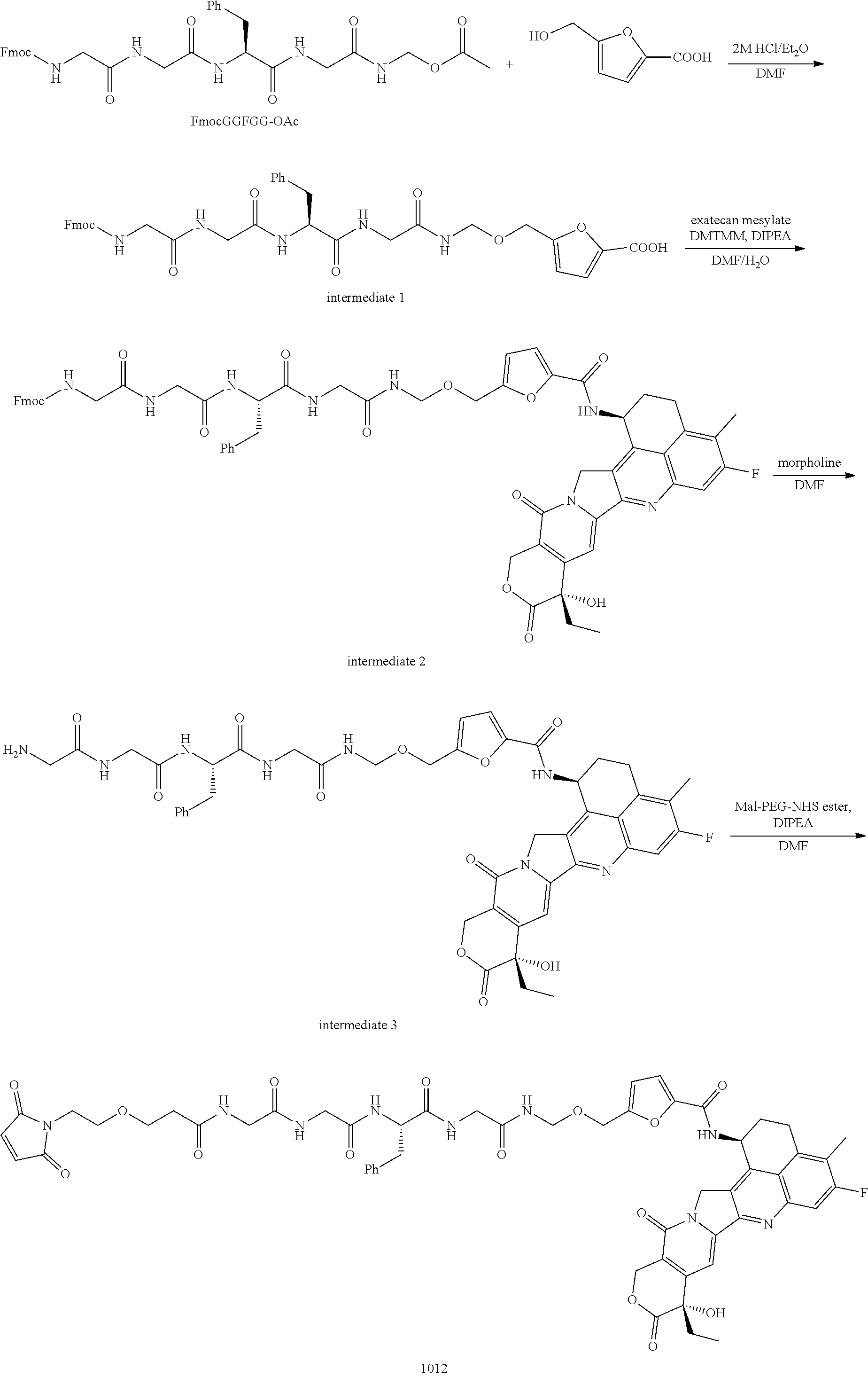

Intermediate 1. To the solution of FmocGGFGG-OAc (1.0 equiv., 50 mg, 0.079 mmol) and 5-(hydroxymethyl)furan-2-carboxylic acid (1.2 equiv., 14 mg, 0.095 mmol) in anhydrous DMF (0.5 ml) was added 2M HCl/Et₂O (70 μL) and the resulting mixture was stirred at room temperature for 2 hours. Volatiles were removed and the residue was purified by reverse-phase flash chromatography (25 g, diol-modified C18, 0→60% ACN/H₂O), giving intermediate 1 as a white solid after lyophilisation (23 mg, 41%). MS calc. for C₃₇H₃₆FN₅O₁₀: 710.25, found: 710.25, [M−H]⁻.

Intermediate 2. The mixture of intermediate 1 (1.05 equiv., 23 mg, 0.032 mmol), exatecan mesylate (1.0 equiv., 16.3 mg, 0.031 mmol) and DMTMM (1.05 equiv., 8.9 mg, 0.032 mmol) was added DMF/water (5:1, 1.2 ml) and diisopropylethylamine (2.1 equiv., 12 μL, 0.065 mmol) and the resulting mixture was stirred at room temperature for 40 minutes, as LC-MS analysis indicated the full consumption of the starting material. The reaction mixture was purified by reverse-phase flash chromatography (25 g, diol-modified C18, 0→75% ACN/H₂O), offering intermediate 2 as a white solid after lyophilisation (27 mg, 77%). MS calc. for C₆₁H₅₈FN₈O₁₃: 1129.41, found: 1129.45, [M+H]⁺.

Intermediate 3. Morpholine (50 μL) was added to the solution of intermediate 2 (1.0 equiv., 27 mg, 0.024 mmol) in anhydrous DMF (1 ml) and the reaction mixture was stirred for 1.5 h at room temperature. LC-MS indicated the full consumption of starting material. The mixture was directly purified by reverse-phase flash chromatography (25 g, diol-modified C18, 0→60% ACN/H₂O), giving the product as a white solid after lyophilisation (15 mg, 69%). MS calc. for C₄₆H₄₈FN₈O₁₁: 907.34, found: 907.35, [M+H]⁺.

Compound 1012. Mal-PEG-NHS ester (1.0 equiv., 0.017 mmol, 5.2 mg) and DIPEA (1.05 equiv., 0.0173 mmol, 3.05 μL) were added to the solution of intermediate 3 (1.0 equiv., 15 mg, 0.017 mmol) in anhydrous DMF (1 ml). The reaction mixture was stirred at room temperature for 1.5 h, as LC-MS indicated the full consumption of starting material. Purification by reverse-phase flash HPLC using a semipreparative column (diol-modified C18, 0→60% ACN/H₂O) offered the desired product as a white solid after lyophilization (11 mg, 60%). MS calc. for C₅₅H₅₇FN₉O₁₅: 1102.40, found: 1102.45, [M+H]⁺. ¹H NMR (500 MHz, DMSO-d₆) δ: 9.06-8.93 (m, 1H), 8.59 (t, J=6.4 Hz, 1H), 8.31 (t, J=5.9 Hz, 1H), 8.16-8.05 (m, 2H), 8.04-7.94 (m, 1H), 7.80 (d, J=10.8 Hz, 1H), 7.30 (s, 1H), 7.27-7.11 (m, 7H), 6.99 (s, 1H), 6.59 (d, J=3.4 Hz, 1H), 6.51 (s, 1H), 5.78-5.63 (m, 1H), 5.38 (s, 2H), 5.28-4.97 (m, 2H), 4.59 (d, J=7.0 Hz, 2H), 4.53-4.37 (m, 3H), 3.84-3.63 (m, 5H), 3.63-3.48 (m, 5H), 3.45 (t, J=5.8 Hz, 2H), 3.29-3.22 (m, 1H), 3.20-3.08 (m, 1H), 3.04 (dd, J=13.9, 4.6 Hz, 1H), 2.79 (dd, J=13.9, 9.6 Hz, 1H), 2.40 (s, 3H), 2.32 (t, J=6.6 Hz, 2H), 2.27-2.19 (m, 2H), 1.96-1.77 (m, 2H), 0.86 (t, J=7.5 Hz, 3H).

### Example 21: Synthesis of Compound 105

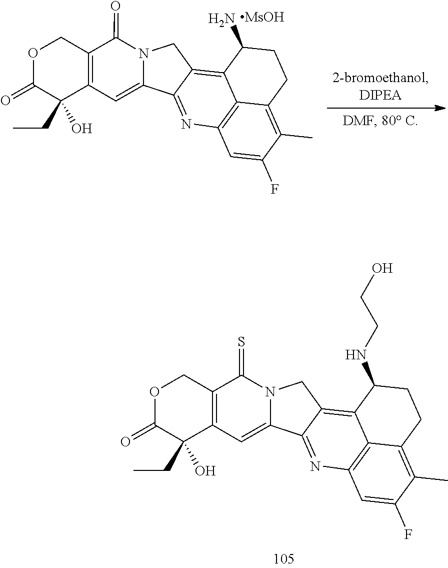 [SMILES: "·MsOH" ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=S)Cc2c-1nc1cc(F)c(C)c3c1c2[C@@H](NCCO)CC3]

Compound 105. To the suspension of exatecan mesylate (30 mg, 0.056 mmol) in DMF (1 mL) were added diisopropylethylamine (3.5 equiv., 0.196 mmol, 34 μL) and 2-bromoethanol (2 equiv., 0.112 mmol, 14 mg, 8 μL) under an argon atmosphere and the mixture was heated to 80° C. for 2 days. LC-MS indicated the full consumption of the starting material. Purification by reverse-phase flash chromatography on semipreparative column (diol-modified C18, 0→50% ACN/1% TFA in H₂O) offered the product (16 mg, 48%) as a white powder after lyophilization. MS calc. for C₂₆H₂₇FN₃O₅: 480.19, found: 480.25, [M+H]⁺. ¹H NMR (400 MHz, DMSO-d₆) δ: 9.03 (s br, 1H), 8.80 (s br, 1H), 7.88 (d, J=10.8 Hz, 1H), 7.34 (s, 1H), 6.57 (s, 1H), 5.57-5.36 (m, 4H), 5.30 (s, 1H), 5.16-5.02 (m, 1H), 3.69 (t, J=5.5 Hz, 2H), 3.31-3.06 (m, 3H), 2.84-2.71 (m, 1H), 2.41 (s, 3H), 2.26-2.10 (m, 1H), 1.99-1.76 (m, 2H), 0.87 (t, J=7.3 Hz, 3H).

### Example 22: Synthesis of Compound 1013

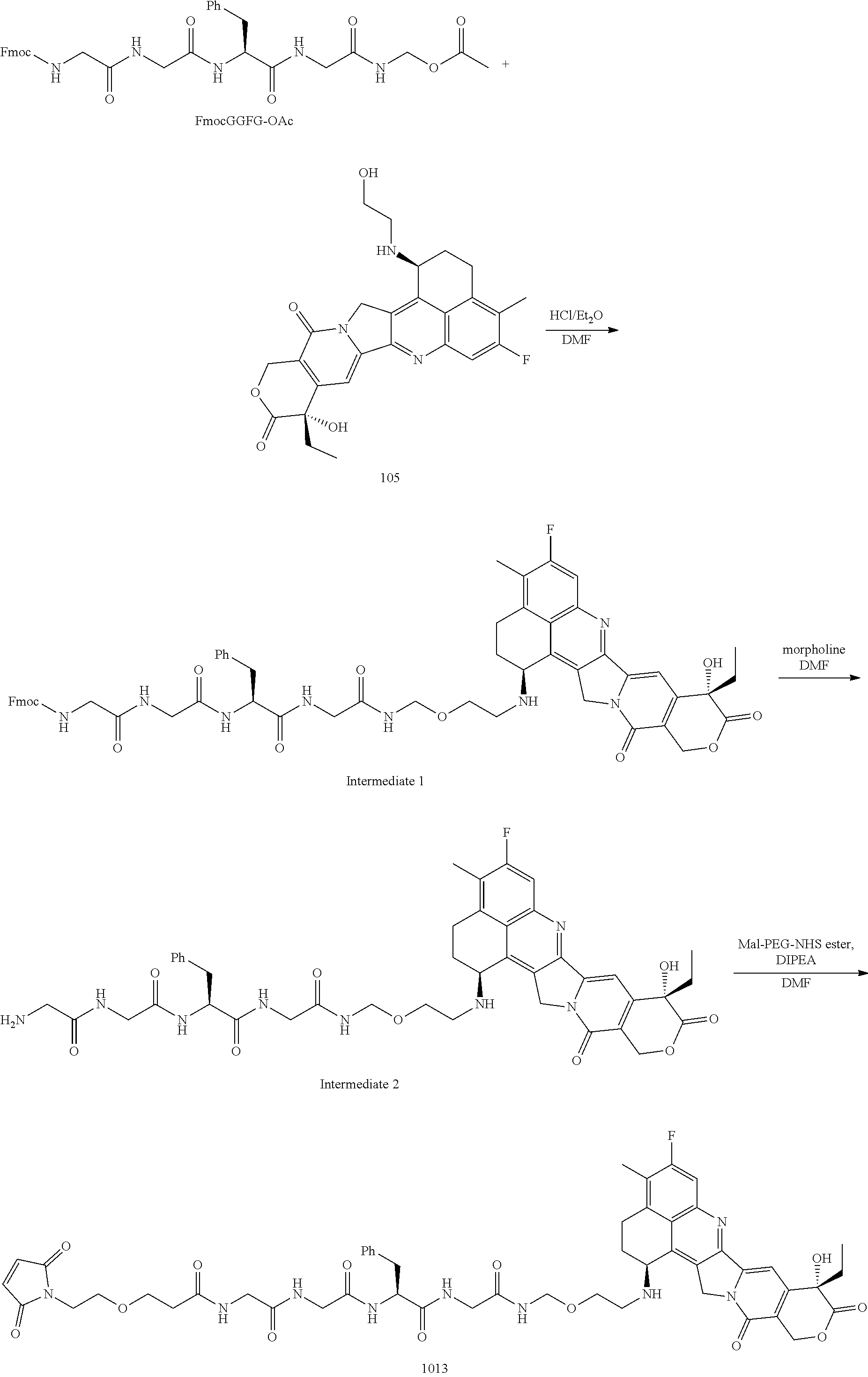

Intermediate 1. The mixture of compound 105 (1.0 equiv., 20 mg, 0.034 mmol) and FmocGGFG-OAc (2.0 equiv., 0.068 mmol, 43 mg) was dissolved in anhydrous DMF (1 mL), followed by the addition of 2M HCl/Et₂O (100 μL). The reaction mixture was stirred at room temperature for 4 h and during that time, several portions (ca. 0.5 equiv. each) of FmocGGFG-OAc were added to the reaction mixture. Then the reaction mixture was purified by reverse-phase flash chromatography (25 g, diol-modified C18, 0→75% ACN/H₂O). Fractions containing the product were lyophilised and the residue (product+co-eluting impurities) was used directly into next step. MS calc. for C₅₇H₅₈FN₈O₁₁: 1049.42, found: 1049.40, [M+H]⁺.

Intermediate 2. Morpholine (80 μL) was added to the solution of intermediate 1 (as obtained in previous step) in anhydrous DMF (2 ml) and the reaction mixture was stirred for 1,5 h at room temperature. LC-MS indicated the full consumption of starting material. The mixture was directly purified by reverse-phase flash chromatography (25 g, diol-modified C18, 0→60% ACN/H₂O), giving the product as a white solid after lyophilization (12 mg, 43%). MS calc. for C₄₂H₄₈FN₈O₉: 827.35, found: 827.30, [M+H]⁺.

Compound 1013. Mal-PEG-NHS ester (1.0 equiv., 0.015 mmol, 4.3 mg) and DIPEA (2.2 equiv., 0.032 mmol, 4.2 mg, 5.7 μL) were added to the solution of intermediate 2 (12 mg, 0.015 mmol) in anhydrous DMF (1 ml). The reaction mixture was stirred at room temperature for 40 minutes, as LC-MS indicated the full consumption of starting material. Purification by reverse-phase flash HPLC using a semipreparative column (diol-modified C18, 0→60% ACN/H₂O) offered the desired product as a white solid after lyophilization (6.3 mg, 41%). MS calc. for C₅₁H₅₇FN₉O₁₃: 1022.41, found: 1022.40, [M+H]⁺. ¹H NMR (500 MHz, DMSO-d₆) δ: 8.65 (t, J=6.7 Hz, 1H), 8.33-8.26 (m, 1H), 8.15-8.07 (m, 2H), 8.03-7.96 (m, 1H), 7.76-7.70 (m, 1H), 7.60 (s, 1H), 7.26-7.18 (m, 5H), 7.18-7.12 (m, 1H), 6.99 (s, 2H), 5.49-5.24 (m, 3H), 4.81 (d, J=11.8 Hz, 1H), 4.71-4.55 (m, 3H), 4.48 (ddd, J=9.6, 8.1, 4.5 Hz, 1H), 4.28 (t, J=4.2 Hz, 1H), 3.81-3.67 (m, 3H), 3.65 (d, J=5.7 Hz, 2H), 3.62-3.47 (m, 6H), 3.47-3.41 (m, 2H), 3.22-3.12 (m, 1H), 3.06-2.90 (m, 2H), 2.87-2.71 (m, 2H), 2.38-2.29 (m, 5H), 2.26-1.98 (m, 4H), 1.26-1.16 (m, 1H), 0.90-0.82 (m, 3H).

### Example 23: Synthesis of Compound 106

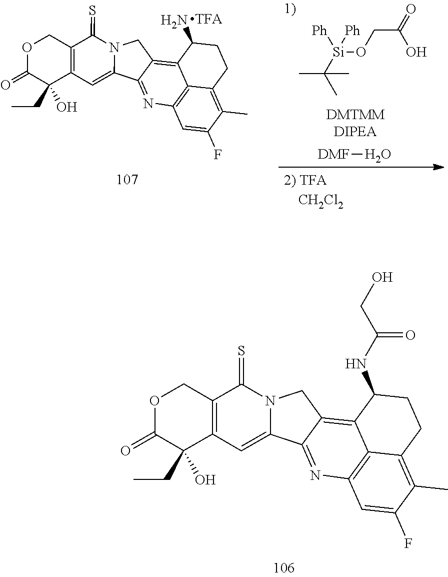

Compound 106. Compound 107 trifluoroacetate (20 mg, 0.0364 mmol), silyl protected glycolic acid (1 equiv., 0.0364 mmol, 11.5 mg), and DMTMM (1 equiv., 0.0364 mmol, 10 mg) were dissolved in 2 mL of a 1:4 water/DMF mixture. DIPEA (20 μL) was added and the reaction mixture was stirred at room temperature for 1 h, as LCMS analysis showed full conversion. The solvents were evaporated under vacuum and the crude reaction mixture was re-dissolved in 1 mL of DCM and 3 mL of TFA. After stirring for 16 h at room temperature, solvents were evaporated and the crude reaction product was re-dissolved in 1 mL of DMF to be directly purified by reverse-phase semipreparative flash chromatography (diol-modified C18, 0→80% ACN/water). The desired product was obtained as a 1:1 mixture of two inseparable isomers, with both forms in equilibrium (10 mg, 54%, orange solid). MS calc. for C₂₆H₂₅FN₃O₅S: 510.15, found: 510.44, [M+H]⁺. ¹H NMR (400 MHz, DMSO-d₆) δ 8.51 (dd, J=15.9, 9.1 Hz, 1H), 7.83-7.74 (m, 2H), 7.63-7.55 (m, 1H), 7.39-7.24 (m, 1H), 6.69 (s, 1H), 5.92 (m, 1H), 5.64 (d, J=6.7 Hz, 1H), 5.56-5.44 (m, 2H), 5.30 (t, J=19.0 Hz, 1H), 4.17-3.99 (m, 1H), 3.26 (m, 1H), 3.19-3.08 (m, 1H), 2.60-2.52 (m, 1H), 2.38 (d, J=1.9 Hz, 3H), 2.21 (d, J=7.2 Hz, 2H), 1.94-1.83 (m, 1H), 0.91-0.81 (m, 3H).

### Example 24: Synthesis of Compound 107

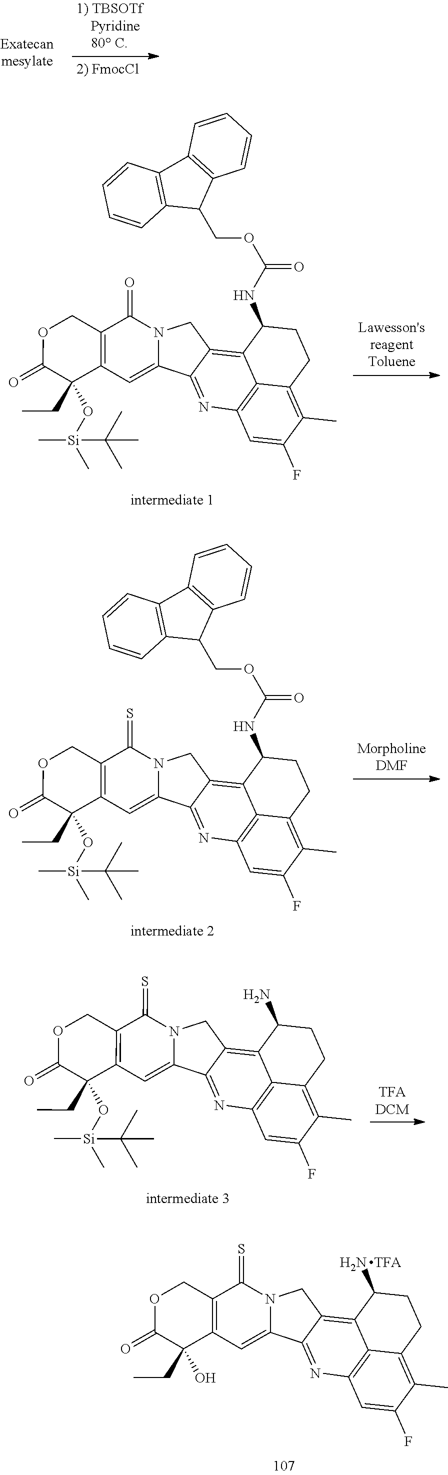 [SMILES: CC[C@@]1(O[Si](C)(C)C(C)(C)C)C(=O)OCc2c1cc1n(c2=O)Cc2c-1nc1cc(F)c(C)c3c1c2[C@@H](NC(=O)OCC1c2ccccc2-c2ccccc21)CC3 ; CC[C@@]1(O[Si](C)(C)C(C)(C)C)C(=O)OCc2c1cc1n(c2=S)Cc2c-1nc1cc(F)c(C)c3c1c2[C@@H](NC(=O)OCC1c2ccccc2-c2ccccc21)CC3 ; CC[C@@]1(O[Si](C)(C)C(C)(C)C)C(=O)OCc2c1cc1n(c2=S)Cc2c-1nc1cc(F)c(C)c3c1c2[C@@H](N)CC3 ; CC[C@@]1(O)C(=O)OCc2c1cc1n(c2=S)Cc2c-1nc1cc(F)c(C)c3c1c2[C@@H](N)CC3]

Intermediate 1. Exatecan mesylate (52 mg, 0.0978 mmol) was dissolved in 3 mL of anhydrous pyridine and heated at 80° C. Tert-Butyldimethylsilyl trifluoromethanesulfonate (10 equiv., 0.978 mmol, 259 mg) was added and the reaction mixture was stirred at 80° C. for 3 h, as LCMS analysis confirmed full conversion into the product. The reaction mixture was allowed to cool to room temperature, and 9-fluorenylmethyloxycarbonyl chloride (2 equiv., 0.196 mmol, 51 mg) was added. The reaction mixture was stirred at room temperature for 2 h. The residue was purified by reverse-phase flash chromatography (diol-modified C18, 0→100% ACN), giving the desired intermediate 1 (45 mg, 60%) as a pale yellow solid. MS calc. for C₄₅H₄₇FN₃O₆Si: 772.32, found: 772.30, [M+H]⁺.

Intermediate 2. The previously prepared intermediate 1 (0.0583 mmol, 45 mg) and Lawesson's reagent (5 equiv., 0.146 mmol, 59 mg) were dissolved in toluene (5 mL) ad the reaction mixture was stirred at 100° C. for 3 h. The solvent was evaporated and the residue was directly purified by reverse-phase flash chromatography (diol-modified C18, 0→100% ACN/water), giving the desired product intermediate 2 (41 mg, 89%) as a yellow solid. MS calc. for C₄₅H₄₇FN₃O₅SSi: 788.30, found: 788.33, [M+H]⁺.

Intermediate 3. The previously prepared intermediate 2 (41 mg, 0.0520 mmol) was dissolved into 2 mL of anhydrous DMF and morpholine (100 μL) was added. The reaction mixture was stirred at room temperature for 1 h. The residue was directly purified by reverse-phase flash chromatography (diol-modified C18, 0→100% CAN in water), giving intermediate 3 (23 mg, 79%) as a bright yellow solid. MS calc. for C₃₀H₃₇FN₃O₃SSi: 566.23, found: 566.45, [M+H]⁺.

Compound 107 TFA. The previously prepared intermediate 3 (23 mg, 0.0407 mmol) was dissolved in 2 mL of anhydrous dichloromethane and 2 mL of trifluoroacetic acid were added. The reaction mixture was stirred at room temperature for 12 hours. Solvents were evaporated under reduced pressure and the crude reaction product was re-dissolved in 2 mL of DMF to be directly purified by reverse-phase flash chromatography (diol-modified C18, 0→100% ACN/water). A second reverse-phase HPLC purification (semipreparative HPLC, diol-modified C18, 0→100% ACN in water) afforded compound 107 trifluoroacetate as a 1:1 mixture of two inseparable isomers, with both forms in equilibrium (11 mg, 60%, bright yellow solid). MS calc. for C₂₄H₂₃FN₃O₃S: 452.14, found: 452.15, [M+H]⁺. ¹H NMR (500 MHz, DMSO-d₆) δ 8.55 (d, J=5.8 Hz, 3H), 7.97-7.91 (m, 1H), 7.84 (s, 1H), 7.32 (d, J=37.9 Hz, 1H), 6.70 (d, J=26.1 Hz, 1H), 6.07-5.91 (m, 2H), 5.71 (d, J=20.1 Hz, 1H), 5.56 (dd, J=16.6, 3.7 Hz, 1H), 3.32 (m, 1H), 3.14 (m, 1H), 2.44 (s, 3H), 1.91 (h, J=6.9 Hz, 2H), 1.26 (q, J=7.1 Hz, 2H), 0.88 (t, J=7.3 Hz, 3H).

### Example 25: Synthesis of Compound 108

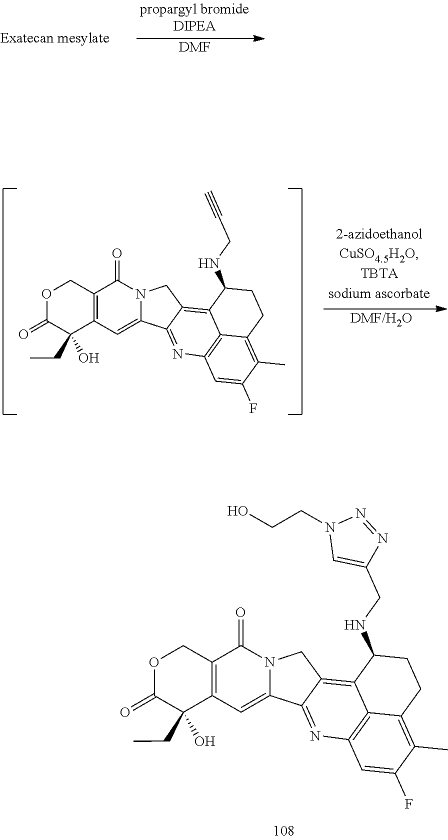

Diisopropylethylamine (2.5 equiv., 0.07 mmol, 9 mg, 13 μL) and propargyl bromide (2.5 equiv., 0.07 mmol, 8.5 mg, 9 μL 80% solution in toluene) were added to the suspension of exatecan mesylate (1.0 equiv., 15 mg, 0.028 mmol) in DMF (0.2 ml) and the resulting solution was stirred for 48 hours. Then, 2-azidoethanol (5.0 equiv., 0.14 mmol, 12 mg, 11 μL), tris(benzyltriazolylmethyl)amine (1.5 equiv., 0.042 mmol, 22 mg), CuSO4·5H2O (1.0 equiv., 0.028 mmol, 140 μL 2M aqueous solution) and sodium ascorbate (2.0 equiv., 0.056 mmol, 56 μL 1M aqueous solution) were successively added to the reaction mixture and the solution was stirred overnight. The crude reaction mixture was directly loaded on column and purified by reverse-phase flash chromatography (25 g, diol-modified C18, 0% to 50% ACN/H₂O), offering compound 108 (12 mg, 77%) as a white powder after lyophilisation. MS calc. for C₂₉H₃₀FN₆O₅: 561.23, found: 561.30, [M+H]⁺. ¹H NMR (400 MHz, DMSO-d₆) δ: 7.97 (s, 1H), 7.72 (d, J=11.0 Hz, 1H), 7.29 (s, 1H), 5.43 (s, 2H), 5.28 (d, J=19.0 Hz, 1H), 5.19 (d, J=19.0 Hz, 1H), 4.39 (t, J=5.5 Hz, 2H), 4.25 (t, J=4.1 Hz, 1H), 3.97 (q, J=13.9 Hz, 2H), 3.80-3.75 (m, 2H), 3.33 (s, 2H), 3.24 (ddd, J=15.8, 10.4, 4.3 Hz, 1H), 3.01 (dt, J=16.8, 4.8 Hz, 1H), 2.39-2.27 (m, 6H), 2.09-1.98 (m, 1H), 1.95-1.78 (m, 2H), 0.87 (t, J=7.3 Hz, 3H).

### Example 26: Synthesis of Compound 1015

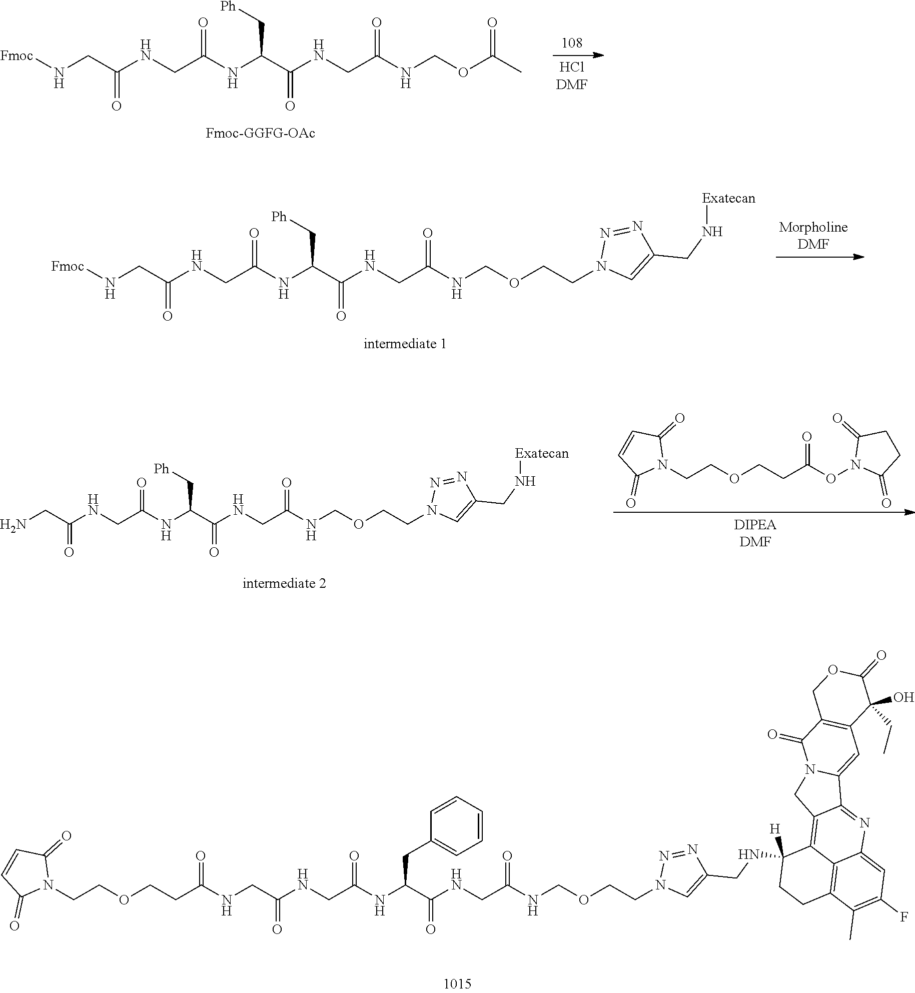

Intermediate 1. Compound 108 (1.0 equiv., 30 mg, 0.0536 mmol) and FmocGGFG-OAc (2.0 equiv., 0.107 mmol, 67 mg) were dissolved in anhydrous DMF (1.5 mL), followed by the addition of 2M HCl/Et₂O (150 μL). The reaction mixture was stirred at room temperature for 4 h and during that time, several portions (ca. 0.5 equiv. each) of FmocGGFG-OAc were added to the reaction mixture. Then the reaction mixture was purified by reverse-phase flash chromatography (25 g, diol-modified C18, 0→75% ACN/H₂O). Fractions containing the product were lyophilised and the residue (product+co-eluting impurities) was used directly into next step. MS calc. for C₆₀H₆₁FN₁₁O₁₁: 1130.45, found: 1130.40, [M+H]⁺.

Intermediate 2. Morpholine (100 μL) was added to the solution of intermediate 1 (as obtained in previous step) in anhydrous DMF (2 ml) and the reaction mixture was stirred for 1 h at room temperature. LC-MS indicated the full consumption of starting material. The mixture was directly purified by reverse-phase flash chromatography (25 g, diol-modified C18, 0→50% ACN/H₂O), giving the product as a white solid after lyophilisation (14 mg, 29% over 2 steps). MS calc. for C₄₅H₅₁FN₁₁O₉: 908.39, found: 908.55, [M+H]⁺.

Compound 1015. Mal-PEG-NHS ester (1 equiv., 0.0154 mmol, 5 mg) and DIPEA (2.5 equiv., 0.039 mmol, 5 mg, 6.6 μL) were added to the solution of intermediate 2 (1 equiv., 14 mg, 0.0154 mmol) in anhydrous DMF (1 ml). The reaction mixture was stirred at room temperature for 30 minutes, as LC-MS indicated the full consumption of starting material. Purification by reverse-phase flash HPLC using a semipreparative column (diol-modified C18, 0 →100% CAN in H₂O) offered the desired product as a white solid after lyophilisation from water-acetonitrile (7 mg, 41%). MS calc. for C₅₄H₆₀FN₁₂O₁₃: 1103.44, found: 1103.61, [M+H]⁺. ¹H NMR (500 MHz, DMSO-d₆) δ 8.54 (m, 1H), 8.30 (m, 1H), 8.12 (m, 2H), 8.01 (m, 1H), 7.97 (s, 1H), 7.73 (m, 1H), 7.65 (s, 1H), 7.29 (s, 1H), 7.25-7.17 (m, 4H), 7.17-7.12 (m, 1H), 7.01 (d, J=5.9 Hz, 1H), 6.99 (s, 2H), 6.51 (s, 1H), 5.43 (s, 1H), 5.34-5.06 (m, 2H), 4.55 (m, 2H), 4.51 (m, 1H), 4.49-4.43 (m, 1H), 4.25 (d, J=17.1 Hz, 1H), 4.10-3.88 (m, 2H), 3.80 (q, J=4.9 Hz, 2H), 3.76-3.63 (m, 5H), 3.45 (t, J=5.9 Hz, 2H), 3.22 (d, J=10.9 Hz, 1H), 3.04-2.98 (m, 2H), 2.87 (t, J=6.0 Hz, 1H), 2.82-2.73 (m, 2H), 2.59 (s, 1H), 2.36 (m, 4H), 2.32 (m, 2H), 2.06-1.97 (m, 2H), 1.86 (m 1H), 1.29-1.21 (m, 2H), 0.86 (m, 3H).

### Example 27: Synthesis of Compound 109

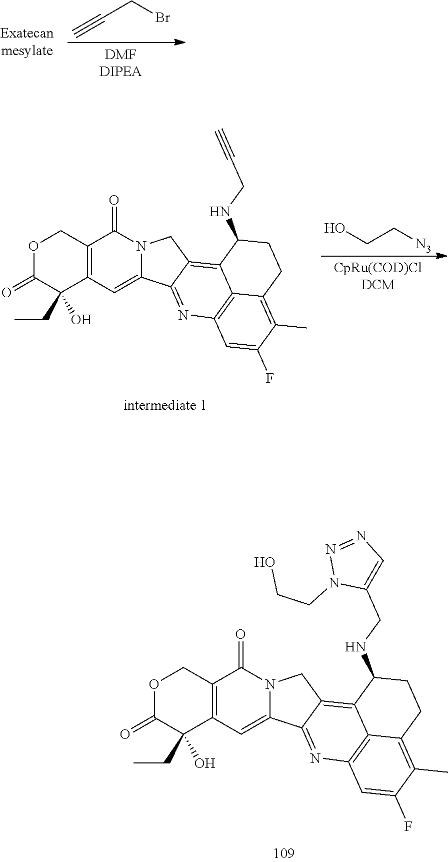

Intermediate 1. Exatecan mesylate (20 mg, 0.0376 mmol) was dissolved in 2 mL of DMF containing diisopropylethylamine (5 equiv., 0.188 mmol, 33 μL). The reaction mixture was stirred at room temperature overnight. The crude reaction mixture was directly loaded on column and purified by reverse-phase flash chromatography, using a column containing 25 g of diol-modified C18, and using a gradient of ACN in water (0% to 60% ACN in H₂O). The desired product was recovered as a white powder, after lyophilization from water (16 mg, 76%). MS calc. for C₂₇H₂₅FN₃O₄: 474.51, found: 474.66, [M+H]⁺.

Compound 109. The previously prepared intermediate 1 (16 mg, 0.0286 mmol) and CpRu(COD)cl (0.1 equiv., 1.3 mg, 0.0029 mmol) were suspended in 10 mL of anhydrous dichloromethane. 2-azidoethanol (3.2 equiv., 0.115 mmol, 10 mg) was added and the mixture was refluxed at 50° C. for 16 hours. The solvent was evaporated and the crude product re-dissolved in DMF (2 mL). The catalyst was filtered off using a 2 μm syringe filter, thus the crude reaction mixture was purified by reverse-phase HPLC, using a semipreparative column containing diol-modified C18, and using a gradient of ACN in water (0% to 80% ACN in H₂O). The desired product was recovered as a white powder, after lyophilization from water (8 mg, 50%). MS calc. for C₂₉H₃₀FN₆O₅: 561.23, found: 561.75, [M+H]⁺. ¹H NMR (400 MHz, DMSO-d₆) δ 7.74 (d, J=11.0 Hz, 1H), 7.69-7.62 (m, 1H), 7.30 (s, 1H), 6.52 (s, 1H), 5.43 (d, J=3.5 Hz, 2H), 5.41-5.28 (m, 1H), 4.44 (m, 2H), 4.30 (t, J=4.6 Hz, 1H), 4.16 (d, J=14.3 Hz, 1H), 4.02 (d, J=14.3 Hz, 1H), 3.76 (t, J=5.5 Hz, 2H), 3.27-3.19 (m, 1H), 3.02 (m, 1H), 2.68 (m, 2H), 2.37 (m, 3H), 2.25 (m, 2H), 2.18-1.99 (m, 1H), 1.86 (m, 2H), 0.86 (dt, J=9.2, 7.3 Hz, 3H).

### Example 28: Synthesis of Compound 110

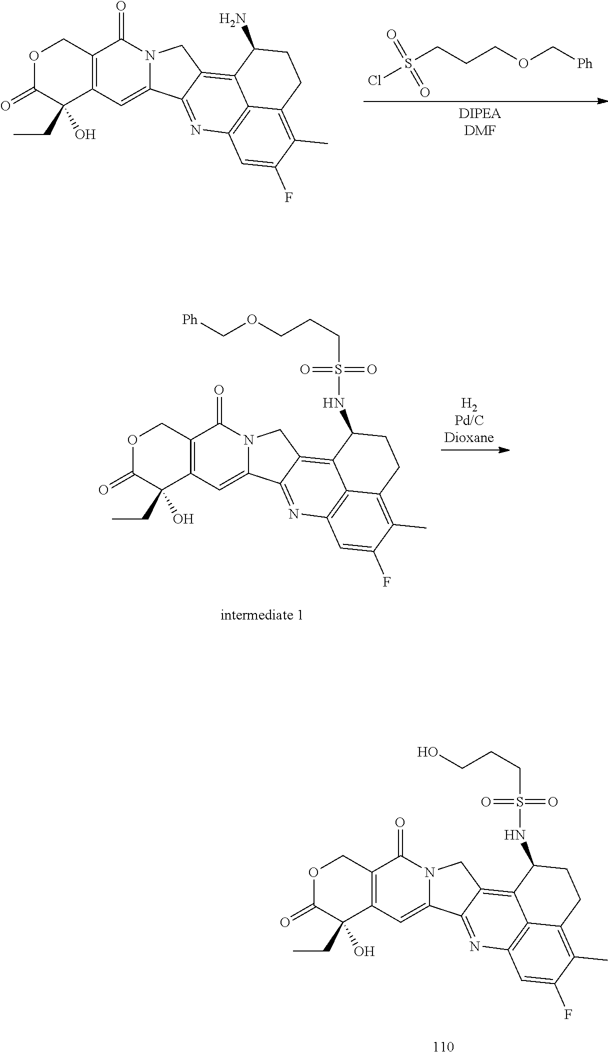 [SMILES: CC[C@@]1(O)C(=O)OCc2c1cc1n(c2=O)Cc2c-1nc1cc(F)c(C)c3c1c2[C@@H](N)CC3 ; O=S(=O)(Cl)CCCOCc1ccccc1 ; CC[C@@]1(O)C(=O)OCc2c1cc1n(c2=O)Cc2c-1nc1cc(F)c(C)c3c1c2[C@@H](NS(=O)(=O)CCCOCc1ccccc1)CC3 ; CC[C@@]1(O)C(=O)OCc2c1cc1n(c2=O)Cc2c-1nc1cc(F)c(C)c3c1c2[C@@H](NS(=O)(=O)CCCO)CC3]

Intermediate 1. Exatecan mesylate (30 mg, 0.066 mmol) and diisopropylethylamine (2.5 equiv., 0.164 mmol, 29 μL) were dissolved in 2 mL of DMF. 3-(Benzyloxy)propane-1-sulfonyl chloride (1.2 equiv., 0.788 mmol, 196 mg) was added and the reaction mixture was stirred for 3 hours at room temperature. Solvents were evaporated under reduced pressure, and the crude reaction mixture was purified by reverse-phase flash chromatography, using a column containing 25 g of diol-modified C18, and using a gradient of ACN in 1% TFA (0→60% ACN in 1% TFA). The desired product was recovered as a white powder, after lyophilization from water (16 mg, 37%). MS calc. for C₃₄H₃₅FN₃O₇S: 648.22, found: 647.99 [M+H]⁺.

Compound 110. The previously synthesized intermediate 1 (16 mg, 0.0247 mmol) was dissolved in 3 mL of dioxane. Pd/C (10% w/w, 5 mg) was suspended in the mixture, and H₂ was bubbled using a balloon into the suspension, while stirring at room temperature for 2 h. The suspension was taken with a syringe and filtrate through 0.2 μm syringe filter. Dioxane was evacuated under reduced pressure and the crude product was redissolved in 2 mL of DMF and purified by reverse-phase flash HPLC, using a semipreparative column containing diol-modified C18, and using a gradient of ACN in water (0→80% ACN in water). The desired product was recovered as a yellow solid, after lyophilization from water-dioxane (6 mg, 44%). MS calc. for C₂₇H₂₉FN₃O₇S: 558.17, found: 558.66 [M+H]⁺. ¹H NMR (400 MHz, DMSO-d₆) δ 7.78 (dd, J=11.0, 6.5 Hz, 1H), 7.31 (s, 1H), 5.46-5.38 (m, 3H), 5.08 (t, J=5.2 Hz, 1H), 3.55 (td, J=6.1, 2.3 Hz, 2H), 3.32-3.28 (m, 2H), 3.16 (dt, J=16.6, 5.9 Hz, 1H), 2.68 (p, J=1.8 Hz, 1H), 2.40-2.35 (m, 4H), 2.33 (m, 2H), 2.26 (q, J=6.2 Hz, 2H), 1.89 (m, 4H), 1.76 (s, 1H), 0.88 (t, J=7.3 Hz, 3H).

### Example 29: Synthesis of Compound 111

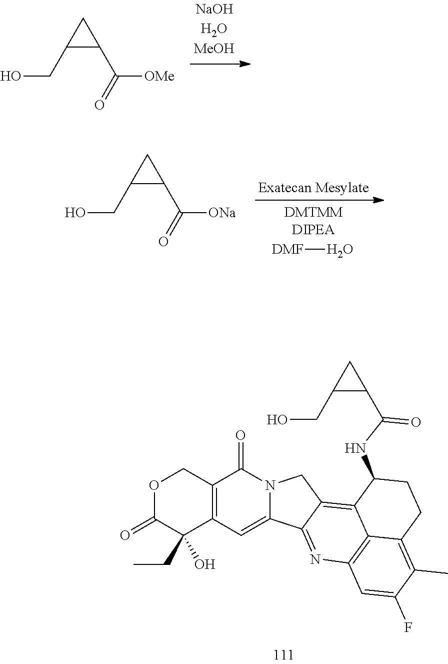

Compound 111. Methyl 2-(hydroxymethyl)cyclopropane-1-carboxylate (25 mg, 0.175 mmol) was dissolved in 1 mL of methanol, and 870 μL of 1M NaOH (1 equiv.) were added. The mixture was stirred at room temperature for 5 h, thus, the solvents were evaporated and the crude product was lyophilized from water. To the obtained solid were added exatecan mesylate (46 mg, 0.5 equiv., 0,874 mmol), DMTMM (48 mg, 1 equiv., 0.175 mmol), and 10 mL of a 4:1 DMF/water mixture. The mixture was stirred at room temperature for 30 min. Solvents were evaporated under reduced pressure to a final volume of approx. 2 mL. The crude reaction mixture was purified by reverse-phase flash chromatography, using a column containing 25 g of diol-modified C18, and using a gradient of ACN in water (0→60% ACN in H₂O). A second purification was performed using a semipreparative column loaded with diol-modified C18, and using a gradient of ACN in water (0→80% ACN in H₂O). Two isomers were separated during the semipreparative purification. The product were recovered separately as white powders, after lyophilization from water/dioxane (42 mg total, 91% calculated from exatecan). MS calc. for C₂₉H₂₉FN₃O₆: 534.20, found: 534.10, [M+H]⁺.

For isomer A: ¹H NMR (400 MHz, DMSO-d₆) δ 8.67 (d, J=8.8 Hz, 1H), 7.78 (d, J=11.0 Hz, 1H), 7.30 (s, 1H), 6.52 (s, 1H), 5.56 (q, J=6.6 Hz, 1H), 5.42 (s, 2H), 5.16 (d, J=2.9 Hz, 2H), 4.63 (t, J=5.5 Hz, 1H), 3.48-3.38 (m, 1H), 3.31-3.25 (m, 2H), 3.21-3.08 (m, 1H), 2,30 (m, 2H), 2.24-2.07 (m, 2H), 1.96-1.77 (m, 2H), 1.58 (d, J=4.4 Hz, 1H), 1.49 (m, 1H), 0.99 (dt, J=8.4, 4.3 Hz, 1H), 0.88 (t, J=7.3 Hz, 3H), 0.71 (m, 1H).

For isomer B (contains 7% of isomer A, based on NMR integrations): ¹H NMR (400 MHz, DMSO-d₆) δ 8.72 (d, J=8.9 Hz, 1H), 7.77 (d, J=10.9 Hz, 1H), 7.32 (s, 1H), 6.53 (s, 1H), 5.56 (m, 2H), 5.44 (s, 2H), 5.31-5.05 (m, 2H), 4.52 (dd, J=6.1, 5.0 Hz, 1H), 3.45 (m 1H), 3.26 (m, 1H), 3.20-3.08 (m, 1H), 2.38 (m, 2H), 2.24-2.00 (m, 2H), 1.87 (m, 2H), 1.76 (s, 1H), 1.60-1.48 (m, 1H), 1.01 (m, 1H), 0.88 (t, J=7.3 Hz, 3H), 0.75 (m, 1H).

### Example 30: Synthesis of Compound 1016

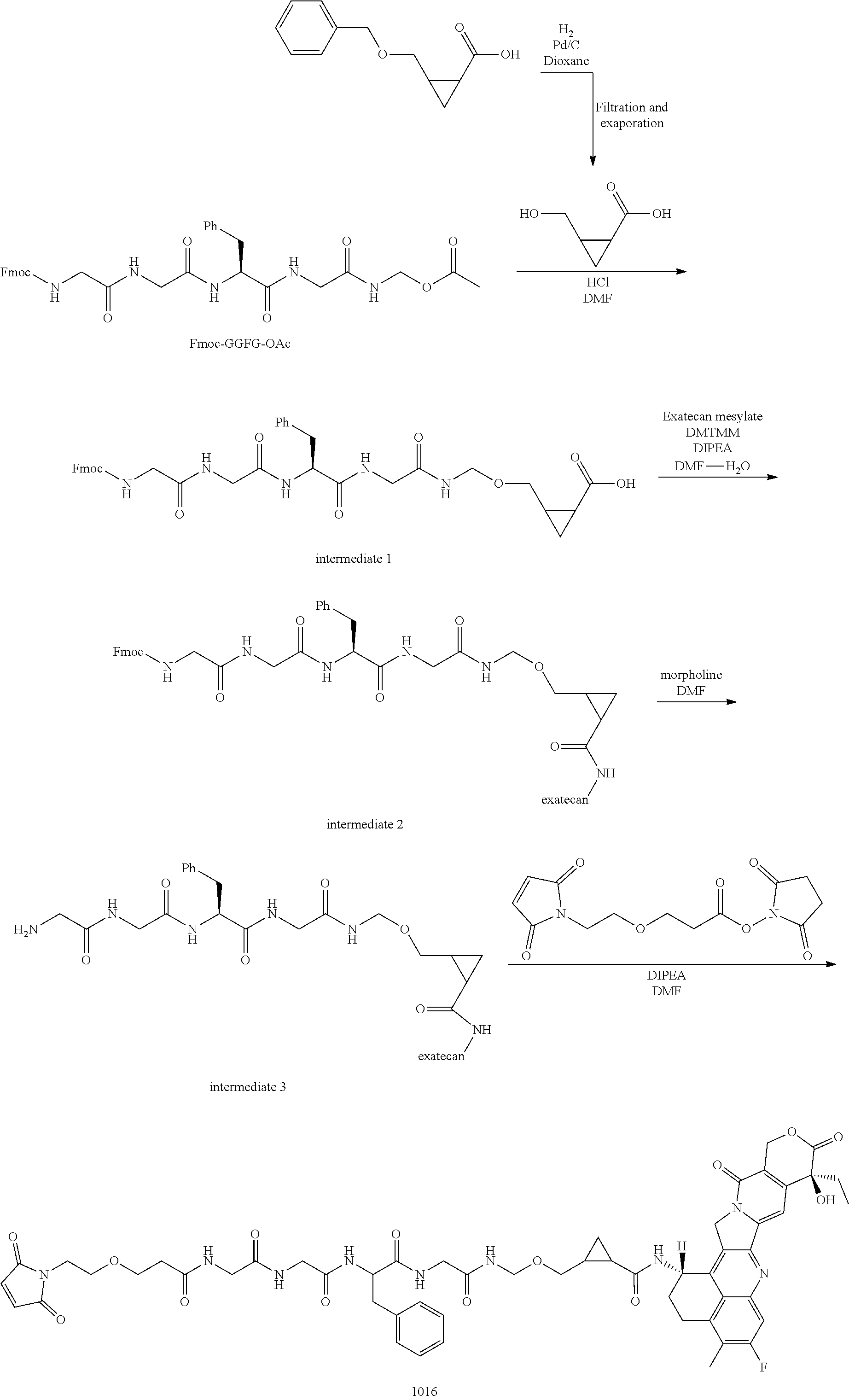

Intermediate 1. 2-((Benzyloxy)methyl)cyclopropane-1-carboxylic acid (27.6 mg, 0.1338 mmol) was dissolved in 3 mL of anhydrous dioxane. Pd/C (10%) was added and hydrogen was bubbled into the solution for 5 h, while stirring at room temperature. The solution was filtered with 0.2 μm syringe filters, and the flask washed with acetonitrile. The filtrate was evaporated, re-dissolved in dioxane and lyophilized overnight. The crude filtrate was re-dissolved in 2 mL of anhydrous DMF, and FmocGGFG-OAc (1 equiv., 0.1338 mmol, 90 mg) was added, followed by 200 μL of a 2M HCl solution in ethyl ether. The reaction mixture was stirred at room temperature for 1 h, to be then directly loaded on column for purification. Purification was performed by reverse-phase flash chromatography (25 g, diol-modified C18, 0 á 75% ACN/H₂O). Fractions containing the product were lyophilized from water (35 mg, 51%). MS calc. for C₃₆H₄₀N₅O₉: 686.28, found: 686.66, [M+H]⁺.

Intermediate 2. To a solution of the previously prepared intermediate 1 (35 mg, 0.0505 mmol) in DMF (2 mL) were added exatecan mesylate (1 equiv., 0.0505 mmol, 27 mg), DMTMM (1.2 equiv., 0.0607 mmol, 17 mg), DIPEA (10 μL) and water (200 μL). The reaction mixture was stirred at room temperature for 30 min, to be then directly loaded on column for purification. Purification was performed by reverse-phase flash chromatography (25 g, diol-modified C18, 0 á 60% ACN/H₂O). Fractions containing the product were lyophilized from water (28 mg, 50%). MS calc. for C₆₀H₆₀FN₈O₁₂: 1103.43, found: 1103.88, [M+H]⁺.

Intermediate 3. The previously prepared intermediate 2 (28 mg, 0.0254 mmol) was dissolved in DMF (2 mL) and morpholine (100 μL) was added. The reaction mixture was stirred at room temperature for 1 h. Purification was performed by reverse-phase HPLC chromatography (semipreparative, diol-modified C18, 0 á 100% ACN/H₂O). Fractions containing the product were lyophilized from water (11 mg, 51%). MS calc. for C₄₅H₅₀FN₈O₁₀: 881.36, found: 881.12, [M+H]⁺.

Compound 1016. The previously prepared intermediate 3 (11 mg, 0.0130 mmol) was dissolved in 2 mL of anhydrous DMF. 2,5-dioxopyrrolidin-1-yl 3-(2-(2,5-dioxo-2,5-dihydro-1H-pyrrol-1-yl)ethoxy)propanoate (1.1 equiv., 0.0143 mmol, 4 mg) and DIPEA (1.1 equiv., 0.0143 mmol, 2.5 μL) were added and the reaction mixture was stirred at room temperature for 1 h. Purification was performed by reverse-phase HPLC chromatography (semipreparative, diol-modified C18, 0 á 100% ACN/H₂O). Fractions containing the product were lyophilized from water (7 mg, 50%). MS calc. for C₅₄H₅₉FN₉O₁₄: 1076.42, found: 1076.56, [M+H]⁺. ¹H NMR (500 MHz, DMSO-d₆) δ 8.80-8.70 (m, 2H), 8.53 (m, 1H), 8.45 (m, 1H), 8.27 (m, 1H), 8.10 (m, 2H), 7.99 (m, 2H), 7.93 (d, J=6.1 Hz, 1H), 7.89 (d, J=8.4 Hz, 1H), 7.80 (m, 2H), 7.34-7.20 (m, 2H), 7.18-7.08 (m, 2H), 6.67 (s, 2H), 6.51 (s, 1H), 5.62 (d, J=10.0 Hz, 1H), 5.55 (s, 2H), 5.43 (t, J=5.7 Hz, 2H), 5.19-5.07 (m, 3H), 5.06-5.02 (m, 1H), 4.74 (d, J=6.0 Hz, 1H), 4.61-4.44 (m, 3H), 3.72 (m, 2H), 3.57 (m, 2H), 3.16 (d, J=8.7 Hz, 1H), 3.08-3.00 (m, 1H), 2.79 (m, 2H), 2.39 (m, 3H), 2.34 (d, J=1.8 Hz, 3H), 2.24-2.07 (m, 2H), 1.96-1.77 (m, 2H), 1.55 (m, 1H), 1.51-1.44 (m, 1H), 0.99 (m, 1H), 0.88 (t, J=7.3 Hz, 3H), 0.83-0.71 (m, 1H).

### Example 31: Synthesis of Compound 1017

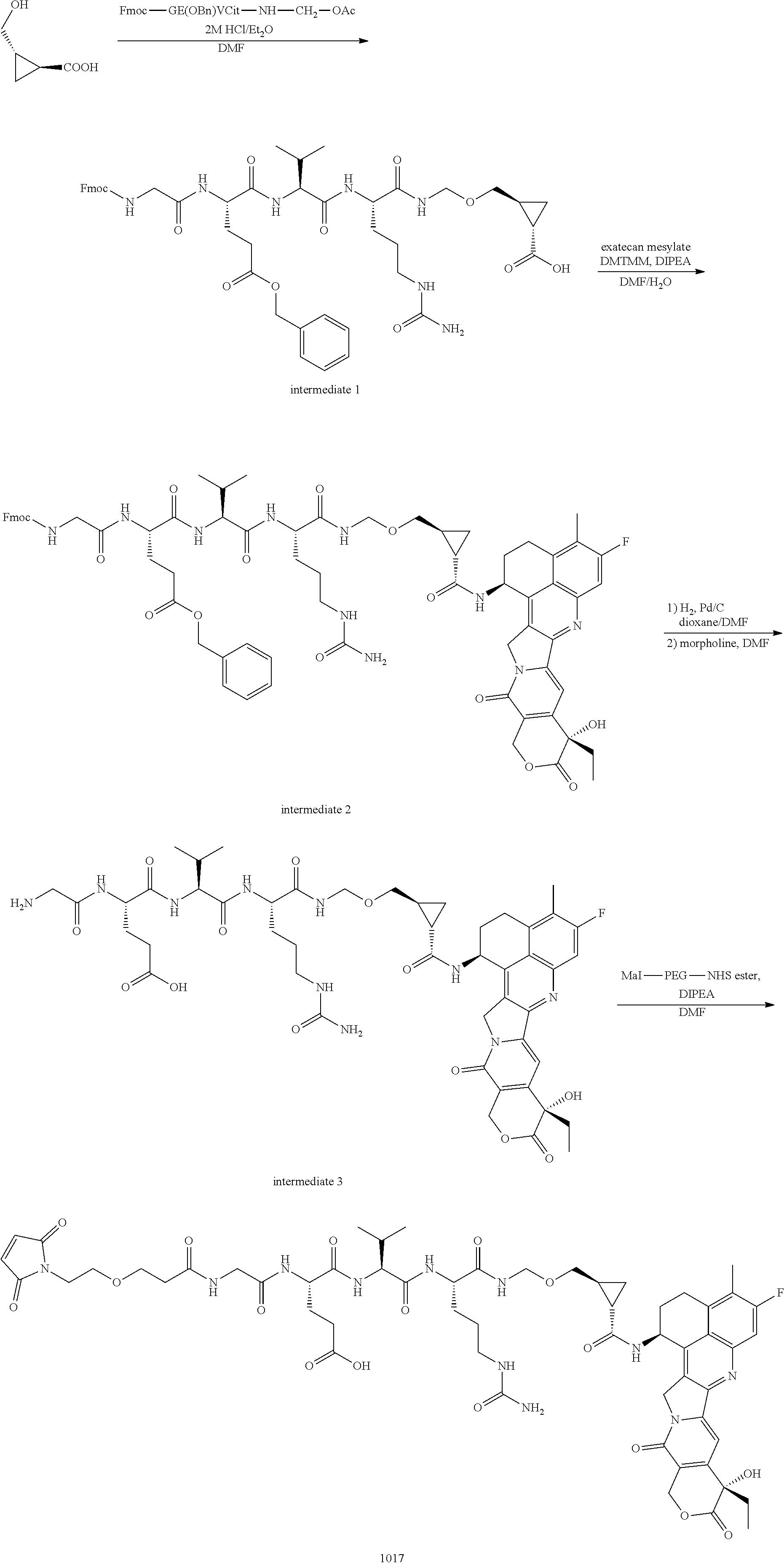

Intermediate 1. To the solution of (1S,2S)-2-(hydroxymethyl)cyclopropane-1-carboxylic acid (1.0 equiv., 5.7 mg, 0.049 mmol), Fmoc-GE(OBn)VCit-NH—CH₂—OAc (1.5 equiv., 62 mg, 0.074 mmol) in anhydrous DMF (0.7 ml) was added 2M HCl/Et₂O (100 μl) and the resulting mixture was stirred at room temperature for 1 hour, as LC-MS analysis indicated the full consumption of the starting material. The reaction mixture was purified by reverse-phase flash chromatography using a semipreparative column (diol-modified C18, 0→75% ACN/0.1% HCl). Fractions containing the product (co-eluting with impurity) were lyophilised to obtain 31 mg of impure intermediate 1, which was used directly into the next step. MS calc. for C₄₆H₅₆FN₇O₁₂: 898.40, found: 898.40, [M−H]⁻.

Intermediate 2. To the mixture of intermediate 1 (1.0 equiv., 31 mg, 0.034 mmol), exatecan mesylate (0.9 equiv., 17 mg, 0.031 mmol) and DMTMM (1.0 equiv., 10 mg, 0.034 mmol) was added DMF/water (5:1, 1.2 ml) and diisopropylethylamine (2.0 equiv., 12 μl, 0.068 mmol) and the resulting mixture was stirred at room temperature for 1 hour, as LC-MS analysis indicated the full consumption of the starting material. The reaction mixture was purified by reverse-phase flash chromatography (25 g, diol-modified C18, 0→75% ACN/H₂O), offering intermediate 2 as a white solid after lyophilisation (25 mg, 39% (2 steps)). MS calc. for C₇₀H₇₈FN₁₀O₁₅: 1317.56, found: 1317.55, [M+H]⁺.

Intermediate 3. To the solution of intermediate 2 (1.0 equiv., 25 mg, 0.019 mmol) in the mixture of dioxane (1.0 ml) and DMF (0.5 ml) was added 10% Pd/C (5 mg) and the reaction mixture was hydrogenated (balloon) for 2 hours at room temperature. LC-MS indicated the full consumption of starting material. The mixture was filtered through the layer of celite and the filtrate was concentrated on rotary evaporator to remove dioxane. Morpholine (40 μl) was then added to the obtained solution and the reaction mixture was stirred at room temperature for 1 hour. Purification by reverse-phase flash chromatography (25 g, diol-modified C18, 0→60% ACN/H₂O) offered the product intermediate 3 as a white solid after lyophilisation (8 mg, 42%). MS calc. for C₄₈H₆₂FN₁₀O₁₃: 1005.43, found: 1005.40, [M+H]⁺.

Compound 1017. Mal-PEG-NHS ester (1.0 equiv., 0.008 mmol, 2.5 mg) and DIPEA (1.05 equiv., 0.008 mmol, 1.5 μL) were added to the solution of intermediate 3 (1.0 equiv., 8 mg, 0.008 mmol) in anhydrous DMF (0.5 ml). The reaction mixture was stirred at room temperature for 1.5 h, as LC-MS indicated the full consumption of starting material. Purification by reverse-phase flash chromatography using a semipreparative column (diol-modified C18, 0→70% ACN/0.1% TFA) offered the desired product as a pale-yellow solid after lyophilisation (5 mg, 52%). MS calc. for C₅₇H₇₁FN₁₁O₁₇: 1200.50, found: 1200.50, [M+H]⁺. ¹H NMR (500 MHz, DMSO-d₆) δ: 12.09 (s br, 1H), 8.71 (d, J=8.7 Hz, 1H), 8.58-8.49 (m, 1H), 8.05 (t, J=5.2 Hz, 1H), 8.01-7.92 (m, 2H), 7.83-7.70 (m, 2H), 7.31 (s, 1H), 7.00 (s, 2H), 6.58 (s br, 1H), 5.92 (s, 1H), 5.62-5.49 (m, 1H), 5.50-5.33 (m, 2H), 5.28-5.12 (m, 2H), 4.60-4.40 (m, 2H), 4.39-4.25 (m, 1H), 4.21-4.02 (m, 2H), 3.77-3.62 (m, 3H), 3.31-3.19 (m, 2H), 3.20-3.09 (m, 1H), 3.04-2.82 (m, 2H), 2.40 (s, 3H), 2.35-2.26 (m, 2H), 2.27-2.10 (m, 4H), 1.99-1.79 (m, 4H), 1.76-1.64 (m, 1H), 1.63-1.41 (m, 4H), 1.40-1.23 (m, 3H), 1.05-0.93 (m, 1H), 0.92-0.84 (m, 3H), 0.84-0.75 (m, 6H), 0.75-0.69 (m, 2H).

### Example 32: Synthesis of Compound 115

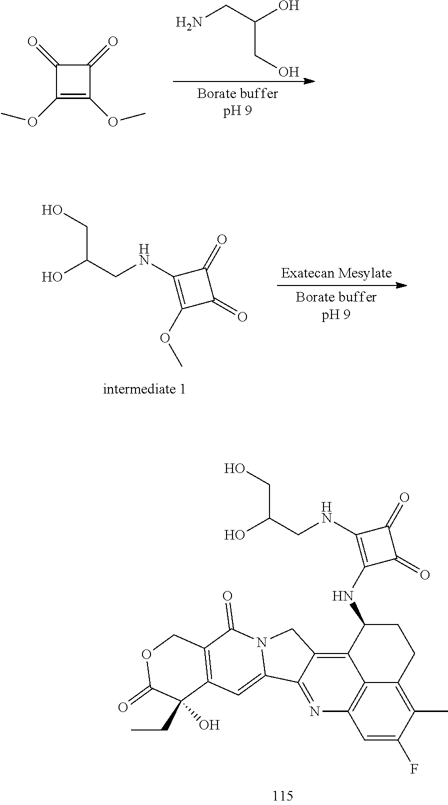

Intermediate 1. 3-Amino-1,2-propanediol (25 mg, 0.274 mmol) and dimethoxysquarate (3 equiv., 0.823 mmol, 117 mg) were suspended in 10 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (33 mg, 57%). MS calc. for C₈H₁₂NO₅: 202.07, found: 202.18, [M+H]⁺.

Compound 115. Exatecan mesylate (20 mg, 0.0377 mmol) and the previously synthesized intermediate 1 (1.5 equiv., 0.0564 mmol, 12 mg) were suspended in 5 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in water (0→50% ACN in H₂O). The desired product was recovered as a white powder, after lyophilization from water (15 mg, 66%). MS calc. for C₃₁H₃₀FN₄O₈: 605.20, found: 605.22, [M+H]⁺. ¹H NMR (400 MHz, DMSO-d₆) δ 8.04 (s, 1H), 7.85 (d, J=10.9 Hz, 1H), 7.32 (s, 1H), 6.53 (s, 1H), 5.79 (s, 1H), 5.42 (s, 2H), 5.39 (s, 2H), 3.74 (s, 2H), 3.55 (s, 1H), 3.52-3.31 (m, 6H), 3.32-3.20 (m, 1H), 2.43 (d, J=1.9 Hz, 3H), 2.34-2.26 (m, 1H), 1.96-1.77 (m, 2H), 0.87 (t, J=7.3 Hz, 3H).

### Example 33: Synthesis of Compound 117

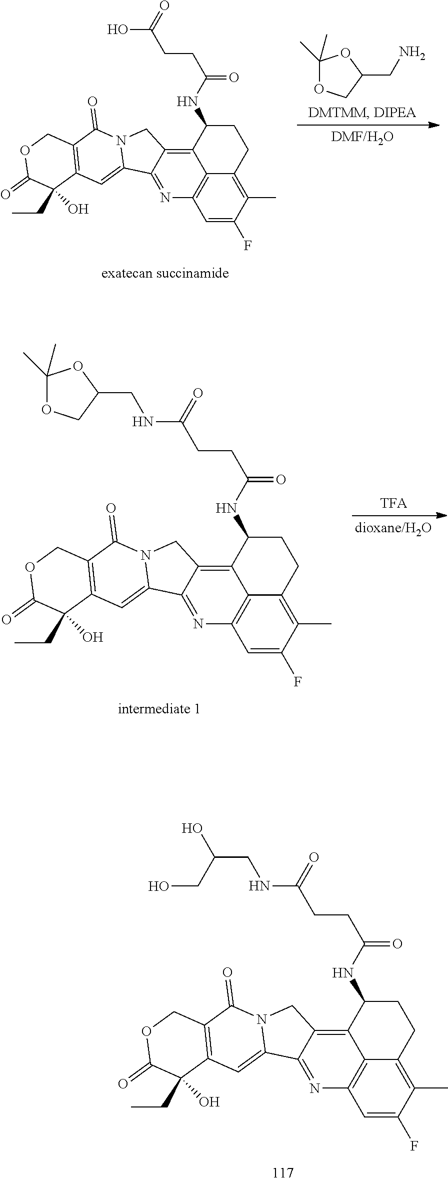

Intermediate 1. To a solution of exatecan succinamide (1 equiv. 16 mg, 0.030 mmol), (2,2-dimethyl-1,3-dioxolan-4-yl)methanamine (3 equiv., 12 mg, 0.090 mmol) and diisopropylethylamine (5 equiv., 0.150 mmol, 26 μL) in DMF/water (5:1, 1 mL) was added DMTMM (1.3 equiv., 13 mg, 0.039 mmol) under an argon atmosphere and the reaction mixture was stirred at room temperature for 0.5 h. LC-MS indicated the full consumption of starting material. Purification of the mixture by reverse-phase flash chromatography (diol-modified C18, 0→75% ACN/H₂O) offered the intermediate 1 (16 mg, 82%) as an off-white powder after lyophilization.

Compound 117. To the solution of intermediate 1 (16 mg, 0.025 mmol) in dioxane/water (1:1, 4 ml) were added 2 drops of TFA and the resulting mixture was stirred at 40° C. for 5 h. LC-MS indicated the full consumption of the intermediate 1 and solvents were evaporated under reduced pressure. The residue was purified by reverse-phase flash chromatography (diol-modified C18, 0→50% ACN/1% TFA in H₂O), giving the product (11 mg, 72%) as a white powder after lyophilization. MS calc. for C₃₁H₃₄FN₄O₈: 609.24, found: 609.25, [M+H]⁺. ¹H NMR (400 MHz, DMSO-d₆) δ: 8.47 (d, J=8.7 Hz, 1H), 7.86-7.81 (m br, 1H), 7.79 (d, J=11.0 Hz, 1H), 7.30 (s, 1H), 6.52 (s, 1H), 5.56 (dt, J=9.0, 4.8 Hz, 1H), 5.42 (s, 2H), 5.22 (d, J=19.1 Hz, 1H), 5.15 (d, J=19.1 Hz, 1H), 4.68 (dd, J=5.0, 2.1 Hz, 1H), 4.47 (t, J=5.7 Hz, 1H), 3.48-3.40 (m, 1H), 3.29-3.22 (m, 2H), 3.20-3.09 (m, 3H), 2.99-2.87 (m, 1H), 2.46-2.33 (m, 6H), 2.21-2.05 (m, 2H), 1.95-1.78 (m, 2H), 0.88 (t, J=7.3 Hz, 3H).

### Example 34: Synthesis of Compound 118

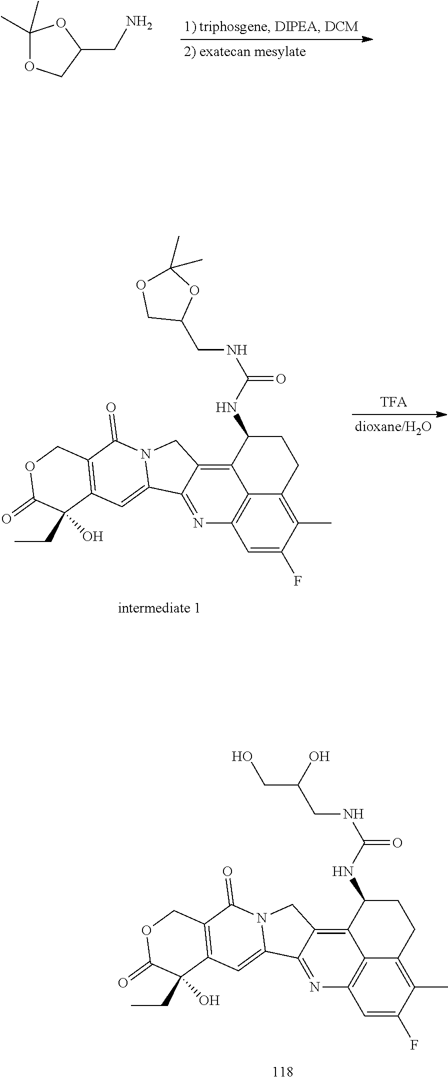

Intermediate 1. To a solution of (2,2-dimethyl-1,3-dioxolan-4-yl)methanamine (2 equiv. 15 mg, 0.114 mmol) and diisopropylethylamine (5 equiv., 0.285 mmol, 50 μL) in dichloromethane (3 mL) was added triphosgene (0.6 equiv., 11 mg, 0.035 mmol) under an argon atmosphere and the reaction mixture was stirred at room temperature for 2 h. Then, the solution of exatecan mesylate (1 equiv., 0.057 mmol, 30 mg) and diisopropylethylamine (1 equiv., 0.057 mmol, 11 μL) in anhydrous DMF (1 mL) was added and the resulting mixture was stirred at r.t. for 2 h. The reaction was quenched by addition of methanol (1 mL) and solvents were evaporated under reduced pressure. The residue was purified by reverse-phase flash chromatography (diol-modified C18, 0→60% ACN/H₂O), giving the intermediate 1 (29 mg, 86%) as an off-white powder after lyophilization and used in the next step.

Compound 118. To the solution of intermediate 1 (29 mg, 0.049 mmol) in dioxane/water (1:1, 4 ml) were added 2 drops of TFA and the resulting mixture was stirred at 40° C. overnight. LC-MS indicated the full consumption of the intermediate 1 and solvents were evaporated under reduced pressure. The residue was purified by reverse-phase flash chromatography (diol-modified C18, 0→50% ACN/1% TFA in H₂O), giving the product Compound 187 (15 mg, 55%) as a white powder after lyophilization. MS calc. for C₂₈H₃₀FN₄O₇: 553.21, found: 553.20, [M+H]⁺. ¹H NMR (400 MHz, DMSO-d₆) δ: 7.76 (d, J=10.9 Hz, 1H), 7.30 (d, J=1.6 Hz, 1H), 6.78 (dd, J=8.9, 6.6 Hz, 1H), 6.52 (d, J=1.1 Hz, 1H), 5.42 (s, 2H), 5.39-5.28 (m, 1H), 5.22 (d, J=19.3, 1H), 4.81 (dd, J=9.3, 4.9 Hz, 1H), 4.57 (q, Jj=5.9, 1H), 3.56-3.43 (m, 1H), 3.38-3.23 (m, 4H), 3.16 (m br, 2H), 3.04-2.91 (m, 1H), 2.38 (s, 3H), 2.24-2.06 (m, 2H), 1.94-1.79 (m, 2H), 0.88 (t, J=7.3 Hz, 3H).

### Example 35: Synthesis of Compound 122

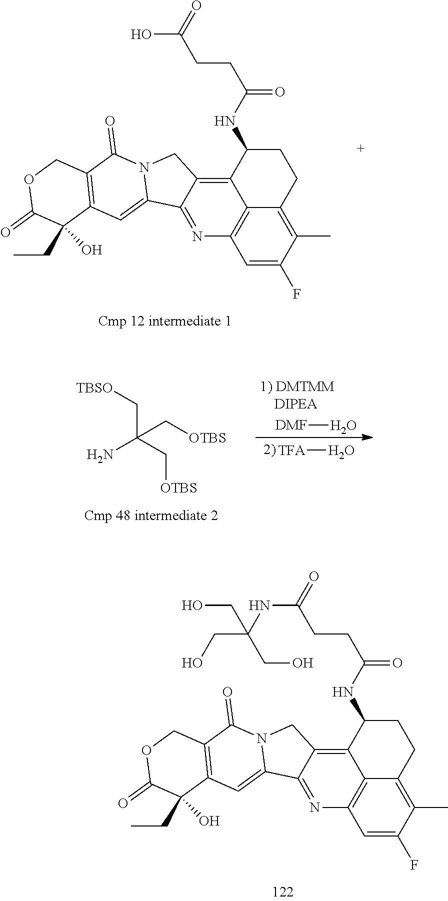

Compound 122. Compound 12 intermediate 1 (20 mg, 0.0373 mmol), Compound 48 intermediate 2 (2 equiv., 0.0747 mmol, 35 mg), DMTMM (2 equiv., 0.0747 mmol, 21 mg) and diisopropyethylamine (20 μL) were dissolved in 2 mL of a 4:1 DMF-water mixture, and the reaction mixture was stirred at room temperature for 1 h. Solvents were evaporated under reduced pressure and the crude product was re-dissolved in 2 mL of DCM. TFA (1 mL) and water (1 mL) were added and the mixture was stirred at room temperature for 1 h. Solvents were evaporated and the crude reaction mixture was purified by reverse-phase flash chromatography, using a column containing 25 g of diol-modified C18, and using a gradient of ACN in 1% TFA (0→40% ACN in 1% TFA). The desired product was recovered as a white powder, after lyophilization from water-acetonitrile (34 mg, 88%). MS calc. for C₃₂H₃₆FN₄O₉: 639.25, found: 639.29 [M+H]⁺. ¹H NMR (400 MHz, DMSO-d₆) δ 8.50 (d, J=8.7 Hz, 1H), 7.99 (s, 1H), 7.81 (d, J=11.0, 1H), 7.32 (d, J=9.0 Hz, 1H), 7.21 (d, J=11.7 Hz, 1H), 6.54 (s, 1H), 5.57 (m, 1H), 5.43 (d, J=1.6 Hz, 2H), 5.21 (m, 2H), 4.20-4.08 (m, 1H), 3.55 (m, 1H), 3.49 (s, 6H), 3.21-3.16 (m, 2H), 2.77-2.65 (m, 1H), 2.43-2.34 (m, 3H), 2.21-2.06 (m, 2H), 1.94-1.79 (m, 2H), 0.88 (td, J=7.4, 3.8 Hz, 3H).

### Example 36: Synthesis of Compound 129

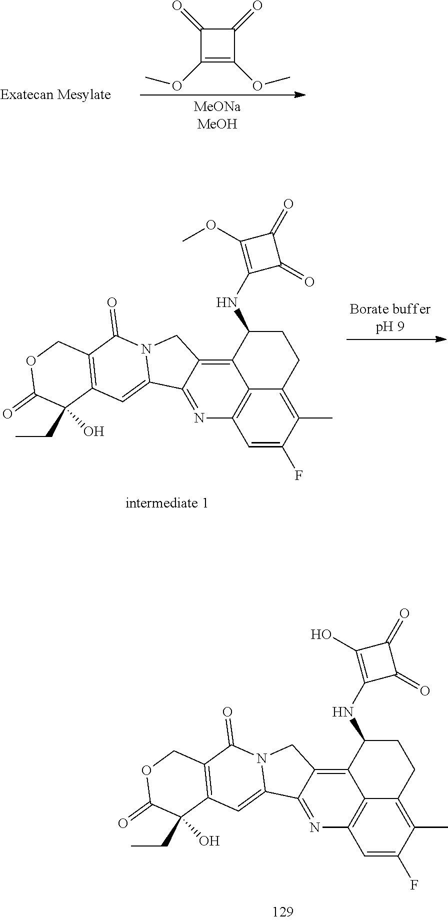

Intermediate 1. Exatecan mesylate (25 mg, 0.0466 mmol), dimethoxysquarate (3 equiv., 0.140 mmol, 20 mg) and sodium methoxide (5 mg) were suspended in 2 mL of anhydrous MeOH, and the mixture was stirred at room temperature for 16 hours. Methanol was evacuated under reduced pressure and the crude reaction product was re-dissolved in 2 mL of DMF. The crude reaction mixture was purified by reverse-phase flash chromatography, using a column containing 25 g of diol-modified C18, and using a gradient of ACN in 1% TFA (0→40% ACN in 1% TFA). The desired product was recovered as a white powder, after lyophilization from water (16 mg, 63%). MS calc. for C₂₉H₂₅FN₃O₇: 546.17, found: 545.98, [M+H]⁺.

Compound 129. The previously prepared Intermediate 1 (16 mg, 0.0293 mmol) was suspended in 10 mL of 1M borate buffer (pH=9), and the mixture was stirred at 55° C. for 16 hours. 2 mL of DMF were added, and solvents were evaporated under reduced pressure to a final volume of approx. 3 mL. The crude reaction mixture was purified by reverse-phase flash chromatography, using a column containing 25 g of diol-modified C18, and using a gradient of ACN in 1% TFA (0→40% ACN in 1% TFA). The desired product was recovered as a white powder, after lyophilization from water (7 mg, 45%). MS calc. for C₂₈H₂₃FN₃O₇: 532.15, found: 532.20, [M+H]⁺. ¹H NMR (400 MHz, DMSO-d₆) δ 9.03 (d, J=8.6 Hz, 1H), 7.81 (d, J=10.9 Hz, 1H), 7.31 (s, 1H), 5.58 (q, J=6.4 Hz, 1H), 5.41 (s, 2H), 5.24 (q, J=18.9 Hz, 2H), 3.99 (bs, 2H), 3.27 (dt, J=16.8, 6.4 Hz, 1H), 3.14 (dt, J=17.0, 5.8 Hz, 1H), 2.40 (d, J=1.9 Hz, 3H), 2.33 (q, J=6.1 Hz, 2H), 1.95-1.81 (m, 2H), 0.87 (t, J=7.3 Hz, 3H).

### Example 37: Synthesis of Compound 130

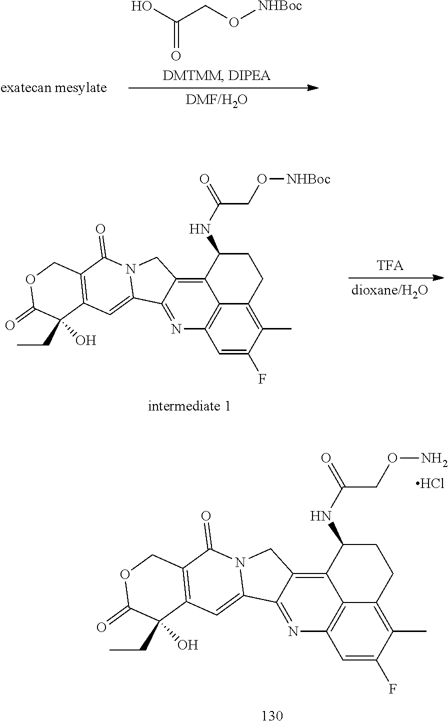 [SMILES: CC(C)(C)OC(=O)NOCC(=O)O ; CC[C@@]1(O)C(=O)OCc2c1cc1n(c2=O)Cc2c-1nc1cc(F)c(C)c3c1c2[C@@H](NC(=O)CONC(=O)OC(C)(C)C)CC3 ; CC[C@@]1(O)C(=O)OCc2c1cc1n(c2=O)Cc2c-1nc1cc(F)c(C)c3c1c2[C@@H](NC(=O)CON)CC3 ; Cl]

Intermediate 1. To the mixture of exatecan mesylate (1 equiv., 40 mg, 0.075 mmol), 2-(((tert-butoxycarbonyl)amino)oxy)acetic acid (1.2 equiv., 18 mg, 0.090 mmol) and DMTMM (1.2 equiv., 25 mg, 0.090 mmol) was added DMF/water (5:1, 2 ml) and diisopropylethylamine (2.2 equiv., 0.165 mmol, 29 μL) and the resulting mixture was stirred at room temperature for 0.5 h. LC-MS indicated the full consumption of starting material. Purification of the mixture by reverse-phase flash chromatography (diol-modified C18, 0→100% ACN/H₂O) offered the intermediate 1 (43 mg, 94%) as a white powder after lyophilization.

Compound 130. Intermediate 1 (43 mg, 0.070 mmol) was dissolved in 4M HCl/dioxane (2 ml) and the mixture was stirred for 1.5 hour at room temperature. The resulting suspension was filtered and the solids were washed with dioxane and Et₂O to give the hydrochloride of compound 130 (35 mg, 90%) as a yellow powder. MS calc. for C₂₆H₂₆FN₄O₆: 509.18, found: 509.20, [M+H]⁺. ¹H NMR (400 MHz, DMSO-d₆) δ: 10.98 (s br, 3H), 8.89 (d, J=8.5 Hz, 1H), 7.81 (d, J=11.0 Hz, 1H), 7.32 (s, 1H), 5.62 (dt, J=8.5, 4.2 Hz, 1H), 5.43 (s, 2H), 5.37-5.24 (m, 2H), 4.63-4.52 (m, 2H), 3.20 (dd, J=7.9, 4.7 Hz, 2H), 2.41 (d, J=1.9 Hz, 3H), 2.32-2.21 (m, 1H), 2.21-2.09 (m, 1H), 1.96-1.77 (m, 2H), 0.87 (t, J=7.3 Hz, 3H).

### Example 38: Synthesis of Compound 1018

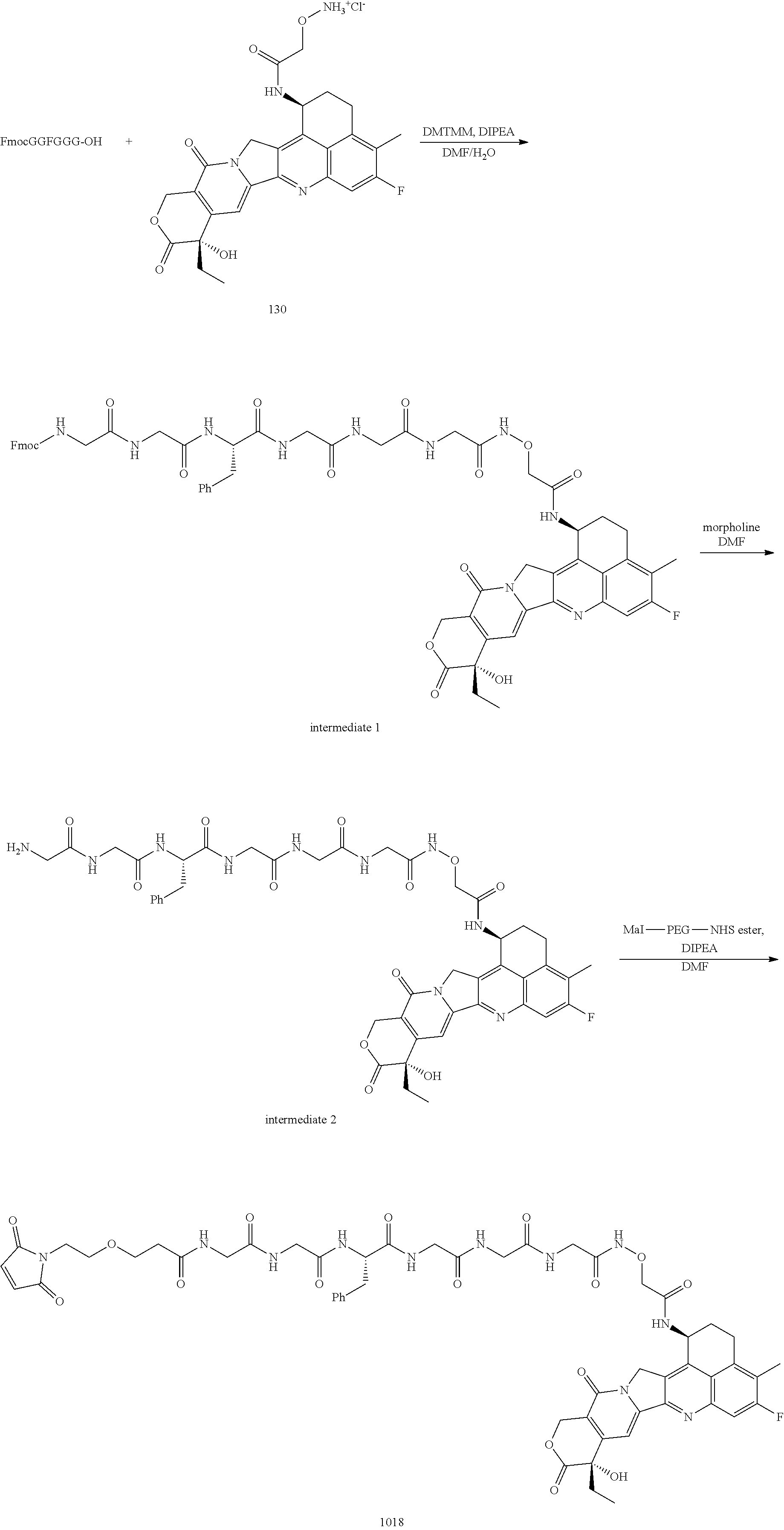

Intermediate 1. The mixture of compound 130 (1.0 equiv., 30 mg, 0.055 mmol), FmocGGFGGG-OH (1.25 equiv., 47 mg, 0.069 mmol) and DMTMM (1.25 equiv., 19 mg, 0.069 mmol) was added DMF/water (5:1, 2.4 ml) and diisopropylethylamine (2.25 equiv., 22 μL, 0.124 mmol) and the resulting mixture was stirred at room temperature for 1 h, as LC-MS analysis indicated the full consumption of the starting material. The reaction mixture was purified by reverse-phase flash chromatography (25 g, diol-modified C18, 0→75% ACN/H₂O), offering intermediate 1 as a white solid after lyophilization (48 mg, 75%). MS calc. for C₆₀H₆₀FN₁₀O₁₄: 1163.43, found: 1163.40, [M+H]⁺.

Intermediate 2. Morpholine (100 μL) was added to the solution of intermediate 1 (1.0 equiv., 48 mg, 0.041 mmol) in anhydrous DMF (1.5 ml) and the reaction mixture was stirred for 1 h at room temperature. LC-MS indicated the full consumption of starting material. The mixture was directly purified by reverse-phase flash chromatography (25 g, diol-modified C18, 0‘→60% ACN/H₂O), giving the product as a white solid after lyophilization (26 mg, 67%). MS calc. for C₄₅H₅₀FN₁₀O₁₂: 941.36, found: 941.40, [M+H]⁺.

Compound 1018. Mal-PEG-NHS ester (1.0 equiv., 0.027 mmol, 8.4 mg) and DIPEA (1.1 equiv., 0.030 mmol, 5.2 μL) were added to the solution of intermediate 2 (1.0 equiv., 26 mg, 0.027 mmol) in anhydrous DMF (1 ml). The reaction mixture was stirred at room temperature for 40 minutes, as LC-MS indicated the full consumption of starting material. DMF was removed and the residue was concentrated from the mixture of 0.1% aq. TFA and ACN. Purification by reverse-phase flash HPLC using a semipreparative column (diol-modified C18, 0→60% ACN/H₂O) offered the desired product as a white solid after lyophilization (17 mg, 55%). MS calc. for C₅₄H₅₉FN₁₁O₁₆: 1136.41, found: 1136.45, [M+H]⁺. ¹H NMR (500 MHz, DMSO-d₆) δ: 11.45 (s, 1H), 8.96-8.68 (m, 1H), 8.26 (t, J=5.8 Hz, 1H), 8.22-8.04 (m, 3H), 8.04-7.93 (m, 2H), 7.87-7.75 (m, 1H), 7.31 (s, 1H), 7.23 (d, J=6.9 Hz, 4H), 7.19-7.12 (m, 1H), 6.99 (s, 1H), 6.59-6.44 (m, 1H), 5.64-5.54 (m, 1H), 5.42 (s, 2H), 5.35-5.10 (m, 2H), 4.50 (td, J=9.8, 9.1, 4.6 Hz, 1H), 4.45-4.28 (m, 2H), 3.79-3.39 (m, 17H), 3.25-3.10 (m, 2H), 3.09-2.99 (m, 1H), 2.88-2.72 (m, 1H), 2.39 (s, 3H), 2.32 (t, J=6.5 Hz, 2H), 2.27-2.02 (m, 2H), 1.96-1.78 (m, 2H), 0.86 (t, J=7.3 Hz, 3H).

### Example 39: Synthesis of Compound 1019

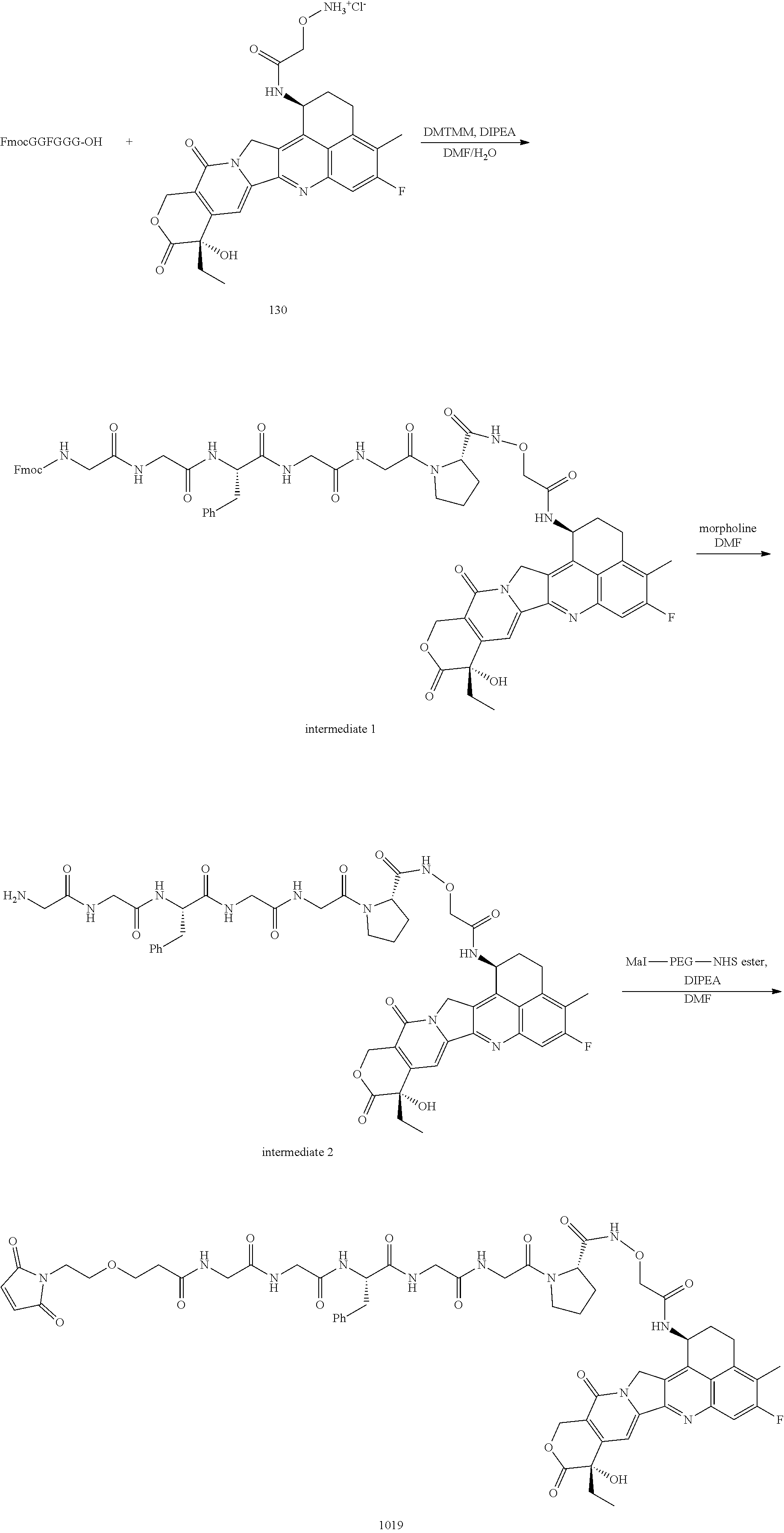

Intermediate 1. The mixture of compound 130 (1.0 equiv., 35 mg, 0.064 mmol), FmocGGFGGP-OH (1.2 equiv., 55 mg, 0.077 mmol) and DMTMM (1.2 equiv., 22 mg, 0.077 mmol) was added DMF/water (5:1, 2.4 ml) and diisopropylethylamine (2.2 equiv., 25 μL, 0.141 mmol) and the resulting mixture was stirred at room temperature for 1 h, as LC-MS analysis indicated the full consumption of the starting material. The reaction mixture was purified by reverse-phase flash chromatography (25 g, diol-modified C18, 0→75% ACN/H₂O), offering intermediate 1 as a white solid after lyophilization (65 mg, 84%). MS calc. for C₆₃H₆₄FN₁₀O₁₄: 1203.46, found: 1203.50, [M+H]⁺.

Intermediate 2. Morpholine (130 μL) was added to the solution of intermediate 1 (1.0 equiv., 65 mg, 0.054 mmol) in anhydrous DMF (1.7 ml) and the reaction mixture was stirred for 1 h at room temperature. LC-MS indicated the full consumption of starting material. The mixture was directly purified by reverse-phase flash chromatography (25 g, diol-modified C18, 0→60% ACN/H₂O), giving the product as a white solid after lyophilization (34 mg, 64%). MS calc. for C₄₈H₅₄FN₁₀O₁₂: 981.39, found: 981.40, [M+H]⁺.

Compound 1019. Mal-PEG-NHS ester (1.0 equiv., 0.035 mmol, 10.8 mg) and DIPEA (1.05 equiv., 0.037 mmol, 6.4 μL) were added to the solution of intermediate 2 (1.0 equiv., 34 mg, 0.035 mmol) in anhydrous DMF (1 ml). The reaction mixture was stirred at room temperature for 1 h, as LC-MS indicated the full consumption of starting material. DMF was removed and the residue was concentrated from the mixture of 0.1% aq. TFA and ACN. Purification by reverse-phase flash HPLC using a semipreparative column (diol-modified C18, 0→60% ACN/H₂O) offered the desired product as a white solid after lyophilization (18 mg, 44%). MS calc. for C₅₇H₆₃FN₁₁O₁₆: 1176.44, found: 1176.45, [M+H]⁺. ¹H NMR (500 MHz, DMSO-d₆) δ: 11.50 (s, 1H), 8.85 (d, J=8.7 Hz, 1H), 8.31-8.25 (m, 1H), 8.16-8.02 (m, 3H), 7.96 (t, J=5.7 Hz, 1H), 7.88-7.77 (m, 2H), 7.74 (t, J=5.2 Hz, 1H), 7.35-7.28 (m, 2H), 7.28-7.18 (m, 5H), 7.18-7.11 (m, 1H), 6.99 (s, 1H), 6.57-6.46 (m, 1H), 5.70-5.58 (m, 1H), 5.43 (s, 2H), 5.35-5.11 (m, 3H), 4.54-4.46 (m, 1H), 4.32 (s, 2H), 4.08-4.03 (m, 1H), 3.90-3.39 (m, 14H), 3.24-3.10 (m, 2H), 3.03 (dd, J=13.9, 4.4 Hz, 1H), 2.77 (dd, J=13.9, 9.8 Hz, 1H), 2.40 (s, 3H), 2.32 (t, J=6.5 Hz, 2H), 2.26-2.11 (m, 2H), 1.97-1.77 (m, 3H), 1.69-1.51 (m, 1H), 0.86 (t, J=7.4 Hz, 3H).

### Example 40: Synthesis of Compound 136

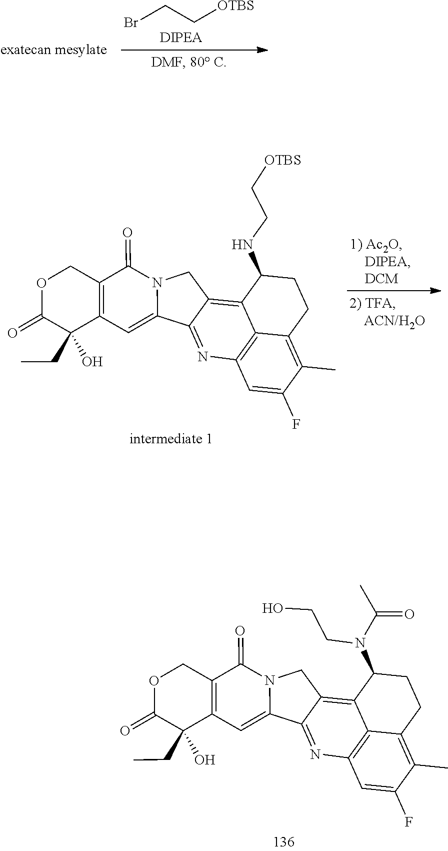 [SMILES: CC(C)(C)[Si](C)(C)OCCBr ; CC[C@@]1(O)C(=O)OCc2c1cc1n(c2=O)Cc2c-1nc1cc(F)c(C)c3c1c2[C@@H](NCCO[Si](C)(C)C(C)(C)C)CC3 ; CC[C@@]1(O)C(=O)OCc2c1cc1n(c2=O)Cc2c-1nc1cc(F)c(C)c3c1c2[C@@H](N(CCO)C(C)=O)CC3]

Intermediate 1. To the suspension of exatecan mesylate (1.0 equiv., 30 mg, 0.056 mmol) in DMF (1.2 mL) were added diisopropylethylamine (4.5 equiv., 0.25 mmol, 45 μL) and (2-bromoethoxy)-tert-butyldimethylsilane (3.3 equiv., 0.19 mmol, 45 mg, 40 μL) under an argon atmosphere and the mixture was heated to 80° C. for 3 days. Purification by reverse-phase flash chromatography (25 g, diol-modified C18, 0→60% ACN/H₂O) offered the product intermediate 1 (12 mg, 36%) as an off-white powder after lyophilization.

Compound 136. Acetic anhydride (2.2 equiv., 0.042, 4.3 mg, 4 μL) and diisopropylethylamine (2.2 equiv., 0.042 mmol, 8 μL) were added to the solution of intermediate 1 (1.0 equiv., 12 mg, 0.02 mmol) in DCM (1.5 ml) and the resulting mixture was stirred at room temperature for 40 hours. Then, volatiles were removed on rotary evaporator and the residue was dissolved in the mixture of water and acetonitrile (1:1, 2 mL) followed by the addition of 2 drops of TFA. The resulting mixture was stirred at room temperature for 2.5 hours, as LC-MS indicated the full consumption of starting material. Purification by reverse-phase flash chromatography using semipreparative column (diol-modified C18, 0→70% ACN/H₂O) offered the product (9 mg, 86%) as a white powder after lyophilization. MS calc. for C₂₈H₂₉FN₃O₆: 522.20, found: 522.20, [M+H]⁺. ¹H NMR (400 MHz, DMSO-d₆, mixture of isomers) δ: 7.84-7.71 (m, 1H), 7.34-7.28 (m, 1H), 6.55-6.48 (m, 1H), 5.64-5.44 (m, 1H), 5.41 (s, 2H), 5.20-5.01 (m, 1H), 4.99-4.81 (m, 2H), 3.69-3.37 (m, 3H), 3.19-2.92 (m, 1H), 2.46-2.30 (m, 4H), 2.31-2.11 (m, 3H), 1.97-1.77 (m, 2H), 0.97-0.78 (m, 3H).

### Example 41: Synthesis of Compound 1022

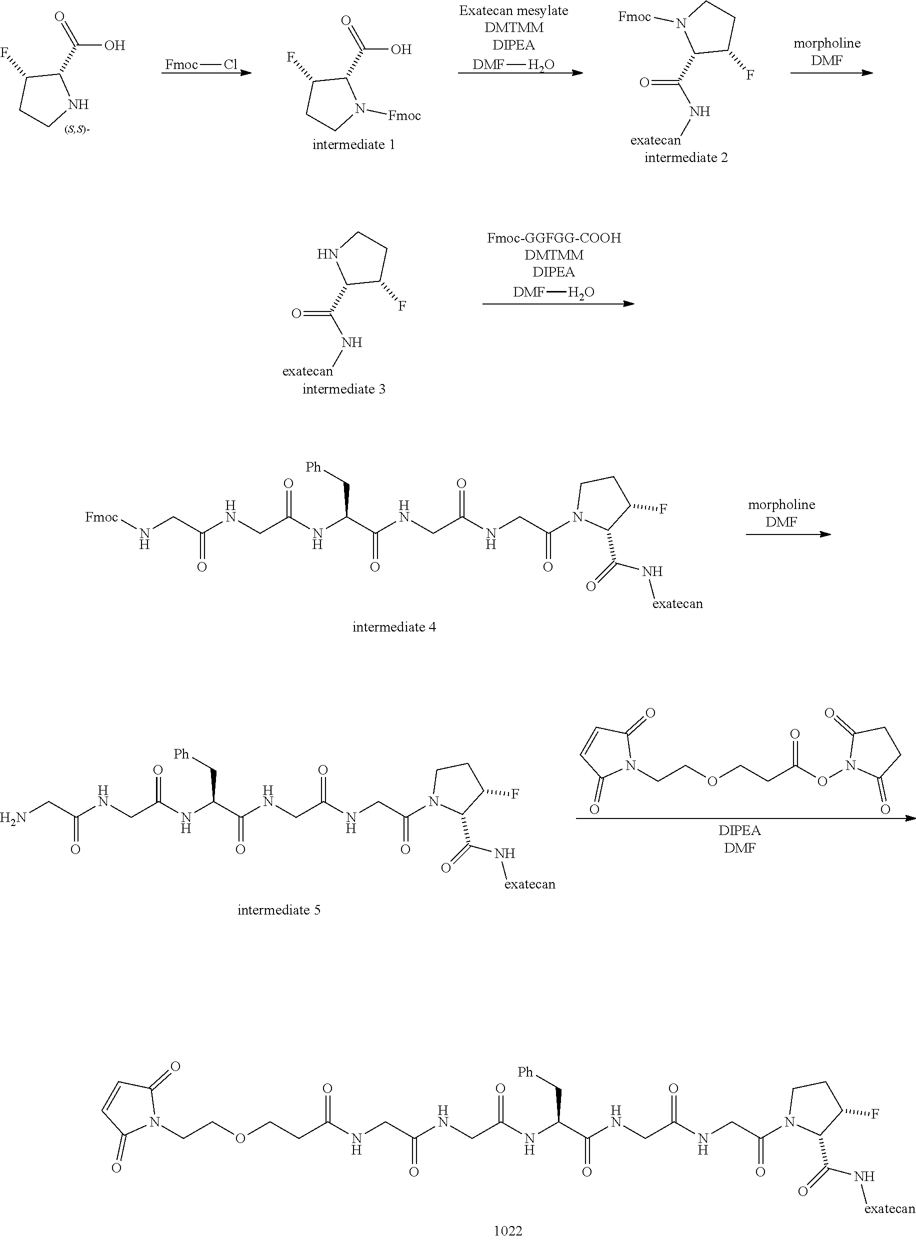

Intermediate 1. (S,S)-3-fluoropyrrolidine-2-carboxylic acid (62.5 mg, 0.47 mmol) was dissolved in 1,4-dioxane (1 mL) and H₂O (3 mL), and cooled to 0° C. K₂CO₃ (162 mg, 1.18 mmol) was added, and then Fmoc-Cl (115 mg, 0.45 mmol) was added. The mixture was stirred at RT overnight and H₂O (10 mL) was added. The mixture acidified with aqueous HCl (1 M) to pH 2-3, and extracted with DCM (2×10 mL). Combined organic layers were dried Na₂SO₄, concentrated to dryness to give the product as a white solid (122 mg, 76% yield). MS calc. for C₂₀H₁₉FNO₄: 356.12, found: 356.22, [M+H]⁺.

Intermediate 2. Exatecan mesylate (30 mg, 0.056 mmol), previously prepared intermediate 1 (5 equiv., 100 mg) and DIPEA (200 μL) were dissolved in a 5:1 mixture of DMF and water (3 mL). DMTMM (5 equiv., 78 mg) was added and the reaction mixture was stirred for 30 min at room temperature, to be then directly loaded on column for purification. Purification was performed by reverse-phase flash chromatography (25 g, diol-modified C18, 0→70% ACN/H₂O). Fractions containing the product were lyophilised from water (35 mg, 81%). MS calc. for C₄₄H₃₉F₂N₄O₆⁺: 789.27, found: 789.43, [M+H]⁺.

Intermediate 3. The previously prepared intermediate 2 (35 mg, 0.044 mmol) was dissolved in DMF (2 mL) and morpholine (100 μL) was added. The reaction mixture was stirred at room temperature for 1 h. Purification was performed by reverse-phase HPLC chromatography (25 g, diol-modified C18, 0→50% ACN/H₂O). Fractions containing the product were lyophilised from water (19 mg, 76%). MS calc. for C₂₉H₂₉F2N₄O₆⁺: 567.20, found: 567.57, [M+H]⁺.

Intermediate 4. The previously prepared intermediate 3 (19 mg, 0.035 mmol), Fmoc-GGFGG-COOH (2 equiv., 47 mg) and DIPEA (150 μL) were dissolved in a 5:1 mixture of DMF and water (3 mL). DMTMM (2 equiv., 19.4 mg) was added and the reaction mixture was stirred for 30 min at room temperature, to be then directly loaded on column for purification. Purification was performed by reverse-phase flash chromatography (semipreparative, diol-modified C18, 0→50% ACN/H₂O). Fractions containing the product were lyophilised from water (32 mg, 80%). MS calc. for C₆₁H₆₀F₂N₉O₁₂⁺: 1149.43, found: 1149.25, [M+H]⁺.

Intermediate 5. The previously prepared intermediate 4 (70 mg, 0.061 mmol) was dissolved in DMF (2 mL) and morpholine (100 μL) was added. The reaction mixture was stirred at room temperature for 1 h. Purification was performed by reverse-phase HPLC chromatography (semipreparative, diol-modified C18, 0→50% ACN/H₂O). Fractions containing the product were lyophilised from water (21 mg, 38%). MS calc. for C₄₆H₅₀F₂N₉O₁₀⁺: 926.36, found: 926.78, [M+H]⁺.

Compound 1022. The previously prepared intermediate 5 (21 mg, 0.023 mmol) was dissolved in 1.5 mL of anhydrous DMF. 2,5-Dioxopyrrolidin-1-yl 3-(2-(2,5-dioxo-2,5-dihydro-1H-pyrrol-1-yl)ethoxy)propanoate (1.1 equiv., 9.6 mg) and DIPEA (10 μL) were added and the reaction mixture was stirred at room temperature for 1 h. Purification was performed by reverse-phase HPLC chromatography (semipreparative, diol-modified C18, 0→50% ACN/H₂O). Fractions containing the product were lyophilised from water (10 mg, 39%). MS calc. for C₅₄H₅₉FN₉O₁₄: 1122.42, found: 1122.45, [M+H]⁺. ¹H NMR (500 MHz, DMSO-d₆) δ 8.50 (d, J=8.5 Hz, 1H), 8.29 (t, J=5.9 Hz, 1H), 8.13-8.07 (m, 2H), 7.97 (t, J=5.7 Hz, 1H), 7.84-7.76 (m, 2H), 7.34 (s, 1H), 7.24 (m, 5H), 7.17 (m, 1H), 7.00 (s, 2H), 6.52 (d, J=4.1 Hz, 1H), 5.55 (dt, J=8.5, 4.2 Hz, 1H), 5.51-5.39 (m, 2H), 5.38-5.32 (m, 1H), 5.26-5.23 (m, 2H), 4.57-4.45 (m, 2H), 4.07 (m, 1H), 3.86 (m, 1H), 3.75 (m, 3H), 3.67 (d, J=5.6 Hz, 2H), 3.61 (d, J=5.7 Hz, 1H), 3.55 (m, 4H), 3.46 (t, J=5.8 Hz, 2H), 3.21-3.14 (m, 1H), 3.04 (m, 2H), 2.79 (m, 1H), 2.45-2.40 (m, 4H), 2.33 (t, J=6.6 Hz, 3H), 2.21 (m, 1H), 2.15 (d, J=5.0 Hz, 1H), 2.11-2.04 (m, 1H), 1.87 (m, 2H), 0.89 (t, J=7.4 Hz, 3H).

### Example 42: Synthesis of Compound 140

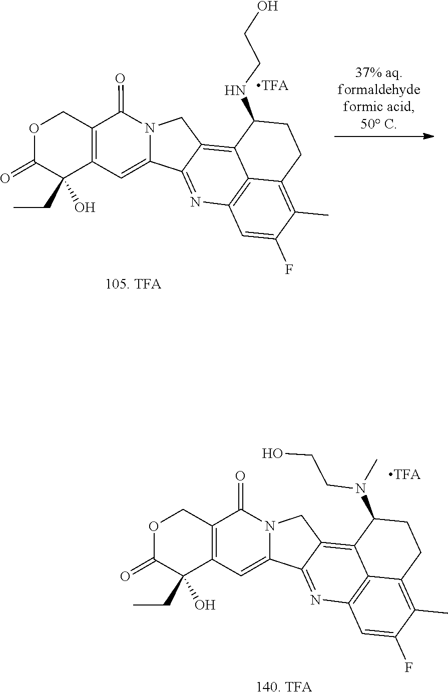 [SMILES: CC[C@@]1(O)C(=O)OCc2c1cc1n(c2=O)Cc2c-1nc1cc(F)c(C)c3c1c2[C@@H](NCCO)CC3 ; CC[C@@]1(O)C(=O)OCc2c1cc1n(c2=O)Cc2c-1nc1cc(F)c(C)c3c1c2[C@@H](N(C)CCO)CC3]

Compound 140 TFA. To the solution of compound 105 trifluoroacetate (1.0 equiv., 14 mg, 0.024 mmol) in formic acid (0.45 ml) was added 37% aqueous formaldehyde (0.12 ml) and the resulting mixture was stirred at 50° C. for 6 hours. Then, water was added, and the mixture was concentrated on rotary evaporator. The residue was purified by reverse-phase flash chromatography using semipreparative column (diol-modified C18, 0→60% ACN/0.1% TFA), giving the trifluoroacetate of compound 141 (4.5 mg, 31%) as a white powder after lyophilisation. MS calc. for C₂₇H₂₉FN₃O₅: 494.21, found: 494.25, [M+H]⁺. ¹H NMR (400 MHz, DMSO-d₆) δ: 7.97-7.73 (m, 1H), 7.34 (s, 1H), 6.74-6.33 (m, 1H), 5.58-5.26 (m, 4H), 5.17-4.88 (m, 1H), 3.87-3.50 (m, 7H), 3.30-2.94 (m, 2H), 2.90-2.59 (m, 1H), 2.39 (s, 3H), 2.30-2.16 (m, 1H), 1.96-1.78 (m, 2H), 0.89 (t, J=7.3 Hz, 3H).

### Example 43: Synthesis of Compound 147

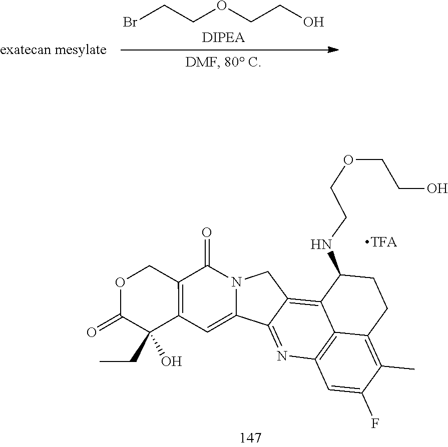 [SMILES: OCCOCCBr ; CC[C@@]1(O)C(=O)OCc2c1cc1n(c2=O)Cc2c-1nc1cc(F)c(C)c3c1c2[C@@H](NCCOCCO)CC3]

Compound 147. To the suspension of exatecan mesylate (1.0 equiv., 40 mg, 0.075 mmol) in DMF (2 mL) were added diisopropylethylamine (3.5 equiv., 0.26 mmol, 45 μL) and 2-(2-bromoethoxy)ethanol (2.0 equiv., 26 mg) under an argon atmosphere and the mixture was heated to 80° C. for 2 days. Purification of the residue by reverse-phase flash chromatography using semipreparative column (diol-modified C18, 0→60% ACN/0.1% TFA) offered the trifluoroacetate of compound 147 (8 mg, 17%) as a yellowish powder after lyophilisation. MS calc. for C₂₈H₃₁FN₃O₆: 524.22, found: 524.25, [M+H]⁺. ¹H NMR (400 MHz, DMSO-d₆) δ: 9.07 (s br, 1H), 8.87 (s br, 1H), 7.88 (d, J=10.7 Hz, 1H), 7.35 (s, 1H), 6.56 (s, 1H), 5.52 (d, J=19.1 Hz, 1H), 5.45 (s, 2H), 5.41 (d, J=19.1 Hz, 1H), 5.09 (s br, 1H), 4.71 (s br, 1H), 3.80-3.64 (m, 2H), 3.63-3.56 (m, 2H), 3.56-3.51 (m, 2H), 3.27-3.10 (m, 2H), 2.83-2.71 (m, 1H), 2.41 (s, 3H), 2.24-2.13 (m, 1H), 1.96-1.79 (m, 2H), 0.87 (t, J=7.3 Hz, 4H).

### Example 44: Synthesis of Compound 148

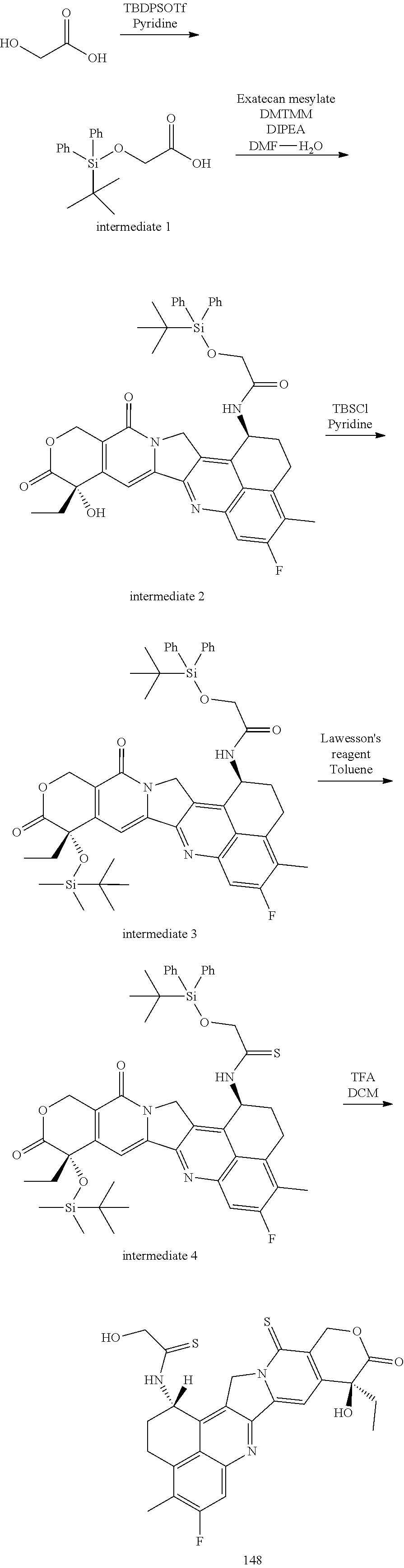

Intermediate 1. Glycolic acid (100 mg, 1.316 mmol) was co-evaporated three times with anhydrous pyridine, to be then dissolved in 2 mL of anhydrous pyridine under an argon atmosphere. Tert-Butyldiphenylsilyl trifluoromethanesulfonate (2 equiv., 2.632 mmol, 723 mg, 556 μL) was added, and the reaction mixture was stirred at room temperature for 12 hours. The reaction mixture was cooled at 0° C. and water (5 mL) was added. The residue was purified by reverse-phase flash chromatography (diol-modified C18, 0→60% ACN/0.1% TFA), giving the intermediate 1 (339 mg, 82%) as a colourless liquid. MS calc. for C₁₈H₂₁O₃Si: 313.12, found: 313.20, [M−H]⁻.

Intermediate 2. Exatecan mesylate (100 mg, 0.188 mmol), the previously prepared intermediate 1 (2 equiv., 0.376 mmol, 118 mg), DMTMM (1.2 equiv, 0.226 mmol, 62 mg), diisopropylethylamine (100 μL) and water (500 μL) were mixed in 4 mL of DMF and the reaction mixture was stirred at room temperature for 1 h. The residue was directly purified by reverse-phase flash chromatography (diol-modified C18, 0→100% ACN/water), giving intermediate 2 (97 mg, 70%) as a yellow solid. MS calc. for C₄₂H₄₃FN₃O₆Si: 732.29, found: 732.33, [M+H]⁺.

Intermediate 3. The previously prepared intermediate 2 (97 mg, 0.132 mmol) was co-evaporated three times with anhydrous pyridine, to be then dissolved in 5 mL of anhydrous pyridine under an argon atmosphere. Tert-Butyldimethyl(chloro)silane (10 equiv., 1.32 mmol, 199 mg) was added, and the reaction mixture was stirred at 80° C. for 48 hours. The residue was directly purified by reverse-phase flash chromatography (diol-modified C18, 0→100% ACN/water), giving intermediate 3 (53 mg, 47%) as a yellow solid. MS calc. for C₄₈H₅₇FN₃O₆Si₂: 846.38, found: 846.12, [M+H]⁺.

Intermediate 4. The previously prepared intermediate 3 (25 mg, 0.0296 mmol) was dissolved in 5 mL of anhydrous toluene, and Lawesson's reagent (4 equiv., 0.0592, 24 mg) was added. The reaction mixture was stirred at 100° C. for 4 hours. The reaction mixture was allowed to reach room temperature, and toluene was evaporated under reduced pressure. The crude reaction product was re-dissolved in 2 mL of DMF and the residue was directly purified by reverse-phase flash chromatography (diol-modified C18, 0→100% ACN/water), giving intermediate 4 (18 mg, 76%) as a yellow solid. MS calc. for C₄₈H₅₇FN₃O₄S₂Si₂: 878.33, found: 878.50, [M+H]⁺.

Compound 148. The previously prepared intermediate 4 (18 mg, 0.0205 mmol) was dissolved in 2 mL of anhydrous dichlorometane, and 1 mL of trifluoroacetic acid was added. The reaction mixture was stirred at room temperature for 2 hours. Solvents were evaporated under reduced pressure and the crude reaction product was re-dissolved in 2 mL of DMF to be directly purified by reverse-phase flash chromatography (diol-modified C18, 0→100% ACN/water). A second reverse-phase HPLC purification (semipreparative HPLC, diol-modified C18, 0→100% ACN in water) afforded as a 1:1 mixture of two inseparable isomers, with both forms in equilibrium (7 mg, 65%, yellow solid). MS calc. for C₂₆H₂₅FN₃O₄S₂: 526.13, found: 526.15, [M+H]⁺. ¹H NMR (500 MHz, DMSO-d₆, mixture of two isomers), δ 10.51 (m, 1H), 7.85 (d, J=10.8 Hz, 1H), 7.81 (d, J=2.9 Hz, 1H), 6.69 (d, J=9.1 Hz, 1H), 6.50 (q, J=9.4, 8.2 Hz, 1H), 6.05 (m, 1H), 5.91 (m, 1H), 5.51 (m, 1H), 5.47-5.31 (m, 2H), 4.60-4.44 (m, 2H), 3.16 (t, J=13.2 Hz, 1H), 2.51 (q, J=1.9 Hz, 2H), 2.40 (s, 3H), 2.28 (m, 1H), 1.96-1.83 (m, J=7.1 Hz, 2H), 0.96-0.84 (m, 3H).

### Example 45: Synthesis of Compound 159

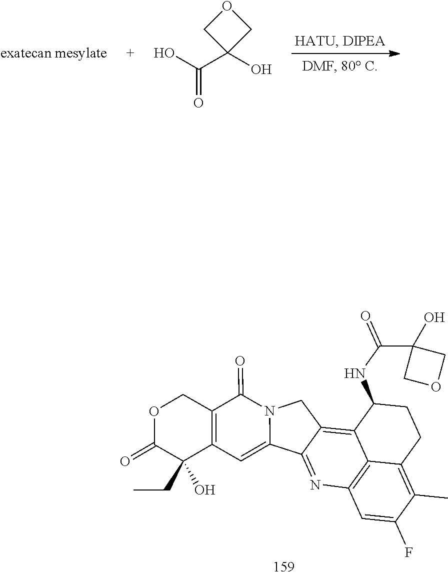 [SMILES: O=C(O)C1(O)COC1 ; CC[C@@]1(O)C(=O)OCc2c1cc1n(c2=O)Cc2c-1nc1cc(F)c(C)c3c1c2[C@@H](NC(=O)C1(O)COC1)CC3]

Compound 159. To the mixture of exatecan mesylate (1.0 eqiuv., 20 mg, 0.038 mmol), (3-hydroxyoxetan-3-yl)carboxylic acid (1.25 eqiuv., 6 mg, 0.048 mmol) and HATU (2.0 eqiuv., 29 mg, 0.076 mmol) was added dry DMF (1.5 mL) and diisopropylethylamine (4.0 equiv., 0.152 mmol, 27 μL) an the resulting solution was stirred under argon atmosphere for 1 hour at room temperature, as LC-MS indicated the full consumption of the starting material. The mixture was directly purified by reverse-phase flash chromatography using semipreparative column (diol-modified C18, 0→70% ACN/H₂O), offering the product (17 mg, 84%) as a white powder after lyophilization. MS calc. for C₂₈H₂₇FN₃O₇: 536.18, found: 536.20, [M+H]⁺. ¹H NMR (400 MHz, DMSO-d₆) δ: 8.52 (d, J=9.1 Hz, 1H), 7.68 (d, J=10.8 Hz, 1H), 7.27 (s, 1H), 6.94 (s, 1H), 6.51 (s, 1H), 5.60 (q, J=7.6 Hz, 1H), 5.47-5.33 (m, 2H), 5.04 (d, J=18.8 Hz, 1H), 4.99 (d, J=6.5 Hz, 1H), 4.94-4.83 (m, 2H), 4.58 (d, J=6.5 Hz, 1H), 4.52 (d, J=6.3 Hz, 1H), 3.27-3.16 (m, 1H), 3.16-3.02 (m, 1H), 2.34 (s, 3H), 2.23-2.13 (m, 2H), 1.93-1.78 (m, 2H), 0.87 (t, J=7.3 Hz, 3H).

### Example 46: Synthesis of Compound 163

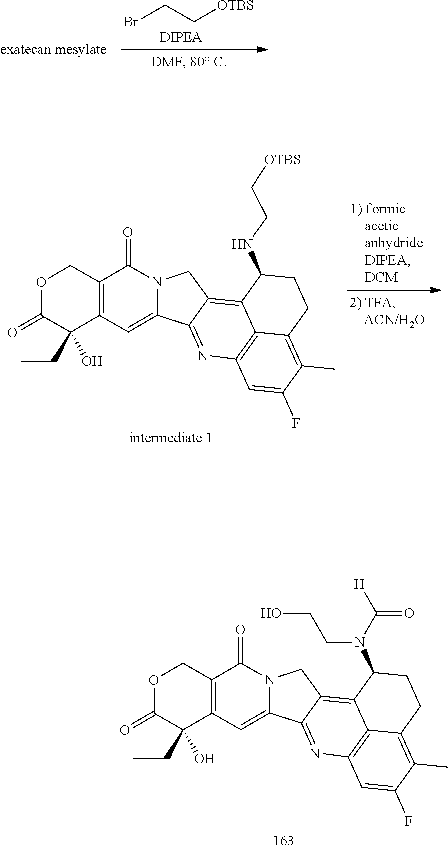 [SMILES: CC(C)(C)[Si](C)(C)OCCBr ; CC[C@@]1(O)C(=O)OCc2c1cc1n(c2=O)Cc2c-1nc1cc(F)c(C)c3c1c2[C@@H](NCCO[Si](C)(C)C(C)(C)C)CC3 ; CC[C@@]1(O)C(=O)OCc2c1cc1n(c2=O)Cc2c-1nc1cc(F)c(C)c3c1c2[C@@H](N(C=O)CCO)CC3]

Intermediate 1. To the suspension of exatecan mesylate (1.0 equiv., 30 mg, 0.056 mmol) in DMF (1.2 mL) were added diisopropylethylamine (4.5 equiv., 0.25 mmol, 45 μL) and (2-bromoethoxy)-tert-butyldimethylsilane (3.3 equiv., 0.19 mmol, 45 mg, 40 μL) under an argon atmosphere and the mixture was heated to 80° C. for 3 days. Purification by reverse-phase flash chromatography (25 g, diol-modified C18, 0→60% ACN/H₂O) offered the product intermediate 1 (13 mg, 39%) as an off-white powder after lyophilization.

Compound 163. Formic acetic anhydride (2.2 equiv., 0.048, 4.2 μL) (prepared according to Huffman, C. W. J. Org. Chem. 1958, 23 (5), 727-729.) and diisopropylethylamine (2.5 equiv., 0.055 mmol, 10 μL) were added to the solution of intermediate 1 (1.0 equiv., 13 mg, 0.022 mmol) in DCM (1.5 ml) and the resulting mixture was stirred at room temperature for 2 hours. Then, volatiles were removed on rotary evaporator and the residue was purified by reverse-phase flash chromatography (25 g, diol-modified C18, 0→80% ACN/H₂O). The fractions containing the TBS protected product were combined, acidified with TFA (50 μL) and evaporated to dryness (2×). Purification of the residue by reverse-phase flash chromatography using semipreparative column (diol-modified C18, 0→70% ACN/H₂O) offered the product (6 mg, 54%) as a white powder after lyophilization. MS calc. for C₂₇H₂₇FN₃O₆: 508.19, found: 508.20, [M+H]⁺. ¹H NMR (400 MHz, DMSO-d₆, mixture of isomers) δ: 8.28 (s, 0.55H), 8.17 (s, 0.45H), 7.78 (dd, J=13.3, 10.8 Hz, 1H), 7.30 (s, 1H), 6.51 (s, 1H), 5.59-5.47 (m, 1H), 5.41 (s, 3H), 5.25 (d, J=18.8 Hz, 0.45H), 5.17-5.00 (m, 1H), 4.97-4.87 (m, 1H), 4.77 (t, J=5.2 Hz, 0.55H), 3.68-3.38 (m, 2H), 3.28-2.96 (m, 2H), 2.46-2.17 (m, 4H), 1.98-1.77 (m, 2H), 0.87 (t, J=7.3 Hz, 3H).

### Example 47: Synthesis of Compound 164

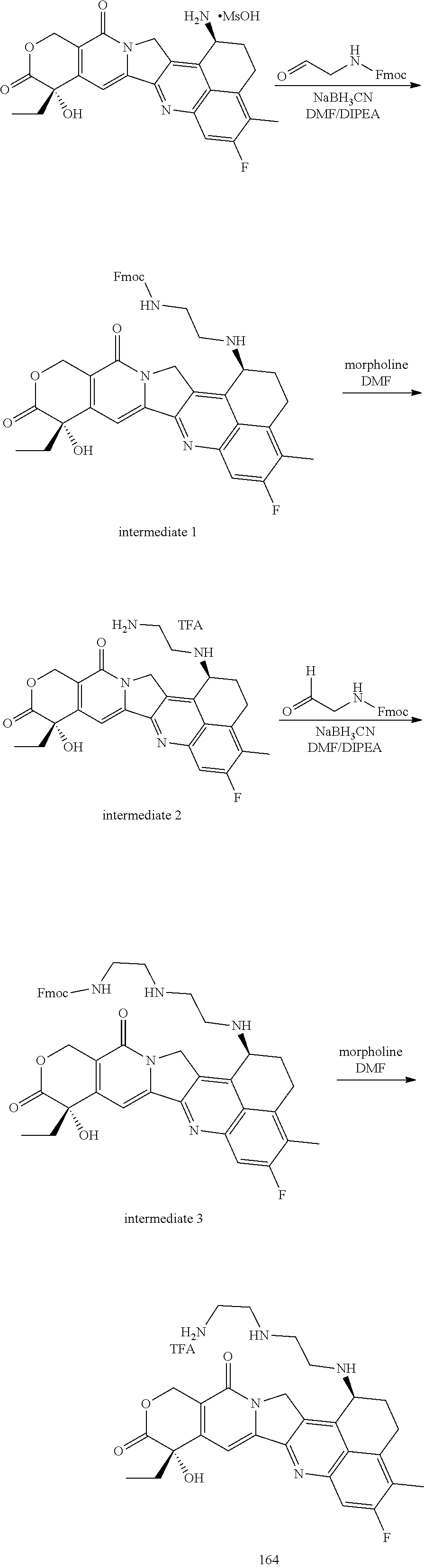

Intermediate 1. Exatecan mesylate (30 mg, 0.056 mmol), N-(Fmoc)-2-aminoacetaldehyde (24 mg, 0.085 mmol) and DIPEA (40 μL) were dissolved in dry DMF (2 mL). The mixture was stirred for 1 hour at 60° C. Then NaBH₃CN (30 mg, 0.47 mmol) was added and the reaction mixture was stirred next 2 hours at 60° C. The mixture was directly purified by reverse-phase flash chromatography (25 g, diol-modified C18, 0→70% ACN/H₂O). Fractions containing the product were lyophilized from water (28 mg, 72%). MS calc. for C₄₁H₃₈FN₄O₆⁺: 701.27, found: 701.43, [M+H]⁺.

Intermediate 2. The previously prepared intermediate 1 (28 mg, 0.04 mmol) was dissolved in DMF (2 mL) and morpholine (100 μL) was added. The reaction mixture was stirred at room temperature for 1 h. Purification was performed by reverse-phase HPLC chromatography (25 g, diol-modified C18, 0→50% ACN/H₂O(TFA)). Fractions containing the product were lyophilized from water (14 mg, 76%). MS calc. for C₂₆H₂₈FN₄O₄⁺: 479.20, found: 479.32, [M+H]⁺.

Intermediate 3. The previously prepared intermediate 2 (17 mg, 0.036 mmol) and N-(Fmoc)-2-aminoacetaldehyde (5 mg, 0.018 mmol) were dissolved in dry DMF (2 mL). The mixture was stirred for 1 hour at 60° C. Then NaBH₃CN (10 mg, 0.16 mmol) was added and the reaction mixture was stirred next 2 hours at 60° C. The mixture was directly purified by reverse-phase flash chromatography (25 g, diol-modified C18, 0→70% ACN/H₂O). Fractions containing the product were lyophilized from water (10 mg, 37%). MS calc. for C₄₃H₄₃FN₅O₆: 744.31, found: 744.45, [M+H]⁺.

Compound 164. The previously prepared intermediate 3 (10 mg, 0.013 mmol) was dissolved in DMF (2 mL) and morpholine (100 μL) was added. The reaction mixture was stirred at room temperature for 1 h. Purification was performed by reverse-phase HPLC chromatography (semipreparative, diol-modified C18, 0→50% ACN/H₂O(TFA)). Fractions containing the product were lyophilised from water (3 mg, 44%). MS calc. for C₂₈H₃₃FN₅O₄⁺: 522.24, found: 522.30, [M+H]⁺. ¹H NMR (500 MHz, DMSO-d₆) δ 7.81 (d, J=10.8 Hz, 1H), 7.35 (s, 1H), 6.57 (s, 2H), 5.45 (s, 2H), 4.46 (s, 1H), 3.90 (m, 1H), 3.77 (m 1H), 3.18-3.14 (m, 2H), 3.12 (d, J=10.2 Hz, 4H), 3.04-2.96 (m, 4H), 2.41 (s, 3H), 2.08 (m, 1H), 1.88 (m, 2H), 0.88 (t, J=7.3 Hz, 3H).

### Example 48: Synthesis of Compound 166

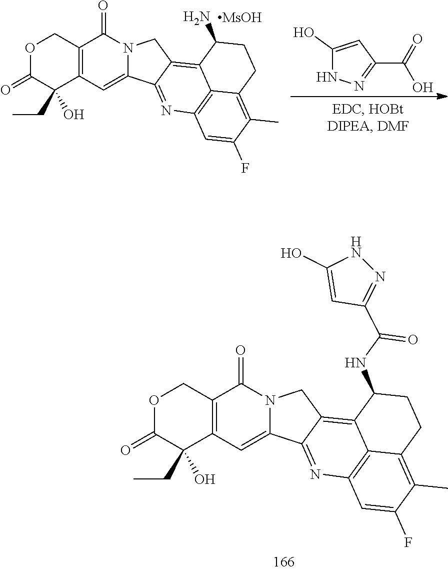 [SMILES: "·MsOH" ; CC[C@@]1(O)C(=O)OCc2c1cc1n(c2=O)Cc2c-1nc1cc(F)c(C)c3c1c2[C@@H](N)CC3 ; O=C(O)c1cc(O)[nH]n1 ; CC[C@@]1(O)C(=O)OCc2c1cc1n(c2=O)Cc2c-1nc1cc(F)c(C)c3c1c2[C@@H](NC(=O)c1cc(O)[nH]n1)CC3]

To DMF (2 mL) were added exatecan mesylate (30 mg, 0.056 mmol), 5-Hydroxy-1H-pyrazole-3-carboxylic acid (11 mg, 0.084 mmol), EDC (22 mg, 0.115 mmol), HOBt (18 mg, 0.117 mmol) and diisopropyethylamine (30 μL). The resulting solution was stirred under argon atmosphere for 16 hours at room temperature. The mixture was directly purified by reverse-phase HPLC chromatography using semipreparative column (diol-modified C18, 0→100% ACN/H₂O). The desired product was obtained as a yellow powder after lyophilisation from water (7 mg, 23%). MS calc. for C₂₈H₂₅FN₅O₆: 546.17, found: 546.25, [M+H]⁺. ¹H NMR (400 MHz, DMSO-d₆) δ 10.15 (s, 1H), 8.79 (d, J=8.7 Hz, 1H), 7.80 (d, J=10.8 Hz, 1H), 7.31 (s, 1H), 6.68 (s, 1H), 6.02 (s, 1H), 5.72 (q, J=6.6 Hz, 1H), 5.39 (s, 2H), 5.17 (s, 2H), 3.27 (m, 1H), 3.19-3.10 (m, 1H), 2.41 (d, J=1.9 Hz, 3H), 2.25 (m, 2H), 1.94-1.81 (m, 2H), 1.77 (s, 1H), 0.87 (t, J=7.3 Hz, 3H).

### Example 49: Synthesis of Compound 167

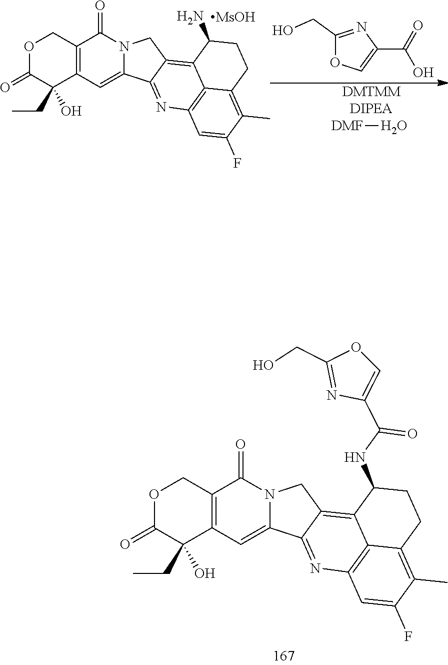

To a 4:1 DMF/water mixture (4 mL) were added exatecan mesylate (20 mg, 0.038 mmol), 2-(hydroxymethyl)oxazole-4-carboxylic acid (1.25 equiv., 7 mg, 0.048 mmol), DMTMM (2.0 equiv., 21 mg, 0.076 mmol) and diisopropylethylamine (20 μL). The resulting solution was stirred for 1 hour at room temperature, as LC-MS indicated the full consumption of the starting material. The mixture was directly purified by reverse-phase HPLC chromatography using semipreparative column (diol-modified C18, 0→100% ACN/H₂O). The desired product was obtained as a white powder after lyophilisation from water (17 mg, 80%). MS calc. for C₂₉H₂₆FN₄O₇: 561.18, found: 561.20, [M+H]⁺. 1H NMR (400 MHz, DMSO-d6) δ 9.01 (d, J=8.8 Hz, 1H), 8.71 (s, 1H), 7.76 (d, J=10.9 Hz, 1H), 7.30 (s, 1H), 6.50 (s, 1H), 5.78 (t, J=6.2 Hz, 1H), 5.73-5.64 (m, 1H), 5.37 (s, 2H), 5.10 (s, 2H), 4.54 (d, J=6.2 Hz, 2H), 3.98 (s, 1H), 3.30-3.23 (m, 1H), 3.17-3.09 (m, 1H), 2.38 (d, J=1.9 Hz, 3H), 2.33-2.19 (m, 1H), 1.95-1.80 (m, 2H), 0.86 (t, J=7.3 Hz, 3H).

### Example 50: Synthesis of Compound 168

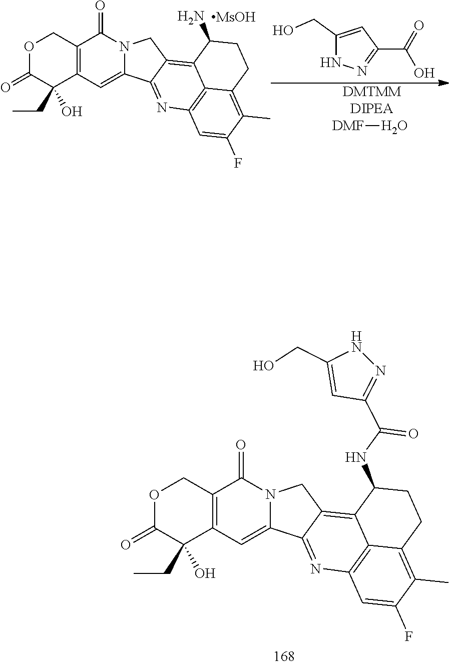

To a 4:1 DMF/water mixture (4 mL) were added exatecan mesylate (20 mg, 0.038 mmol), 5-(hydroxymethyl)-1H-pyrazole-3-carboxylic acid (1.25 eqiuv., 7 mg, 0.048 mmol), DMTMM (2.0 equiv., 21 mg, 0.076 mmol) and diisopropyethylamine (20 μL). The resulting solution was stirred under argon atmosphere for 1 hour at room temperature, as LC-MS indicated the full consumption of the starting material. The mixture was directly purified by reverse-phase HPLC chromatography using semipreparative column (diol-modified C18, 0→100% ACN/H₂O). The desired product was obtained as a white powder after lyophilisation from water (14 mg, 66%). MS calc. for C₂₉H₂₇FN₅O₆: 560.19, found: 560.30, [M+H]⁺. ¹H NMR (400 MHz, DMSO-d₆) δ 13.25-13.20 (m, 1H), 8.85 (d, J=8.9 Hz, 1H), 7.76 (d, J=10.9 Hz, 1H), 7.29 (s, 1H), 6.62 (d, J=1.9 Hz, 1H), 6.51 (d, J=3.4 Hz, 1H), 5.69 (q, J=7.6 Hz, 1H), 5.38 (d, J=12.2 Hz, 2H), 5.28-5.01 (m, 3H), 4.54 (d, J=5.6 Hz, 2H), 3.29-3.01 (m, 1H), 2.43-2.36 (m, 4H), 2.29-2.22 (m, 2H), 1.95-1.74 (m, 2H), 0.86 (t, J=7.3 Hz, 3H).

### Example 51: Synthesis of Compound 175

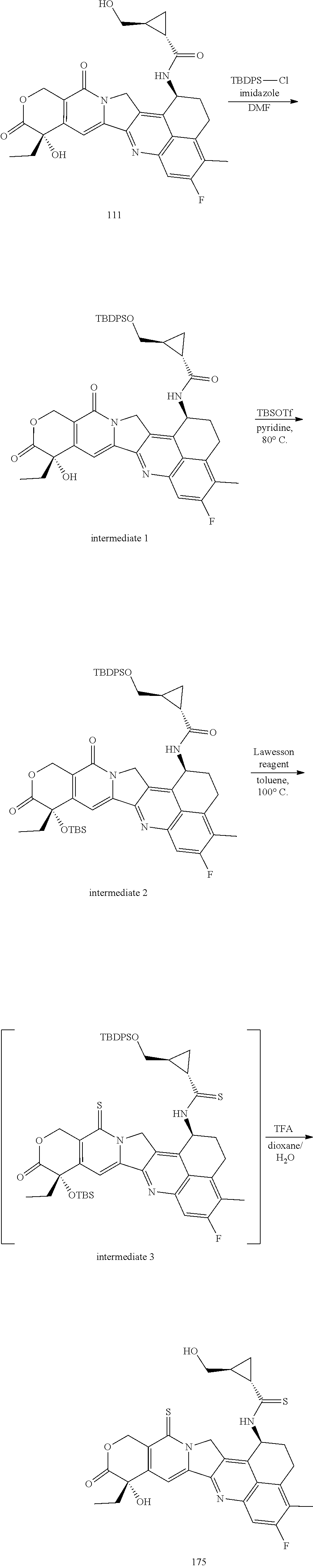

Intermediate 1. To the solution of compound 11 (1.0 equiv., 52 mg, 0.097 mmol) and imidazole (2.5 equiv., 0.243 mmol, 17 mg) in DMF (2 mL) was added tert-butyl(chloro)diphenylsilane (2.5 equiv., 0.243 mmol, 68 mg, 63 μL) under an argon atmosphere and the mixture was stirred at room temperature for 4 hours. Purification by reverse-phase flash chromatography (25 g, diol-modified C18, 0→80% ACN/H₂O) offered the product intermediate 1 (61 mg, 81%) as an off-white powder after lyophilisation.

Intermediate 2. To the solution of intermediate 1 (1.0 equiv., 61 mg, 0.078 mmol) in dry pyridine (3 mL) was added tert-butyldimethylsilyl trifluoromethanesulfonate (14.0 equiv., 1.09 mmol, 287 mg, 250 μL) under an argon atmosphere and the mixture was heated to 80° C. for 5 hours. Then, it was concentrated on rotary evaporator and the residue was purified by reverse-phase flash chromatography (25 g, diol-modified C18, 0→100% ACN/H₂O), giving the product intermediate 2 (56 mg, 81%) as a white powder after lyophilization.

Compound 175. To the mixture of intermediate 2 (1.0 equiv., 56 mg, 0.063 mmol) and Lawesson reagent (5.0 equiv., 0.316 mmol, 128 mg) was added anhydrous toluene (8 ml) and the resulting mixture was heated to 100° C. for 6 hours. The progress of the reaction was monitored by LC-MS. More Lawesson reagent was added (2.0 equiv., 0.126 mmol, 52 mg) and the mixture was heated to 100° C. for another 3 hours. Then, volatiles were removed on rotary evaporator and the residue was purified by reverse-phase flash chromatography (25 g, diol-modified C18, 0→100% ACN/H₂O). The fractions containing the intermediate 3 were combined and concentrated on rotary evaporator. The residue was dissolved in dioxane (2 ml), followed by the addition of water (1 ml) and trifluoroacetic acid (1 ml). The resulting mixture was stirred at room temperature for 40 hours and the progress of the reaction was monitored by LC-MS. Then, solvents were removed on rotary evaporator and the residue was purified by reverse-phase flash chromatography using semipreparative column (diol-modified C18, 0→70% ACN/H₂O), giving the product (12 mg, 34%) as a yellow powder after lyophilization. MS calc. for C₂₉H₂₉FN₃O₄S₂: 566.16, found: 566.20, [M+H]⁺. ¹H NMR (500 MHz, DMSO-d₆) δ: 10.62 (dd, J=15.6, 8.6 Hz, 1H), 7.85 (d, J=10.8 Hz, 1H), 7.80 (d, J=2.8 Hz, 1H), 6.68 (s, 1H), 6.53-6.41 (m, 1H), 5.91 (dd, J=16.6, 3.3 Hz, 1H), 5.55-5.45 (m, 1H), 5.46-5.30 (m, 2H), 3.71 (dd, J=11.2, 4.6 Hz, 1H), 3.50 (dd, J=11.5, 5.3 Hz, 1H), 3.31-3.10 (m, 2H), 2.40 (s, 3H), 2.36-2.17 (m, 1H), 2.17-2.06 (m, 1H), 2.04-1.82 (m, 3H), 1.54-1.34 (m, 1H), 1.10-0.93 (m, 1H), 0.86 (t, J=7.3 Hz, 3H).

### Example 52: Synthesis of Compound 176

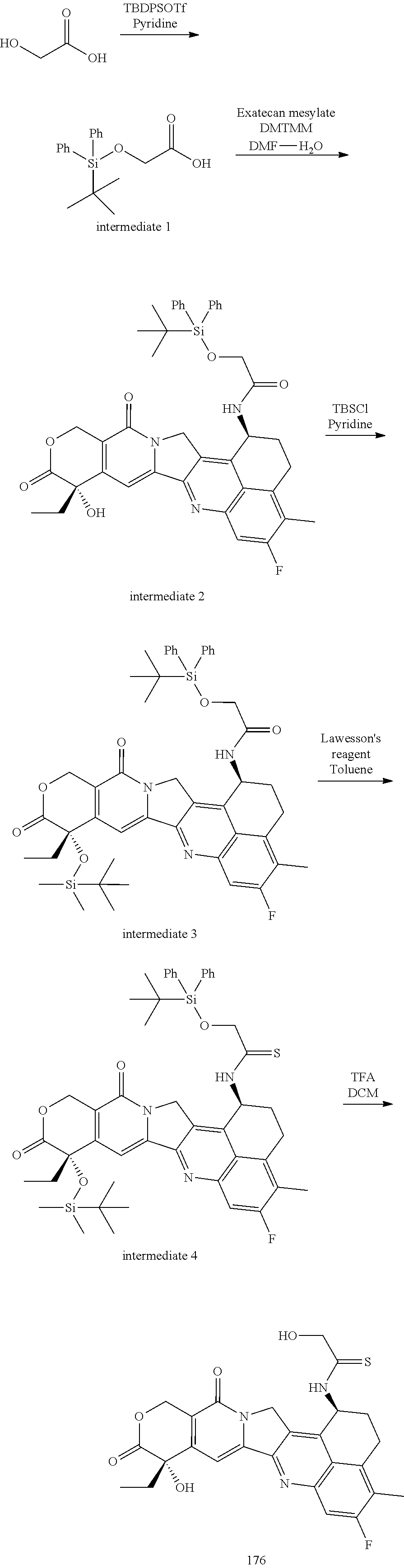

Intermediate 1. Glycolic acid (100 mg, 1.316 mmol) was co-evaporated three times with anhydrous pyridine, to be then dissolved in 2 mL of anhydrous pyridine under an argon atmosphere. Tert-Butyldiphenylsilyl trifluoromethanesulfonate (2 equiv., 2.632 mmol, 723 mg, 556 μL) was added, and the reaction mixture was stirred at room temperature for 12 hours. The reaction mixture was cooled at 0° C. and water (5 mL) was added. The residue was purified by reverse-phase flash chromatography (diol-modified C18, 0→60% ACN/0.1% TFA), giving the intermediate 1 (339 mg, 82%) as a colourless liquid. MS calc. for C₁₈H₂₁O₃Si: 313.12, found: 313.20, [M−H]⁻.

Intermediate 2. Exatecan mesylate (100 mg, 0.188 mmol), the previously prepared intermediate 1 (2 equiv., 0.376 mmol, 118 mg), DMTMM (1.2 equiv, 0.226 mmol, 62 mg), diisopropylethylamine (100 μL) and water (500 μL) were mixed in 4 mL of DMF and the reaction mixture was stirred at room temperature for 1 h. The residue was directly purified by reverse-phase flash chromatography (diol-modified C18, 0→100% ACN/water), giving the intermediate 2 (97 mg, 70%) as a yellow solid. MS calc. for C₄₂H₄₃FN₃O₆Si: 732.29, found: 732.33, [M+H]⁺.

Intermediate 3. The previously prepared intermediate 2 (97 mg, 0.132 mmol) was co-evaporated three times with anhydrous pyridine, to be then dissolved in 5 mL of anhydrous pyridine under an argon atmosphere. Tert-Butyldimethyl(chloro)silane (10 equiv., 1.32 mmol, 199 mg) was added, and the reaction mixture was stirred at 80° C. for 48 hours. The residue was directly purified by reverse-phase flash chromatography (diol-modified C18, 0→100% ACN/water), giving the intermediate 3 (53 mg, 47%) as a yellow solid. MS calc. for C₄₈H₅₇FN₃O₆Si₂: 846.38, found: 846.12, [M+H]⁺.

Intermediate 4. The previously prepared intermediate 3 (25 mg, 0.0296 mmol) was dissolved in 5 mL of anhydrous toluene, and Lawesson's reagent (1 equiv., 0.0296, 6 mg) was added. The reaction mixture was stirred at 100° C. for 4 hours. The reaction mixture was allowed to reach room temperature, and toluene was evaporated under reduced pressure. The crude reaction product was re-dissolved in 2 mL of DMF and the residue was directly purified by reverse-phase flash chromatography (diol-modified C18, 0→100% ACN/water), giving the intermediate 4 (22 mg, 86%) as a yellow solid. MS calc. for C₄₈H₅₇FN₃O₅SSi₂: 862.35, found: 862.40, [M+H]⁺.

Compound 176. The previously prepared intermediate 4 (22 mg, 0.0255 mmol) was dissolved in 1 mL of anhydrous dichlorometane, and 2 mL of trifluoroacetic acid was added. The reaction mixture was stirred at room temperature for 16 hours. Solvents were evaporated under reduced pressure and the crude reaction product was re-dissolved in 1 mL of DMF to be directly purified by reverse-phase flash chromatography (diol-modified C18, 0→80% ACN/water). A second reverse-phase HPLC purification (semipreparative HPLC, diol-modified C18, 0→100% ACN in water) afforded compound 177 as a 1:1 mixture of two inseparable isomers, with both forms in equilibrium (11 mg, 84%, orange solid). MS calc. for C₂₆H₂₅FN₃O₅S: 510.15, found: 510.15, [M+H]⁺. ¹H NMR (401 MHz, DMSO-d₆) δ 10.40 (d, J=8.8 Hz, 1H), 7.81 (d, J=10.9 Hz, 1H), 7.32 (s, 1H), 6.53 (s, 1H), 6.44-6.38 (m, 1H), 5.98 (t, J=5.8 Hz, 1H), 5.42 (s, 2H), 5.12 (d, J=3.4 Hz, 2H), 4.40 (d, J=5.8 Hz, 2H), 3.23-3.13 (m, 2H), 2.41 (d, J=1.9 Hz, 3H), 2.30-2.19 (m, 1H), 1.86 (dq, J=14.2, 7.1 Hz, 2H), 1.24 (s, 1H), 0.87 (t, J=7.3 Hz, 3H).

### Example 53: Synthesis of Compound 188

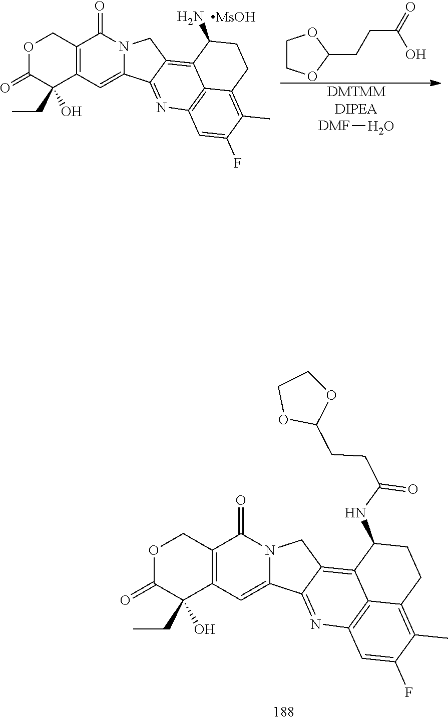

To a 4:1 DMF/water mixture (4 mL) were added exatecan mesylate (20 mg, 0.0376 mmol), 3-(1,3-dioxolan-2-yl)propanoic acid (2 eqiuv., 0.0753 mmol, 11 mg), DMTMM (1.5 equiv., 0.0564 mmol, 16 mg) and diisopropylethylamine (20 μL). The resulting solution was stirred for 1 hour at room temperature, as LC-MS indicated the full consumption of the starting material. The mixture was directly purified by reverse-phase HPLC chromatography using a semipreparative column (diol-modified C18, 0→100% ACN/H₂O). The desired product was obtained as a white powder after lyophilisation (19 mg, 89%). MS calc. for C₃₀H₃₁FN₃O₇: 564.21, found: 564.34, [M+H]⁺. ¹H NMR (400 MHz, DMSO-d₆) δ 8.46 (d, J=8.7 Hz, 1H), 7.78 (d, J=10.9 Hz, 1H), 7.30 (s, 1H), 6.52 (s, 1H), 5.56 (dt, J=9.3, 5.0 Hz, 1H), 5.43 (s, 2H), 5.31-5.11 (m, 2H), 4.83 (t, J=4.5 Hz, 1H), 3.89-3.80 (m, 2H), 3.79-3.69 (m, 2H), 3.24-3.10 (m, 2H), 2.40 (d, J=1.9 Hz, 3H), 2.26 (t, J=7.6 Hz, 2H), 2.14 (q, J=7.3, 6.6 Hz, 2H), 1.98-1.77 (m, 4H), 0.88 (t, J=7.3 Hz, 3H).

### Example 54: Synthesis of Compound 1002

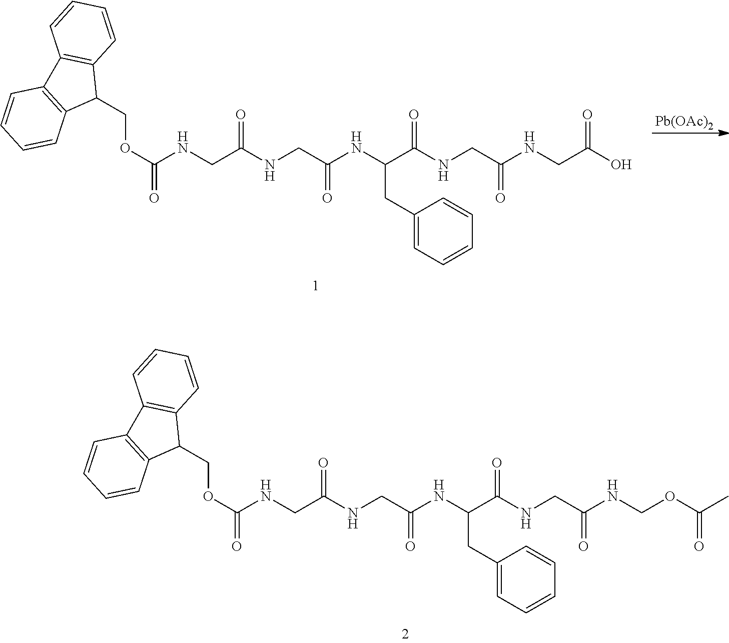 [SMILES: O=C(O)CNC(=O)CNC(=O)C(Cc1ccccc1)NC(=O)CNC(=O)CNC(=O)OCC1c2ccccc2-c2ccccc21 ; CC(=O)OCNC(=O)CNC(=O)C(Cc1ccccc1)NC(=O)CNC(=O)CNC(=O)OCC1c2ccccc2-c2ccccc21 ; "Pb(OAc)2"]

Intermediate 1 is dissolved in anhydrous DMF and magnetically stirred in a flask and copper (II) acetate acetic acid and lead tetraacetate were added. The flask is heated in a 60° C. oil bath for 20 min. The oil bath is removed and the reaction mixture is allowed to cool to room temperature. The mixture is purified on a C18 RP column to yield 260 intermediate 2.

### Step 2:

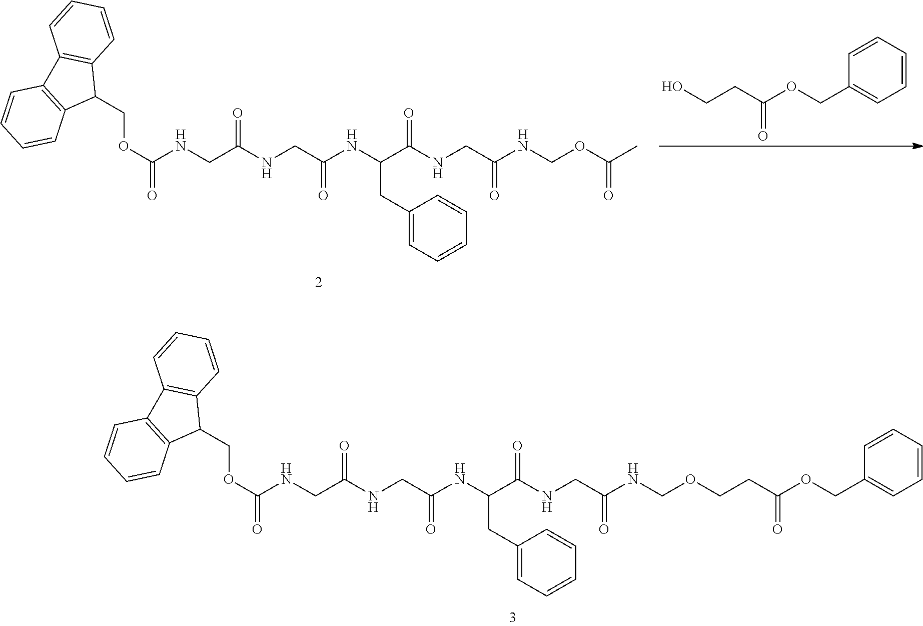 [SMILES: CC(=O)OCNC(=O)CNC(=O)C(Cc1ccccc1)NC(=O)CNC(=O)CNC(=O)OCC1c2ccccc2-c2ccccc21 ; O=C(CCO)OCc1ccccc1 ; O=C(CNC(=O)OCC1c2ccccc2-c2ccccc21)NCC(=O)NC(Cc1ccccc1)C(=O)NCC(=O)NCOCCC(=O)OCc1ccccc1]

Intermediate 2 and benzyl 3-hydroxypropionate are suspended in a cold solution of 20% TFA in dichloromethane and stirred at room temperature for 60 min. The solvent is evaporated, and the residue is purified on a C18 RP to give intermediate 3.

### Step 3:

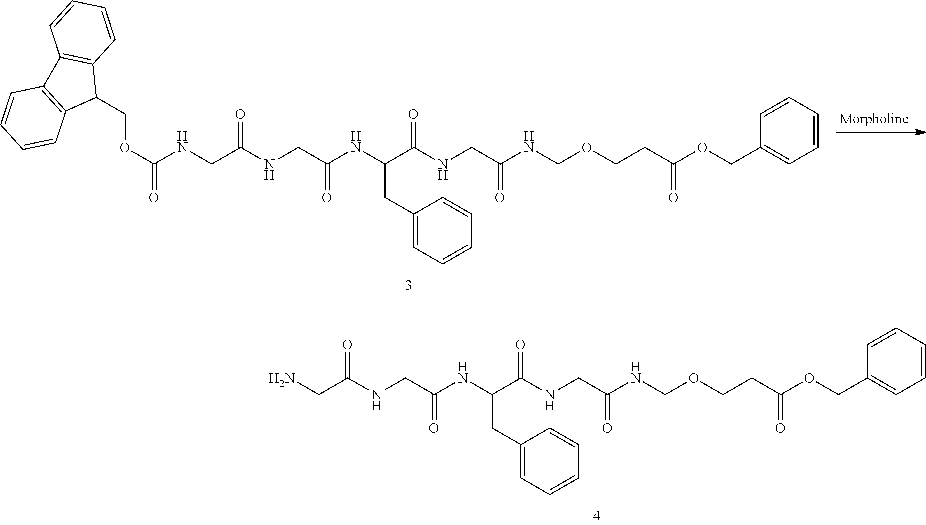 [SMILES: O=C(CNC(=O)OCC1c2ccccc2-c2ccccc21)NCC(=O)NC(Cc1ccccc1)C(=O)NCC(=O)NCOCCC(=O)OCc1ccccc1 ; NCC(=O)NCC(=O)NC(Cc1ccccc1)C(=O)NCC(=O)NCOCCC(=O)OCc1ccccc1]

Morpholine is added to a stirred solution of intermediate 3 in DMF. After 1.5 h the reaction mixture is loaded onto a C18 RP column and eluted to give intermediate 4.

### Step 4:

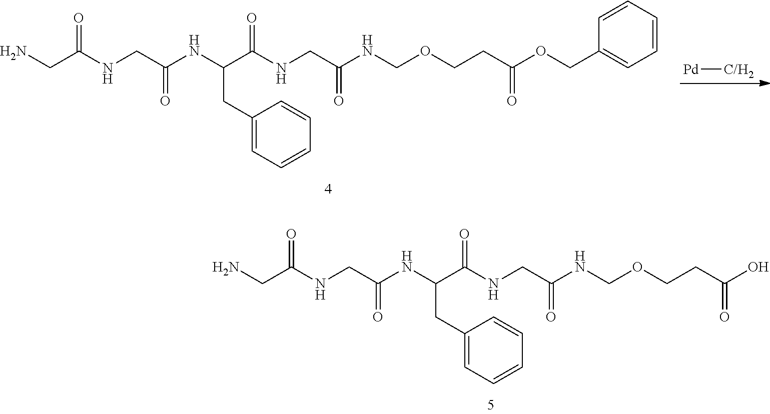

To a solution of intermediate 4 in 5:95 deionized water:methanol (35 mL) is added 10% palladium on carbon (0.09 g). The mixture is hydrogenated at 30 PSI H₂ for 80 min, filtered and evaporated under vacuum to afford intermediate 5.

### Step 5

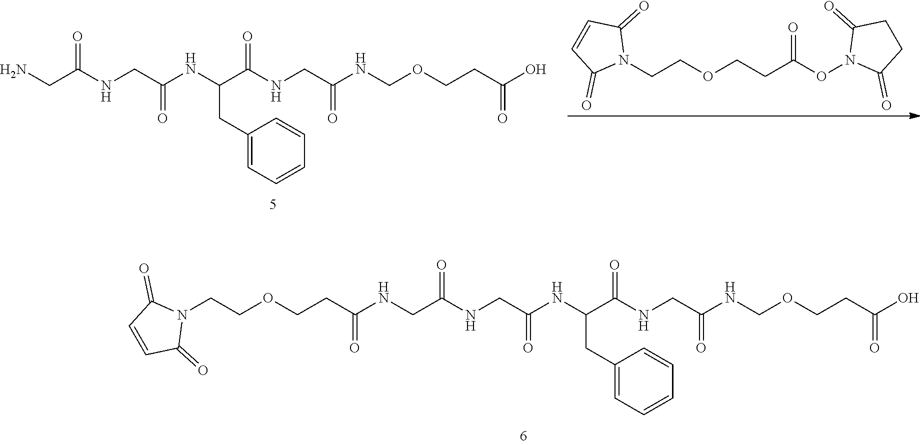

To a stirred solution of intermediate 5 in anhydrous DMF is added DIPEA and Mal-PEG1-NHS ester. The mixture is stirred for 30 min at RT, then applied to a C18 RP column and eluted to give intermediate 6.

### Step 6:

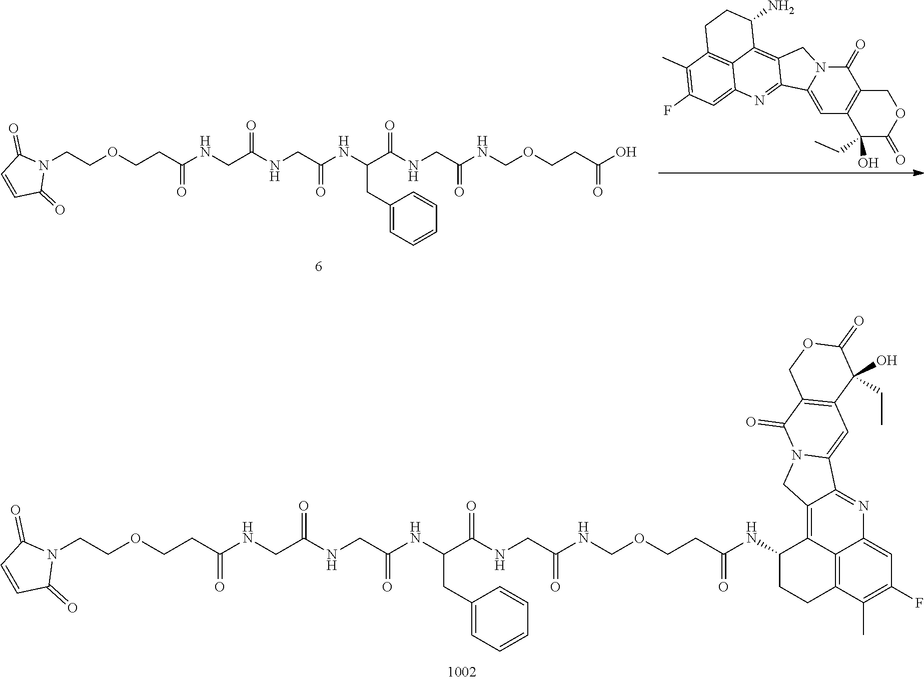 [SMILES: O=C(O)CCOCNC(=O)CNC(=O)C(Cc1ccccc1)NC(=O)CNC(=O)CNC(=O)CCOCCN1C(=O)C=CC1=O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CCOCNC(=O)CNC(=O)C(Cc1ccccc1)NC(=O)CNC(=O)CNC(=O)CCOCCN1C(=O)C=CC1=O)CC3]

A solution of exatecan mesylate DMTMM, triethylamine and intermediate 6 in 20% DMF/water is stirred at 37° C. for 30 minutes. The reaction mixture is cooled to RT and purified by silica gel column chromatography give compound 1002.

### Step 7:

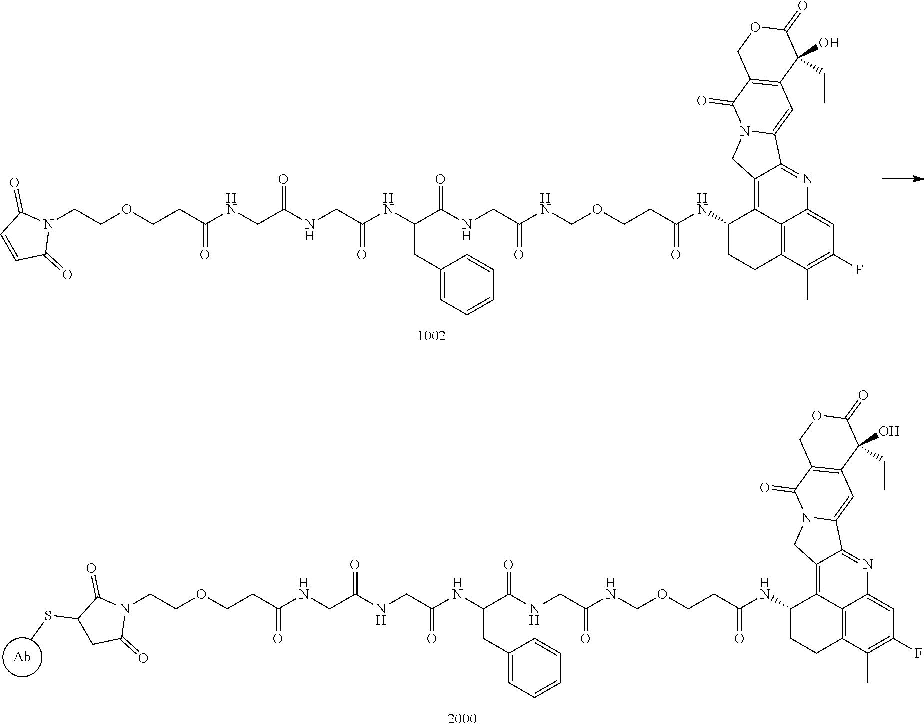

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1002 dissolved in a —40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2000.

### Example 55: Synthesis of Compound 1003

### Step 1:

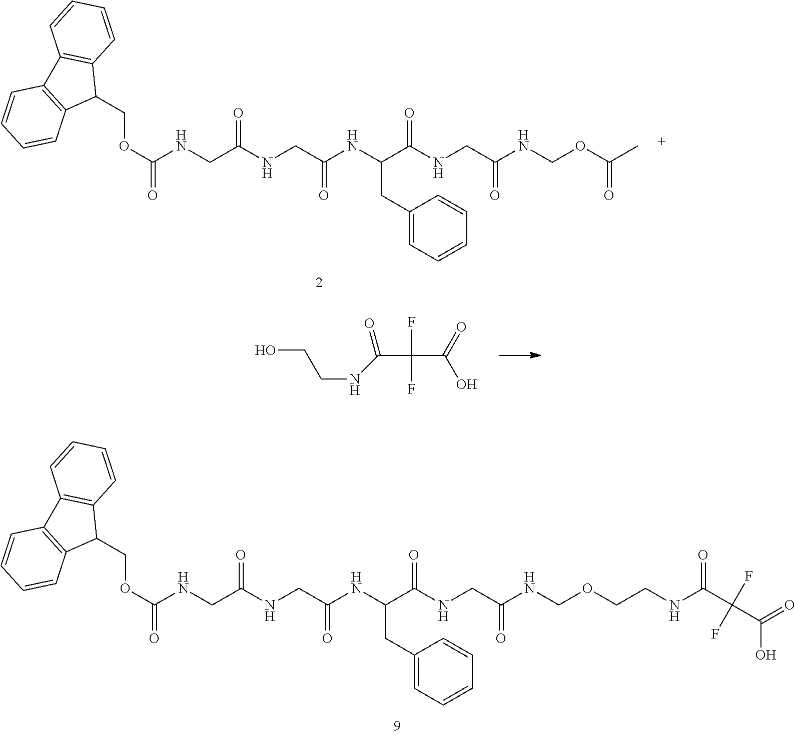 [SMILES: CC(=O)OCNC(=O)CNC(=O)C(Cc1ccccc1)NC(=O)CNC(=O)CNC(=O)OCC1c2ccccc2-c2ccccc21 ; O=C(O)C(F)(F)C(=O)NCCO ; O=C(CNC(=O)OCC1c2ccccc2-c2ccccc21)NCC(=O)NC(Cc1ccccc1)C(=O)NCC(=O)NCOCCNC(=O)C(F)(F)C(=O)O]

A suspension of intermediate 2 from Example 54 and 2,2-difluoro-3-((2-hydroxyethyl)amino)-3-oxopropanoic acid in 20% TFA/DCM is stirred at rt for 60 min. The solvent is evaporated and the residue is purified by reverse phase column chromatography to give intermediate 9.

### Step 2:

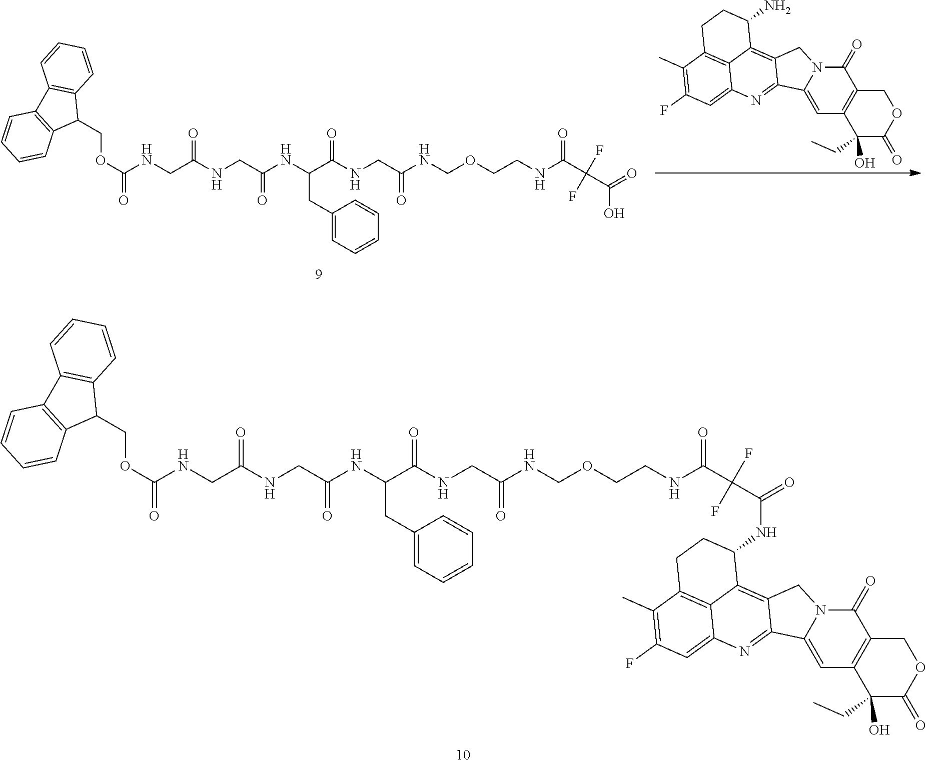 [SMILES: O=C(CNC(=O)OCC1c2ccccc2-c2ccccc21)NCC(=O)NC(Cc1ccccc1)C(=O)NCC(=O)NCOCCNC(=O)C(F)(F)C(=O)O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CNC(=O)OCC1c2ccccc2-c2ccccc21)CC3]

A solution of exatecan mesylate, DMTMM, triethylamine and intermediate 9 in 20% DMF/water is stirred at 35° C. for 30 minutes. The reaction mixture is purified by silica gel column chromatography to give intermediate 10.

### Step 3:

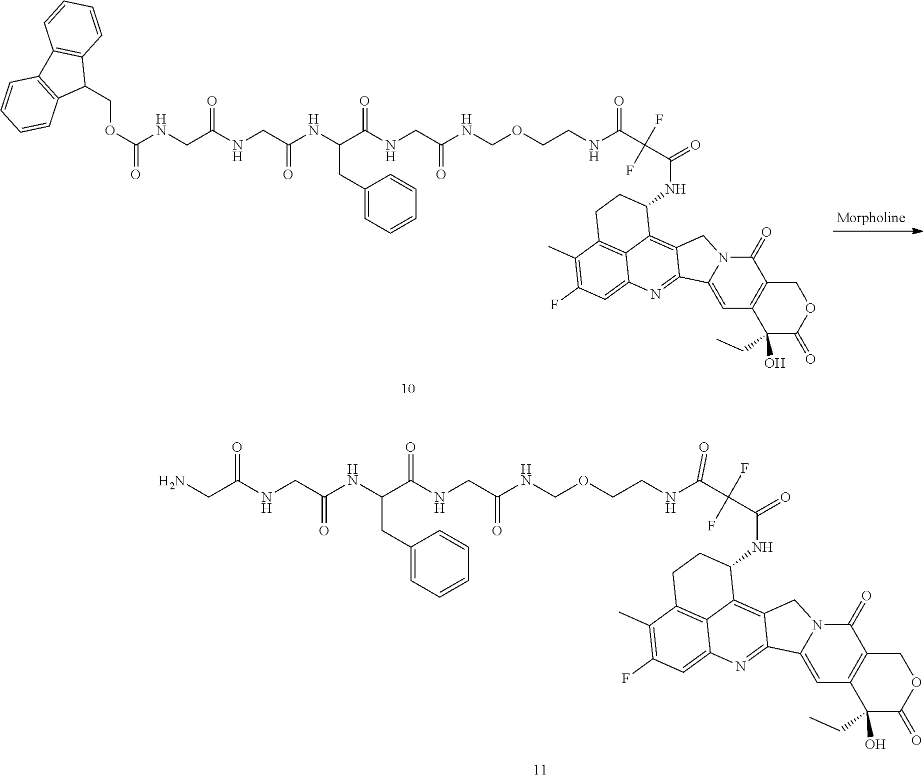 [SMILES: CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CNC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CN)CC3]

Morpholine is added to a stirred solution of intermediate 10 in DMF. After 1.5 h the reaction mixture is loaded onto a C18 RP column and eluted to give intermediate 11.

### Step 4:

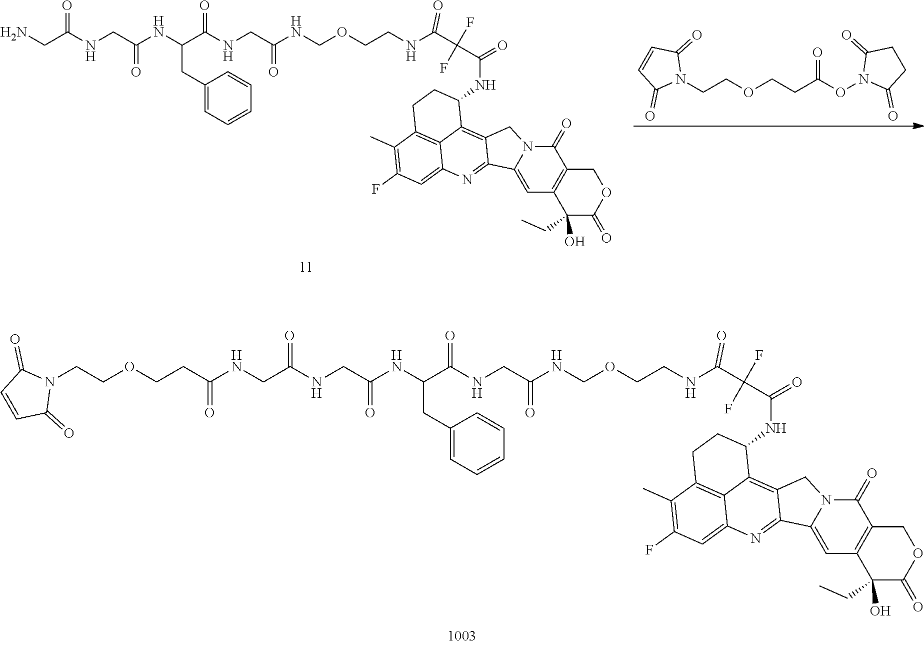 [SMILES: CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CN)CC3 ; O=C(CCOCCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)C(F)(F)C(=O)NCCOCNC(=O)CNC(=O)C(Cc1ccccc1)NC(=O)CNC(=O)CNC(=O)CCOCCN1C(=O)C=CC1=O)CC3]

To a stirred solution of intermediate 11 in anhydrous DMF is added DIPEA and Mal-PEG1-NHS ester. The mixture is stirred for 30 min at RT, and purified by reverse phase column chromatography to give compound 1003.

### Step 5:

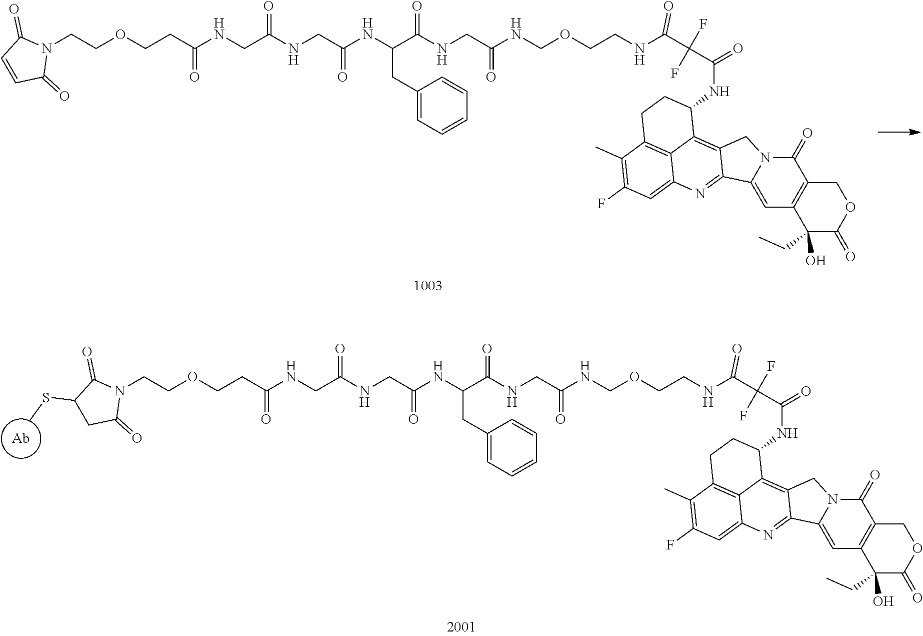

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1003 dissolved in a ˜40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2001.

### Example 56: Synthesis of Compound 1004

### Step 1:

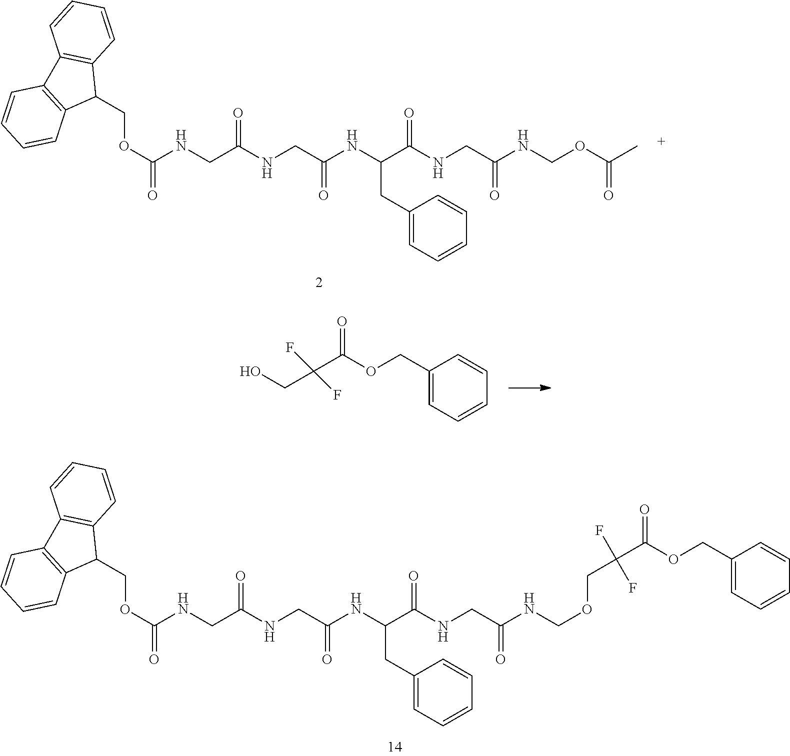 [SMILES: CC(=O)OCNC(=O)CNC(=O)C(Cc1ccccc1)NC(=O)CNC(=O)CNC(=O)OCC1c2ccccc2-c2ccccc21 ; O=C(OCc1ccccc1)C(F)(F)CO ; O=C(CNC(=O)OCC1c2ccccc2-c2ccccc21)NCC(=O)NC(Cc1ccccc1)C(=O)NCC(=O)NCOCC(F)(F)C(=O)OCc1ccccc1]

A suspension of intermediate 2 from Example 54 and benzyl 2,2-difluoro-3-hydroxypropanoate is stirred at rt for 60 min. The solvent is evaporated and the residue is purified by reverse phase column chromatography to give intermediate 14.

### Step 2:

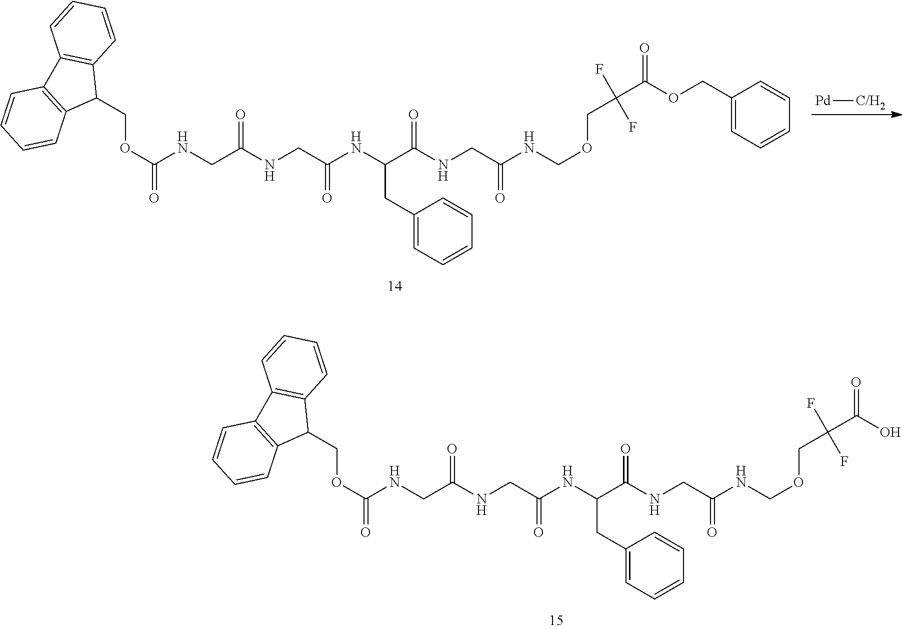

To a solution of intermediate 14 in 5:95 deionized water:methanol (35 mL) is added 10% palladium on carbon (0.09 g). The mixture is hydrogenated at 30 PSI H₂ for 80 min, filtered and evaporated under vacuum to afford intermediate 15.

### Step 3:

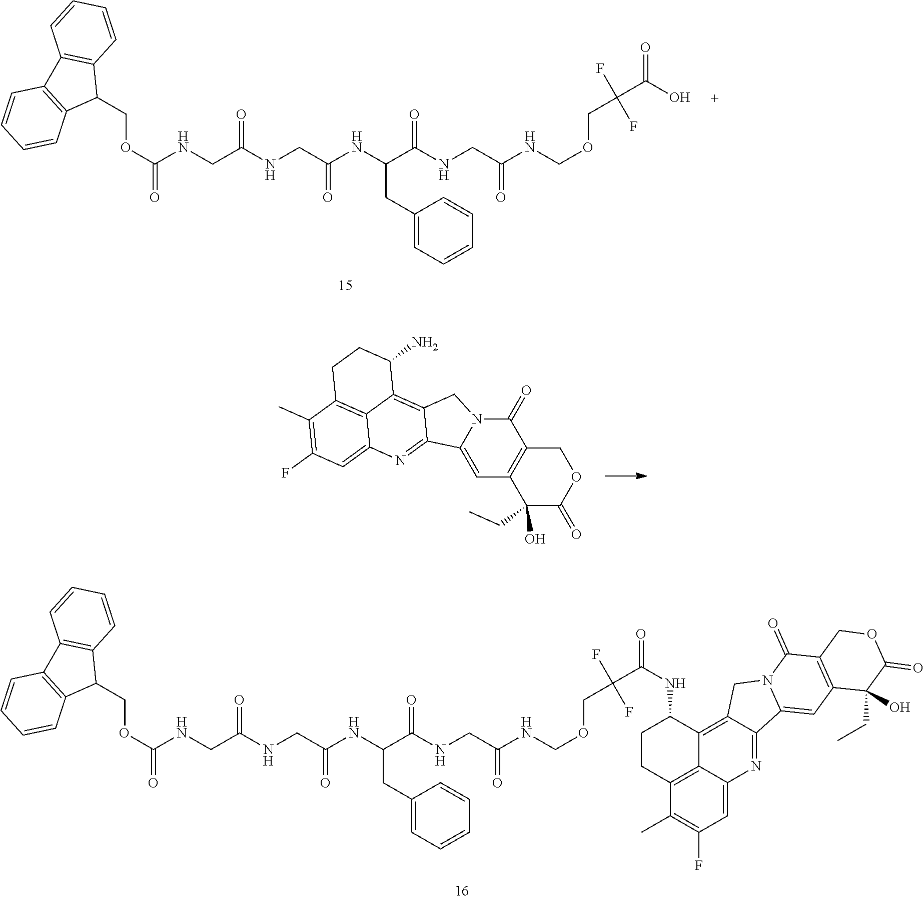 [SMILES: O=C(CNC(=O)OCC1c2ccccc2-c2ccccc21)NCC(=O)NC(Cc1ccccc1)C(=O)NCC(=O)NCOCC(F)(F)C(=O)O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)C(F)(F)COCNC(=O)CNC(=O)C(Cc1ccccc1)NC(=O)CNC(=O)CNC(=O)OCC1c2ccccc2-c2ccccc21)CC3]

A solution of exatecan mesylate, DMTMM, triethylamine and intermediate 15 in 20% DMF/water is stirred at 35° C. for 30 minutes. The reaction mixture is purified by silica gel column chromatography to give intermediate 16.

### Step 4:

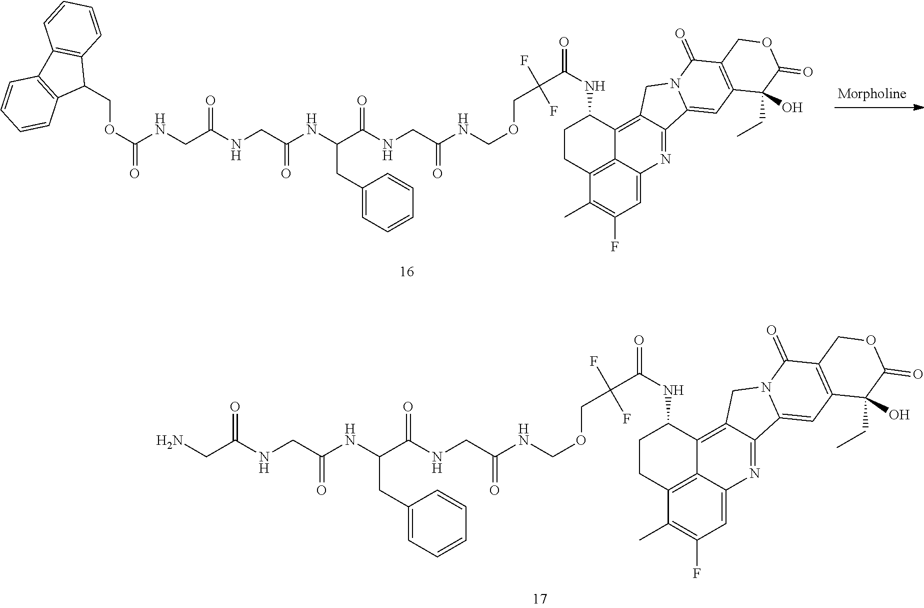 [SMILES: CC[C@@]1(O)C(=O)OCc2c1cc1n(c2=O)Cc2c-1nc1cc(F)c(C)c3c1c2[C@@H](NC(=O)C(F)(F)COCNC(=O)CNC(=O)C(Cc1ccccc1)NC(=O)CNC(=O)CNC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](NC(=O)C(F)(F)COCNC(=O)CNC(=O)C(Cc1ccccc1)NC(=O)CNC(=O)CN)CC3]

Morpholine is added to a stirred solution of intermediate 16 in DMF. After 1.5 h the reaction mixture is loaded onto a C18 RP column and eluted to give intermediate 17.

### Step 5:

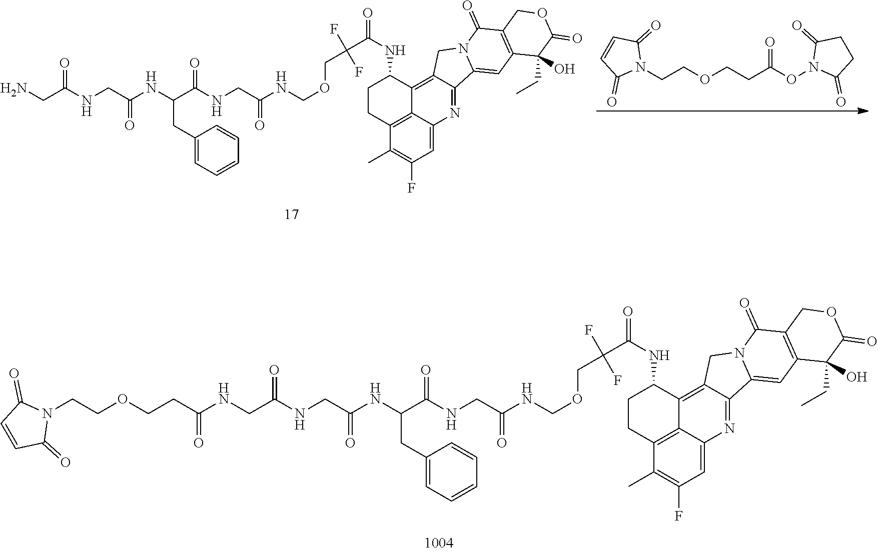

To a stirred solution of intermediate 17 in anhydrous DMF is added DIPEA and Mal-PEG1-NHS ester. The mixture is stirred for 30 min at RT, and purified by reverse phase column chromatography to give compound 1004.

### Step 6:

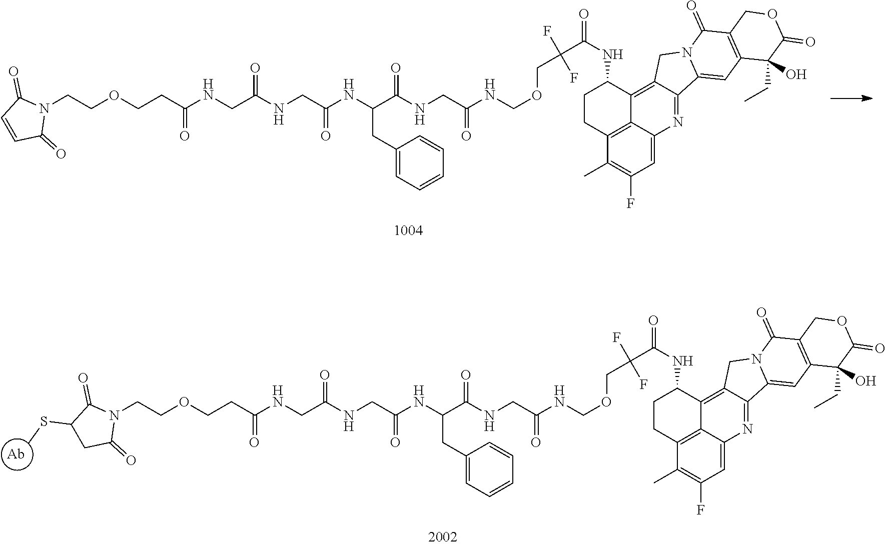

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1004 dissolved in a ˜40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2002.

### Example 57: Synthesis of Compound 1020

### Step 1:

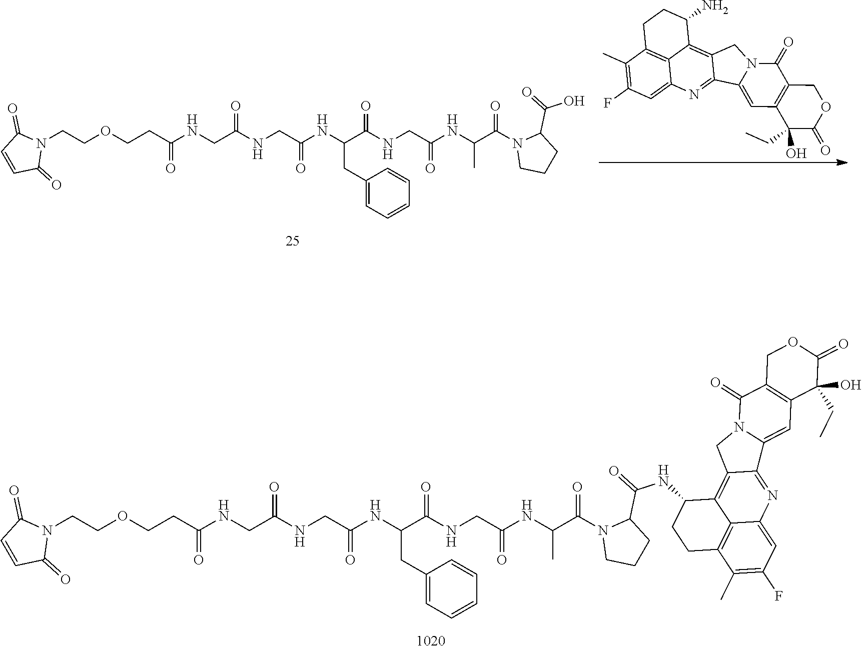 [SMILES: CC(NC(=O)CNC(=O)C(Cc1ccccc1)NC(=O)CNC(=O)CNC(=O)CCOCCN1C(=O)C=CC1=O)C(=O)N1CCCC1C(=O)O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)C1CCCN1C(=O)C(C)NC(=O)CNC(=O)C(Cc1ccccc1)NC(=O)CNC(=O)CNC(=O)CCOCCN1C(=O)C=CC1=O)CC3]

A solution of exatecan mesylate, DMTMM, triethylamine and intermediate 25 in 20% DMF/water is stirred at 35° C. for 30 minutes. The reaction mixture is purified by silica gel column chromatography to give compound 1020.

### Step 2:

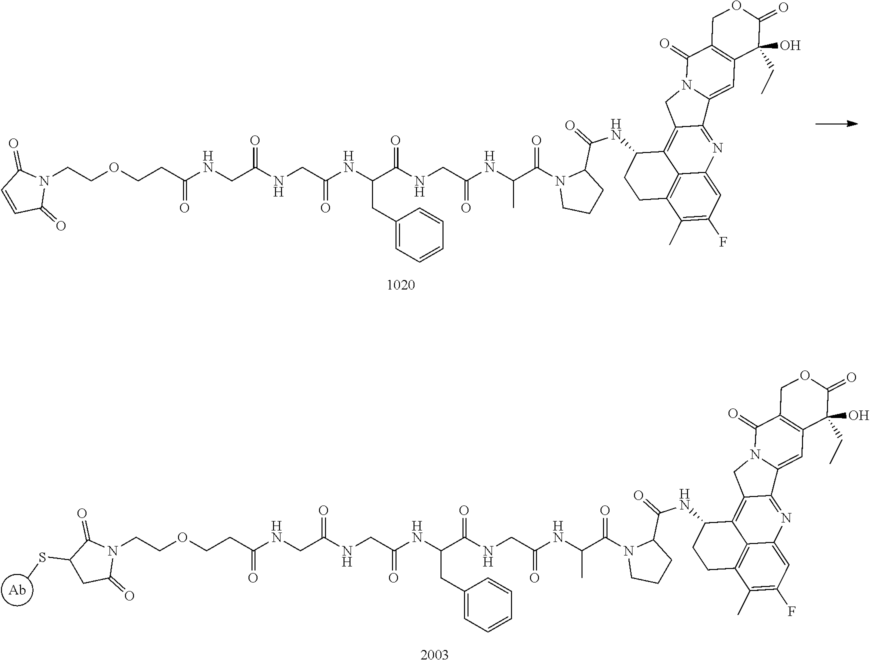

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1020 dissolved in a ˜40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2003.

### Example 58: Synthesis of Compound 1021

### Step 1:

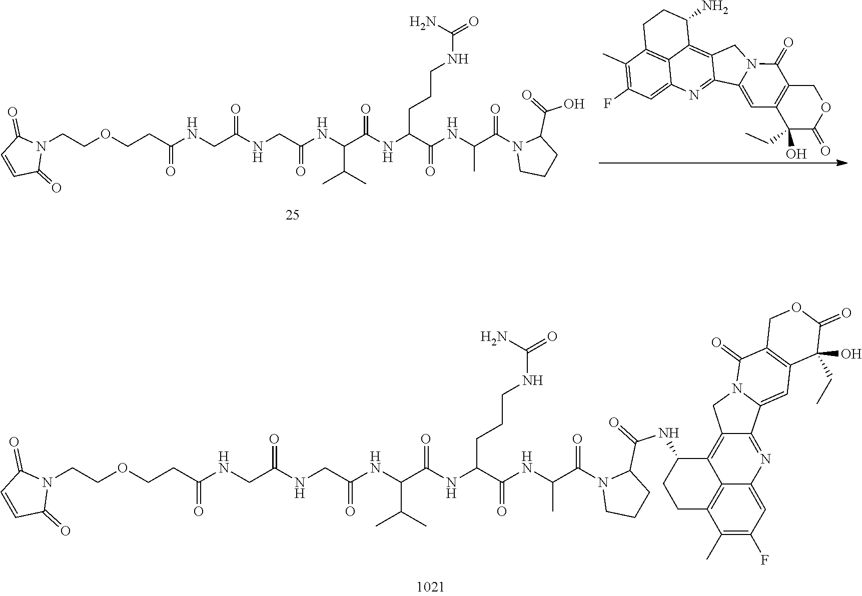 [SMILES: CC(NC(=O)C(CCCNC(N)=O)NC(=O)C(NC(=O)CNC(=O)CNC(=O)CCOCCN1C(=O)C=CC1=O)C(C)C)C(=O)N1CCCC1C(=O)O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)C1CCCN1C(=O)C(C)NC(=O)C(CCCNC(N)=O)NC(=O)C(NC(=O)CNC(=O)CNC(=O)CCOCCN1C(=O)C=CC1=O)C(C)C)CC3]

A solution of exatecan mesylate, DMTMM, triethylamine and intermediate 27 in 20% DMF/water is stirred at 35° C. for 30 minutes. The reaction mixture is purified by silica gel column chromatography to give compound 1021.

### Step 2:

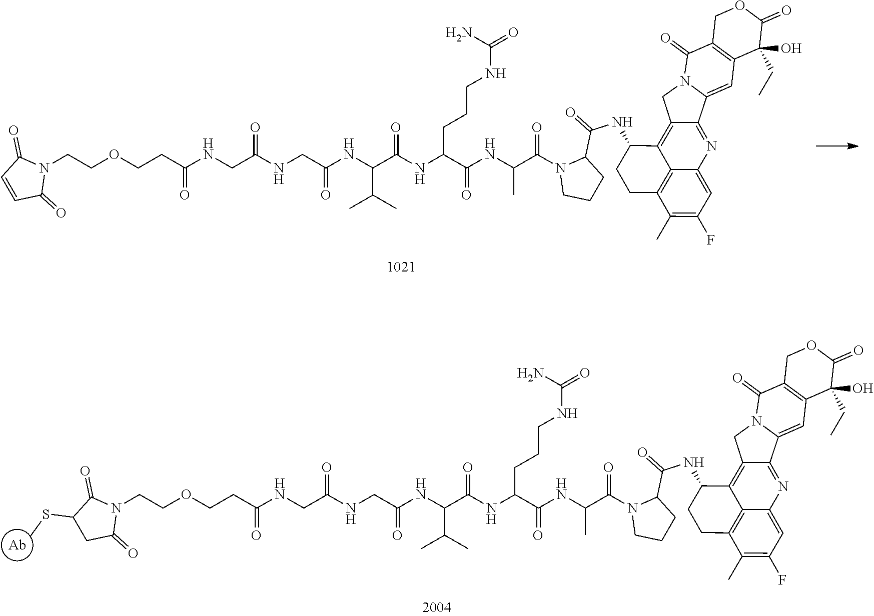

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1020 dissolved in a ˜40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2004.

### Example 59: Synthesis of Compound 1006

### Step 1:

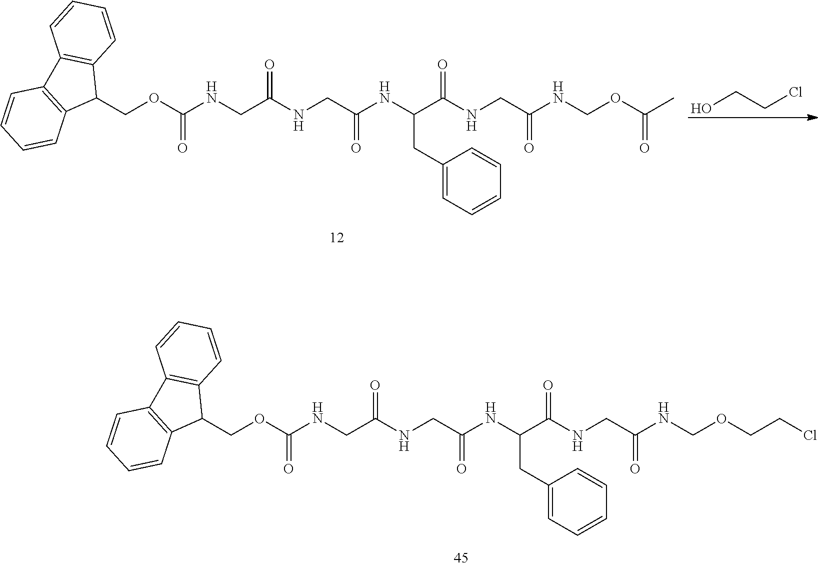

Intermediate 12 and chloroethanol are suspended in 20% TFA in dichloromethane (12 mL) and stirred at room temperature for 60 min. The solvent is evaporated and the residue is purified by reverse phase column chromatography to give intermediate 45

### Step 2:

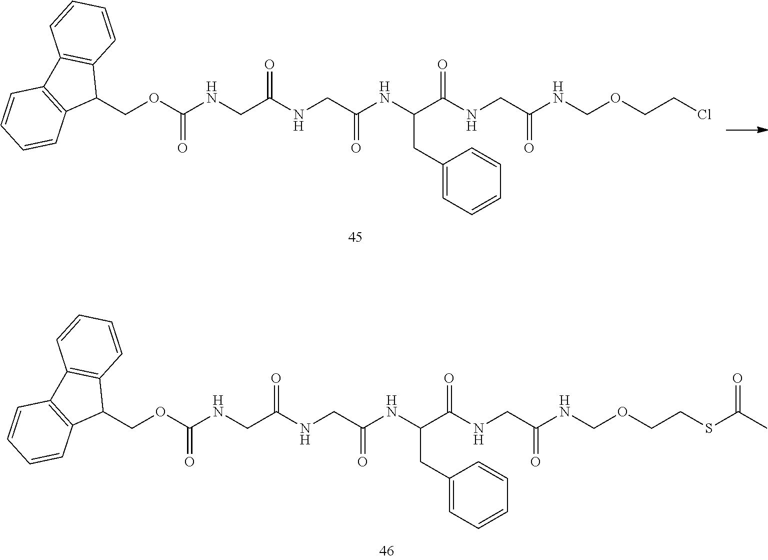 [SMILES: O=C(CNC(=O)OCC1c2ccccc2-c2ccccc21)NCC(=O)NC(Cc1ccccc1)C(=O)NCC(=O)NCOCCCl ; CC(=O)SCCOCNC(=O)CNC(=O)C(Cc1ccccc1)NC(=O)CNC(=O)CNC(=O)OCC1c2ccccc2-c2ccccc21]

To a stirred solution of intermediate 45 and tetrabutylammonium iodide in DMF at 0° C. was added powdered potassium thiol acetate. The reaction mixture is gradually warmed to rt and stirred overnight. The reaction mixture is quenched with water, and the residue purified by reverse phase column chromatography to give intermediate 46.

### Step 3:

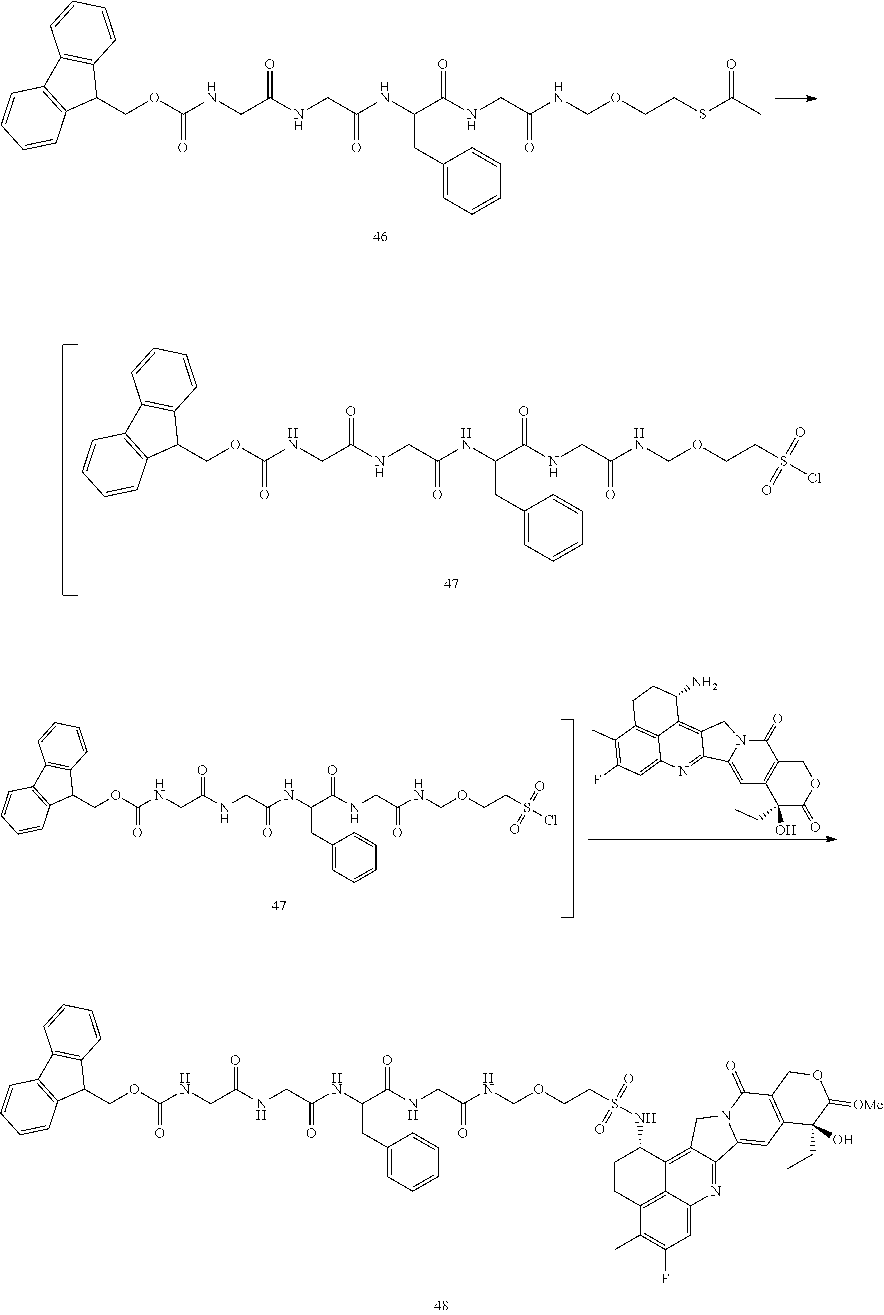

To a solution of intermediate 46 in acetonitrile at 10° C. was added a solution of NCS in 4:1 CH₃CN/2N HCl dropwise. The reaction mixture is stirred at 10° C. for 2 h and dried over molecular sieves overnight at 10° C. The solvent is evaporated, the residue diluted with dry acetonitrile, and evaporated again. After 3 cycles of dilution and evaporation, the intermediate 47 is dissolved in dry THF, cooled in an ice bath, and a solution of exatecan and triethanolamine in 5 ml of dry ethyl acetate is added. The reaction mixture is stirred at ice bath temperature for 2 hours and then at RT overnight. The solvent is evaporated, and the residue purified by reverse phase column chromatography to give intermediate 48.

### Step 4:

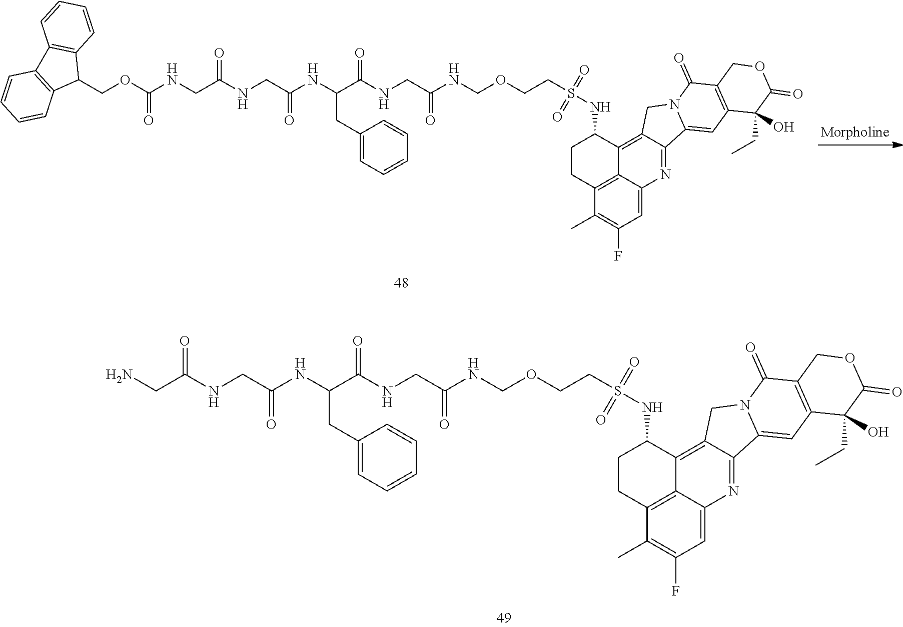 [SMILES: CC[C@@]1(O)C(=O)OCc2c1cc1n(c2=O)Cc2c-1nc1cc(F)c(C)c3c1c2[C@@H](NS(=O)(=O)CCOCNC(=O)CNC(=O)C(Cc1ccccc1)NC(=O)CNC(=O)CNC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](NS(=O)(=O)CCOCNC(=O)CNC(=O)C(Cc1ccccc1)NC(=O)CNC(=O)CN)CC3]

Morpholine is added to a stirred solution of intermediate 16 in DMF. After 1.5 h the reaction mixture is loaded onto a C18 RP column and eluted to give intermediate 17.

### Step 5:

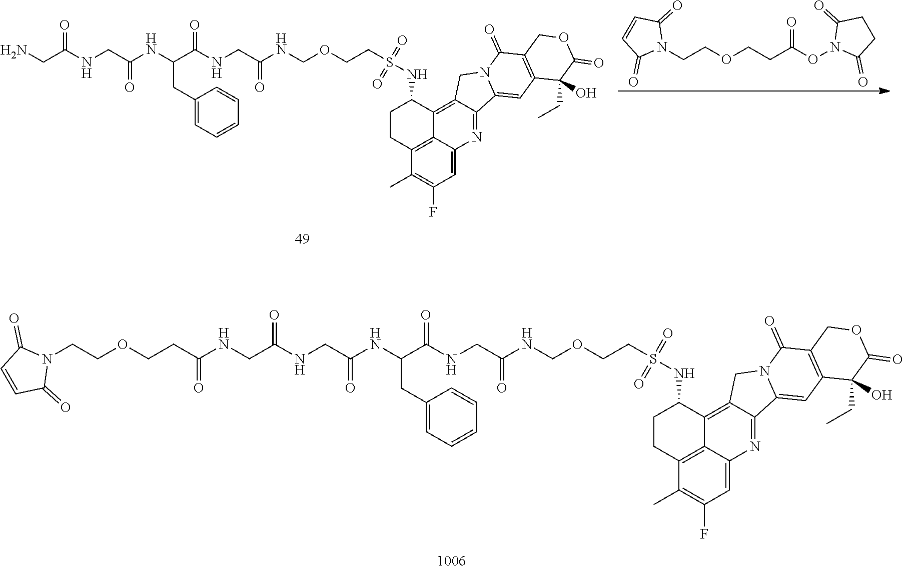

To a stirred solution of intermediate 49 in anhydrous DMF is added DIPEA and Mal-PEG1-NHS ester. The mixture is stirred for 30 min at RT, and purified by reverse phase column chromatography to give compound 1006.

### Step 6:

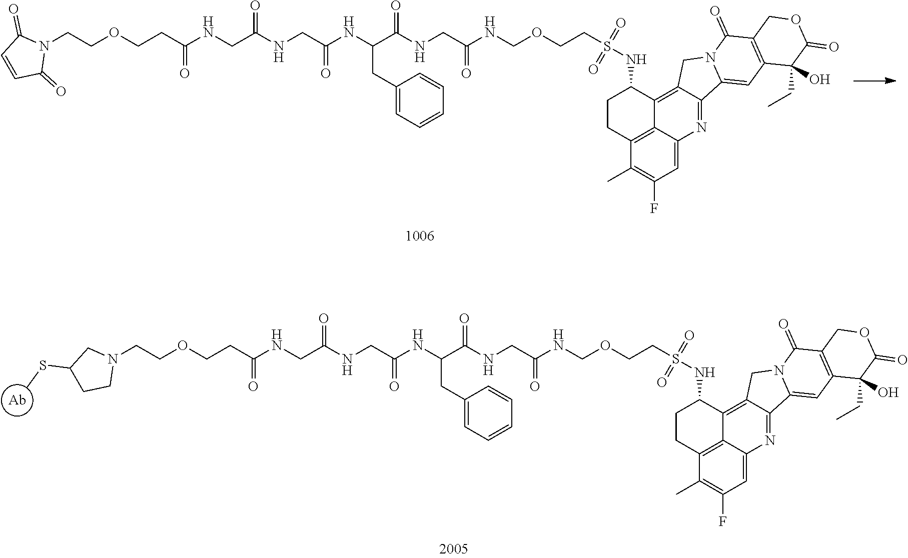

To a 5 mg/ml solution of anti-huTrop2 antibody is added TCEP in 50 mM EPPS (pH 7.4, containing 5 mM EDTA). After stirring at 37° C. for 90 minutes, the mixture is then cooled to ˜25° C., and the conjugation reaction is initiated by adding 12 equivalents of compound 1006 dissolved in a ˜40° C. solution of 50 mM EPPS (pH 7.4 containing 20% DMSO). The reaction mixture is stirred at RT for 2 h, then mixed with 10 mM acetate, 10% sucrose, 0.01% Tween-20 pH 5.0 and concentrated. The resulting mixture is purified by size exclusion column chromatography to afford compound 2005.

### Example 60: Synthesis of Compound 1009

### Step 1:

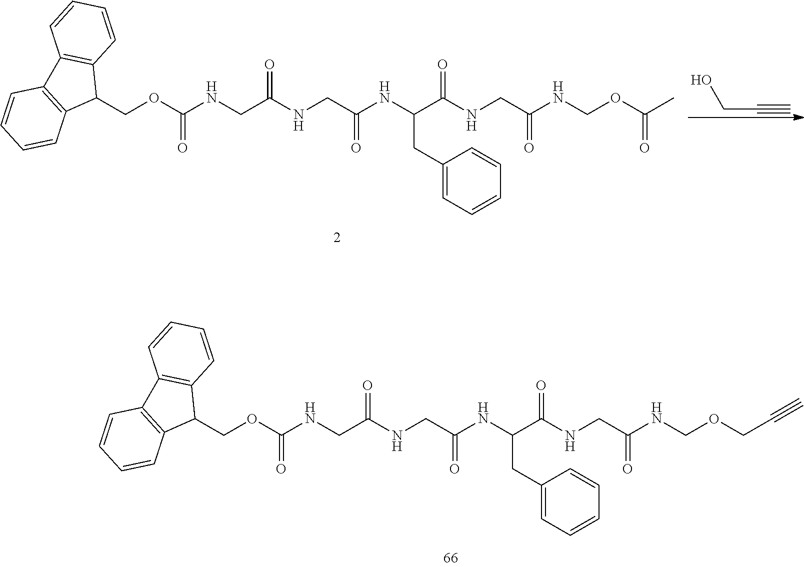

Intermediate 2 from Example 54 propargyl alcohol (192.6 mg, 3.44 mmol) are suspended in a cold solution of 20% TFA in dichloromethane and stirred at room temperature for 60 min. The solvent is evaporated, and the residue purified by reverse phase column chromatography to give intermediate 66.

### Step 2:

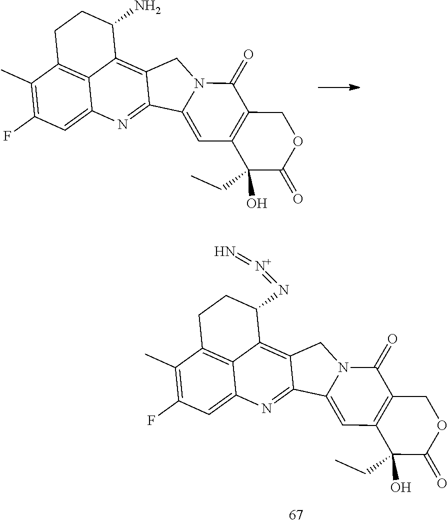 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](N=[N+]=N)CC3]

To a solution of exatecan in methanol was added imidazole-1-sulfonyl azide, HCl salt, K₂CO₃, and copper sulfate pentahydrate dissolved in water. The reaction mixture is stirred at 37° C. for 18 hours and the residue purified by column chromatography to provide intermediate 67.

### Step 3:

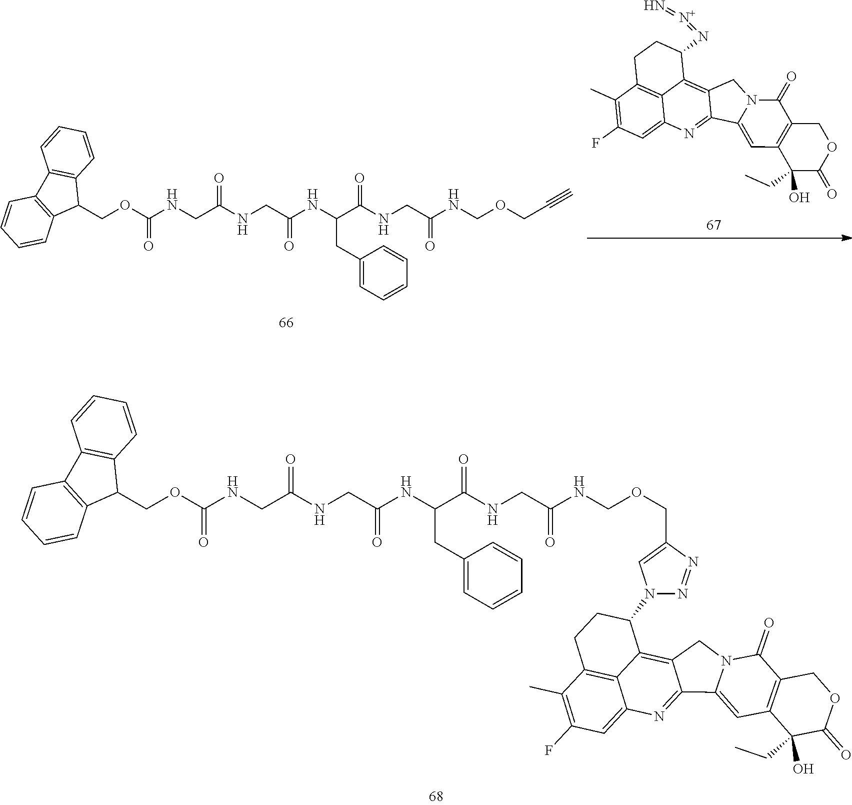 [SMILES: C#CCOCNC(=O)CNC(=O)C(Cc1ccccc1)NC(=O)CNC(=O)CNC(=O)OCC1c2ccccc2-c2ccccc21 ; CC[C@@]1(O)C(=O)OCc2c1cc1n(c2=O)Cc2c-1nc1cc(F)c(C)c3c1c2[C@@H](N=[N+]=N)CC3 ; CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CNC(=O)OCC2c4ccccc4-c4ccccc42)nn1)CC3]

To a suspension of azide 67 in DMSO is added intermediate compound 66. Bis(triphenylphosphine)copper(I) acetate and BTTAA are added, and the reaction mixture is stirred at rt 2 hours and at 40° C. for an additional 5 hours. The solvent is evaporated, and the residue purified by reverse phase column chromatography to give intermediate 68.

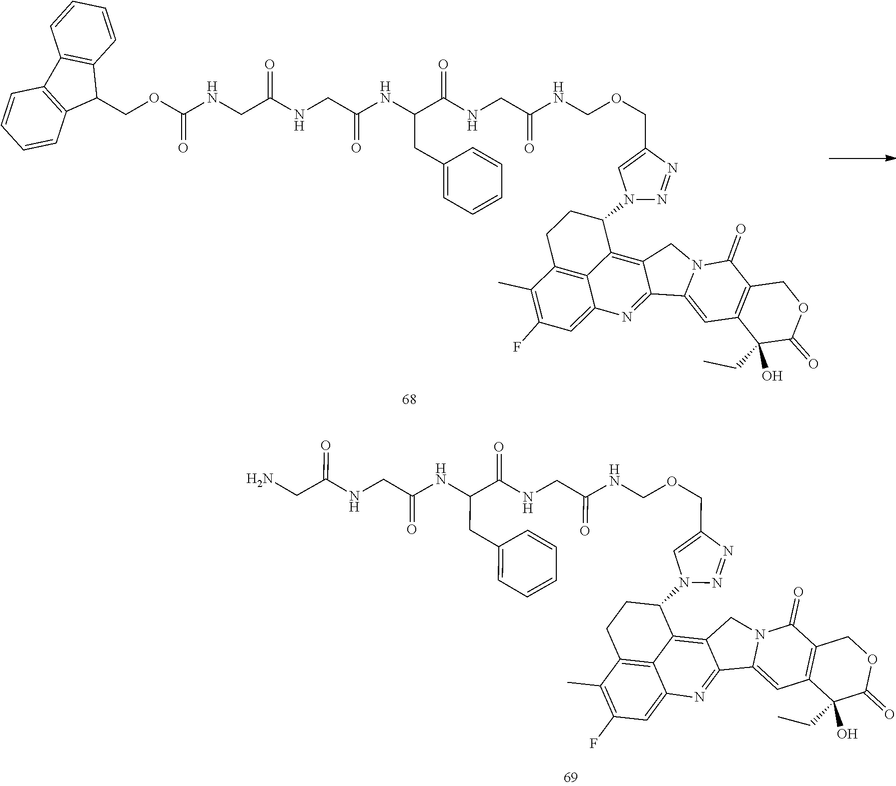 [SMILES: CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CNC(=O)OCC2c4ccccc4-c4ccccc42)nn1)CC3 ; CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CN)nn1)CC3]

Morpholine is added to a stirred solution of intermediate 68 in DMF. After 1.5 h the reaction mixture is loaded onto a C18 RP column and eluted to give intermediate 69.

### Step 5:

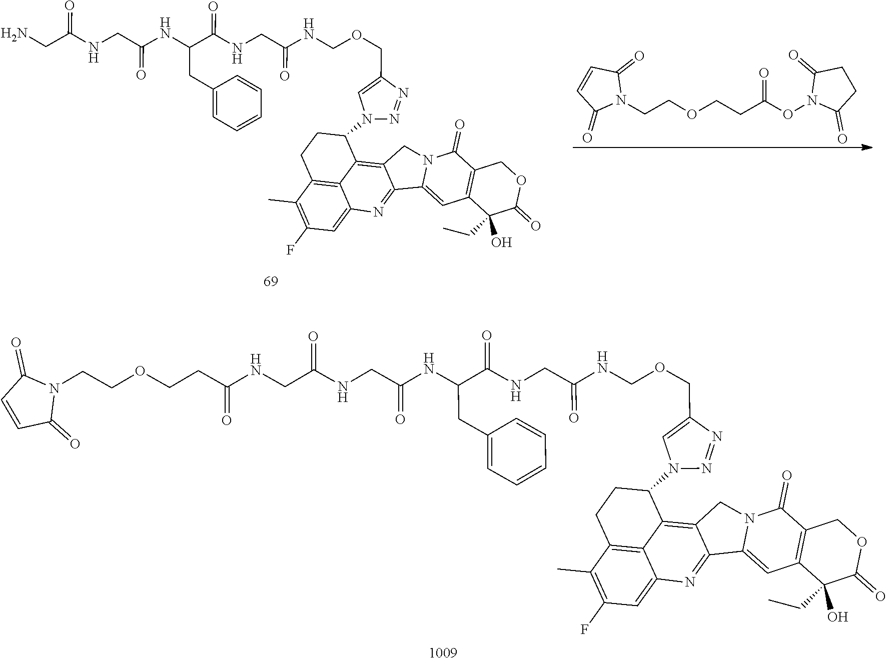 [SMILES: CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CN)nn1)CC3 ; O=C(CCOCCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](n1cc(COCNC(=O)CNC(=O)C(Cc2ccccc2)NC(=O)CNC(=O)CNC(=O)CCOCCN2C(=O)C=CC2=O)nn1)CC3]

To a stirred solution of intermediate 69 in anhydrous DMF is added DIPEA and Mal-PEG1-NHS ester. The mixture is stirred for 30 min at RT, and purified by reverse phase column chromatography to give compound 1009.

### Example 61: Synthesis of Compound 1011

### Step 1:

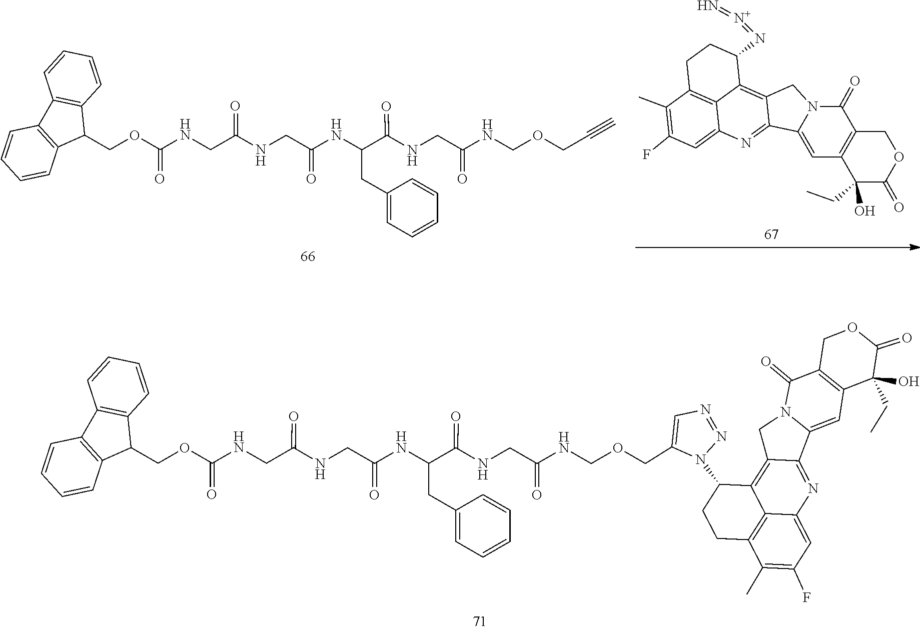 [SMILES: C#CCOCNC(=O)CNC(=O)C(Cc1ccccc1)NC(=O)CNC(=O)CNC(=O)OCC1c2ccccc2-c2ccccc21 ; CC[C@@]1(O)C(=O)OCc2c1cc1n(c2=O)Cc2c-1nc1cc(F)c(C)c3c1c2[C@@H](N=[N+]=N)CC3 ; CC[C@@]1(O)C(=O)OCc2c1cc1n(c2=O)Cc2c-1nc1cc(F)c(C)c3c1c2[C@@H](n1nncc1COCNC(=O)CNC(=O)C(Cc1ccccc1)NC(=O)CNC(=O)CNC(=O)OCC1c2ccccc2-c2ccccc21)CC3]

Chloro(pentamethylcyclopentadienyl)(cyclooctadiene)ruthenium (II), azide 67, and intermediate 66 are dissolved in acetonitrile. The reaction mixture is stirred at RT for 2 hours and then at 40° C. for additional 4 hours. The solvent is evaporated, and the residue purified by reverse phase column chromatography to give intermediate 71.

### Step 2:

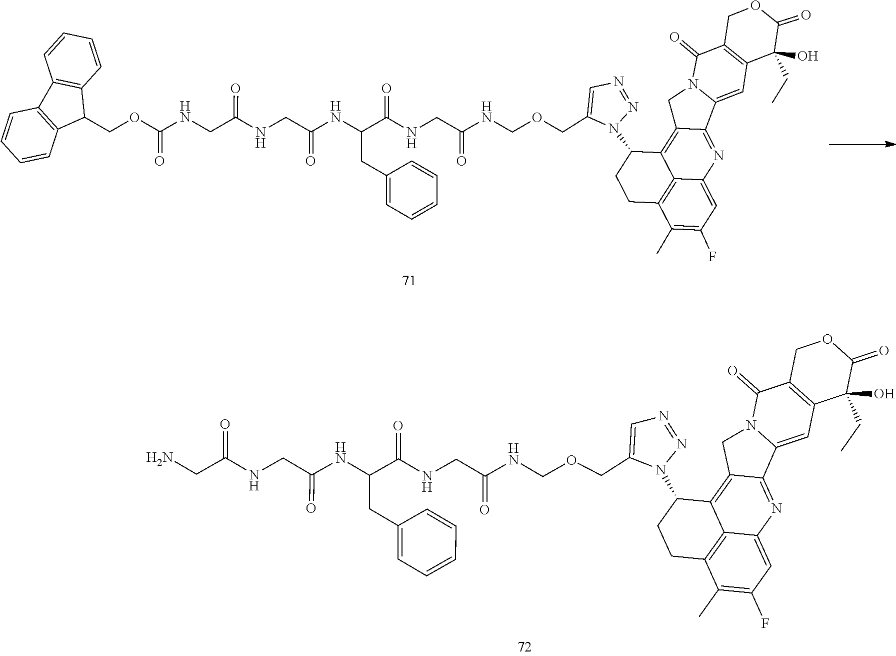 [SMILES: CC[C@@]1(O)C(=O)OCc2c1cc1n(c2=O)Cc2c-1nc1cc(F)c(C)c3c1c2[C@@H](n1nncc1COCNC(=O)CNC(=O)C(Cc1ccccc1)NC(=O)CNC(=O)CNC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](n1nncc1COCNC(=O)CNC(=O)C(Cc1ccccc1)NC(=O)CNC(=O)CN)CC3]

Morpholine is added to a stirred solution of intermediate 68 in DMF. After 1.5 h the reaction mixture is loaded onto a C18 RP column and eluted to give intermediate 72.

### Step 3:

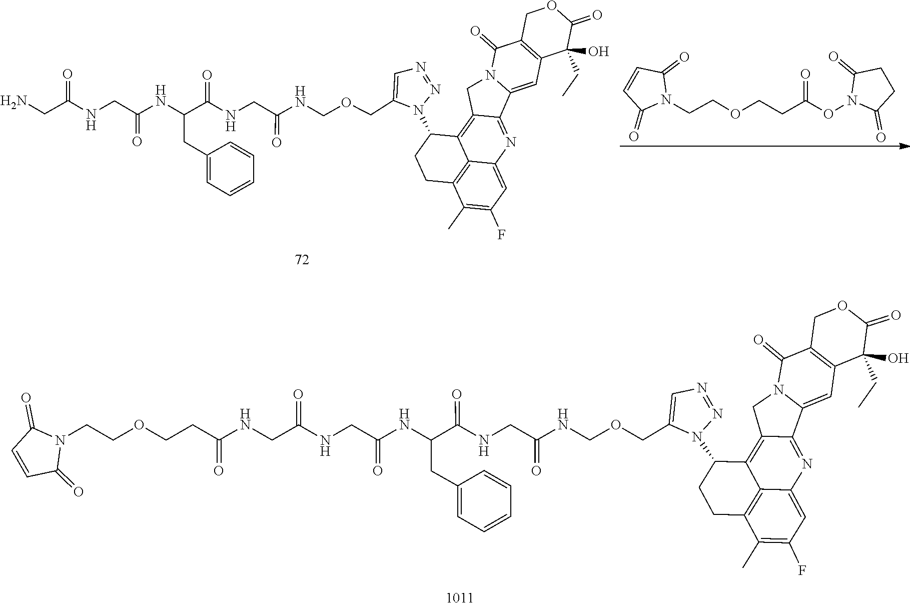 [SMILES: CC[C@@]1(O)C(=O)OCc2c1cc1n(c2=O)Cc2c-1nc1cc(F)c(C)c3c1c2[C@@H](n1nncc1COCNC(=O)CNC(=O)C(Cc1ccccc1)NC(=O)CNC(=O)CN)CC3 ; O=C(CCOCCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](n1nncc1COCNC(=O)CNC(=O)C(Cc1ccccc1)NC(=O)CNC(=O)CNC(=O)CCOCCN1C(=O)C=CC1=O)CC3]

To a stirred solution of intermediate 69 in anhydrous DMF is added DIPEA and Mal-PEG1-NHS ester. The mixture is stirred for 30 min at RT, and purified by reverse phase column chromatography to give compound 1011.

### Example 62: Synthesis of Compound 1023

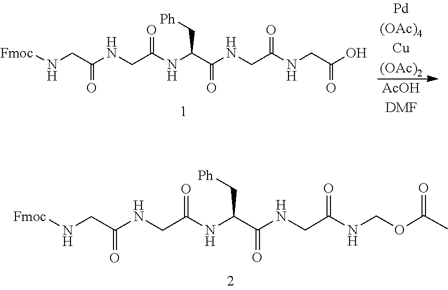

Intermediate 2. To a solution of compound 1 (350 mg, mmol) in DMF (3 mL) were added in order Cu(OAc)₂, AcOH and Pb(OAc)₄. The reaction mixture was stirred at 60° C. for 40 min, and then allowed to cool down at room temperature. The crude reaction mixture was directly load on C18 column (40 g C18) and eluted using a gradient of ACN in water (0→60% ACN in water). Fractions containing the desired product were merged and solvents were partially evaporated under reduced pressure to a final volume of approx. 10 mL, to be then lyophilized. The desired product was obtained as a white powder (285 mg, 80% HPLC purity). MS calc. for C₃₃H₃₅N5NaO₈: 652.24, found: 652.42 [M+Na]⁺.

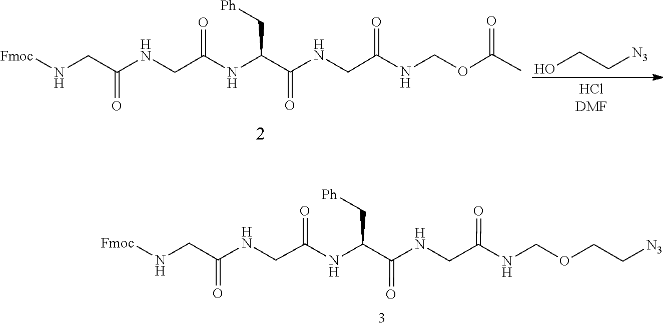

Intermediate 3. Intermediate 2 (150 mg, 0.238 mmol) was dissolved in DMF (2 mL). Azido ethanol (2 equiv., 0.457 mmol, 40 mg) was added, followed by 100 μL of a 2M solution of HCl in dioxane. The reaction mixture was stirred at room temperature for 1 hour. The crude reaction mixture was directly load on C18 column (40 g C18) and eluted using a gradient of ACN in water (0→60% ACN in water). Fractions containing the desired product were merged and solvents were partially evaporated under reduced pressure to a final volume of approx. 10 mL, to be then lyophilized. The desired product was obtained as a white powder (94 mg, 65% HPLC purity). MS calc. for C₃₃H₃₆N₈NaO₇: 679.26, found: 679.28 [M+Na]⁺.

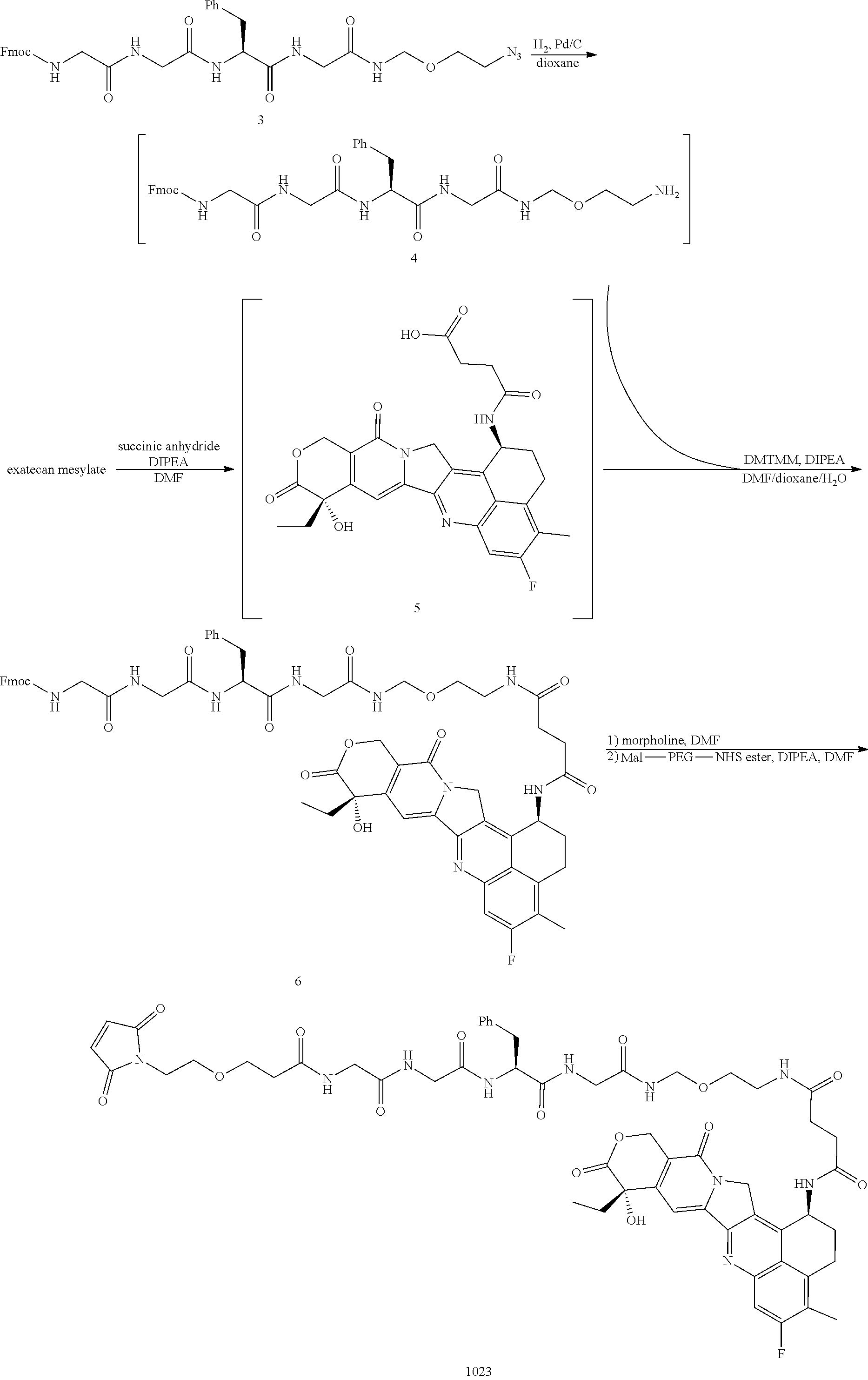

Intermediate 4. To the solution of Intermediate 3 (1.0 equiv., 26 mg, 0.040 mmol) in dioxane (1.5 ml) was added Pd/C (10% w/w, 5 mg) and the resulting suspension was hydrogenated reaction mixture was hydrogenated (balloon) for 1.5 hour at room temperature. LC-MS analysis confirmed the full consumption of the starting material. The suspension was filtered and the desired product was obtained as a solution in dioxane, which was used directly into next step. MS calc. for C₃₃H₃₉N₆O₇: 631.29, found: 631.30, [M+H]⁺.

Intermediate 6. To the mixture of exatecan mesylate (1.0 equiv., 20 mg, 0.037 mmol) and succinic anhydride (1.1 equiv., 4.1 mg, 0.041 mmol) was added DMF (0.5 mL) and diisopropylethylamine (2.2 equiv., 14 μL, 0.082 mmol) and the mixture was stirred for 30 minutes at room temperature. Then, the solution of Intermediate 4 in dioxane (obtained in the previous step) was added to the reaction mixture, followed by the addition of DMTMM (1.0 equiv., 11 mg, 0.037 mmol), diisopropylethylamine (1.0 equiv., 7 μL, 0.037 mmol) and water (0.25 mL), and the resulting solution was stirred 1 hour at room temperature. The mixture was concentrated on rotary evaporator and the residue was purified by reverse-phase flash chromatography (25 g, diol-modified C18, 0→60% ACN/0.1% aq. TFA) to obtain the desired product (15 mg, 35%) as a pale-yellow solid after lyophilization. MS calc. for C₆₁H₆₃FN₉O₁₃: 1148.45, found: 1148.45, [M+H]⁺.

Compound 1023. Morpholine (35 μL) was added to the solution of Intermediate 6 (1.0 equiv., 15 mg, 0.013 mmol) in DMF (1 mL) and the reaction mixture was stirred for 1.5 hour at room temperature. Then, DMF and excess morpholine were evaporated using rotary evaporator. The residue was re-dissolved in DMF (0.5 mL), followed by the addition of Mal-PEG-NHS ester (1.1 equiv., 4.4 mg, 0.014 mmol) and diisopropylethylamine (1.1 equiv., 2.5 μL, 0.014 mmol), and the resulting solution was stirred for 1 hour at room temperature. Purification by reverse-phase flash chromatography using a semipreparative column (diol-modified C18, 0→70% ACN/0.1% aq. TFA) offered the desired product (7 mg, 48%) as a pale-yellow solid after lyophilization. MS calc. for C₅₂H₆₂FN₁₀O₁₅: 1121.44, found: 1121.45, [M+H]⁺.

### Example 63: Synthesis of Compound 71

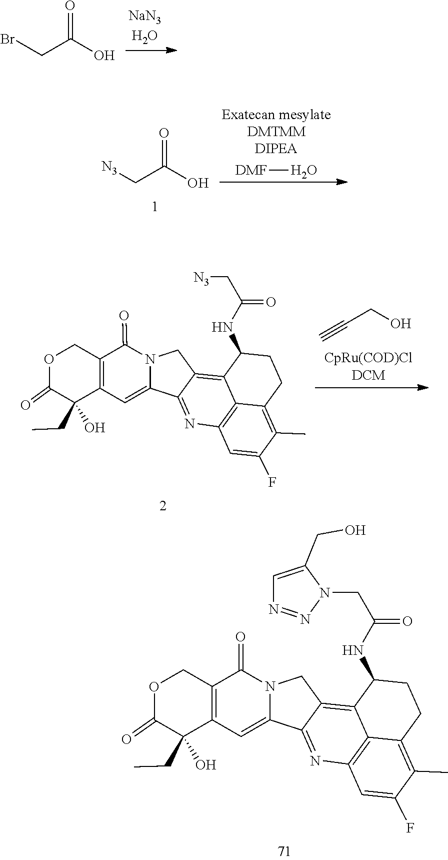

Intermediate 1. Bromoacetic acid (0.715 g, 10 mmol) was dissolved in 5 mL of water, then sodium azide (0.696 g, 5 mmol) was added and the solution was stirred at room temperature overnight. The solution was acidified with HCl until pH=1, then the desired product was extracted with diethyl ether. The solvent was dried over Na₂SO₄, filtered, and then evaporated, affording the reaction product which was used into the next step without any further purification.

Intermediate 2. Exatecan mesylate (100 mg, 0.188 mmol), 2-azidoacetic acid (1.1 equiv., 0.207 mmol, 21 mg), DMTMM (1.3 equiv., 0.244 mmol, 68 mg) and DIPEA (50 μL) were dissolved into 5 mL of a 4:1 DMF/water mixture. The reaction mixture was stirred at room temperature for 1 hour. The mixture was directly purified by reverse-phase flash chromatography (diol-modified C18, 25 g, 0→100% ACN in water). The desired product was obtained as a white powder after lyophilization (79 mg, 74%). MS calc. for C₂₆H₂₄FN₆O₅: 519.18, found: 519.41, [M+H]⁺.

Compound 71. The previously prepared intermediate 2 (10 mg, 0.0193 mmol), propargyl alcohol (1.2 equiv., 0.0232 mmol, 1.35 μL), and the catalyst CpRu(COD)Cl(10%, 0.7 mg) were suspended in anhydrous DCM (2 mL) under an argon atmosphere. The mixture was stirred at 40 C for 16 hours. The crude reaction product was directly purified by reverse-phase HPLC chromatography (semipreparative diol-modified C18, 0→100% ACN in water). The desired product was obtained as a yellowish powder after lyophilization from water-ACN (9 mg, 90%). MS calc. for C₂₉H₂₈FN₆O₆: 575.21, found: 575.14, [M+H]⁺.

### Example 64: Synthesis of Compound 72

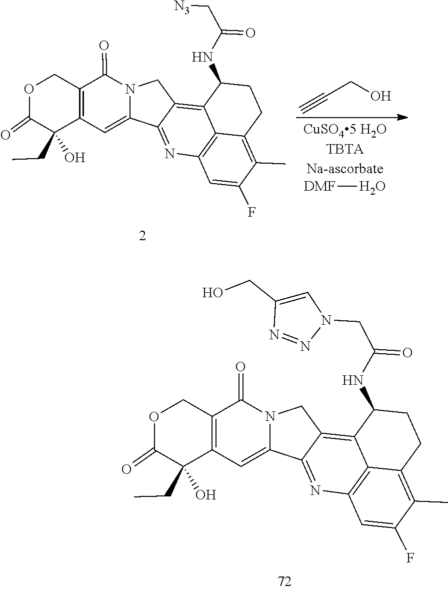

Compound 72. The previously prepared intermediate 2 of Example 63 (10 mg, 0.0193 mmol), propargyl alcohol (1.2 equiv., 0.0232 mmol, 1.35 μL), sodium ascorbate (0.2 equiv., 0.00386 mmol, 2 M in water, 1.93 μL), copper sulphate pentahydrate (0.1 equiv., 0.00193 mmol, 1 M in water, 1.93 μL) and TBTA (0.15 equiv., 0.0029 mmol, 1.5 mg) were dissolved in 2 mL of a 4:1 mixture of DMF/water. The reaction mixture was stirred at room temperature for 2 hours. The crude reaction product was directly purified by reverse-phase HPLC chromatography (semipreparative diol-modified C18, 0→100% ACN in water). The desired product was obtained as a yellowish powder after lyophilization from water-ACN (10 mg, 95%). MS calc. for C₂₉H₂₈FN₆O₆: 575.21, found: 575.55, [M+H]⁺.

### Example 65: Synthesis of Compound 73

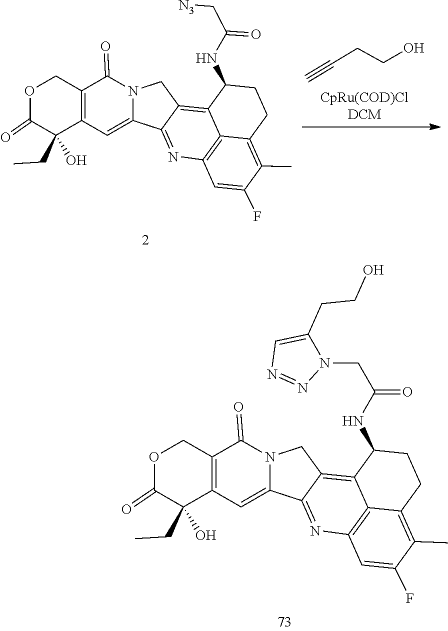

Compound 73. The previously prepared intermediate 2 of Example 63 (10 mg, 0.0193 mmol), 3-butyn-1-ol (1.2 equiv., 0.0232 mmol, 1.50 μL), and the catalyst CpRu(COD)C1 (10%, 0.7 mg) were suspended in anhydrous DCM (2 mL) under an argon atmosphere. The mixture was stirred at 40 C for 16 hours. The crude reaction product was directly purified by reverse-phase HPLC chromatography (semipreparative diol-modified C18, 0→100% ACN in water). The desired product was obtained as a yellowish powder after lyophilization from water-ACN (6 mg, 54%). MS calc. for C₃₀H₃₀FN₆O₆: 589.22, found: 589.23, [M+H]⁺.

### Example 66: Synthesis of Compound 74

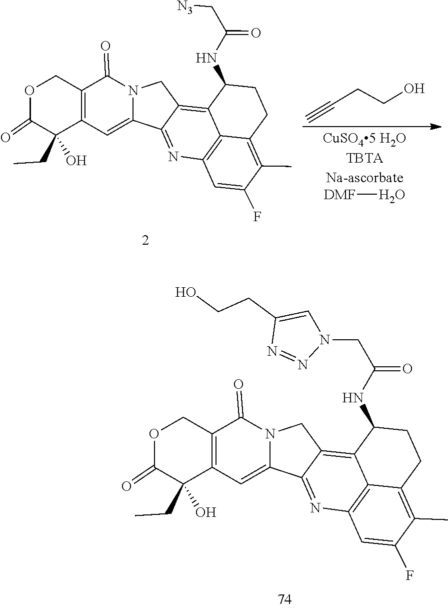

Compound 74. The previously prepared intermediate 2 of Example 63 (10 mg, 0.0193 mmol), 3-butyn-1-ol (1.2 equiv., 0.0232 mmol, 1.50 μL), sodium ascorbate (0.2 equiv., 0.00386 mmol, 2 M in water, 1.93 μL), copper sulphate pentahydrate (0.1 equiv., 0.00193 mmol, 1 M in water, 1.93 μL) and TBTA (0.15 equiv., 0.0029 mmol, 1.5 mg) were dissolved in 2 mL of a 4:1 mixture of DMF/water. The reaction mixture was stirred at room temperature for 2 hours. The crude reaction product was directly purified by reverse-phase HPLC chromatography (semipreparative diol-modified C18, 0→100% ACN in water). The desired product was obtained as a white powder after lyophilization from water-ACN (7 mg, 62%). MS calc. for C₃₀H₃₀FN₆O₆: 589.22, found: 589.05, [M+H]⁺.

### Example 67: Synthesis of Compound 173

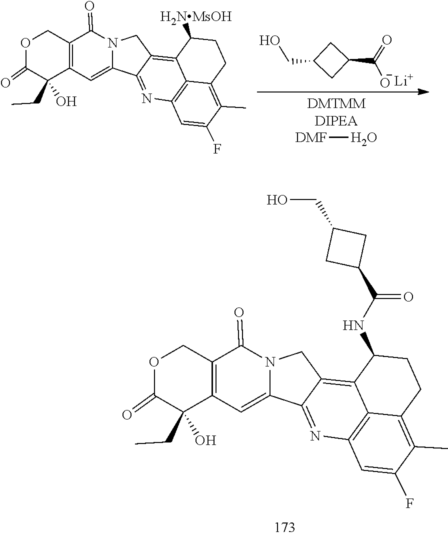

To a 4:1 DMF/water mixture (4 mL) were added exatecan mesylate (20 mg, 0.038 mmol), trans-3-hydroxymethylcyclobutane-1-carboxylic acid, lithium salt (1.25 eqiuv., 6.6 mg, 0.048 mmol), DMTMM (2.0 equiv., 21 mg, 0.076 mmol) and diisopropylethylamine (10 μL). The resulting solution was stirred for 1 hour at room temperature, as LC-MS indicated the full consumption of the starting material. The mixture was directly purified by reverse-phase HPLC chromatography using a semipreparative column (diol-modified C18, 0→100% ACN/1% TFA). The desired product was obtained as a white powder after lyophilization (16 mg, 77%). MS calc. for C₃₀H₃₁FN₃O₆: 548.22, found: 548.31, [M+H]⁺.

### Example 68: Kinetic Solubility of Compounds Based on Turbidity

### Turbidity-Based-Aqueous Solubility (Kinetic Solubility) Procedure

In-vitro kinetic solubilities of the compounds in PBS pH 7.4 buffer at 25° C. were determined by diluting compounds from 100% dimethyl sulfoxide (DMSO) into PBS buffer and measuring absorbance at 490, 590 and 650 nm. Stock concentrations in 100% DMSO were provided at 1-6 mM in 100% DMSO. Kinetic solubilities were determined by diluting test compounds from 100% DMSO into PBS pH 7.4 buffer, as duplicate, 10 point 2-fold serial dilution starting at 100× dilution of stock DMS solution into PBS buffer in a clear, flat bottom polystyrene assay plates. Total assay volume was 200 microliters. Solutions were mixed by plate shaking, incubated for 30 m at 25° C., and absorbance was read at 490, 590 and 650 nm. 10 (v/v) DMSO in PBS buffer was used as a blank. For each test compound, average optical density (GD) at each concentration was determined by averaging the blank corrected sum of absorption at 490 nm, 590 nm and 650 nm, over duplicate measurements. Turbidity threshold optical density value was set as a sum of mean plus two standard deviations of the mean of absorbances at 490, 590 and 650 nm for 1% (v/v) DMSO in PBS pH 7.4 buffer. Highest soluble concentration (micromolar) corresponded to the highest concentration at which average optical density was below optical density value set for turbidity threshold (Table 4). Amiodarone and propranolol were used as low and high solubility controls respectively.

TABLE 4

| | Highest Soluble | Highest |
|---|---|---|
| | Concentration | Concentration |
| Compound | (μM) | Tested (μM) |
| Amiodarone | 0 | 100 |
| Propranolol | 100 | 100 |
| Compound 18 | 13 | 13 |
| Compound 66 | 21 | 21 |
| Compound 105 | 25 | 25 |
| Compound 109 | 17 | 17 |
| Compound 110 | 18 | 18 |
| Compound 52 | 25 | 25 |
| Compound 115 | 23 | 23 |
| Compound 117 | 30 | 30 |
| Compound 118 | 30 | 30 |
| Compound 129 | 22 | 22 |
| Compound 2 | 15 | 31 |
| Compound 16 | 22 | 22 |
| Compound 22 | 23 | 23 |
| Compound 42 | 8 | 16 |
| Compound 108 | 10 | 20 |
| Compound 12 | 17 | 17 |
| Compound 48 | 28 | 28 |
| Compound 79 | 30 | 30 |
| Compound 100 | 29 | 29 |
| Compound 103 | 7 | 27 |
| Compound 122 | 26 | 26 |
| Compound 136 | 23 | 23 |
| Compound 148 | 3 | 25 |
| Compound 111-A | 23 | 23 |
| Compound 111-B | 30 | 30 |
| Compound 147 | 21 | 21 |
| Compound 107 | 15 | 15 |
| Compound 164 | 10 | 10 |
| Compound 58 | 37 | 37 |
| Compound 168 | 37 | 37 |
| Compound 175 | 8 | 33 |
| Compound 140 | 10 | 20 |
| Compound 163 | 24 | 24 |
| Compound 176 Rotamer A | 28 | 28 |
| Compound 176 Rotamer B | 29 | 29 |
| Compound 159 | 27 | 54 |
| Compound 167 | 42 | 42 |
| Compound 106 | 10 | 20 |
| Compound 188 | 6 | 62 |
| Compound 83 | 44 | 44 |
| Compound 166 | 22 | 22 |
| Dxd | 21 | 21 |
| Exatecan | 24 | 24 |
| SN-38 | 4 | 18 |

### Example 69. Characterization of Compound Polarity by Retention Times in Reverse Phase Liquid Chromatography

Retention times in reverse phase liquid chromatography were determined by two independent experiments. First experiment, HPLC-MS analyses were performed on Shimadzu UFLC-MS-2020 system with ESI. Column: Acquity UPLC BEH C18 1.7 μm, 2.1×50 mm. Solvent A: 0.1% formic acid in water; Solvent B: 0.1% formic acid in acetonitrile. Gradient: 0% B 0.8 min., 0% B to 100% B 4.2 min., 100% B 3 minutes at. Total flow 0.6 ml/min. Total time of the method 10 min. UV-Vis spectra were recorded with a Shimadzu SPD-M20A Prominence diode array detector, in the range 200-800 nm. Second experiment, lyophilized compounds were dissolved in dry DMSO at 2 mM, aliquots frozen and stored at −80° C. Compounds dissolved in DMSO were analyzed using RP-HPLC (UV/VIS, MS ELSD) Agilent 1100 platform with 1200 DAD and SofTA ELSD detectors, and the Agilent 6150 MS system. Shimadzu 3.0 mm×30 mm XR ODS 2.2 μm column was run at 50° C., 1.5 mL/min. Solvent A: 0.1% Formic acid in water, Solvent B: 0.08% Formic acid in methanol-Gradient: 5%-100% B in 3.0 min, 100% solvent B for 0.3 min. Retention times of compounds in reverse phase liquid chromatography using both methods are shown in Table 5.

TABLE 5
Characterization of compound polarity by two
independent determinations of retention times (RT) by
RP-HPLC in methanol or acetonitrile gradients and
determination of compounds purity by UV/VIS
and Evaporative Light Scattering (ELS).

| | RT* | RT** | UV % | ELS % |
|---|---|---|---|---|
| DXD | 3.41 | 2.5 | 98.2 | 100 |
| Exatecan | 2.929 | 1.73 | 99 | 97.6 |
| SN-38 | 3.982 | 2.46 | 100 | 100 |
| Compound 2 | 3.321 | 2.38 | 100 | 100 |
| Compound 12 | 3.309 | 2.44 | 96.6 | 98.02 |
| Compound 16 | 3.316 | 2.45 | 97.3 | 100 |
| Compound 18 | 3.241 | 2.32 | 100 | 100 |
| Compound 22 | 3.493 | 2.56 | 95.6 | 97.33 |
| Compound 42 | 3.394 | 2.49 | 79.7 | 87.3 |
| Compound 48 | 3.512 | 2.5 | 100 | 100 |
| Compound 52 | 3.492 | 2.55 | 99 | 99.38 |
| Compound 58 | 3.441 | 2.45 | 99.4 | 100 |
| Compound 66 | 4.43 | 2.45 | 91.6 | 94.49 |
| Compound 71 | 3.374 | nc | nc | nc |
| Compound 72 | 3.333 | nc | nc | nc |
| Compound 73 | 3.351 | nc | nc | nc |
| Compound 74 | 3.342 | nc | nc | nc |
| Compound 79 | 3.484 | 2.5 | 100 | 100 |
| Compound 83 | 3.452 | 2.44 | 100 | 99.53 |
| Compound 100 | 3.427 | 2.35 | 88.4 | 100 |
| Compound 103 | 3.525 | 2.56 | 97.9 | 100 |
| Compound 105 | 2.939 | 1.71 | 83.6 | 92.37 |
| Compound 106a | 3.569 | 2.55 | 90.1 | 90.06 |
| Compound 106b | 3.642 | 2.67 | 90.1 | 90.06 |
| Compound 107a | 3.044 | 1.82 | 95.2 | 100 |
| Compound 107b | 3.171 | 2.02 | 95.2 | 100 |
| Compound 108 | 3.505 | 1.9 | 95.6 | 100 |
| Compound 109 | 4.081 | 2.37 | 87.9 | 87.74 |
| Compound 110 | 4.042 | 2.47 | 92.4 | 97.98 |
| Compound 111-A | 3.578 | 2.44 | 100 | 100 |
| Compound 111-B | 3.659 | 2.51 | 96.4 | 100 |
| Compound 115 | 3.259 | 2.35 | 98.7 | 99.23 |
| Compound 117 | 3.278 | 2.42 | 100 | 100 |
| Compound 118 | 3.333 | 2.46 | 98.1 | 99.5 |
| Compound 122 | 3.288 | 2.39 | 96.3 | 72.17 |
| Compound 129 | 3.298 | 2.87 | 100 | 100 |
| Compound 130 | 3.594 | 2.32 | 59.2 | 58.92 |
| Compound 136 | 3.405 | 2.45 | 98.7 | 99.32 |
| Compound 140 | 3.16 | 2.28 | 92.9 | 98.77 |
| Compound 147 | 3.016 | 1.7 | 100 | 100 |
| Compound 148a | 3.96 | 2.9 | 100 | 99.18 |
| Compound 148b | 3.405 | 3.05 | 100 | 99.18 |
| Compound 159 | 3.443 | 2.45 | 100 | 100 |
| Compound 163 | 3.501 | 2.38 | 96.5 | 100 |
| Compound 164 | 2.928 | 1.55 | 83 | 96.69 |
| Compound 166 | 3.436 | 2.45 | 100 | 99.92 |
| Compound 167 | 3.449 | 2.49 | 100 | 99.78 |
| Compound 168 | 3.431 | 2.44 | 98.3 | 100 |
| Compound 173 | 3.467 | 2.92 | 100 | 100 |
| Compound 175 | 4.049 | 3.01 | 100 | 100 |
| Compound 176C | 3.686 | 2.72 | 96.2 | 100 |
| Compound 176D | 3.775 | 2.87 | 93.5 | 99.21 |
| Compound 188 | 3.557 | 2.54 | 100 | 99.58 |

nc-data not collected
*-HPLC; solvent A: water + 0.1% formic acid; solvent B: acetonitrile + 0.1% formic acid.
Gradient: 0% B 0.8 min., 0% B to 100% B 4.2 min., 100% B 3 minutes
**-HPLC; solvent A: water + 0.1% formic acid; solvent B: methanol + 0.1% formic acid.-Gradient: 5%-100% B in 3.0 min, 100% solvent B for 0.3 min.
RT-RP HPLC retention time (min)
a, b conformers not isolated
A, B stereoisomers separated or synthesized from stereochemically pure building blocks
C, D separated and purified conformers

### Example 70: In Vitro Cytotoxicity Assay

### Cancer Cell Lines

Human tumor cell lines, SK-BR-3, NCI-H292, HT-29, MCF-7, NCI-N87, and FaDu were obtained from ATCC. NCI-H292, HT-29, MCF-7, and NCI-N87 cells were cultured in RPMI-1640 media (Gibco, Life Technologies) supplemented with 10% v/v heat inactivated FBS (Corning), FaDu cells were maintained in DMEM media (Gibco, Life Technologies) supplemented with 10% v/v heat inactivated FBS (Corning). SK-BR-3 cells were maintained in McCoys 5A medium (Gibco, Life Technologies) supplemented with 10% v/w heat inactivated FBS (Corning) at 37° C. in a humidified incubator containing 5% CO2.

### Compound Preparation

Lyophilized compounds were dissolved in 100% dry DMSO, aliquots frozen and stored at −80° C. Concentration and purity of compound stock solutions in DMSO were determined by RP-HPLC Agilent 1100 platform with 1200 DAD and SofTA ELSD detectors, and the Agilent 6150 MS system (UV/VIS, ELSD purity and MS compound identity confirmation). Shimadzu 3.0 mm×30 mm XR ODS 2.2 μm column was run at 50° C., 1.5 mL/min. Solvent A: 0.1% Formic acid in water, Solvent B: 0.08% Formic acid in methanol-Gradient: 5%-100% B in 3.0 min, 100% solvent B for 0.3 min. Concentration of compounds in 100% DMSO determined by ELS ranged from 1-6 mM. UV-VIS and ELS determined compound purity is shown in Table 5.

### Cytotoxicity Assay

Cells were plated in 96-well white flat-bottomed plates (Corning) at 2.0×10³ cells per well in 100 μL culture medium. After incubation for 24 hours, test compounds were added at a range of concentrations as ten point serial dilution as duplicates or triplicates. Following further incubation for 6 days 37° C., 5% CO2, cell viability was assessed with the use of a CellTiter-Glo Luminescent Cell Viability Assay (Promega). Luminescence was measured using the GloMax instrument (Promega). Luminescence values were plotted against log concentration of test compounds, and the IC50 values were calculated by GraphPad Prism 9 as best-fit values using four parameter dose-response curve fit, with R squared values ranging from 0.97-0.999. For a subset of payloads, each treatment was independently repeated two to eight times, and IC50 values were averaged. Table 6 shows mean of IC50 values and standard deviations (stdev) for treatments repeated as two to eight independent experiments. Standard deviation (stdev) is shown as (N/A) for treatment performed once.

TABLE 6
Cytotoxicity IC50 (nmol/L) of exatecan derivatives for multiple tumor cell lines.

| Compound | SKBR3 | HT29 | NCI-H292 | MCF7 | NCI-N87 | FaDu | SKOV3 | BXPC3 |
|---|---|---|---|---|---|---|---|---|
| DXD | 1.3 | 6.9 | 3.8 | 2.7 | 4.7 | 0.9 | 8.6 | 2.3 |
| stdev | 0.6 | 3.7 | 2.5 | 1.3 | 3.0 | 0.6 | N/A | N/A |
| Exatecan | 0.5 | 1.0 | 1.3 | 0.4 | 1.2 | 0.6 | 0.7 | 0.3 |
| stdev | 0.3 | 0.8 | 1.1 | 0.2 | 0.7 | 0.2 | N/A | N/A |
| SN-38 | 1.8 | 5.1 | 2.5 | 4.3 | 4.2 | 2.2 | 3.2 | 0.5 |
| stdev | 2.0 | 3.2 | 1.6 | 3.7 | 2.7 | 1.9 | N/A | N/A |
| Compound 2 | 75.4 | 180.3 | 96.0 | 41.4 | 111.3 | 23.4 | 124.2 | 29.8 |
| stdev | 47.3 | 112.6 | 40.3 | 17.8 | 79.6 | 10.1 | N = 1 | N/A |
| Compound 12 | 30.5 | 318.0 | 68.6 | 43.9 | 92.0 | 16.4 | 150.6 | 45.6 |
| stdev | 19.6 | 170.0 | 55.6 | 20.4 | 99.8 | 4.6 | N/A | N/A |
| Compound 16 | 166.3 | 1137.4 | 290.3 | 140.3 | 735.6 | 72.0 | 340.9 | 148.9 |
| stdev | 81.8 | 746.2 | 255.4 | 29.5 | 851.1 | 26.2 | N/A | N/A |
| Compound 18 | 65.7 | 178.8 | 82.1 | 40.7 | 158.2 | 42.9 | 166.8 | 44.9 |
| stdev | 35.9 | 125.4 | 30.9 | 1.1 | 66.4 | 1.6 | N/A | N/A |
| Compound 22 | 1.2 | 5.7 | 2.7 | 2.3 | 2.6 | 0.7 | 1.9 | 0.7 |
| stdev | 0.3 | 3.0 | 1.7 | 2.0 | 0.7 | 0.2 | N/A | N/A |
| Compound 42 | 5.2 | 10.2 | 2.4 | 11.8 | 12.0 | 2.9 | 19.5 | 5.6 |
| stdev | N/A | 7.1 | N/A | N/A | 10.5 | N/A | N/A | N/A |
| Compound 48 | 12.6 | 63.6 | 27.1 | 11.8 | 43.0 | 2.8 | 77.8 | 20.3 |
| stdev | N/A | N/A | N/A | N/A | N/A | N/A | N/A | N/A |
| Compound 52 | 2.5 | 23.3 | 6.8 | 5.9 | 10.1 | 4.2 | 12.9 | 3.4 |
| stdev | 0.8 | 10.0 | 1.7 | 2.2 | 3.8 | 1.1 | N/A | N/A |
| Compound 58 | 12.1 | 177.2 | 60.5 | 32.0 | 49.4 | 6.3 | | |
| stdev | N/A | N/A | N/A | N/A | N/A | N/A | | |
| Compound 66 | 7.3 | 78.1 | 24.7 | 23.9 | 30.0 | 7.9 | 43.3 | 9.8 |
| stdev | 1.4 | 32.4 | 7.2 | 5.6 | 9.5 | 2.1 | N/A | N/A |
| Compound 79 | 5.9 | 31.7 | 14.0 | 9.8 | 15.5 | 2.3 | 25.3 | 5.5 |
| stdev | 1.7 | 16.9 | 7.6 | 7.8 | 3.8 | 0.7 | 2.0 | 0.1 |
| Compound 83 | 25.6 | 254.8 | 121.3 | 42.2 | 158.8 | 16.5 | | |
| stdev | 6.2 | 71.2 | 49.7 | 21.1 | 61.5 | 5.1 | | |
| Compound 100 | 36.0 | 93.4 | 228.4 | 28.8 | 78.6 | 7.0 | 90.5 | 26.9 |
| stdev | N/A | N/A | N/A | N/A | N/A | N/A | N/A | N/A |
| Compound 103 | 6.9 | 14.4 | 6.0 | 4.6 | 17.2 | 1.5 | 26.5 | 6.4 |
| stdev | 1.9 | 8.3 | 2.6 | 1.3 | 15.9 | 0.5 | 2.6 | 0.6 |
| Compound 105 | 0.9 | 2.1 | 1.7 | 1.4 | 1.6 | 1.3 | 1.8 | 0.6 |
| stdev | 0.3 | 1.4 | 1.0 | 1.6 | 0.7 | 0.8 | N/A | N/A |
| Compound 106 | 0.4 | 1.1 | 0.4 | 0.2 | 0.9 | 0.0 | | |
| stdev | 0.2 | 0.5 | 0.2 | 0.2 | 0.5 | 0.0 | | |
| Compound 107 | 0.1 | 0.4 | 0.6 | 0.2 | 0.2 | 0.1 | | |
| stdev | 0.0 | 0.1 | 0.2 | 0.1 | 0.1 | 0.0 | | |
| Compound 108 | 2.9 | 13.2 | 8.1 | 4.4 | 8.3 | 2.4 | 6.6 | 2.3 |
| stdev | 0.5 | 10.0 | 4.6 | 3.5 | 4.8 | 0.4 | N/A | N/A |
| Compound 109 | 7.3 | 56.8 | 26.1 | 12.8 | 29.3 | 8.3 | 32.4 | 10.8 |
| stdev | 0.4 | 21.9 | 1.2 | 9.5 | 0.4 | 0.2 | N/A | N/A |
| Compound 110 | 7.6 | 28.6 | 18.8 | 22.4 | 18.2 | 15.6 | 59.2 | 15.7 |
| stdev | N/A | N/A | N/A | N/A | N/A | N/A | N/A | N/A |
| Compound 111-A | 6.0 | 34.4 | 18.0 | 16.7 | 13.1 | 1.9 | 24.5 | 5.6 |
| stdev | 2.2 | 24.4 | 12.9 | 11.0 | 4.2 | 0.5 | N/A | N/A |
| Compound 111-B | 2.1 | 6.3 | 4.5 | 3.8 | 5.2 | 1.1 | | |
| stdev | 0.5 | 1.2 | 1.1 | 3.0 | 2.1 | 0.4 | N/A | |
| Compound 115 | 62.8 | 155.0 | 127.8 | 47.0 | 127.2 | 56.1 | 173.7 | 55.0 |
| stdev | 10.0 | 59.0 | 6.1 | N/A | 12.7 | 27.0 | N/A | N/A |
| Compound 117 | 474.4 | 2913.8 | 887.7 | 342.1 | 1446.9 | 233.8 | 1089.0 | 412.0 |
| stdev | 27.4 | 399.6 | 105.8 | 7.8 | 251.9 | 2.5 | N/A | N/A |
| Compound 118 | 23.9 | 290.9 | 88.4 | 45.9 | 96.6 | 17.5 | 96.7 | 52.7 |
| stdev | 9.4 | 182.1 | 4.1 | 12.9 | 32.0 | 8.5 | N/A | N/A |
| Compound 122 | 657.2 | 3396.5 | 1022.4 | 205.4 | 2695.9 | 121.0 | 818.6 | 226.9 |
| stdev | 80.8 | 3179.9 | 966.8 | 27.7 | 1080.6 | 96.6 | N/A | 18.6 |
| Compound 129 | 168.6 | 281.8 | 207.5 | 79.3 | 300.2 | 92.9 | 264.6 | 133.9 |
| stdev | 109.2 | 202.2 | 97.3 | 51.6 | 213.0 | 43.4 | N/A | N/A |
| Compound 136 | 23.7 | 36.8 | 16.3 | 18.4 | 40.2 | 5.2 | 98.4 | 24.9 |
| stdev | 3.0 | 9.4 | 4.0 | 4.0 | 2.5 | 3.7 | 3.3 | N/A |
| Compound 140 | 0.8 | 1.2 | 3.0 | 0.2 | 1.5 | 0.3 | | |
| stdev | 0.6 | 0.3 | 2.2 | N/A | 0.6 | 0.2 | | |
| Compound 147 | 2.7 | 7.1 | 9.4 | 3.1 | 8.5 | 3.0 | | |
| stdev | 0.3 | 1.7 | 6.3 | 2.7 | 3.2 | 0.4 | | |
| Compound 148 | 0.3 | 0.9 | 1.1 | 0.1 | 0.2 | 0.0 | 0.2 | 0.1 |
| stdev | 0.1 | 1.4 | 2.0 | 0.1 | 0.2 | 0.0 | 0.1 | 0.2 |
| Compound 159 | 5.8 | 64.2 | 21.6 | 9.8 | 22.4 | 2.0 | | |
| stdev | N/A | N/A | N/A | N/A | N/A | N/A | | |
| Compound 163 | 9.8 | 65.0 | 22.3 | 10.9 | 29.4 | 7.8 | | |
| stdev | 0.6 | 4.7 | 2.6 | 1.2 | 0.7 | 0.6 | | |
| Compound 164 | 10.8 | 32.7 | 30.3 | 5.2 | 22.8 | 8.8 | | |
| stdev | 1.2 | 11.8 | 25.2 | 1.0 | 2.9 | 1.3 | | |
| Compound 166 | 118.0 | 663.0 | 322.2 | 162.5 | 409.5 | 93.8 | | |
| stdev | 9.8 | 222.4 | 145.7 | 62.3 | 117.1 | 21.2 | | |
| Compound 167 | 3.5 | 43.5 | 23.0 | 8.9 | 34.0 | 3.0 | | |
| stdev | 1.3 | 4.5 | 1.8 | 5.4 | 3.7 | 0.7 | | |
| Compound 168 | 32.7 | 627.4 | 77.9 | 19.4 | 102.8 | 9.9 | | |
| stdev | 1.2 | N/A | 2.9 | 1.4 | 4.7 | 1.0 | | |
| Compound 175-B | 0.5 | 1.6 | 1.5 | 0.6 | 1.5 | 0.3 | | |
| stdev | 0.4 | 0.9 | 0.5 | 0.7 | 1.4 | 0.1 | | |
| Compound 176 | 0.7 | 4.3 | 4.5 | 7.8 | 2.1 | 0.9 | | |
| stdev | 0.1 | 0.0 | 0.2 | 5.0 | 0.0 | 0.7 | | |
| Compound 188 | 3.4 | 13.0 | 6.6 | 4.9 | 9.0 | 1.5 | | |
| stdev | 2.1 | 8.2 | 3.3 | 1.6 | 5.5 | 0.8 | | |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound represented by Formula I: 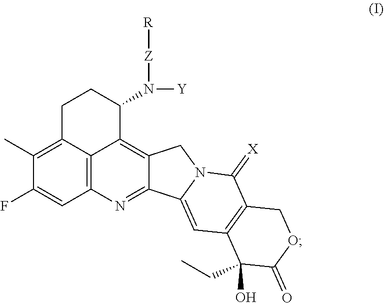
or a pharmaceutically acceptable salt or stereoisomer thereof, wherein:
X is selected from the group consisting of O;
Z is a bond; Y is selected from the group consisting of hydrogen, —C₁₋₃alkyl, —CHO, and —C(O)—C₁₋₃alkyl; and R is selected from the group consisting of R², R³, and R⁴; or
Y and Z, together with the nitrogen to which they are attached, are joined together to form a 5-6 membered heteroaryl optionally substituted by one, two or three substituents, each independently selected from R^{Z}; R is bonded to the heteroaryl; and R is R⁶; or
—N(Y)—Z—R is selected from the group consisting of: 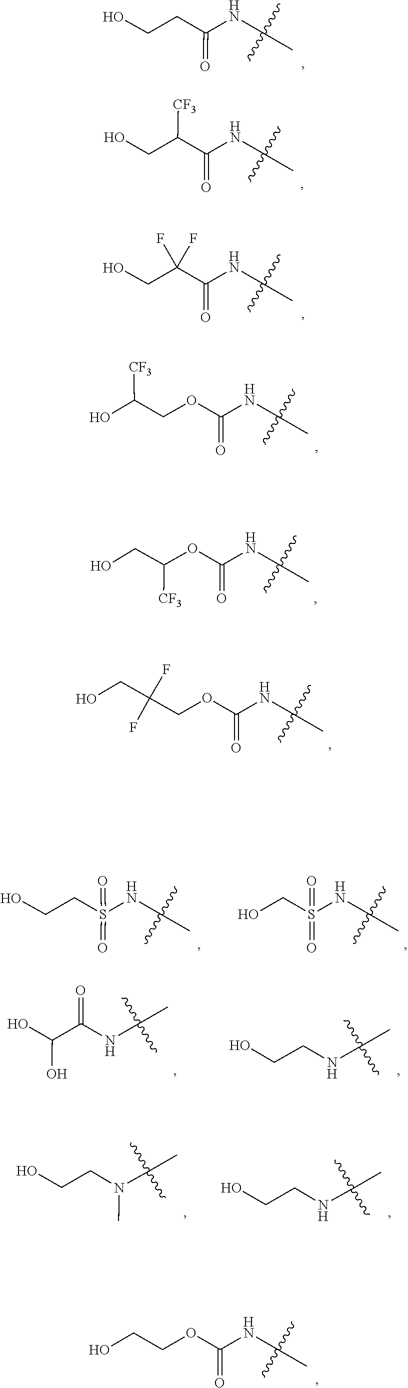 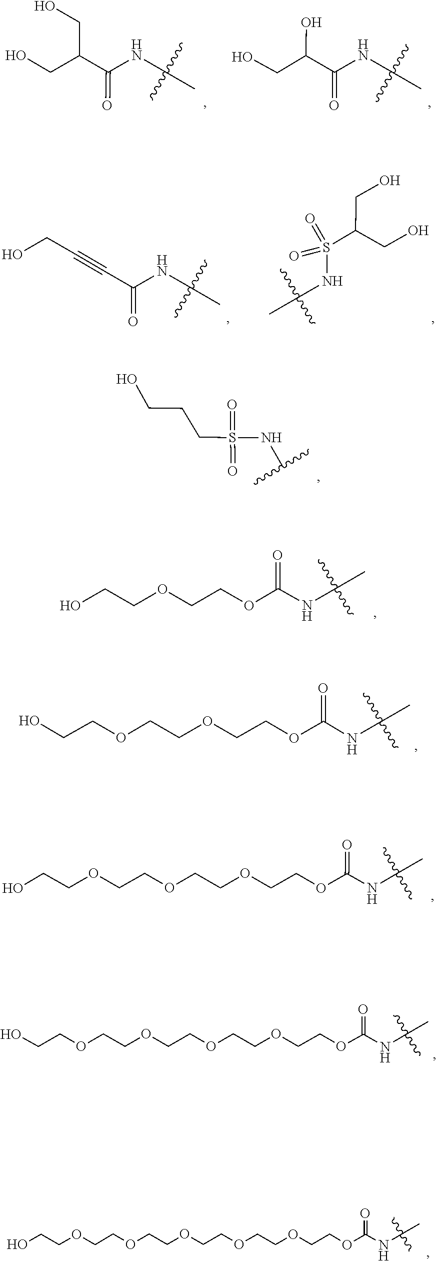 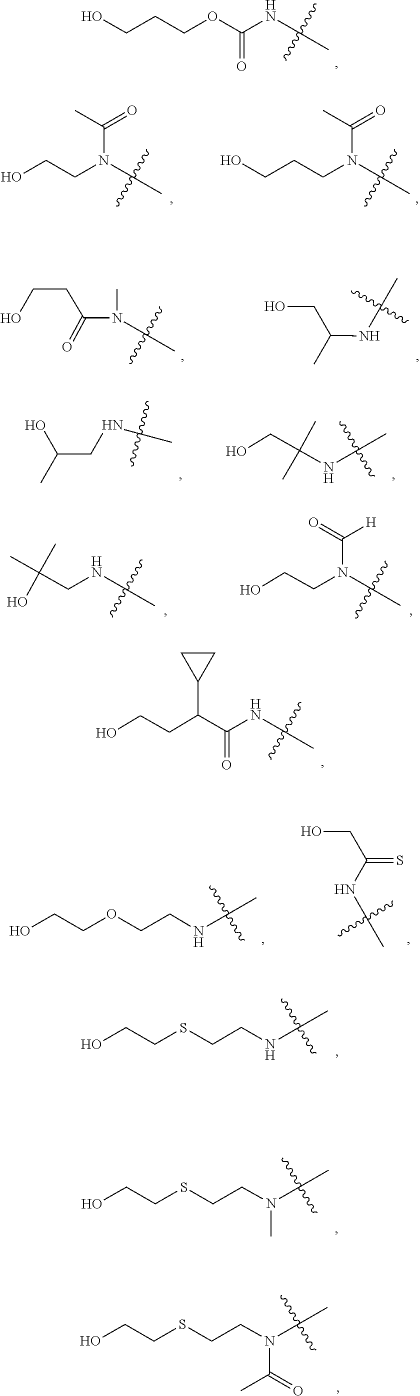 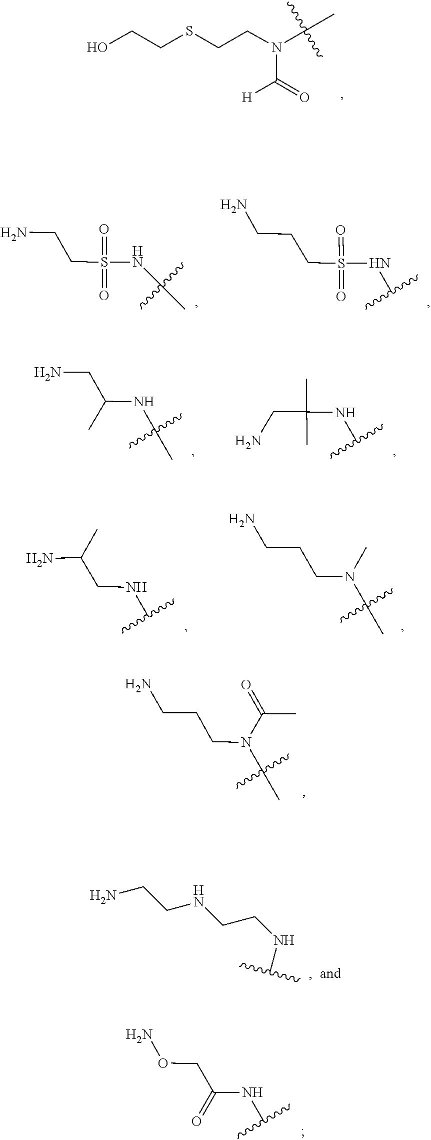
R² is selected from the group consisting of —C(O)—NR^{a}—C₁₋₃alkyl, —C(O)—C₀₋₃alkyl-C(O)—NR^{a}—C₁₋₃alkyl, —C(O)—C₁₋₃alkyl-NR^{a}—C₁₋₃alkyl, —S(O)₂—C₁₋₃alkyl-NR^{a}—C(O)—C₁₋₃alkyl, and —C(O)NR^{a}—[(CH₂)₂—O]₁₋₁₀—C₂alkyl; wherein R² is substituted by hydroxyl and optionally substituted by one or more additional substituents each independently selected from R²²;
R²² is independently selected for each occurrence from the group consisting of halogen, hydroxyl, —C₁₋₃alkyl-OH, and —C₁₋₃haloalkyl;
R³ is selected from the group consisting of —C(O)—C₀₋₃alkyl-R³⁰, —C(O)—C₀₋₃alkyl-O—C₁₋₃alkyl-R³⁰, —C₀₋₃alkyl-R³⁰, and —C₁₋₃alkyl-O—C₁₋₃alkyl-R³⁰; wherein the alkyl if present may optionally be substituted by one or more substituents each independently selected from the group consisting of halogen and —C₁₋₃haloalkyl;
R³⁰ is selected from the group consisting of 5-6 membered heteroaryl and 4-10 membered heterocyclyl having one, two or three heteroatoms, each independently selected from the group consisting of N, NR³¹, and O; wherein R³⁰ is optionally substituted on one or more available carbons by one or more substituents each independently selected from R³³;
R³¹ is independently selected for each occurrence from the group consisting of hydrogen, —C₁₋₃alkyl, —C₁₋₃alkyl-OH, —CH(OH)CH₂OH, —CHO, and —C(O)—C₁₋₃alkyl;
R³³ is independently selected for each occurrence from the group consisting of —C₁₋₃alkyl-OH, halogen, hydroxyl, oxo, and —C₁₋₃haloalkyl;
R⁴ is selected from the group consisting of; 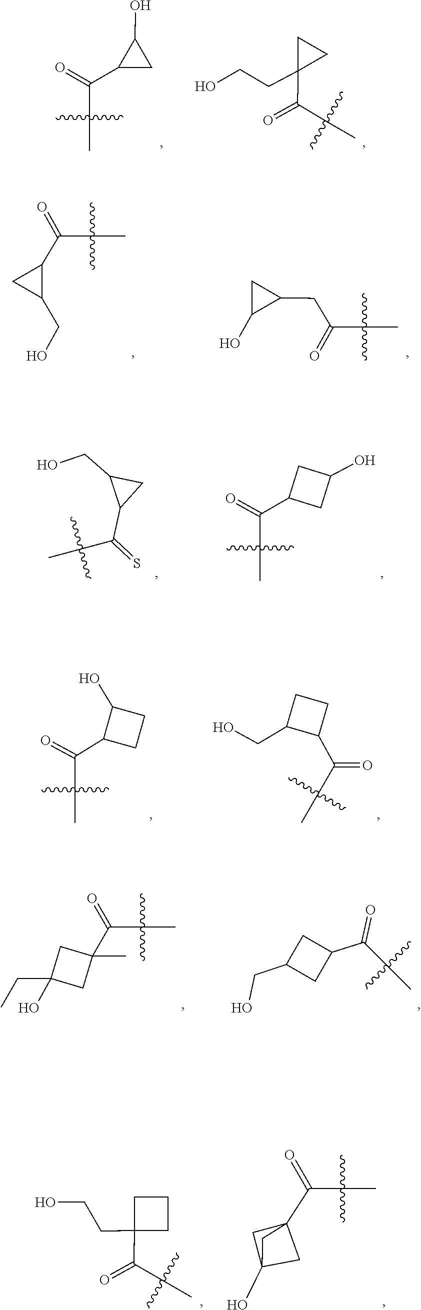 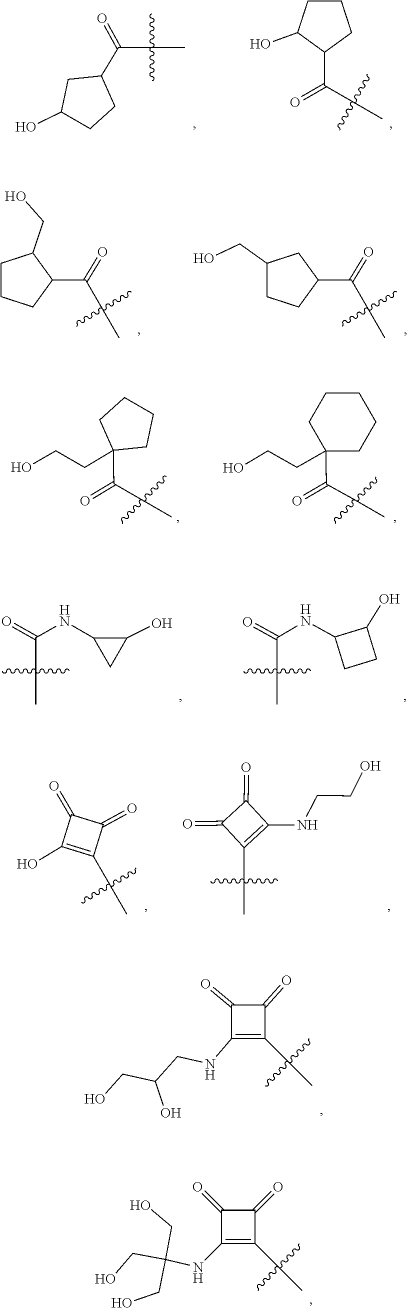 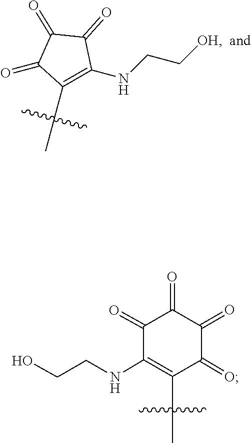
R⁶ is —C₁₋₃alkyl substituted by hydroxyl and optionally substituted by one or more additional substituents each independently selected from R⁶⁶;
R⁶⁶ is independently selected for each occurrence from the group consisting of halogen and —C₁₋₃haloalkyl;
R^{Z} is selected from the group consisting of halogen, —C₁₋₃alkyl and —C₁₋₃alkyl-OH; and
R^{a} and R^{b} are each independently selected for each occurrence from the group consisting of the group consisting of hydrogen, —C₁₋₃alkyl-OH, and —C₁₋₃haloalkyl-OH.

2.-3. (canceled)

4. The compound of claim 1, wherein Y is selected from the group consisting of hydrogen, —CH₃, —CHO, and —COCH₃.

5.-9. (canceled)

10. The compound of claim 1, wherein Y is hydrogen.

11. The compound of claim 1, wherein R² is selected from the group consisting of —C(O)—NH—C₂alkyl, —C(O)—NH—C₃alkyl, —C(O)—C(O)—NH—C₂alkyl, —C(O)—C(O)—NH—C₃alkyl, —C(O)—C₁alkyl-C(O)—NH—C₂alkyl, —C(O)—C₂alkyl-C(O)—NH—C₂alkyl, —C(O)—C₂alkyl-C(O)—NH—C₃alkyl, —S(O)₂-C₂alkyl-NH—C(O)—C₁alkyl, —S(O)₂—C₂alkyl-NH—C(O)—C₂alkyl, and —C(O)NH—[(CH₂)₂—O]₁₋₂—C₂alkyl; and wherein R² is substituted by hydroxyl and optionally substituted by one or more additional substituents one or more additional substituents each independently selected from R²².

12. The compound of claim 1, wherein R²² is selected from the group consisting of fluoro, hydroxyl, —CH₂—OH, and —CF₃.

13. The compound of claim 1, wherein —N(Y)—Z—R is selected from the group consisting of:

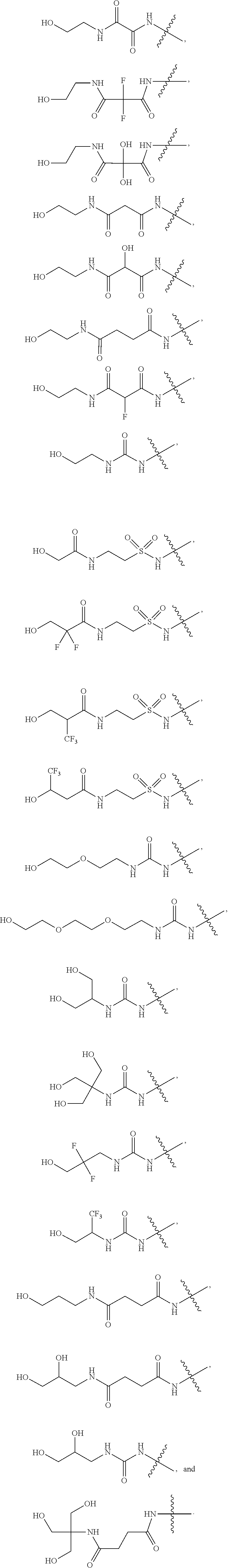

14.-15. (canceled)

16. The compound of claim 1, wherein R³ is selected from the group consisting of:
—C(O)-triazolyl, —C(O)—C₁alkyl-triazolyl, —C(O)—C₂alkyl-triazolyl, —C(O)—C₃alkyl-triazolyl, —C₁alkyl-triazolyl, —C₂alkyl-triazolyl, —C₃alkyl-triazolyl, —C(O)—O—C₁alkyl-triazolyl, —C(O)—O—C₂alkyl-triazolyl, —C(O)—C₁alkyl-O—C₂alkyl-triazolyl, —C(O)—C₂alkyl-O—C₁alkyl-triazolyl, —C(O)—C₂alkyl-O—C₂alkyl-triazolyl, —C₂alkyl-O—C₁alkyl-triazolyl, and —C₂alkyl-O—C₂alkyl-triazolyl; wherein:
alkyl for each occurrence may optionally be substituted by one, two or three substituents each independently selected from the group consisting of fluoro and —CF₃;
triazolyl is substituted on an available nitrogen, if present, by a substituent selected from the group consisting of hydrogen, —C₁₋₃alkyl, and —C₁₋₂alkyl-OH; and
triazolyl may optionally be substituted on an available carbon by a substituent selected from the group consisting of chloro, fluoro, and —C₁₋₂alkyl-OH.

17. The compound of claim 1, wherein R³ is selected from the group consisting of:

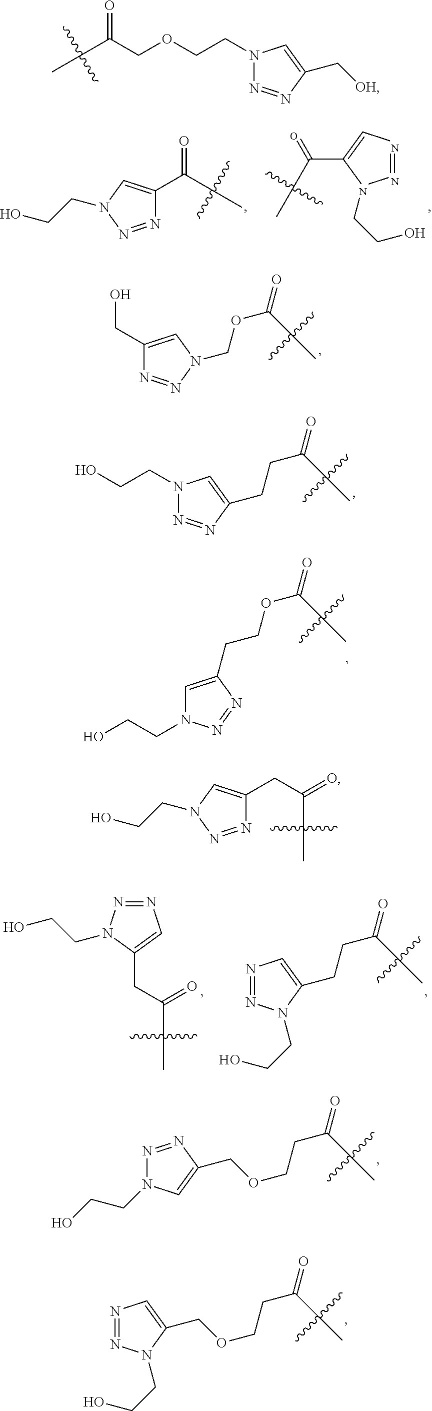

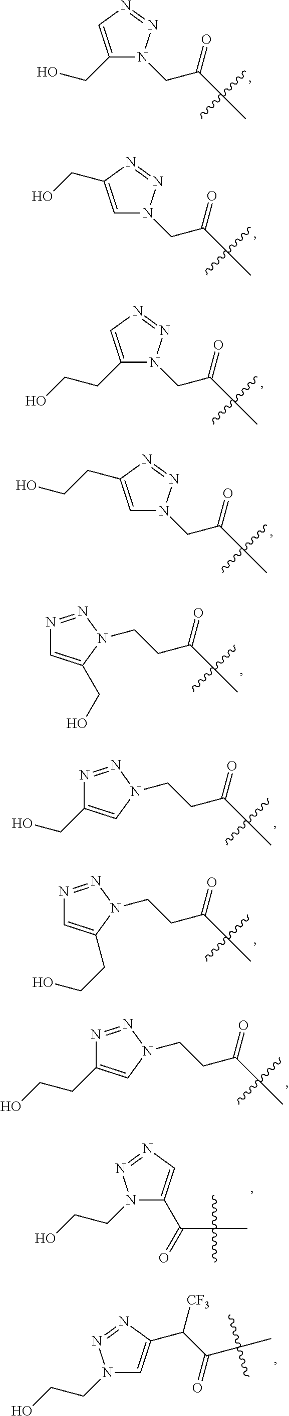

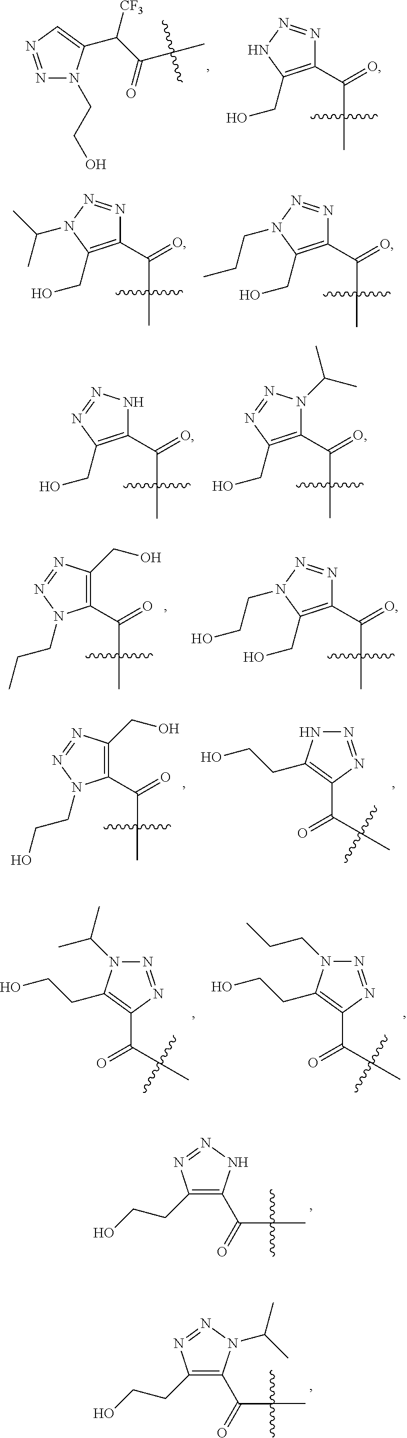

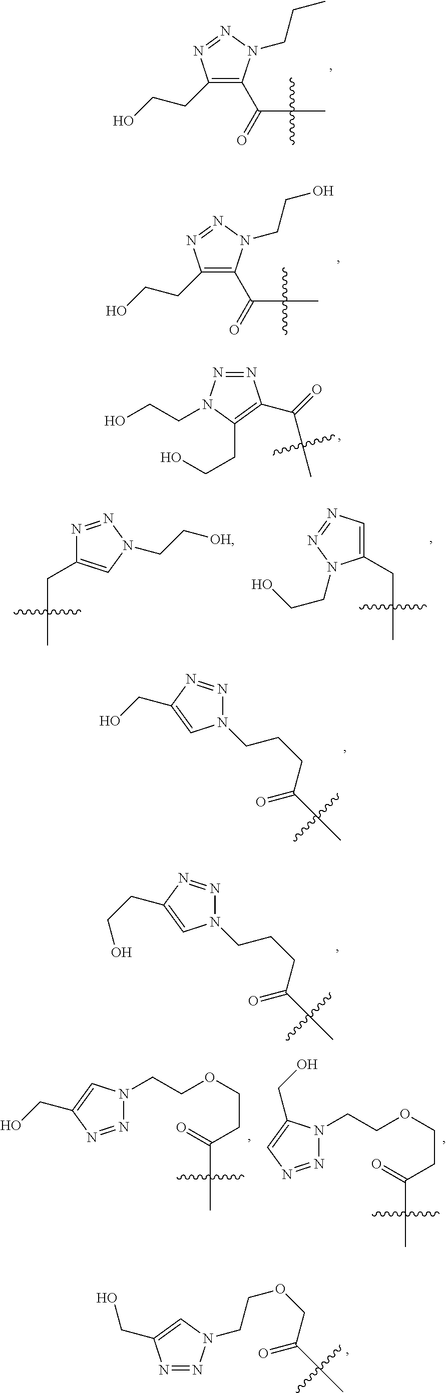

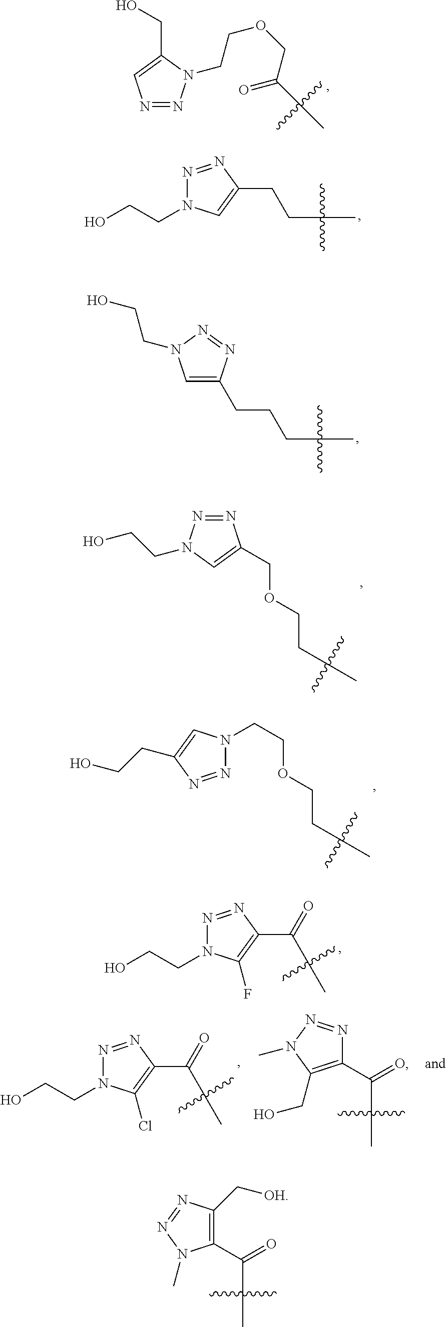

18. The compound of claim 1, wherein R³ is selected from the group consisting of: —C(O)-furanyl, —C₁alkyl-furanyl, —C(O)-oxazolyl, and —C(O)-pyrrazolyl; wherein R³ is substituted by a substituent selected from the group consisting of hydroxyl and —C₁₋₂alkyl-OH.

19. The compound of claim 1, wherein R³ is selected from the group consisting of:

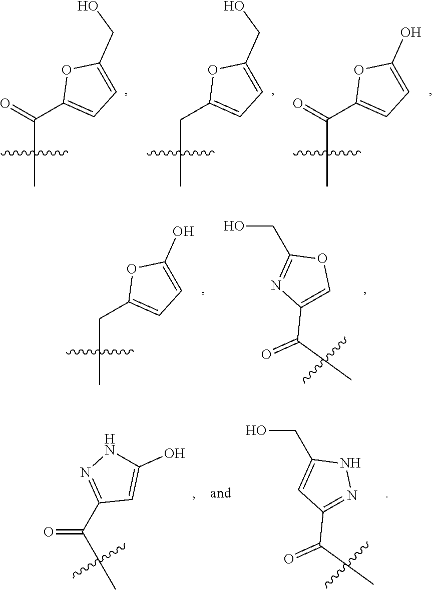

20. The compound of claim 1, wherein R³ is selected from the group consisting of:

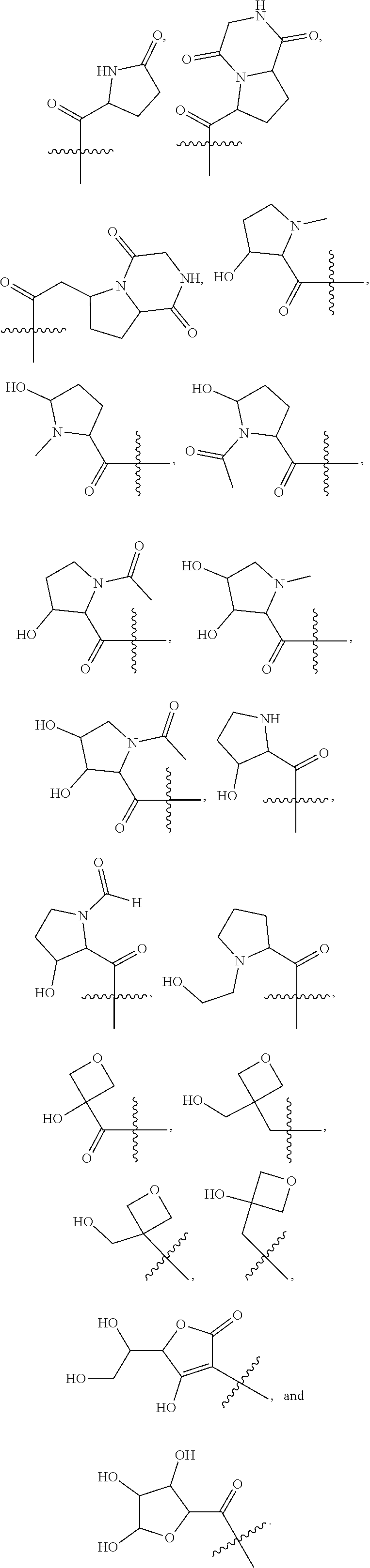

21.-28. (canceled)

29. The compound of claim 1, Y and Z, together with the nitrogen to which they are attached, are joined together to form a triazolyl selected from the group consisting of:

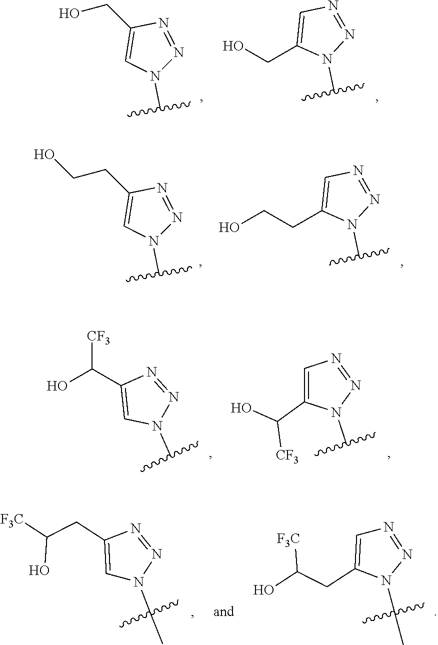

30.-34. (canceled)

35. A compound selected from the group consisting of: 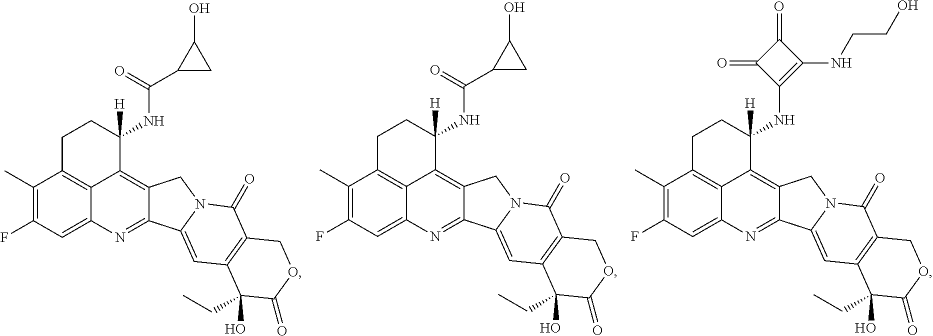 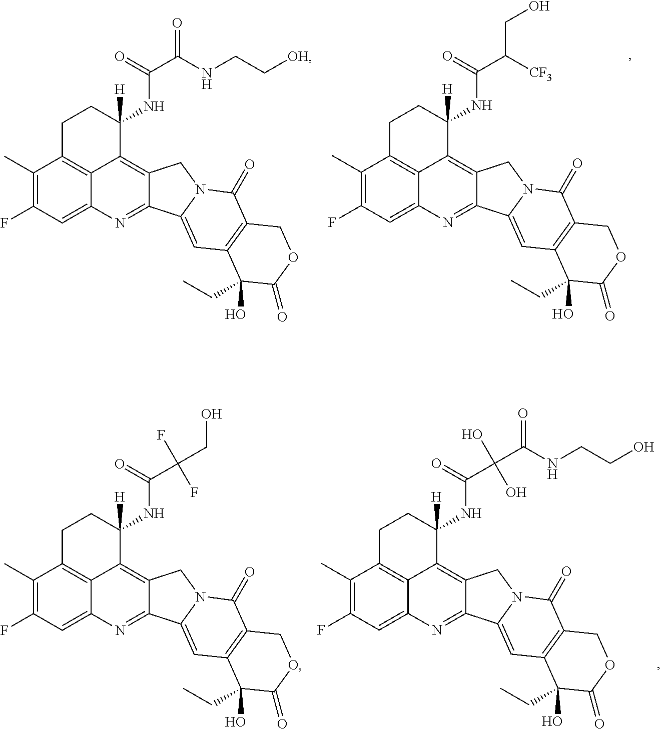 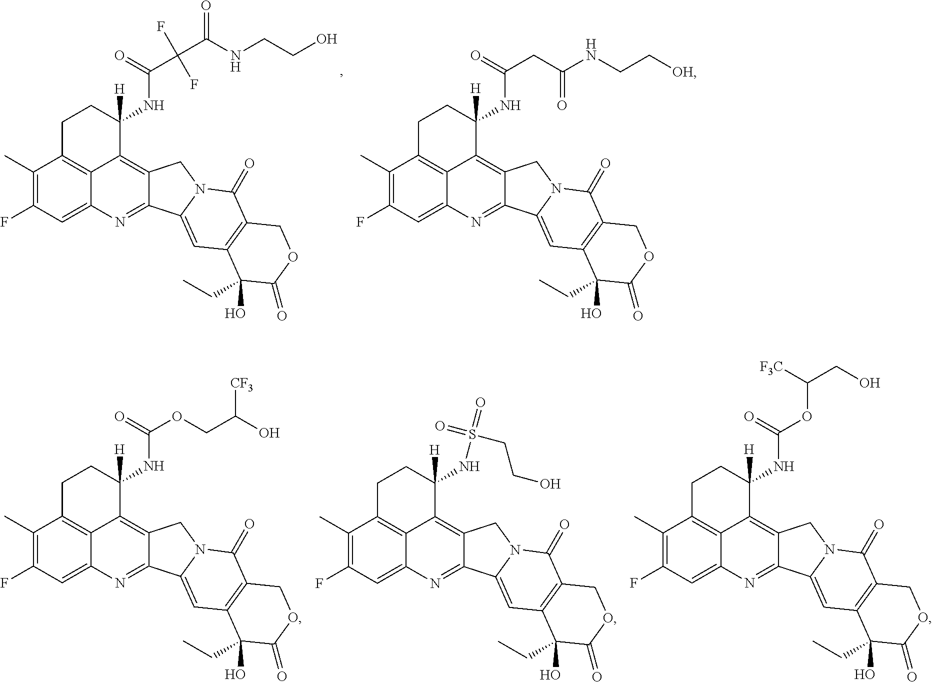 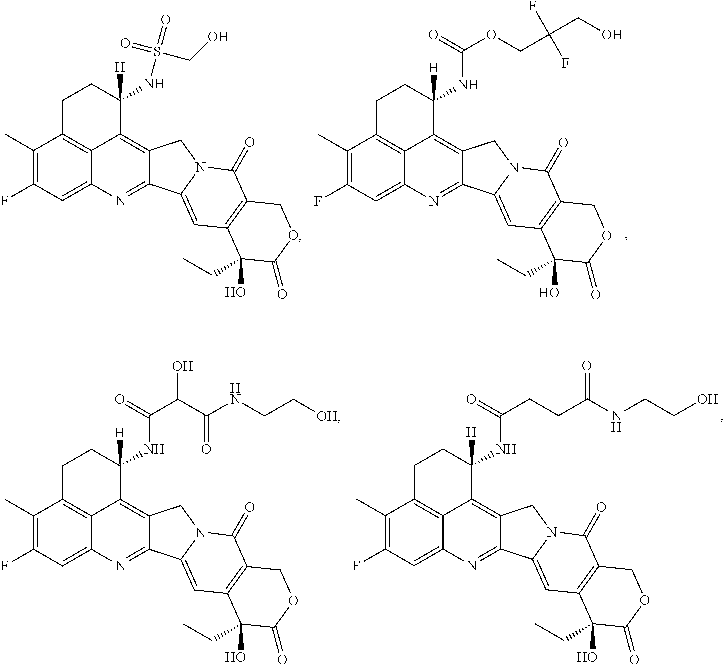 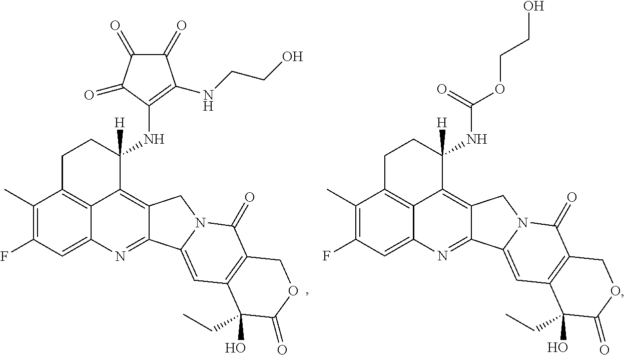 [SMILES: CC[C@@]1(O)C(=O)OCc2c1cc1n(c2=O)Cc2c-1nc1cc(F)c(C)c3c1c2[C@@H](Nc1c(NCCO)c(=O)c(=O)c1=O)CC3 ; CC[C@@]1(O)C(=O)OCc2c1cc1n(c2=O)Cc2c-1nc1cc(F)c(C)c3c1c2[C@@H](NC(=O)OCCO)CC3] 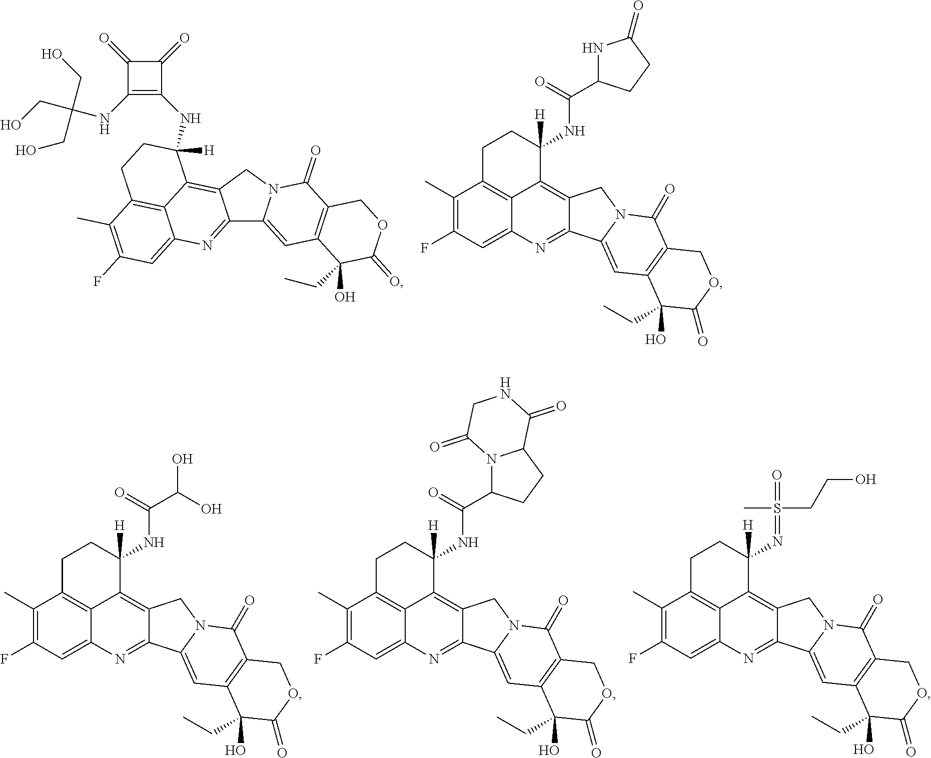 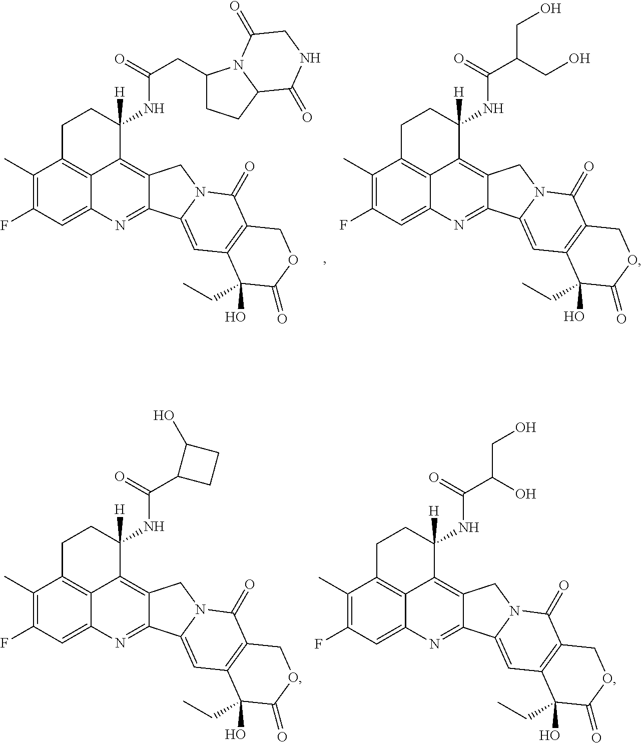 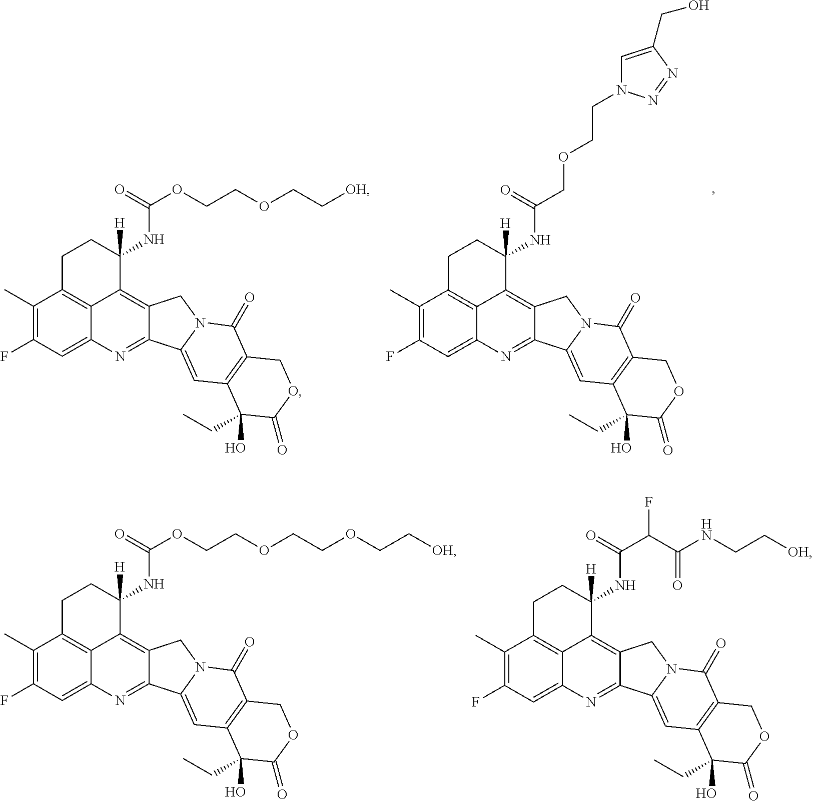 [SMILES: CC[C@@]1(O)C(=O)OCc2c1cc1n(c2=O)Cc2c-1nc1cc(F)c(C)c3c1c2[C@@H](NC(=O)OCCOCCO)CC3 ; CC[C@@]1(O)C(=O)OCc2c1cc1n(c2=O)Cc2c-1nc1cc(F)c(C)c3c1c2[C@@H](NC(=O)COCCn1cc(CO)nn1)CC3 ; CC[C@@]1(O)C(=O)OCc2c1cc1n(c2=O)Cc2c-1nc1cc(F)c(C)c3c1c2[C@@H](NC(=O)OCCOCCOCCO)CC3 ; CC[C@@]1(O)C(=O)OCc2c1cc1n(c2=O)Cc2c-1nc1cc(F)c(C)c3c1c2[C@@H](NC(=O)C(F)C(=O)NCCO)CC3] 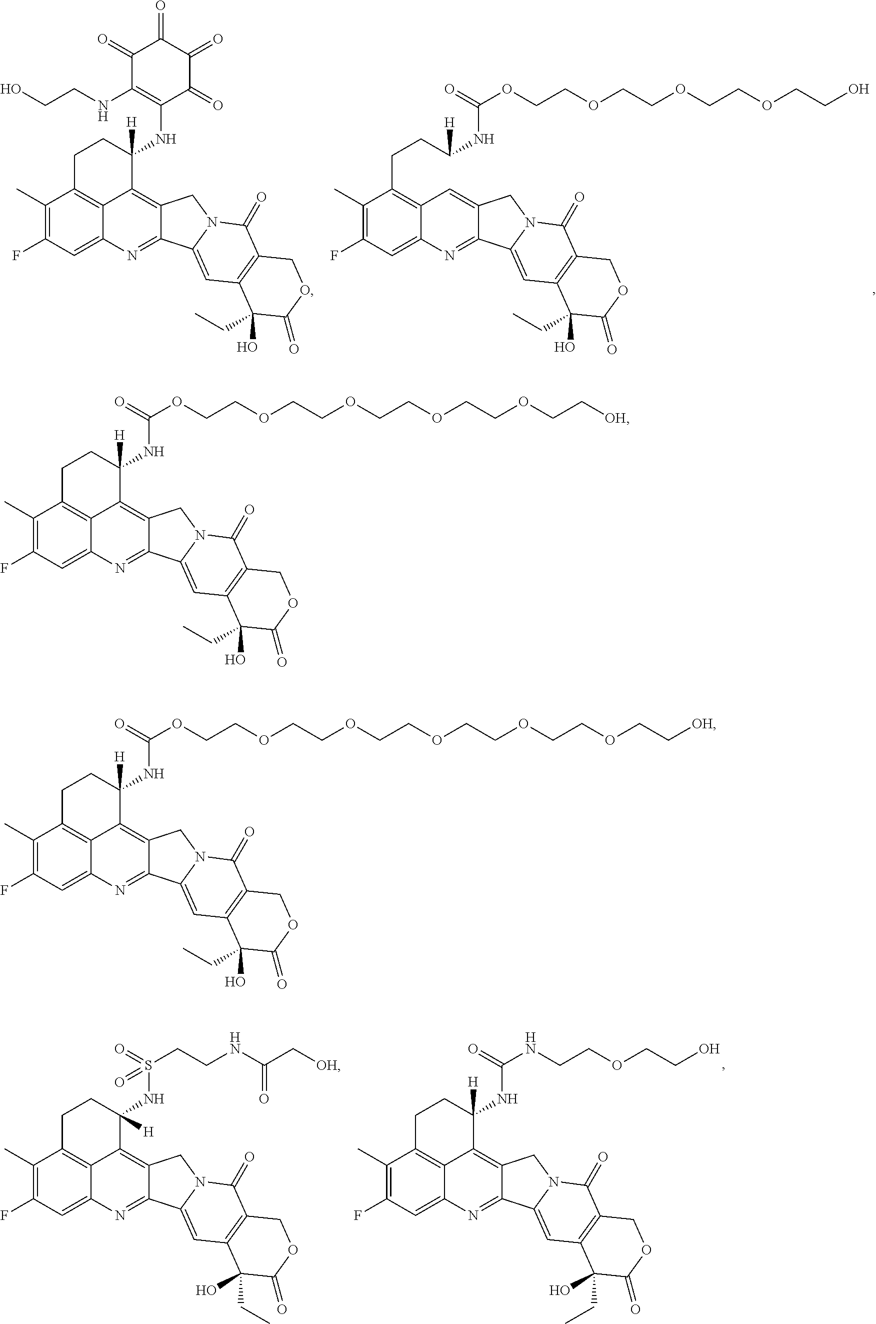 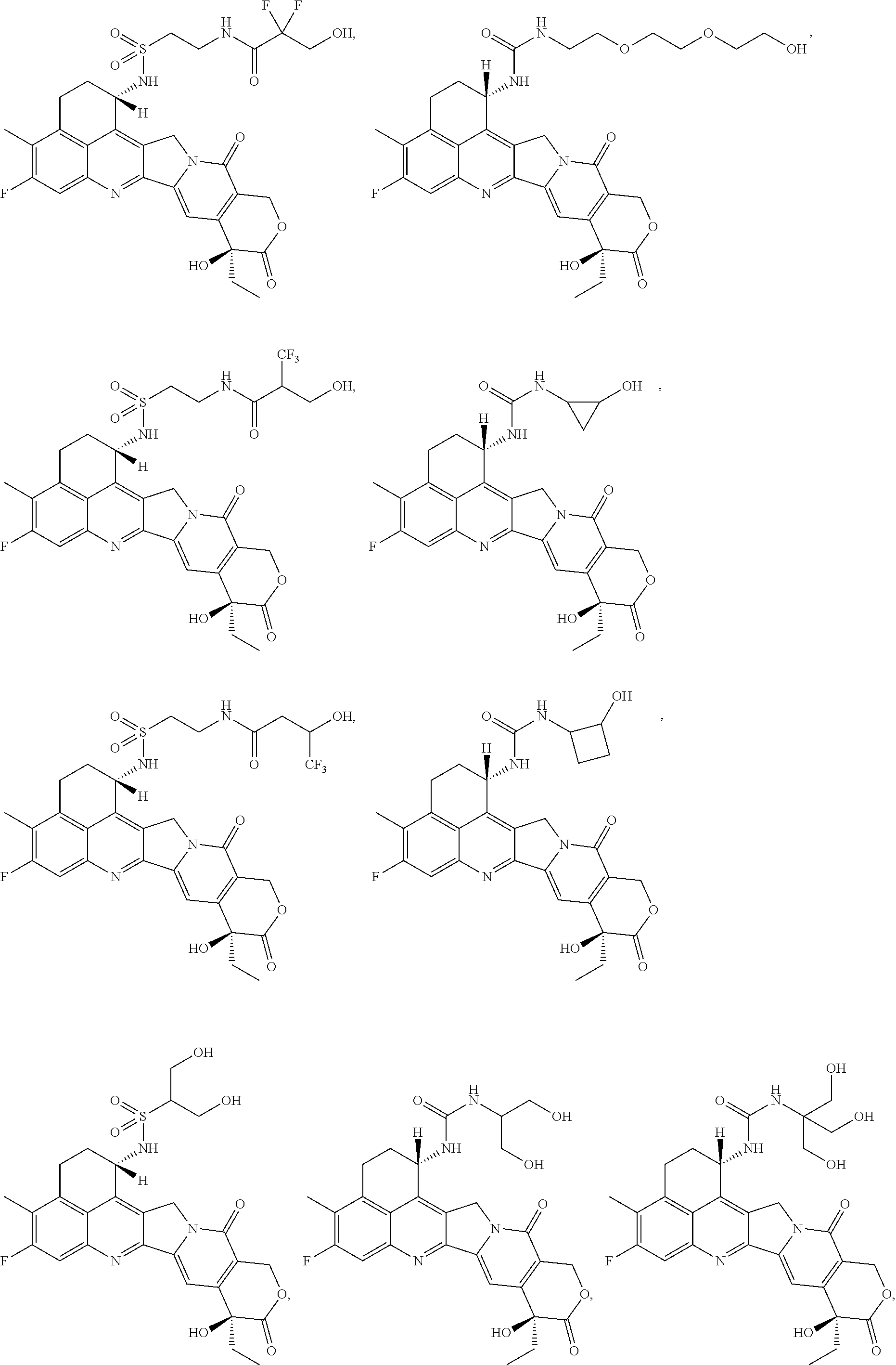 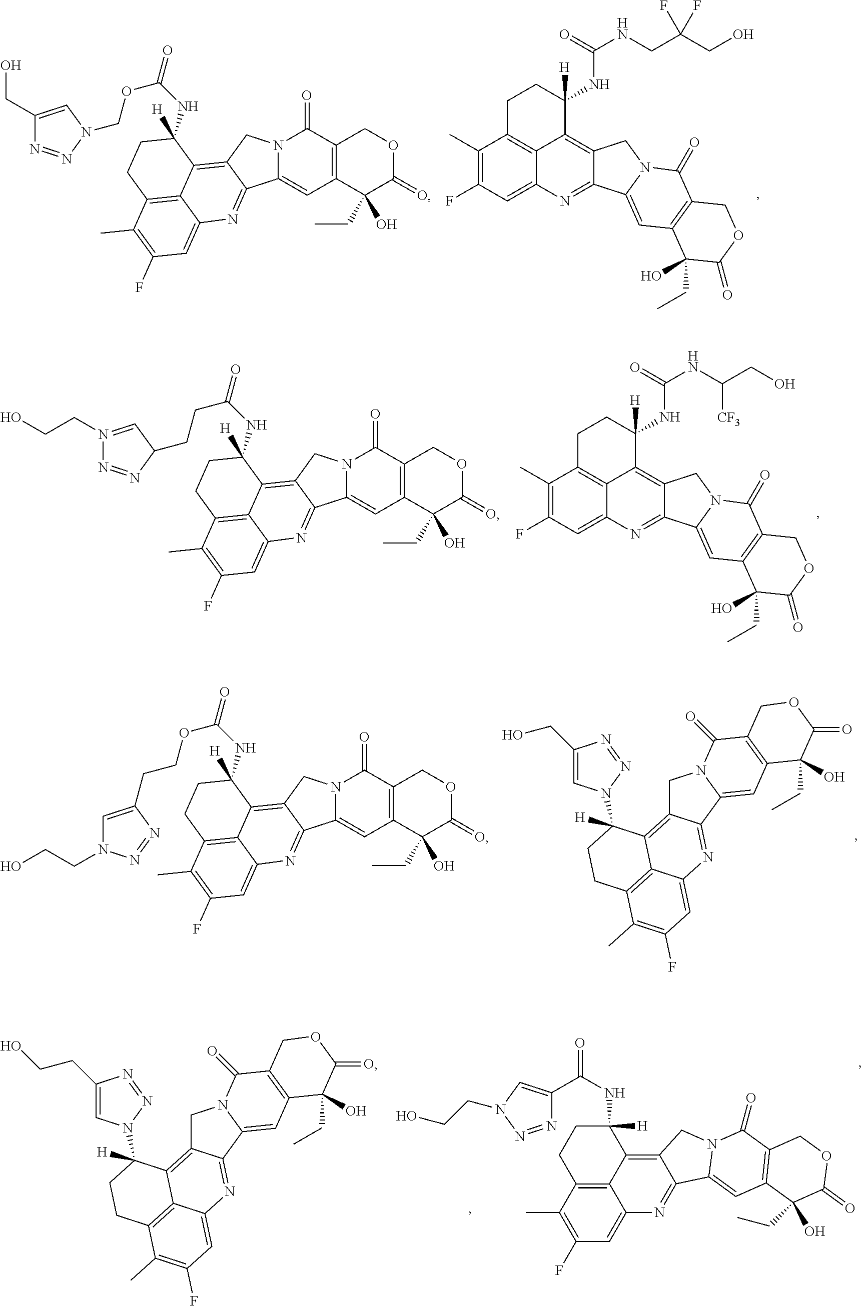 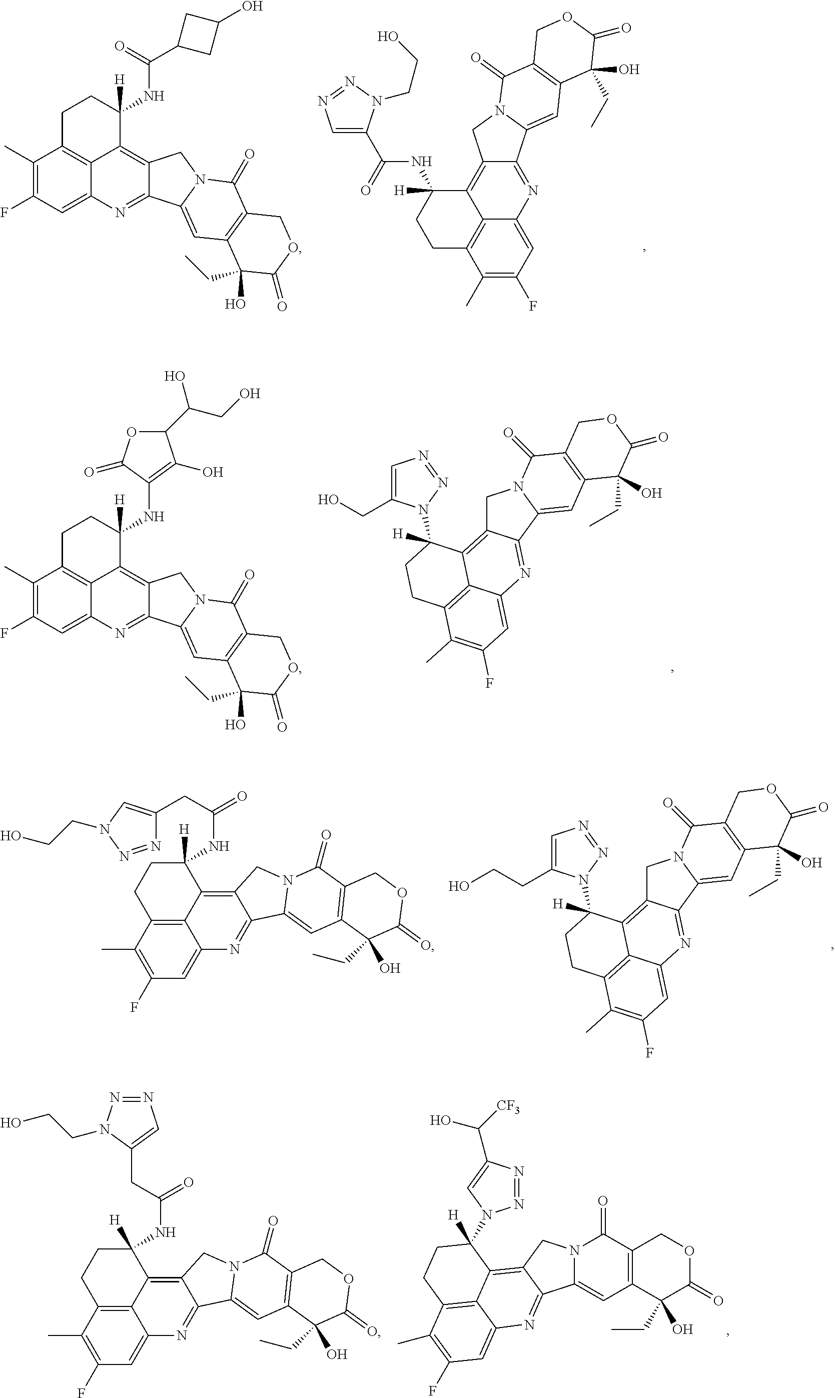 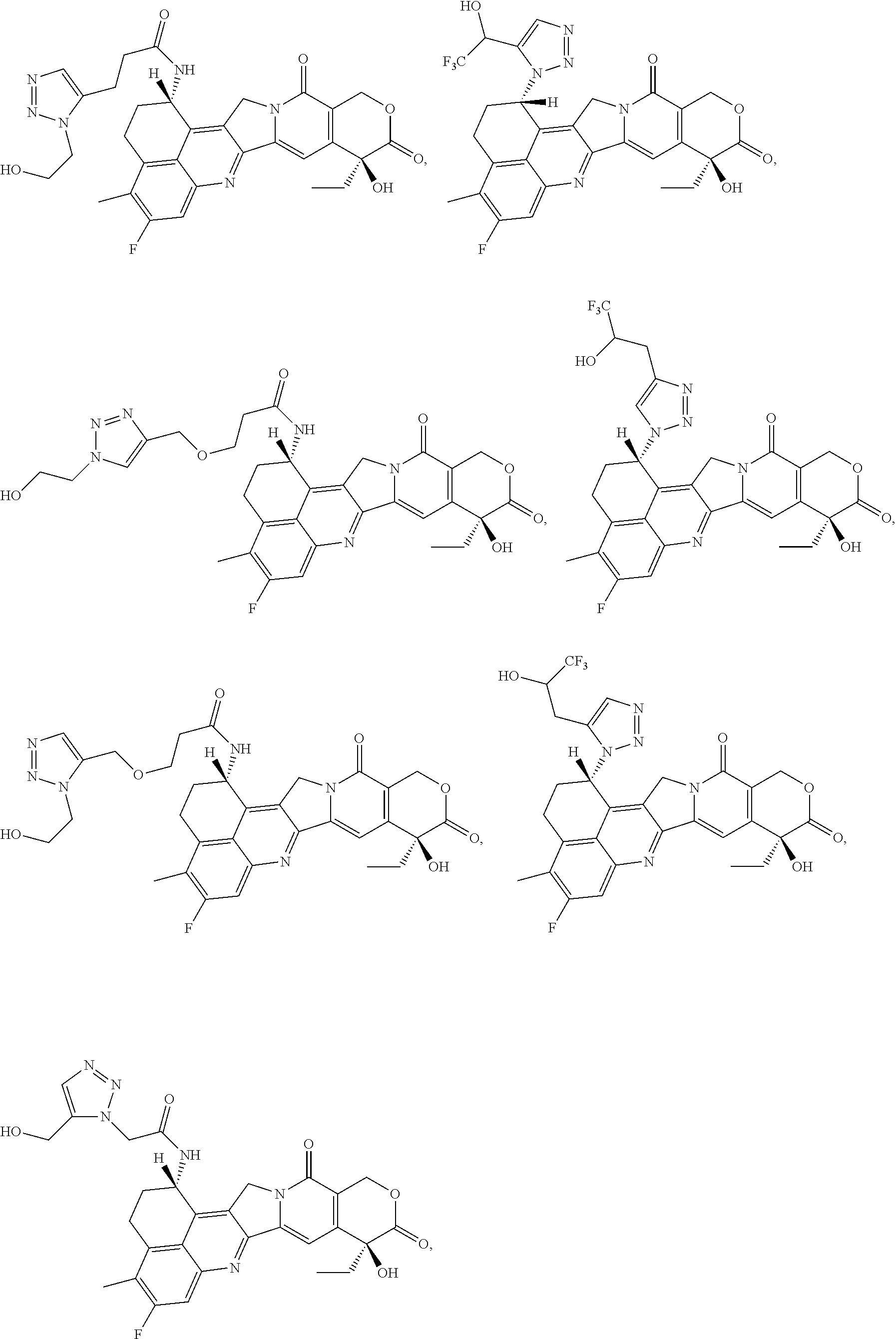 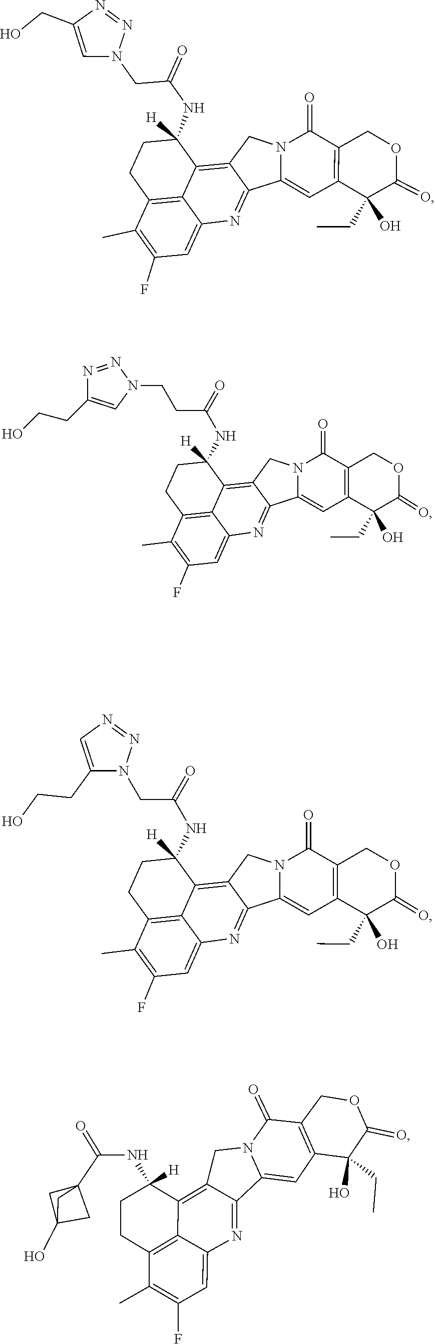 [SMILES: CC[C@@]1(O)C(=O)OCc2c1cc1n(c2=O)Cc2c-1nc1cc(F)c(C)c3c1c2[C@@H](NC(=O)Cn1cc(CO)nn1)CC3 ; CC[C@@]1(O)C(=O)OCc2c1cc1n(c2=O)Cc2c-1nc1cc(F)c(C)c3c1c2[C@@H](NC(=O)CCn1cc(CCO)nn1)CC3 ; CC[C@@]1(O)C(=O)OCc2c1cc1n(c2=O)Cc2c-1nc1cc(F)c(C)c3c1c2[C@@H](NC(=O)Cn1nncc1CCO)CC3 ; CC[C@@]1(O)C(=O)OCc2c1cc1n(c2=O)Cc2c-1nc1cc(F)c(C)c3c1c2[C@@H](NC(=O)C12CC(O)(C1)C2)CC3] 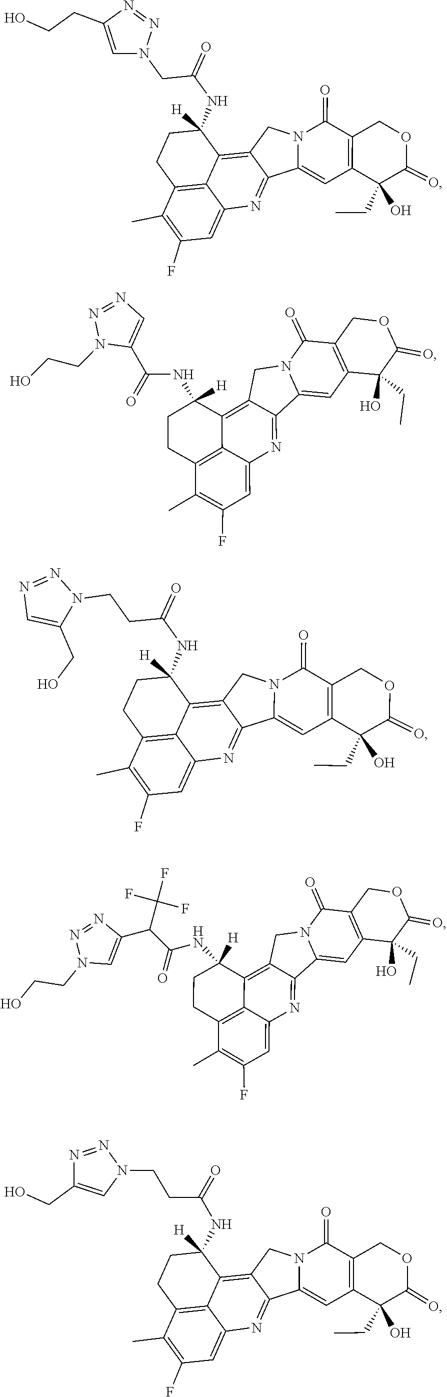 [SMILES: CC[C@@]1(O)C(=O)OCc2c1cc1n(c2=O)Cc2c-1nc1cc(F)c(C)c3c1c2[C@@H](NC(=O)Cn1cc(CCO)nn1)CC3 ; CC[C@@]1(O)C(=O)OCc2c1cc1n(c2=O)Cc2c-1nc1cc(F)c(C)c3c1c2[C@@H](NC(=O)c1cnnn1CCO)CC3 ; CC[C@@]1(O)C(=O)OCc2c1cc1n(c2=O)Cc2c-1nc1cc(F)c(C)c3c1c2[C@@H](NC(=O)CCn1nncc1CO)CC3 ; CC[C@@]1(O)C(=O)OCc2c1cc1n(c2=O)Cc2c-1nc1cc(F)c(C)c3c1c2[C@@H](NC(=O)C(c1cn(CCO)nn1)C(F)(F)F)CC3 ; CC[C@@]1(O)C(=O)OCc2c1cc1n(c2=O)Cc2c-1nc1cc(F)c(C)c3c1c2[C@@H](NC(=O)CCn1cc(CO)nn1)CC3] 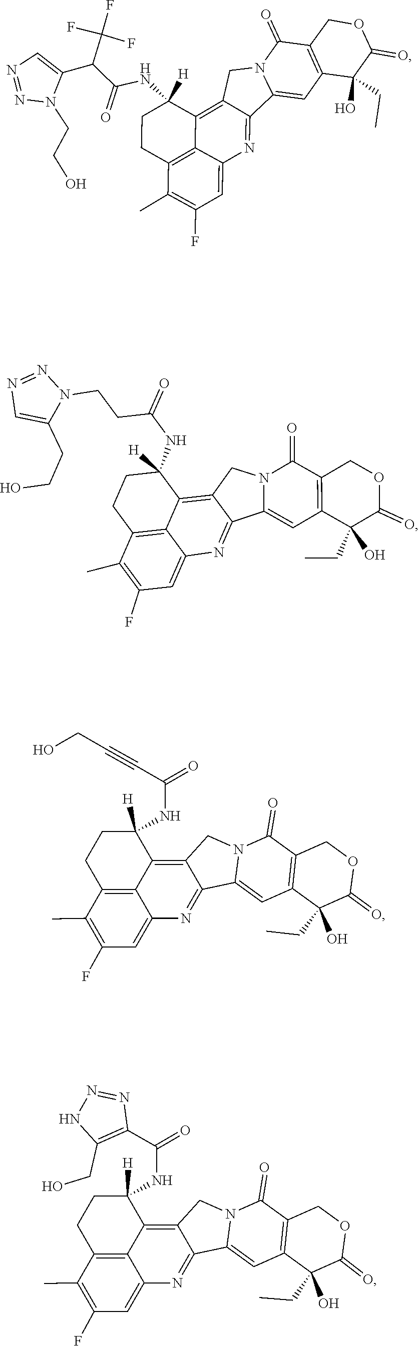 [SMILES: CC[C@@]1(O)C(=O)OCc2c1cc1n(c2=O)Cc2c-1nc1cc(F)c(C)c3c1c2[C@@H](NC(=O)C(c1cnnn1CCO)C(F)(F)F)CC3 ; CC[C@@]1(O)C(=O)OCc2c1cc1n(c2=O)Cc2c-1nc1cc(F)c(C)c3c1c2[C@@H](NC(=O)CCn1nncc1CCO)CC3 ; CC[C@@]1(O)C(=O)OCc2c1cc1n(c2=O)Cc2c-1nc1cc(F)c(C)c3c1c2[C@@H](NC(=O)C#CCO)CC3 ; CC[C@@]1(O)C(=O)OCc2c1cc1n(c2=O)Cc2c-1nc1cc(F)c(C)c3c1c2[C@@H](NC(=O)c1nn[nH]c1CO)CC3] 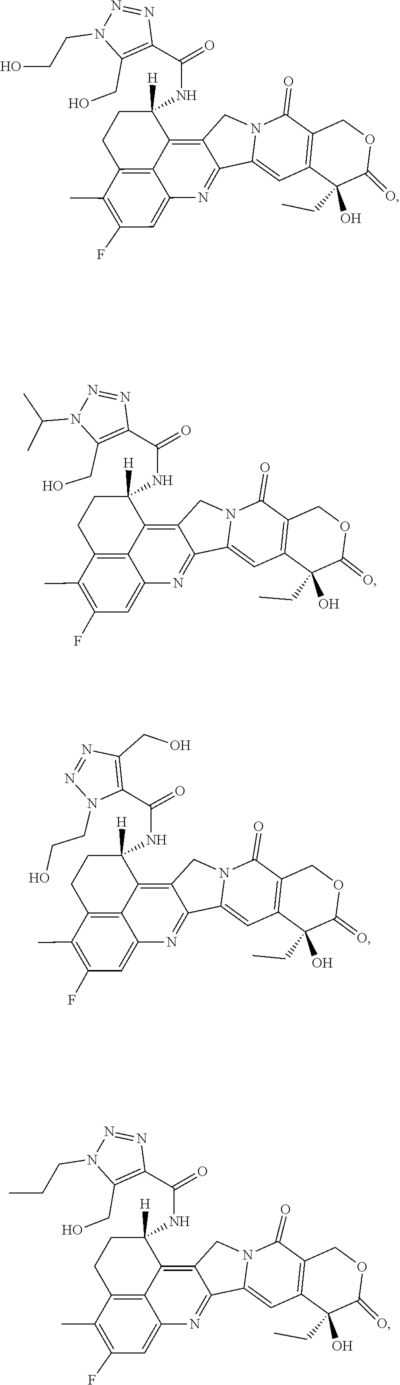 [SMILES: CC[C@@]1(O)C(=O)OCc2c1cc1n(c2=O)Cc2c-1nc1cc(F)c(C)c3c1c2[C@@H](NC(=O)c1nnn(CCO)c1CO)CC3 ; CC[C@@]1(O)C(=O)OCc2c1cc1n(c2=O)Cc2c-1nc1cc(F)c(C)c3c1c2[C@@H](NC(=O)c1nnn(C(C)C)c1CO)CC3 ; CC[C@@]1(O)C(=O)OCc2c1cc1n(c2=O)Cc2c-1nc1cc(F)c(C)c3c1c2[C@@H](NC(=O)c1c(CO)nnn1CCO)CC3 ; CCCn1nnc(C(=O)N[C@H]2CCc3c(C)c(F)cc4nc5c(c2c34)Cn2c-5cc3c(c2=O)COC(=O)[C@]3(O)CC)c1CO] 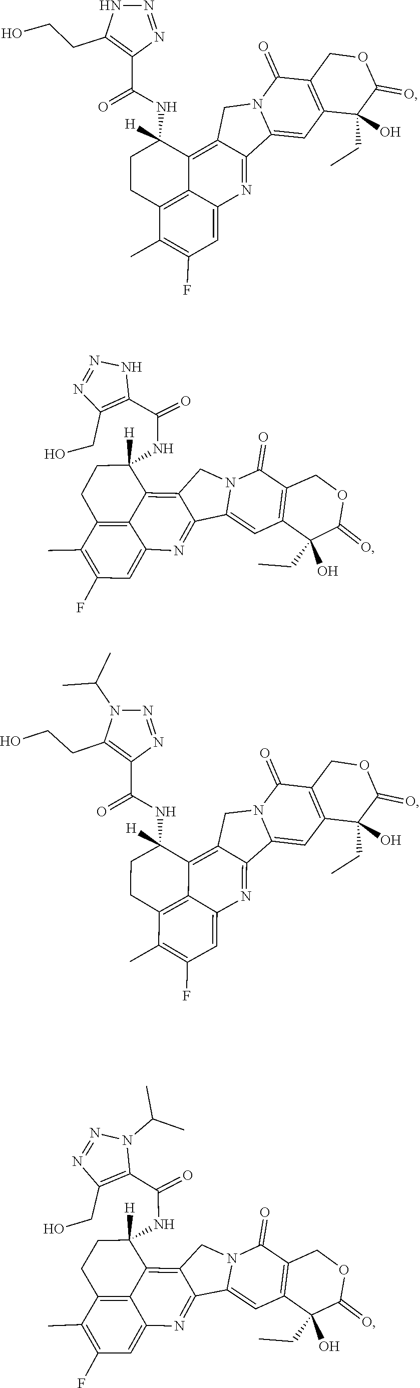 [SMILES: CC[C@@]1(O)C(=O)OCc2c1cc1n(c2=O)Cc2c-1nc1cc(F)c(C)c3c1c2[C@@H](NC(=O)c1nn[nH]c1CCO)CC3 ; CC[C@@]1(O)C(=O)OCc2c1cc1n(c2=O)Cc2c-1nc1cc(F)c(C)c3c1c2[C@@H](NC(=O)c1[nH]nnc1CO)CC3 ; CC[C@@]1(O)C(=O)OCc2c1cc1n(c2=O)Cc2c-1nc1cc(F)c(C)c3c1c2[C@@H](NC(=O)c1nnn(C(C)C)c1CCO)CC3 ; CC[C@@]1(O)C(=O)OCc2c1cc1n(c2=O)Cc2c-1nc1cc(F)c(C)c3c1c2[C@@H](NC(=O)c1c(CO)nnn1C(C)C)CC3] 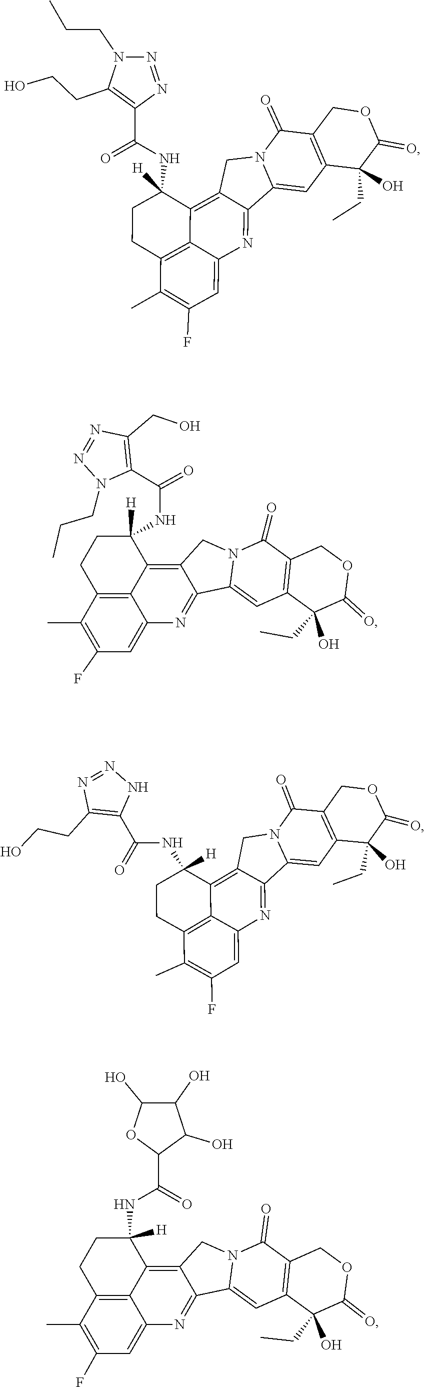 [SMILES: CCCn1nnc(C(=O)N[C@H]2CCc3c(C)c(F)cc4nc5c(c2c34)Cn2c-5cc3c(c2=O)COC(=O)[C@]3(O)CC)c1CCO ; CCCn1nnc(CO)c1C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)c1[nH]nnc1CCO)CC3 ; CC[C@@]1(O)C(=O)OCc2c1cc1n(c2=O)Cc2c-1nc1cc(F)c(C)c3c1c2[C@@H](NC(=O)C1OC(O)C(O)C1O)CC3] 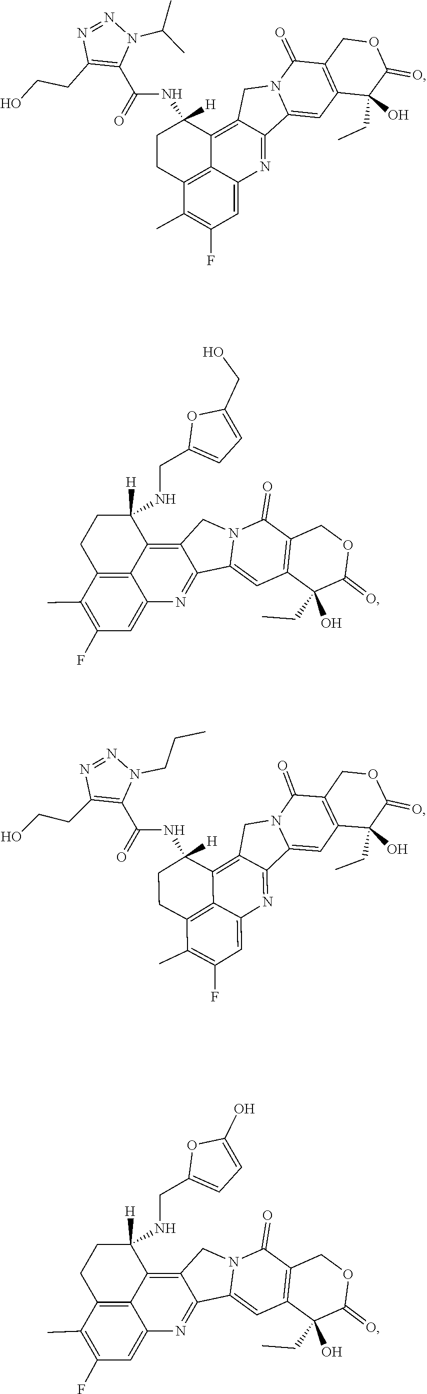 [SMILES: CC[C@@]1(O)C(=O)OCc2c1cc1n(c2=O)Cc2c-1nc1cc(F)c(C)c3c1c2[C@@H](NC(=O)c1c(CCO)nnn1C(C)C)CC3 ; CC[C@@]1(O)C(=O)OCc2c1cc1n(c2=O)Cc2c-1nc1cc(F)c(C)c3c1c2[C@@H](NCc1ccc(CO)o1)CC3 ; CCCn1nnc(CCO)c1C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NCc1ccc(O)o1)CC3] 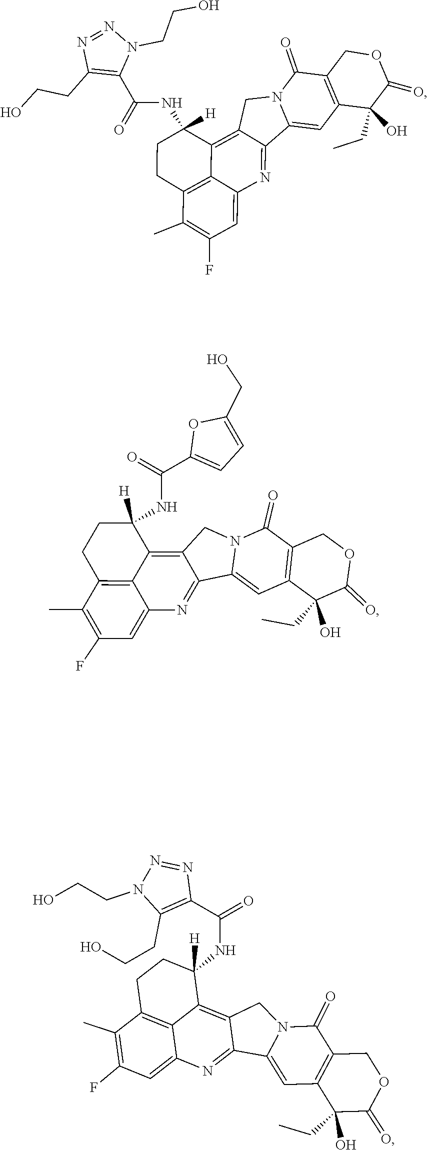 [SMILES: CC[C@@]1(O)C(=O)OCc2c1cc1n(c2=O)Cc2c-1nc1cc(F)c(C)c3c1c2[C@@H](NC(=O)c1c(CCO)nnn1CCO)CC3 ; CC[C@@]1(O)C(=O)OCc2c1cc1n(c2=O)Cc2c-1nc1cc(F)c(C)c3c1c2[C@@H](NC(=O)c1ccc(CO)o1)CC3 ; CC[C@@]1(O)C(=O)OCc2c1cc1n(c2=O)Cc2c-1nc1cc(F)c(C)c3c1c2[C@@H](NC(=O)c1nnn(CCO)c1CCO)CC3] 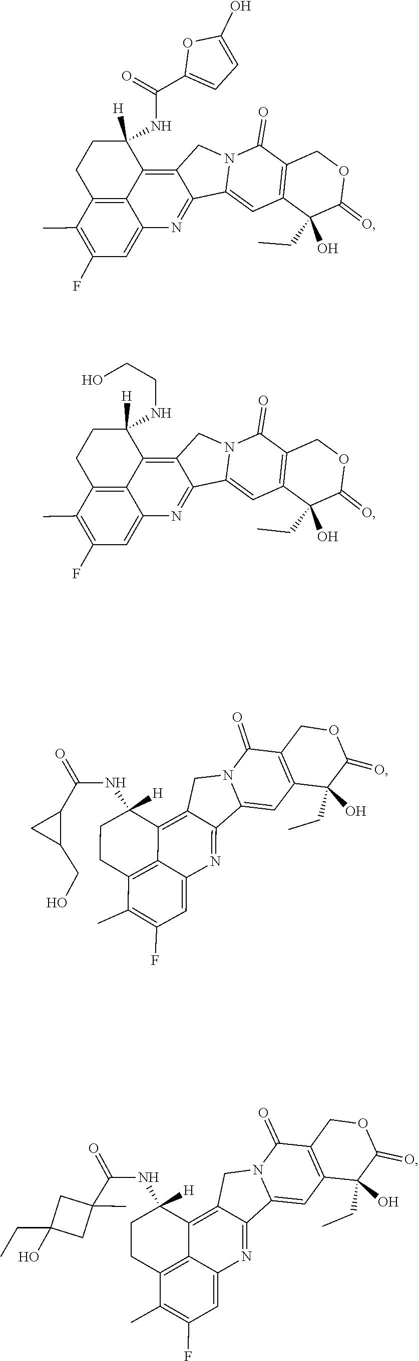 [SMILES: CC[C@@]1(O)C(=O)OCc2c1cc1n(c2=O)Cc2c-1nc1cc(F)c(C)c3c1c2[C@@H](NC(=O)c1ccc(O)o1)CC3 ; CC[C@@]1(O)C(=O)OCc2c1cc1n(c2=O)Cc2c-1nc1cc(F)c(C)c3c1c2[C@@H](NCCO)CC3 ; CC[C@@]1(O)C(=O)OCc2c1cc1n(c2=O)Cc2c-1nc1cc(F)c(C)c3c1c2[C@@H](NC(=O)C1CC1CO)CC3 ; CCC1(O)CC(C)(C(=O)N[C@H]2CCc3c(C)c(F)cc4nc5c(c2c34)Cn2c-5cc3c(c2=O)COC(=O)[C@]3(O)CC)C1] 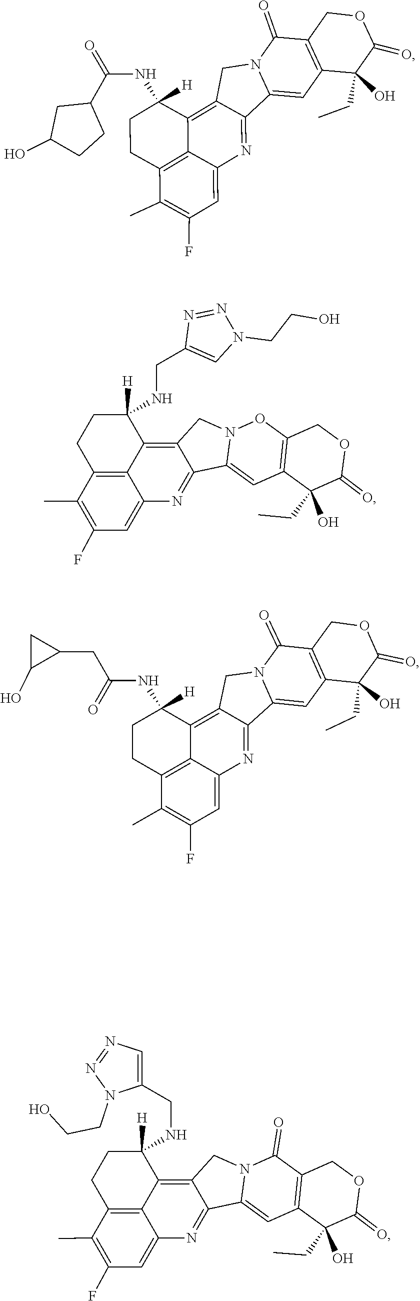 [SMILES: CC[C@@]1(O)C(=O)OCc2c1cc1n(c2=O)Cc2c-1nc1cc(F)c(C)c3c1c2[C@@H](NC(=O)C1CCC(O)C1)CC3 ; CC[C@@]1(O)C(=O)OCC2=C1C=C1c3nc4cc(F)c(C)c5c4c(c3CN1O2)[C@@H](NCc1cn(CCO)nn1)CC5 ; CC[C@@]1(O)C(=O)OCc2c1cc1n(c2=O)Cc2c-1nc1cc(F)c(C)c3c1c2[C@@H](NC(=O)CC1CC1O)CC3 ; CC[C@@]1(O)C(=O)OCc2c1cc1n(c2=O)Cc2c-1nc1cc(F)c(C)c3c1c2[C@@H](NCc1cnnn1CCO)CC3] 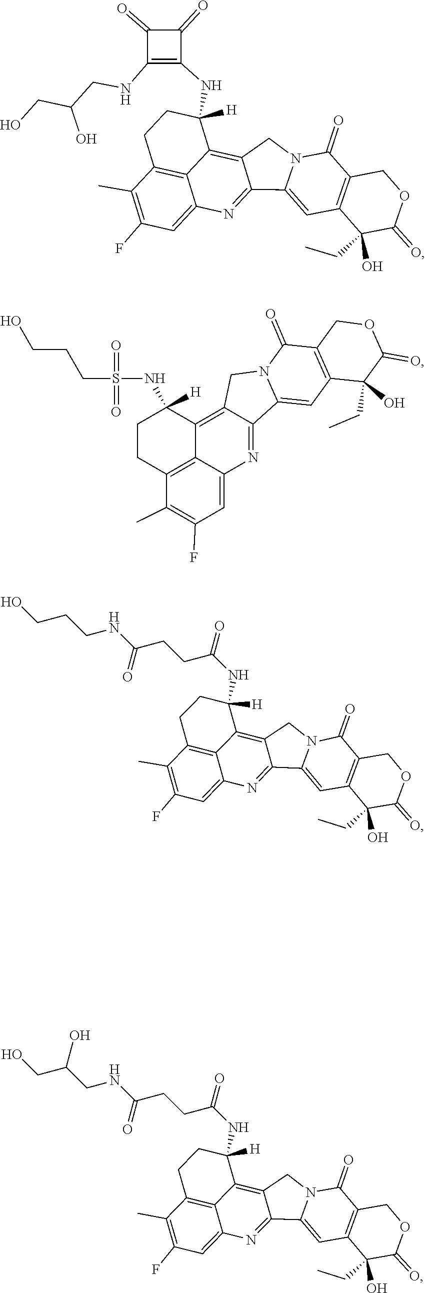 [SMILES: CC[C@@]1(O)C(=O)OCc2c1cc1n(c2=O)Cc2c-1nc1cc(F)c(C)c3c1c2[C@@H](Nc1c(NCC(O)CO)c(=O)c1=O)CC3 ; CC[C@@]1(O)C(=O)OCc2c1cc1n(c2=O)Cc2c-1nc1cc(F)c(C)c3c1c2[C@@H](NS(=O)(=O)CCCO)CC3 ; CC[C@@]1(O)C(=O)OCc2c1cc1n(c2=O)Cc2c-1nc1cc(F)c(C)c3c1c2[C@@H](NC(=O)CCC(=O)NCCCO)CC3 ; CC[C@@]1(O)C(=O)OCc2c1cc1n(c2=O)Cc2c-1nc1cc(F)c(C)c3c1c2[C@@H](NC(=O)CCC(=O)NCC(O)CO)CC3] 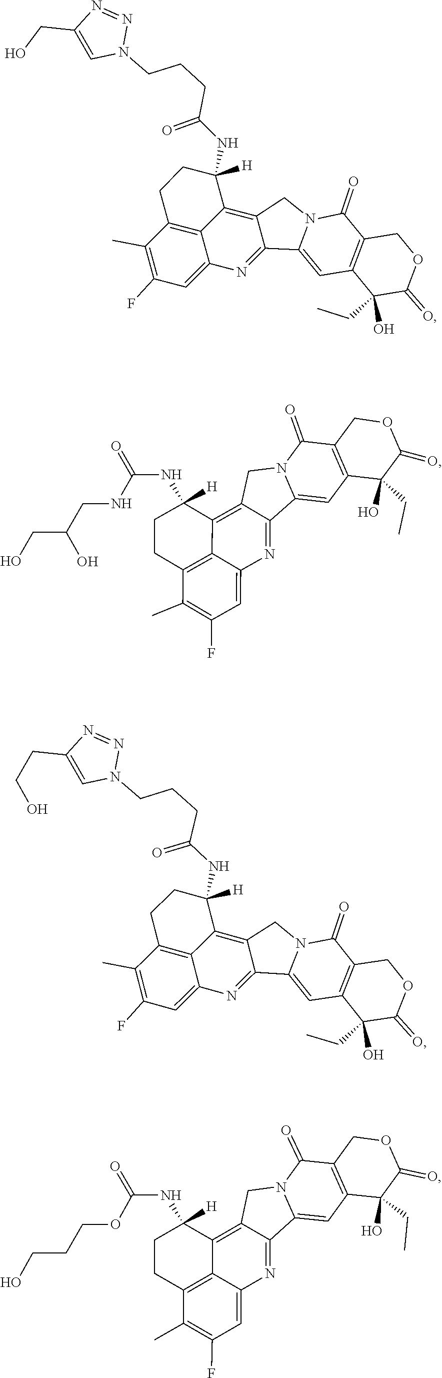 [SMILES: CC[C@@]1(O)C(=O)OCc2c1cc1n(c2=O)Cc2c-1nc1cc(F)c(C)c3c1c2[C@@H](NC(=O)CCCn1cc(CO)nn1)CC3 ; CC[C@@]1(O)C(=O)OCc2c1cc1n(c2=O)Cc2c-1nc1cc(F)c(C)c3c1c2[C@@H](NC(=O)NCC(O)CO)CC3 ; CC[C@@]1(O)C(=O)OCc2c1cc1n(c2=O)Cc2c-1nc1cc(F)c(C)c3c1c2[C@@H](NC(=O)CCCn1cc(CCO)nn1)CC3 ; CC[C@@]1(O)C(=O)OCc2c1cc1n(c2=O)Cc2c-1nc1cc(F)c(C)c3c1c2[C@@H](NC(=O)OCCCO)CC3] 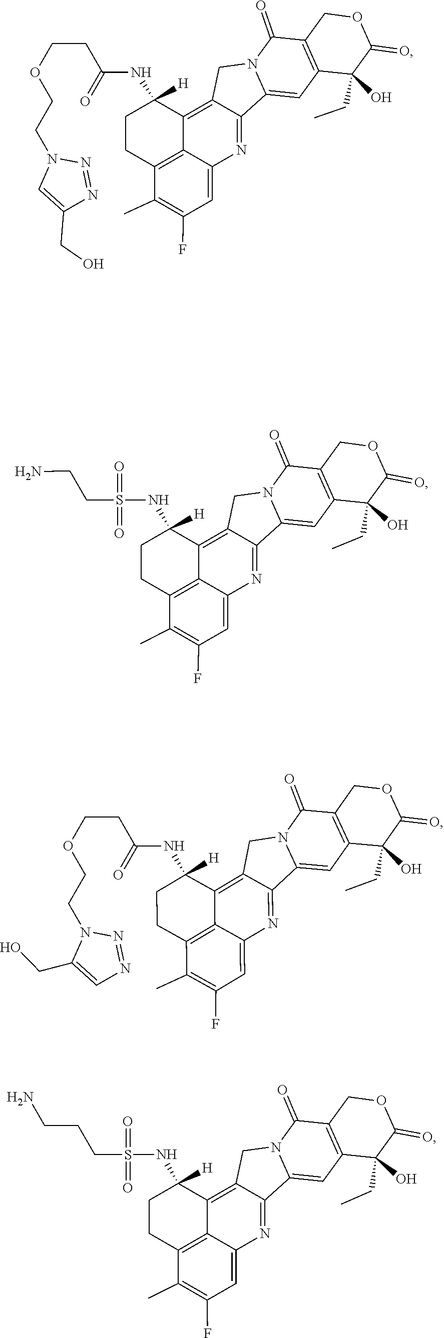 [SMILES: CC[C@@]1(O)C(=O)OCc2c1cc1n(c2=O)Cc2c-1nc1cc(F)c(C)c3c1c2[C@@H](NC(=O)CCOCCn1cc(CO)nn1)CC3 ; CC[C@@]1(O)C(=O)OCc2c1cc1n(c2=O)Cc2c-1nc1cc(F)c(C)c3c1c2[C@@H](NS(=O)(=O)CCN)CC3 ; CC[C@@]1(O)C(=O)OCc2c1cc1n(c2=O)Cc2c-1nc1cc(F)c(C)c3c1c2[C@@H](NC(=O)CCOCCn1nncc1CO)CC3 ; CC[C@@]1(O)C(=O)OCc2c1cc1n(c2=O)Cc2c-1nc1cc(F)c(C)c3c1c2[C@@H](NS(=O)(=O)CCCN)CC3] 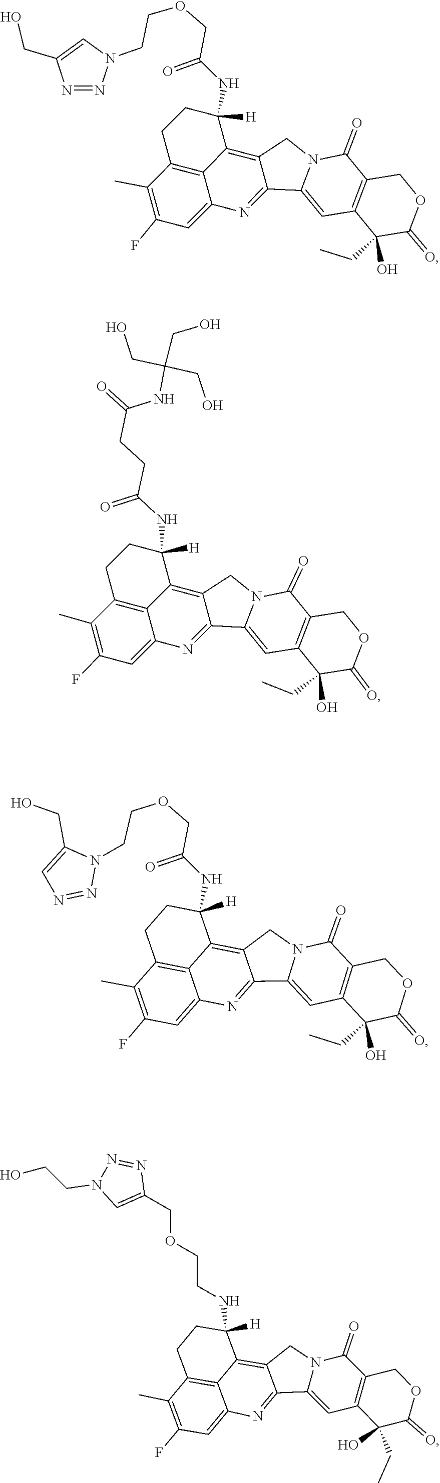 [SMILES: CC[C@@]1(O)C(=O)OCc2c1cc1n(c2=O)Cc2c-1nc1cc(F)c(C)c3c1c2[C@@H](NC(=O)COCCn1cc(CO)nn1)CC3 ; CC[C@@]1(O)C(=O)OCc2c1cc1n(c2=O)Cc2c-1nc1cc(F)c(C)c3c1c2[C@@H](NC(=O)CCC(=O)NC(CO)(CO)CO)CC3 ; CC[C@@]1(O)C(=O)OCc2c1cc1n(c2=O)Cc2c-1nc1cc(F)c(C)c3c1c2[C@@H](NC(=O)COCCn1nncc1CO)CC3 ; CC[C@@]1(O)C(=O)OCc2c1cc1n(c2=O)Cc2c-1nc1cc(F)c(C)c3c1c2[C@@H](NCCOCc1cn(CCO)nn1)CC3] 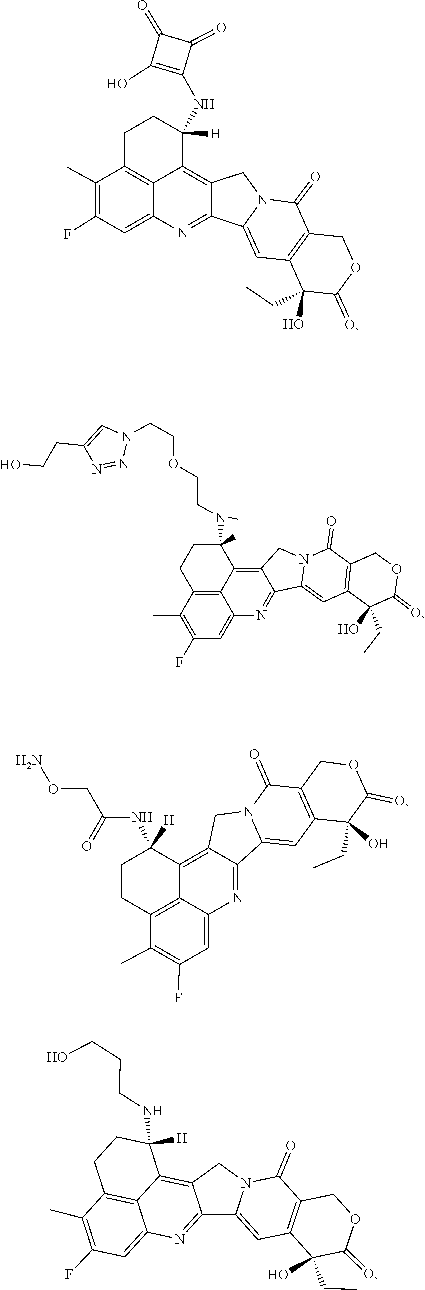 [SMILES: CC[C@@]1(O)C(=O)OCc2c1cc1n(c2=O)Cc2c-1nc1cc(F)c(C)c3c1c2[C@@H](Nc1c(O)c(=O)c1=O)CC3 ; CCN(CCOCCn1cc(CCO)nn1)[C@@]1(C)CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)CON)CC3 ; CC[C@@]1(O)C(=O)OCc2c1cc1n(c2=O)Cc2c-1nc1cc(F)c(C)c3c1c2[C@@H](NCCCO)CC3] 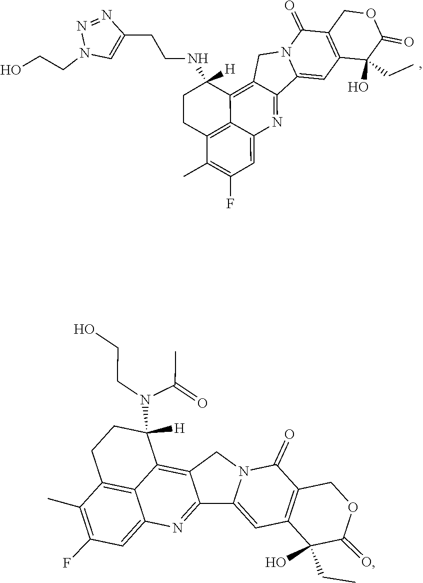 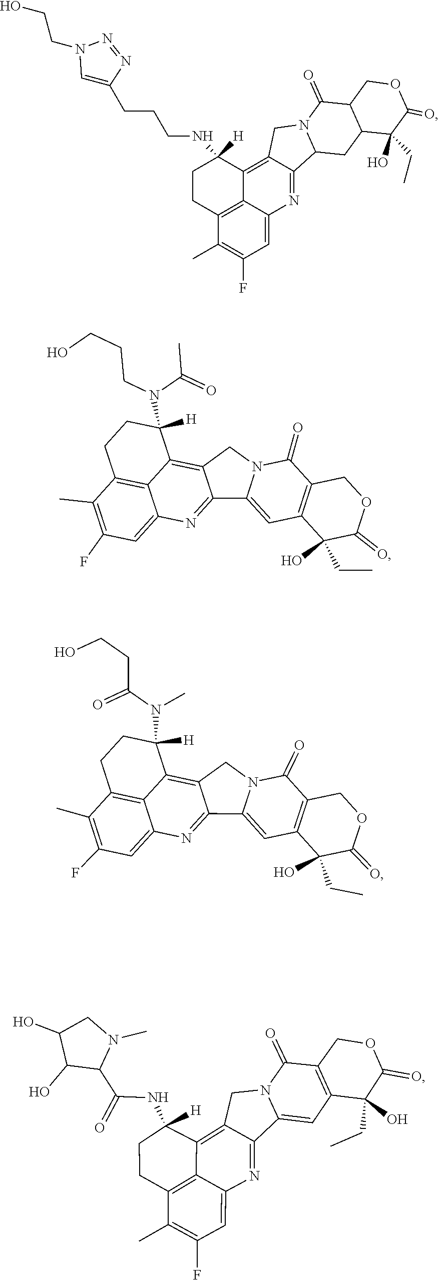 [SMILES: CC[C@@]1(O)C(=O)OCC2C(=O)N3Cc4c(nc5cc(F)c(C)c6c5c4[C@@H](NCCCc4cn(CCO)nn4)CC6)C3CC21 ; CC[C@@]1(O)C(=O)OCc2c1cc1n(c2=O)Cc2c-1nc1cc(F)c(C)c3c1c2[C@@H](N(CCCO)C(C)=O)CC3 ; CC[C@@]1(O)C(=O)OCc2c1cc1n(c2=O)Cc2c-1nc1cc(F)c(C)c3c1c2[C@@H](N(C)C(=O)CCO)CC3 ; CC[C@@]1(O)C(=O)OCc2c1cc1n(c2=O)Cc2c-1nc1cc(F)c(C)c3c1c2[C@@H](NC(=O)C1C(O)C(O)CN1C)CC3] 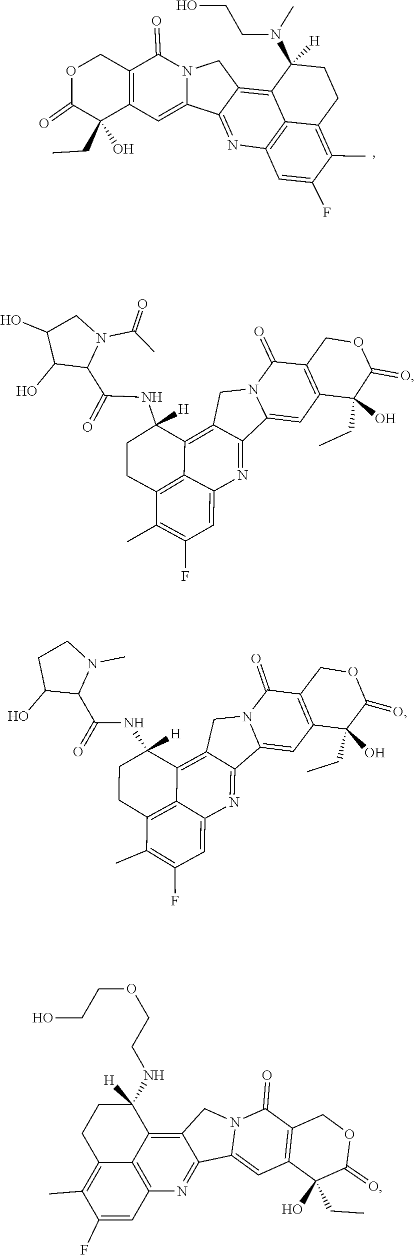 [SMILES: CC[C@@]1(O)C(=O)OCc2c1cc1n(c2=O)Cc2c-1nc1cc(F)c(C)c3c1c2[C@@H](N(C)CCO)CC3 ; CC[C@@]1(O)C(=O)OCc2c1cc1n(c2=O)Cc2c-1nc1cc(F)c(C)c3c1c2[C@@H](NC(=O)C1C(O)C(O)CN1C(C)=O)CC3 ; CC[C@@]1(O)C(=O)OCc2c1cc1n(c2=O)Cc2c-1nc1cc(F)c(C)c3c1c2[C@@H](NC(=O)C1C(O)CCN1C)CC3 ; CC[C@@]1(O)C(=O)OCc2c1cc1n(c2=O)Cc2c-1nc1cc(F)c(C)c3c1c2[C@@H](NCCOCCO)CC3] 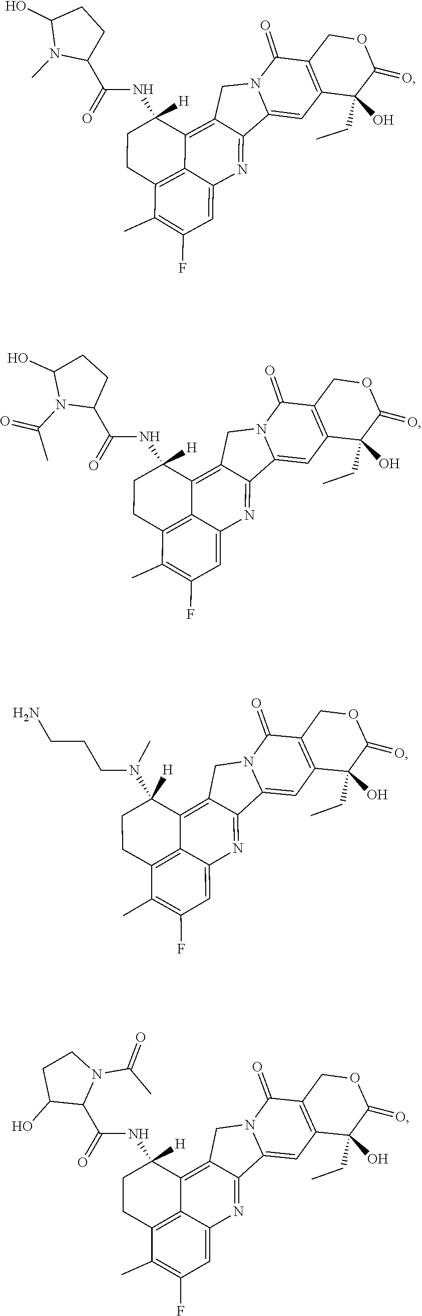 [SMILES: CC[C@@]1(O)C(=O)OCc2c1cc1n(c2=O)Cc2c-1nc1cc(F)c(C)c3c1c2[C@@H](NC(=O)C1CCC(O)N1C)CC3 ; CC[C@@]1(O)C(=O)OCc2c1cc1n(c2=O)Cc2c-1nc1cc(F)c(C)c3c1c2[C@@H](NC(=O)C1CCC(O)N1C(C)=O)CC3 ; CC[C@@]1(O)C(=O)OCc2c1cc1n(c2=O)Cc2c-1nc1cc(F)c(C)c3c1c2[C@@H](N(C)CCCN)CC3 ; CC[C@@]1(O)C(=O)OCc2c1cc1n(c2=O)Cc2c-1nc1cc(F)c(C)c3c1c2[C@@H](NC(=O)C1C(O)CCN1C(C)=O)CC3] 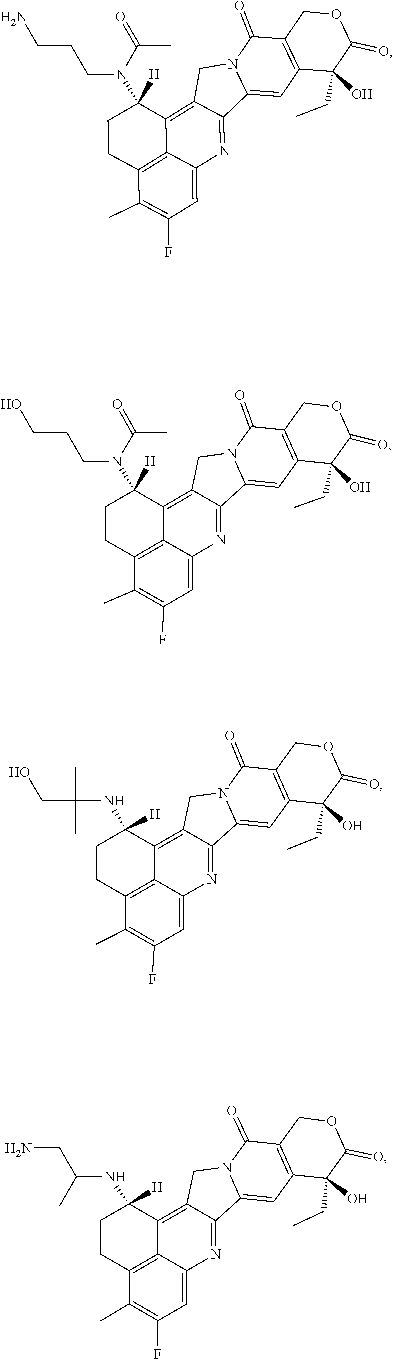 [SMILES: CC[C@@]1(O)C(=O)OCc2c1cc1n(c2=O)Cc2c-1nc1cc(F)c(C)c3c1c2[C@@H](N(CCCN)C(C)=O)CC3 ; CC[C@@]1(O)C(=O)OCc2c1cc1n(c2=O)Cc2c-1nc1cc(F)c(C)c3c1c2[C@@H](N(CCCO)C(C)=O)CC3 ; CC[C@@]1(O)C(=O)OCc2c1cc1n(c2=O)Cc2c-1nc1cc(F)c(C)c3c1c2[C@@H](NC(C)(C)CO)CC3 ; CC[C@@]1(O)C(=O)OCc2c1cc1n(c2=O)Cc2c-1nc1cc(F)c(C)c3c1c2[C@@H](NC(C)CN)CC3] 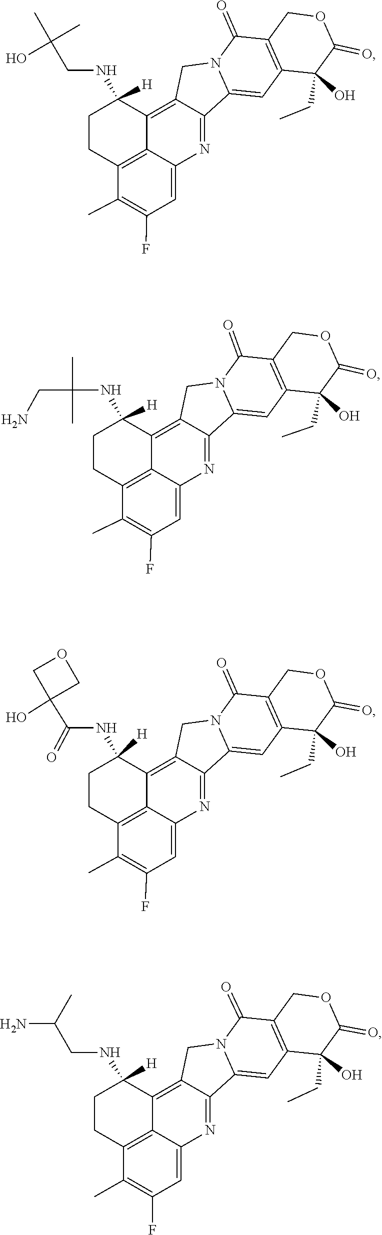 [SMILES: CC[C@@]1(O)C(=O)OCc2c1cc1n(c2=O)Cc2c-1nc1cc(F)c(C)c3c1c2[C@@H](NCC(C)(C)O)CC3 ; CC[C@@]1(O)C(=O)OCc2c1cc1n(c2=O)Cc2c-1nc1cc(F)c(C)c3c1c2[C@@H](NC(C)(C)CN)CC3 ; CC[C@@]1(O)C(=O)OCc2c1cc1n(c2=O)Cc2c-1nc1cc(F)c(C)c3c1c2[C@@H](NC(=O)C1(O)COC1)CC3 ; CC[C@@]1(O)C(=O)OCc2c1cc1n(c2=O)Cc2c-1nc1cc(F)c(C)c3c1c2[C@@H](NCC(C)N)CC3] 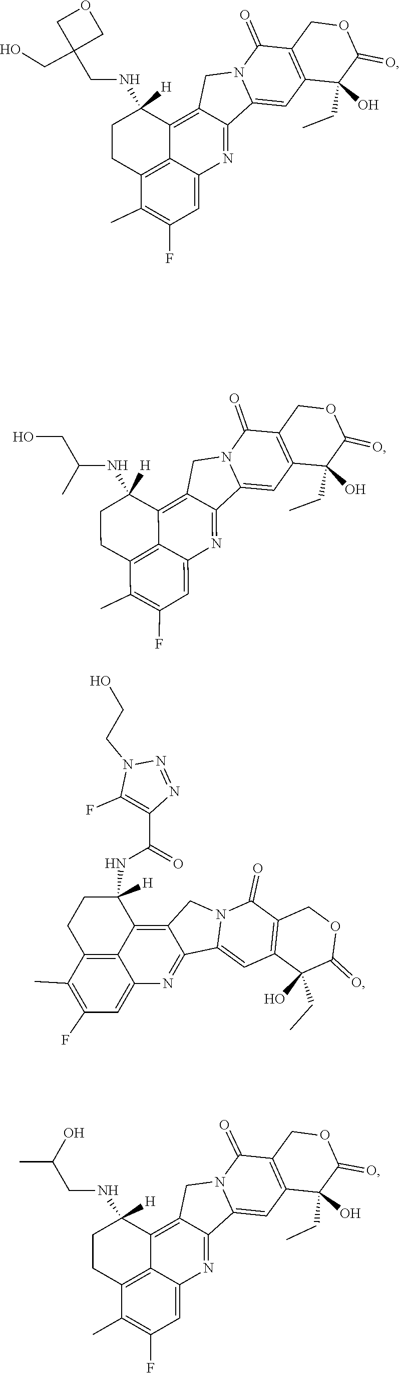 [SMILES: CC[C@@]1(O)C(=O)OCc2c1cc1n(c2=O)Cc2c-1nc1cc(F)c(C)c3c1c2[C@@H](NCC1(CO)COC1)CC3 ; CC[C@@]1(O)C(=O)OCc2c1cc1n(c2=O)Cc2c-1nc1cc(F)c(C)c3c1c2[C@@H](NC(C)CO)CC3 ; CC[C@@]1(O)C(=O)OCc2c1cc1n(c2=O)Cc2c-1nc1cc(F)c(C)c3c1c2[C@@H](NC(=O)c1nnn(CCO)c1F)CC3 ; CC[C@@]1(O)C(=O)OCc2c1cc1n(c2=O)Cc2c-1nc1cc(F)c(C)c3c1c2[C@@H](NCC(C)O)CC3] 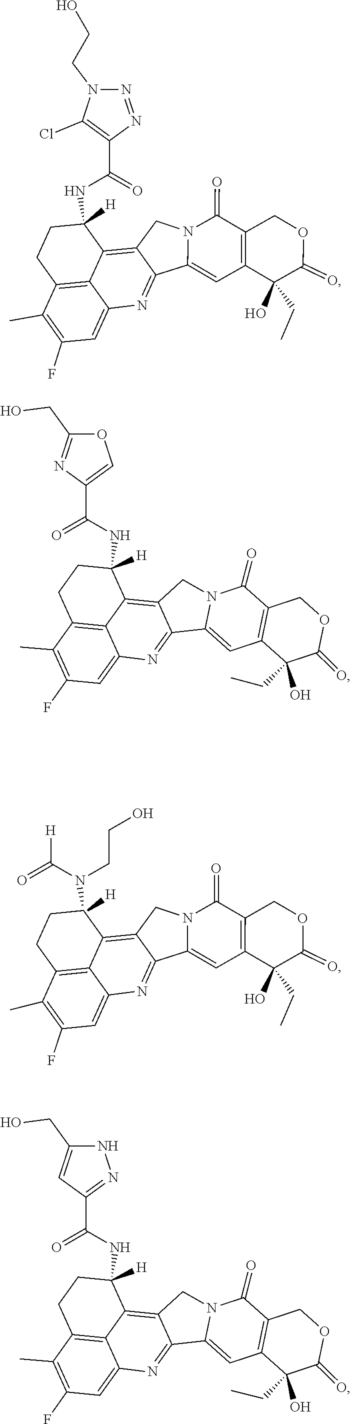 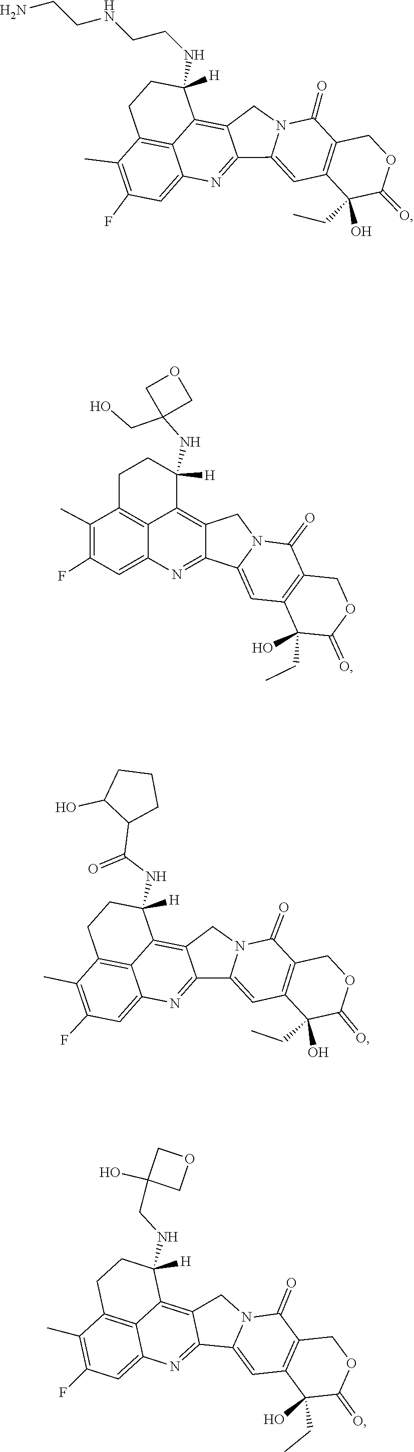 [SMILES: CC[C@@]1(O)C(=O)OCc2c1cc1n(c2=O)Cc2c-1nc1cc(F)c(C)c3c1c2[C@@H](NCCNCCN)CC3 ; CC[C@@]1(O)C(=O)OCc2c1cc1n(c2=O)Cc2c-1nc1cc(F)c(C)c3c1c2[C@@H](NC1(CO)COC1)CC3 ; CC[C@@]1(O)C(=O)OCc2c1cc1n(c2=O)Cc2c-1nc1cc(F)c(C)c3c1c2[C@@H](NC(=O)C1CCCC1O)CC3 ; CC[C@@]1(O)C(=O)OCc2c1cc1n(c2=O)Cc2c-1nc1cc(F)c(C)c3c1c2[C@@H](NCC1(O)COC1)CC3] 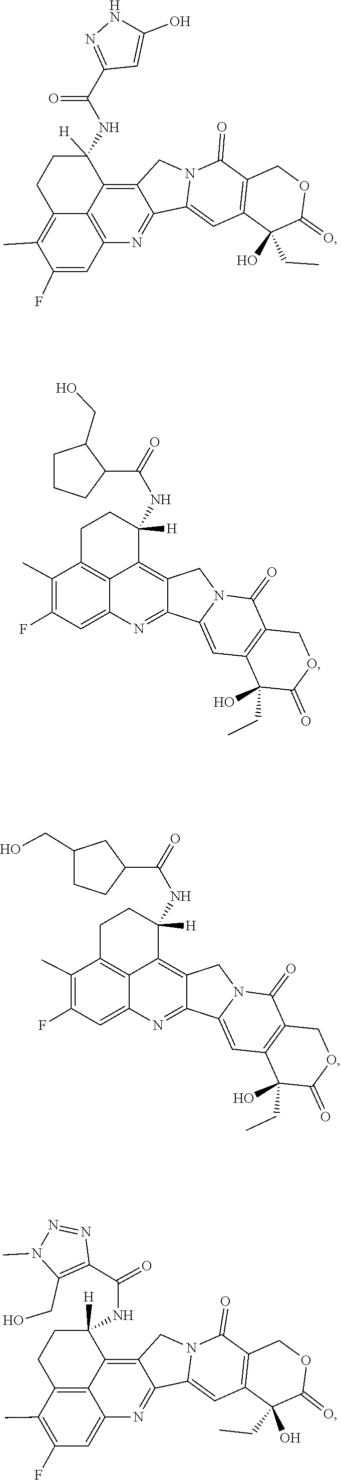 [SMILES: CC[C@@]1(O)C(=O)OCc2c1cc1n(c2=O)Cc2c-1nc1cc(F)c(C)c3c1c2[C@@H](NC(=O)c1cc(O)[nH]n1)CC3 ; CC[C@@]1(O)C(=O)OCc2c1cc1n(c2=O)Cc2c-1nc1cc(F)c(C)c3c1c2[C@@H](NC(=O)C1CCCC1CO)CC3 ; CC[C@@]1(O)C(=O)OCc2c1cc1n(c2=O)Cc2c-1nc1cc(F)c(C)c3c1c2[C@@H](NC(=O)C1CCC(CO)C1)CC3 ; CC[C@@]1(O)C(=O)OCc2c1cc1n(c2=O)Cc2c-1nc1cc(F)c(C)c3c1c2[C@@H](NC(=O)c1nnn(C)c1CO)CC3] 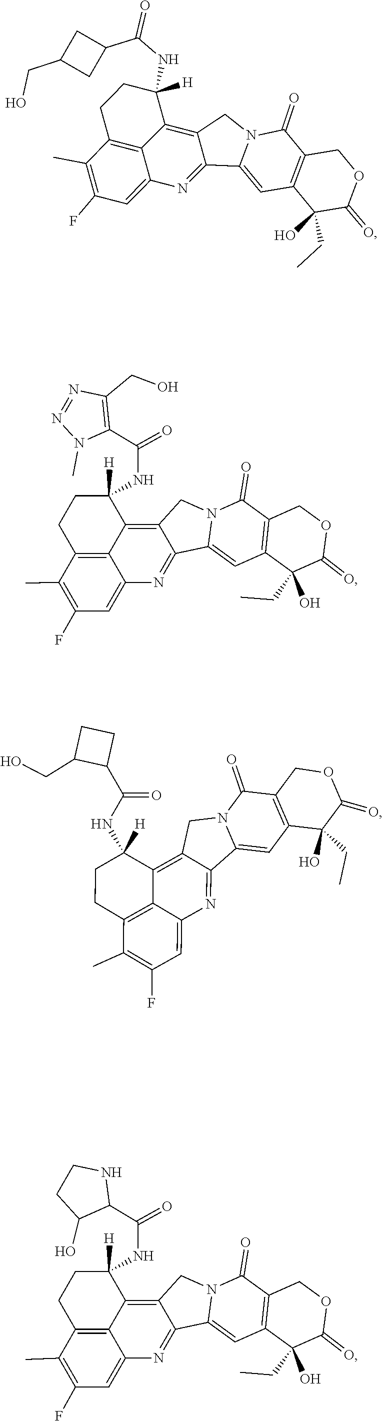 [SMILES: CC[C@@]1(O)C(=O)OCc2c1cc1n(c2=O)Cc2c-1nc1cc(F)c(C)c3c1c2[C@@H](NC(=O)C1CC(CO)C1)CC3 ; CC[C@@]1(O)C(=O)OCc2c1cc1n(c2=O)Cc2c-1nc1cc(F)c(C)c3c1c2[C@@H](NC(=O)c1c(CO)nnn1C)CC3 ; CC[C@@]1(O)C(=O)OCc2c1cc1n(c2=O)Cc2c-1nc1cc(F)c(C)c3c1c2[C@@H](NC(=O)C1CCC1CO)CC3 ; CC[C@@]1(O)C(=O)OCc2c1cc1n(c2=O)Cc2c-1nc1cc(F)c(C)c3c1c2[C@@H](NC(=O)C1NCCC1O)CC3] 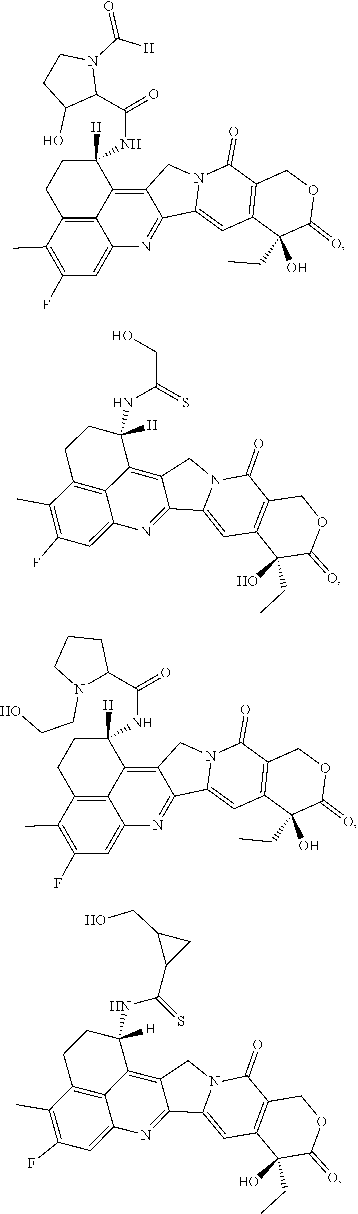 [SMILES: CC[C@@]1(O)C(=O)OCc2c1cc1n(c2=O)Cc2c-1nc1cc(F)c(C)c3c1c2[C@@H](NC(=O)C1C(O)CCN1C=O)CC3 ; CC[C@@]1(O)C(=O)OCc2c1cc1n(c2=O)Cc2c-1nc1cc(F)c(C)c3c1c2[C@@H](NC(=S)CO)CC3 ; CC[C@@]1(O)C(=O)OCc2c1cc1n(c2=O)Cc2c-1nc1cc(F)c(C)c3c1c2[C@@H](NC(=O)C1CCCN1CCO)CC3 ; CC[C@@]1(O)C(=O)OCc2c1cc1n(c2=O)Cc2c-1nc1cc(F)c(C)c3c1c2[C@@H](NC(=S)C1CC1CO)CC3] 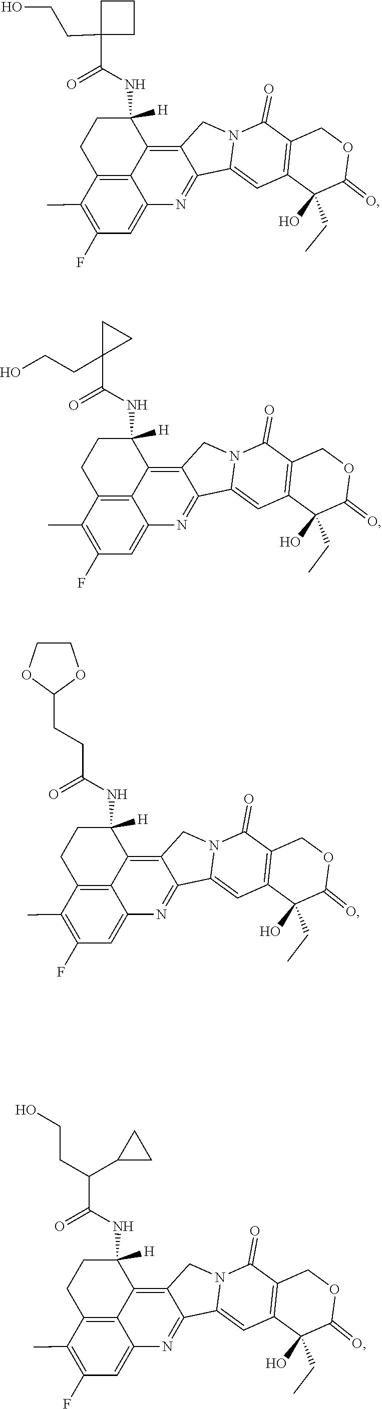 [SMILES: CC[C@@]1(O)C(=O)OCc2c1cc1n(c2=O)Cc2c-1nc1cc(F)c(C)c3c1c2[C@@H](NC(=O)C1(CCO)CCC1)CC3 ; CC[C@@]1(O)C(=O)OCc2c1cc1n(c2=O)Cc2c-1nc1cc(F)c(C)c3c1c2[C@@H](NC(=O)C1(CCO)CC1)CC3 ; CC[C@@]1(O)C(=O)OCc2c1cc1n(c2=O)Cc2c-1nc1cc(F)c(C)c3c1c2[C@@H](NC(=O)CCC1OCCO1)CC3 ; CC[C@@]1(O)C(=O)OCc2c1cc1n(c2=O)Cc2c-1nc1cc(F)c(C)c3c1c2[C@@H](NC(=O)C(CCO)C1CC1)CC3] 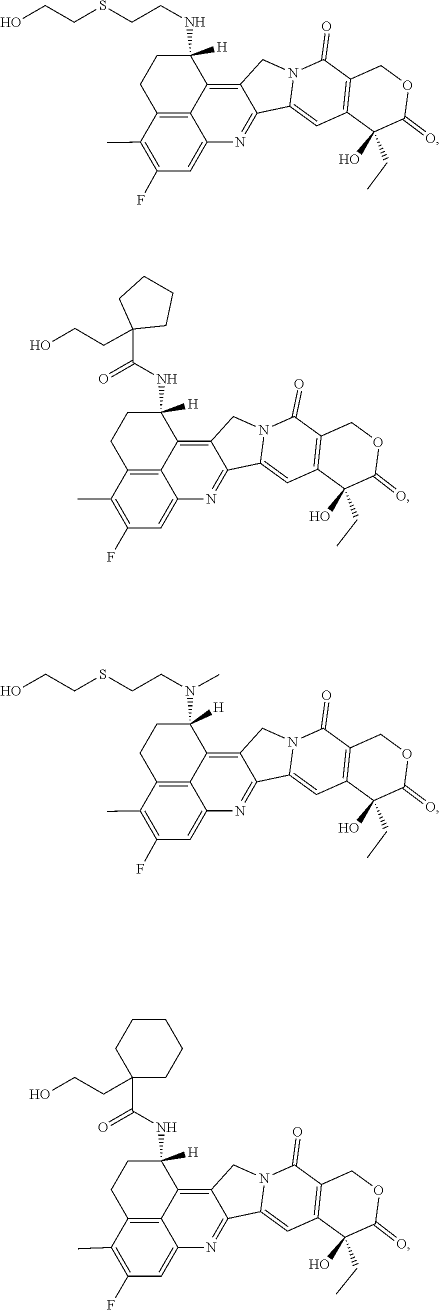 [SMILES: CC[C@@]1(O)C(=O)OCc2c1cc1n(c2=O)Cc2c-1nc1cc(F)c(C)c3c1c2[C@@H](NCCSCCO)CC3 ; CC[C@@]1(O)C(=O)OCc2c1cc1n(c2=O)Cc2c-1nc1cc(F)c(C)c3c1c2[C@@H](NC(=O)C1(CCO)CCCC1)CC3 ; CC[C@@]1(O)C(=O)OCc2c1cc1n(c2=O)Cc2c-1nc1cc(F)c(C)c3c1c2[C@@H](N(C)CCSCCO)CC3 ; CC[C@@]1(O)C(=O)OCc2c1cc1n(c2=O)Cc2c-1nc1cc(F)c(C)c3c1c2[C@@H](NC(=O)C1(CCO)CCCCC1)CC3] 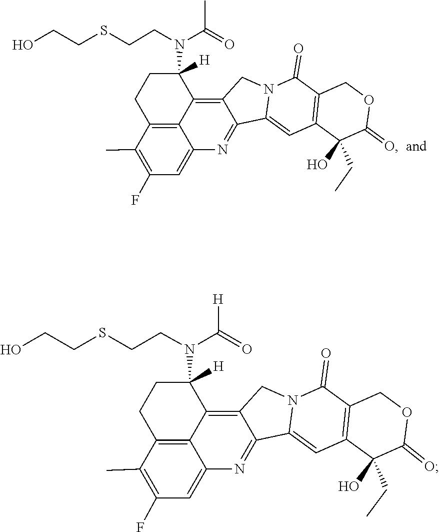
or a pharmaceutically acceptable salt or stereoisomer of any of the foregoing.

36.-60. (canceled)

61. A compound represented by Formula IIA or Formula IIB: 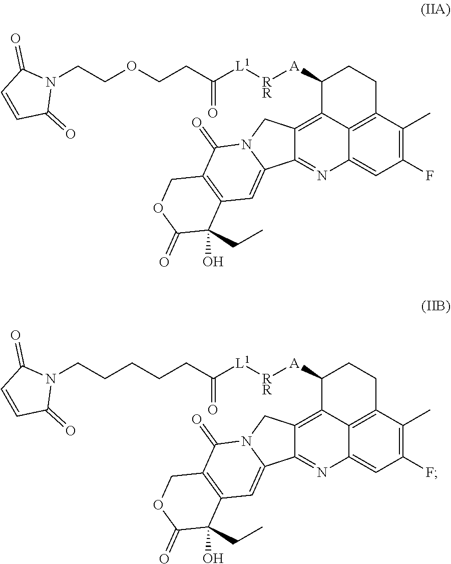
or a pharmaceutically acceptable salt or stereoisomer thereof, wherein:
A is NH or triazolyl;
L¹ is selected from the group consisting of 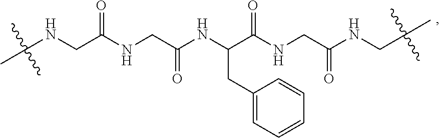 [SMILES: *CNC(=O)CNC(=O)C(Cc1ccccc1)NC(=O)CNC(=O)CN*] 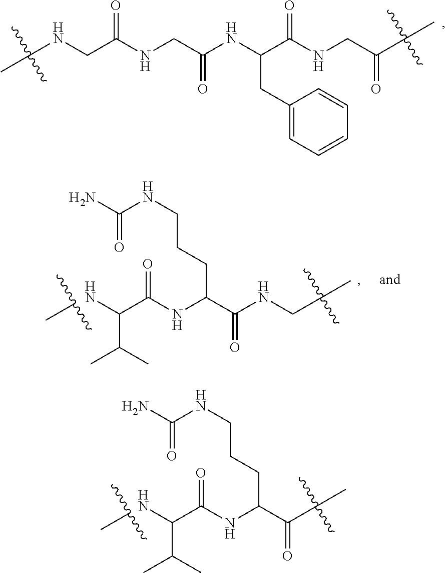
and
RR is an alkoxy or amino moiety formed from L¹ and a hydroxy or —NH₂ moiety of R of claim 1.

62. (canceled)

63. The compound of claim 61, wherein the compound is selected from the group consisting of:

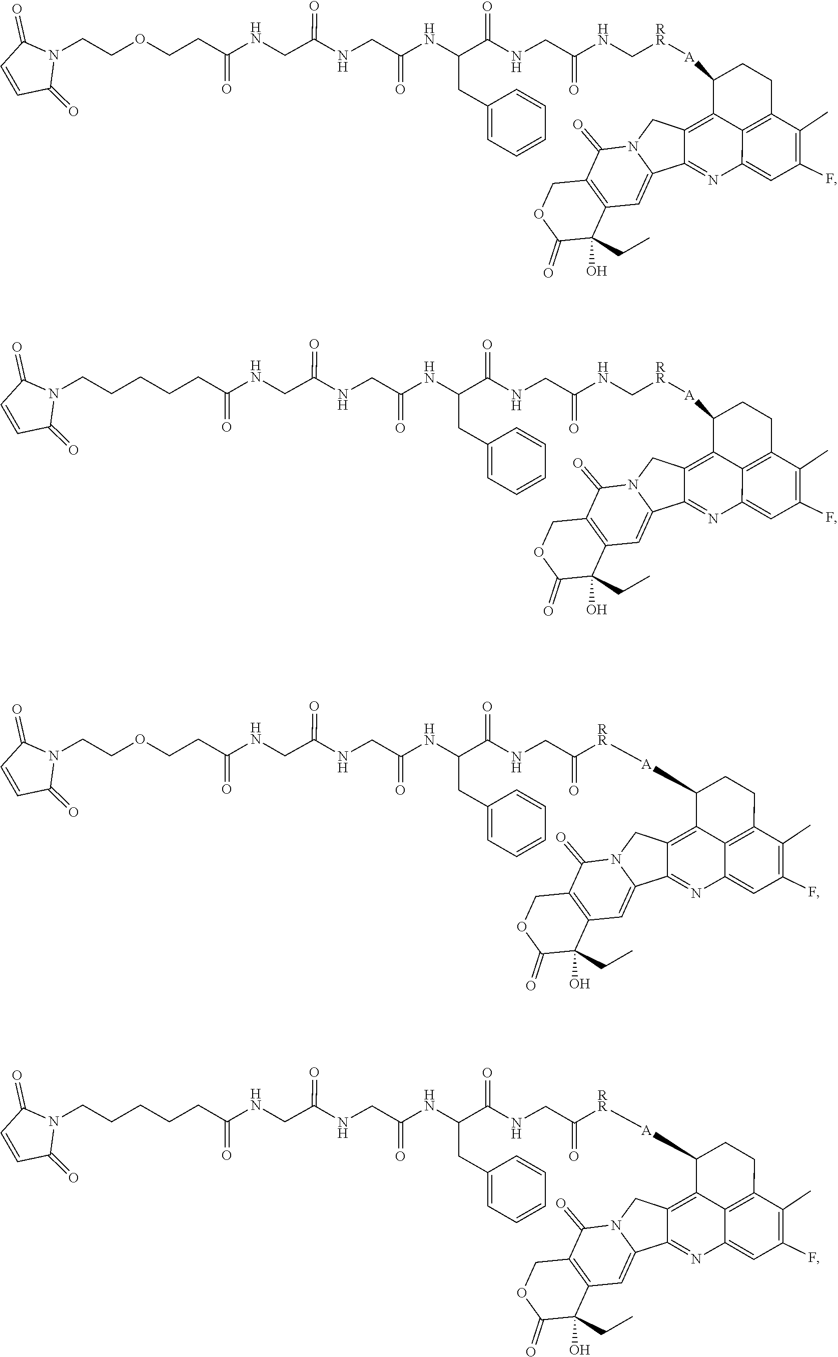

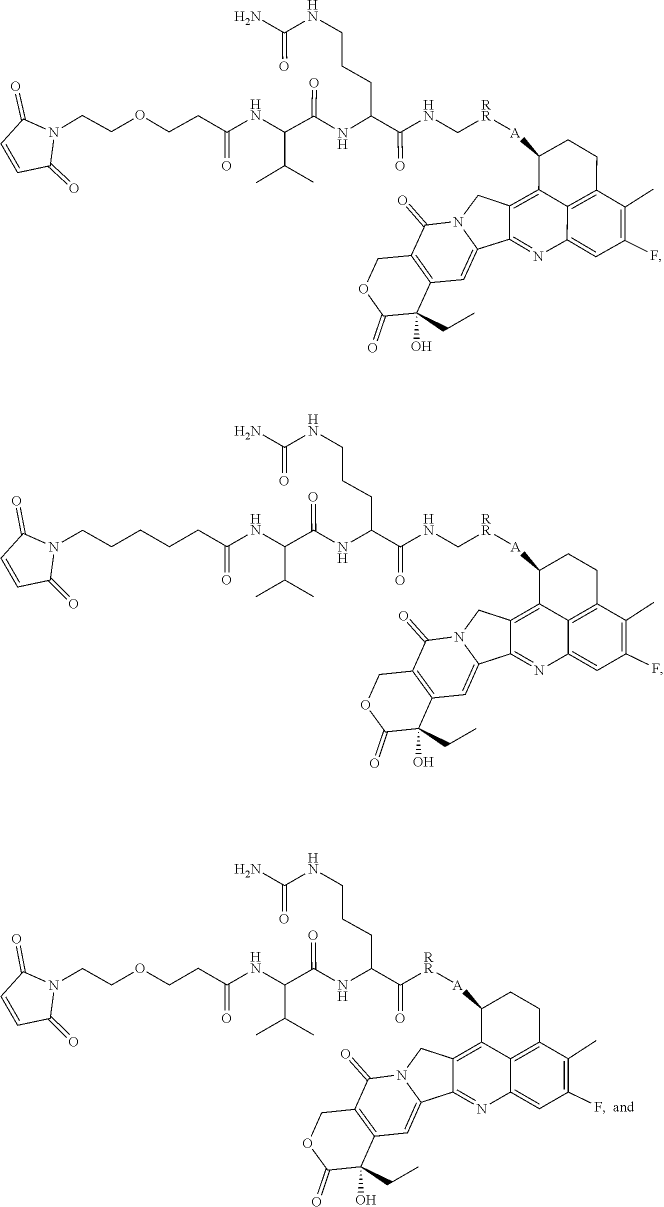

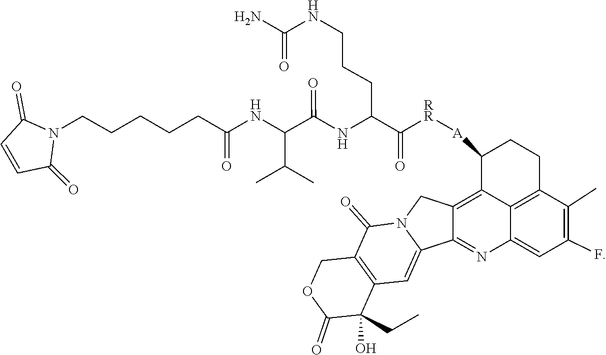

64. A compound selected from the group consisting of: 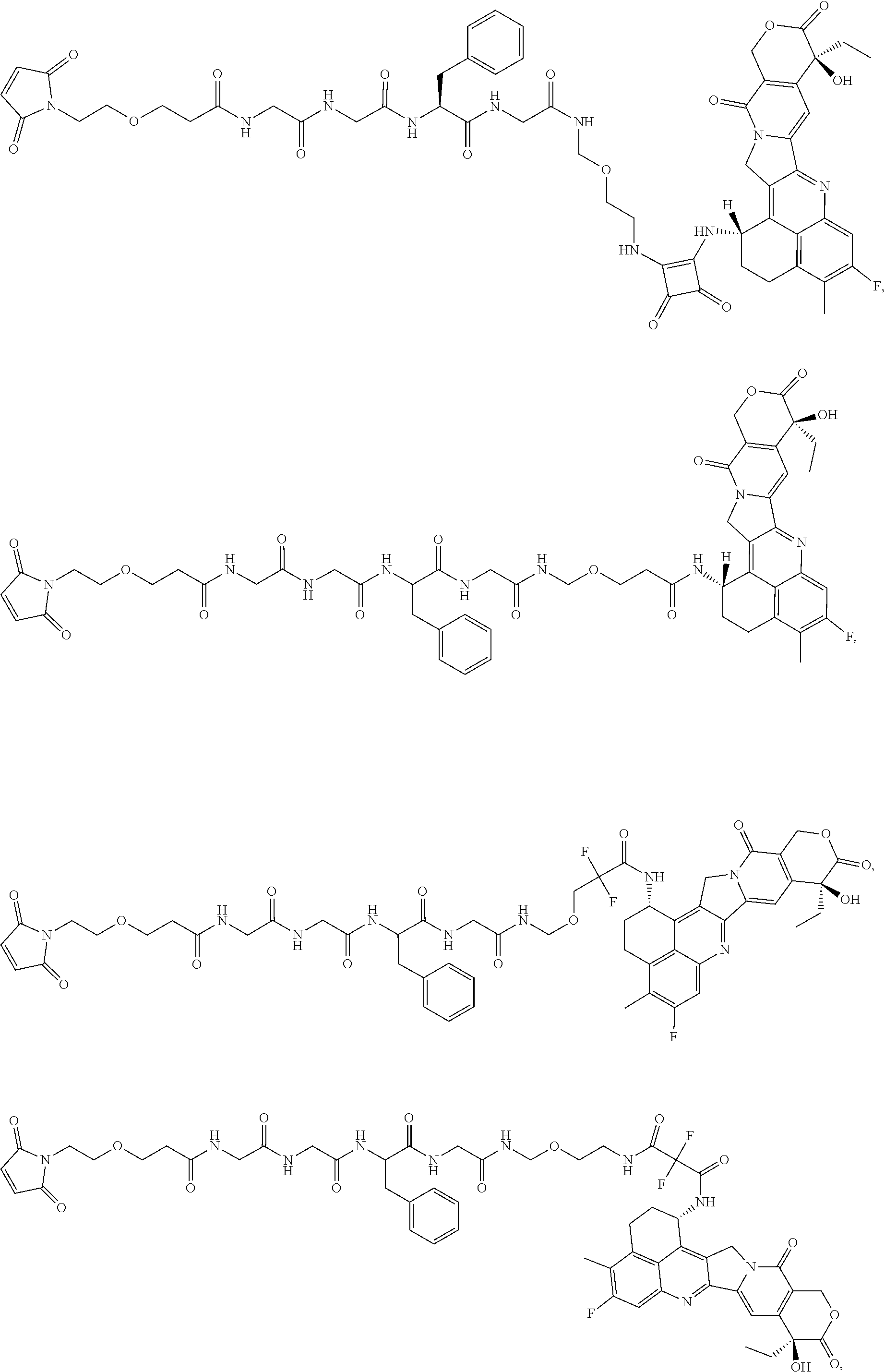 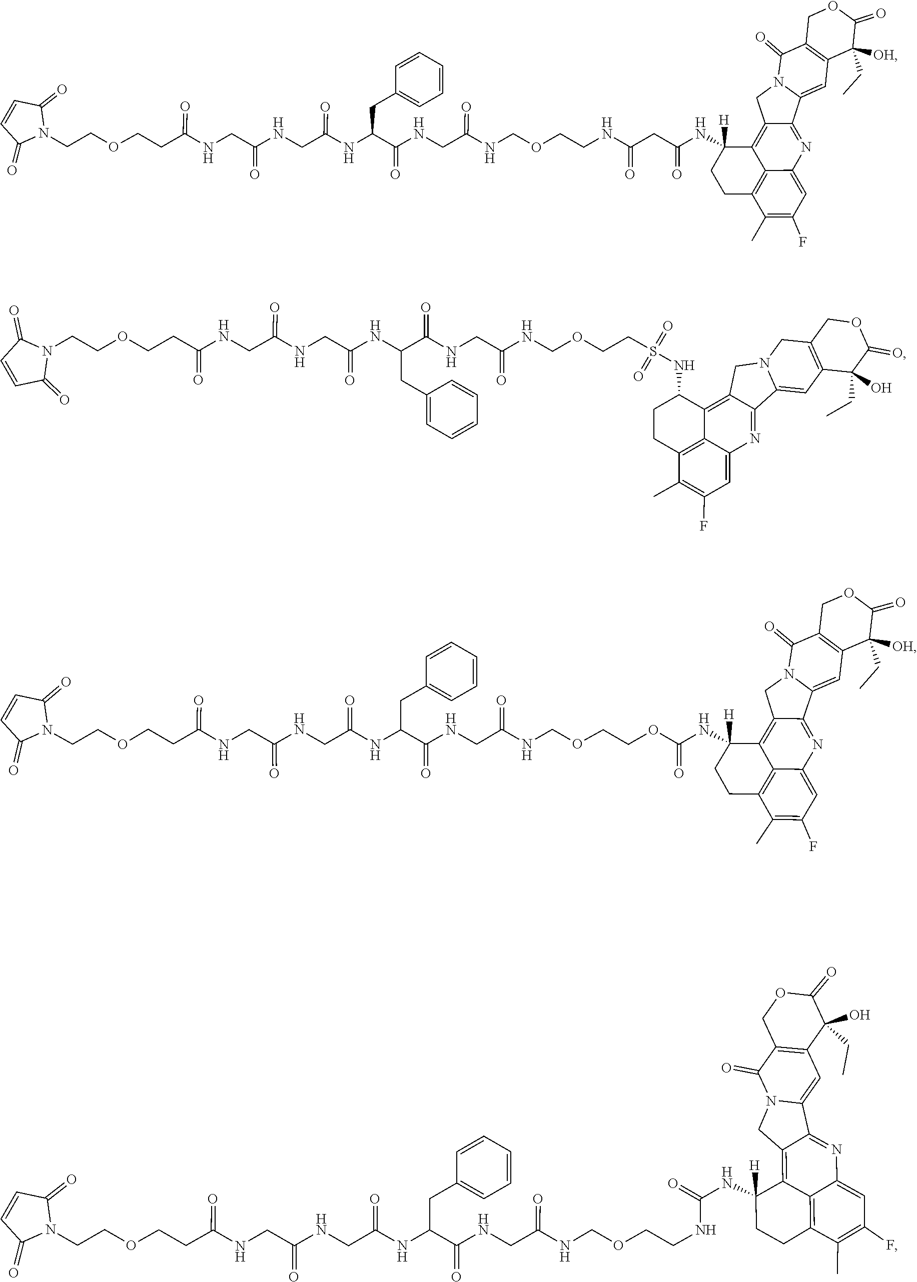 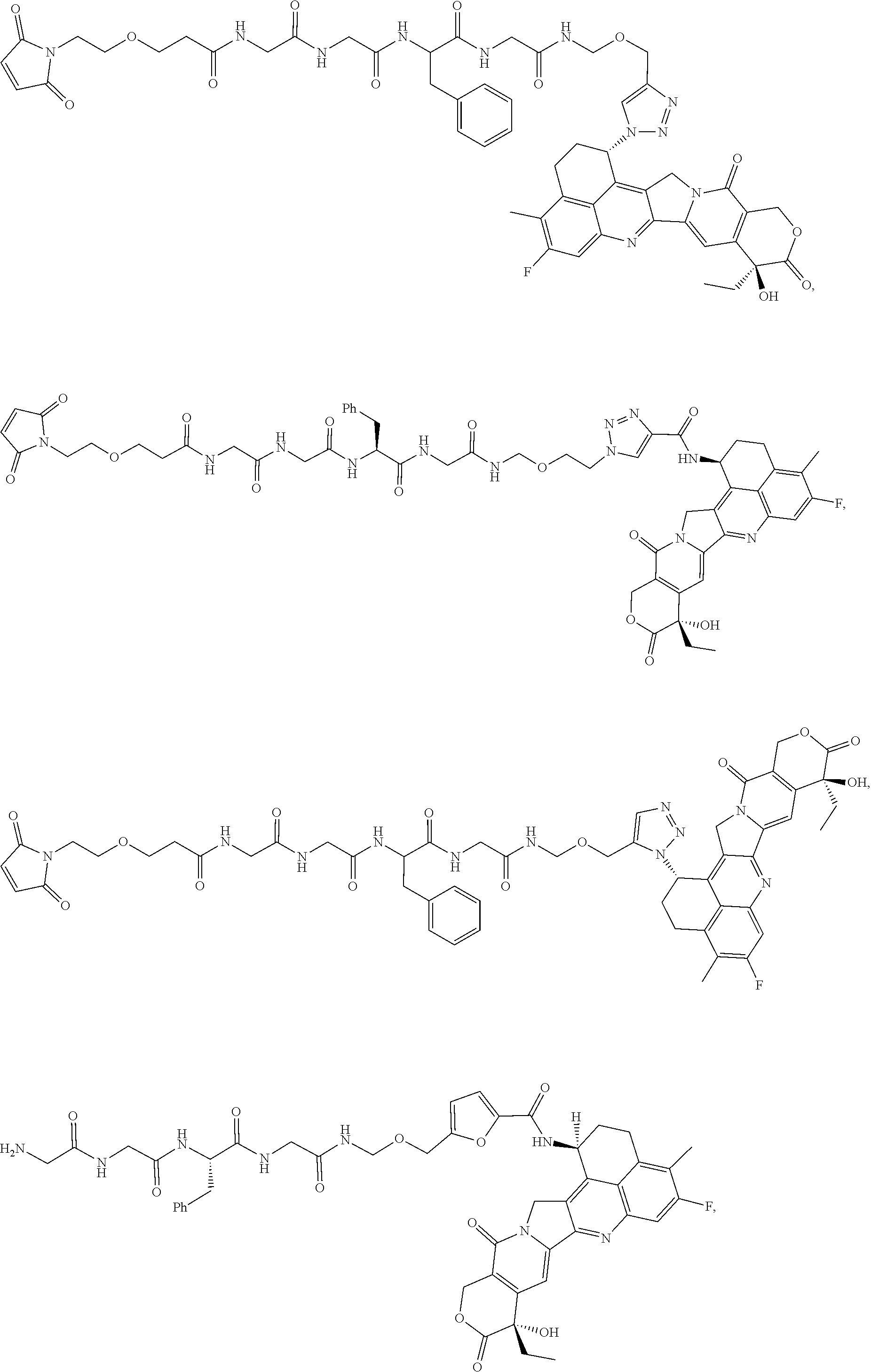 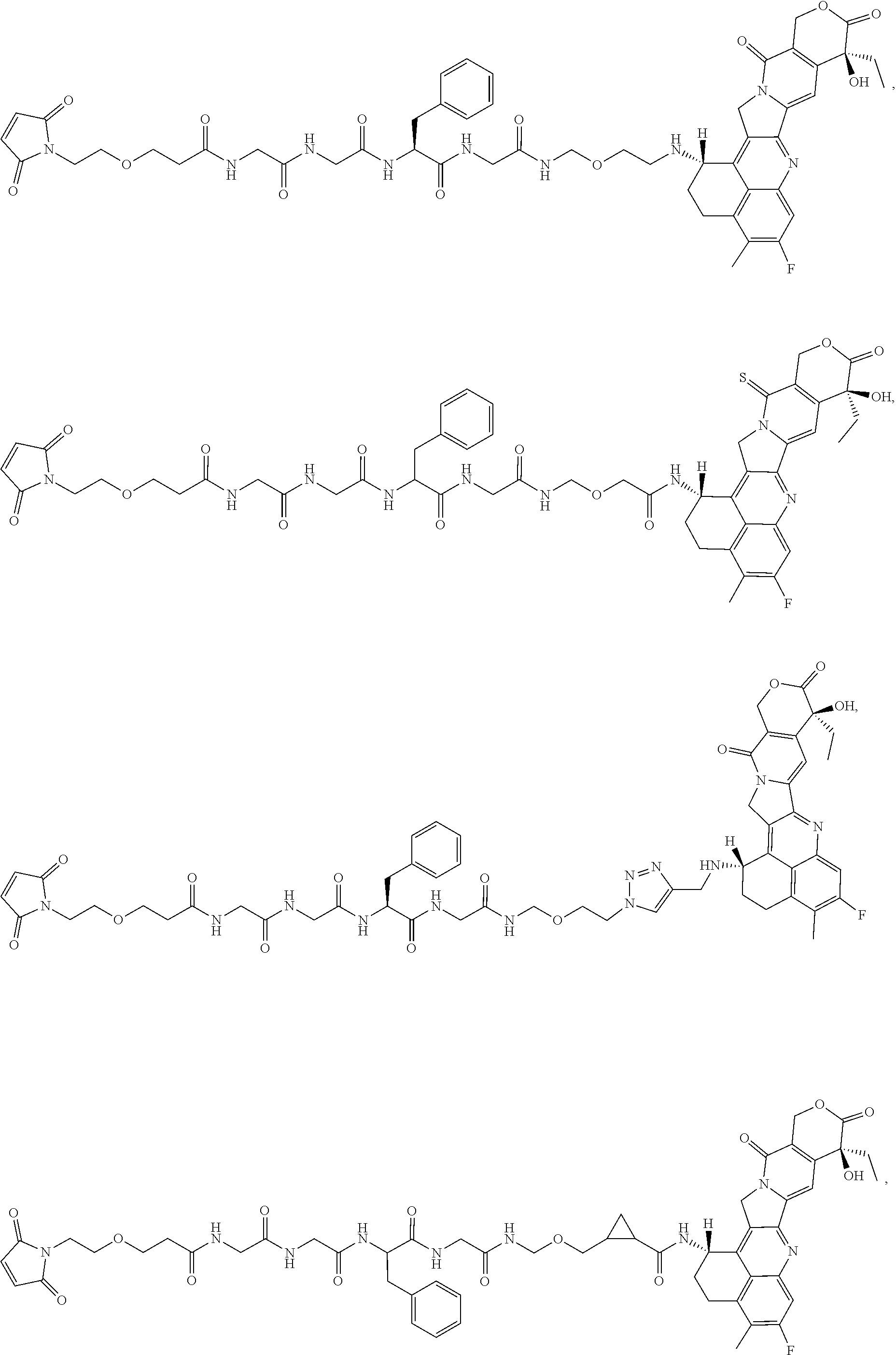 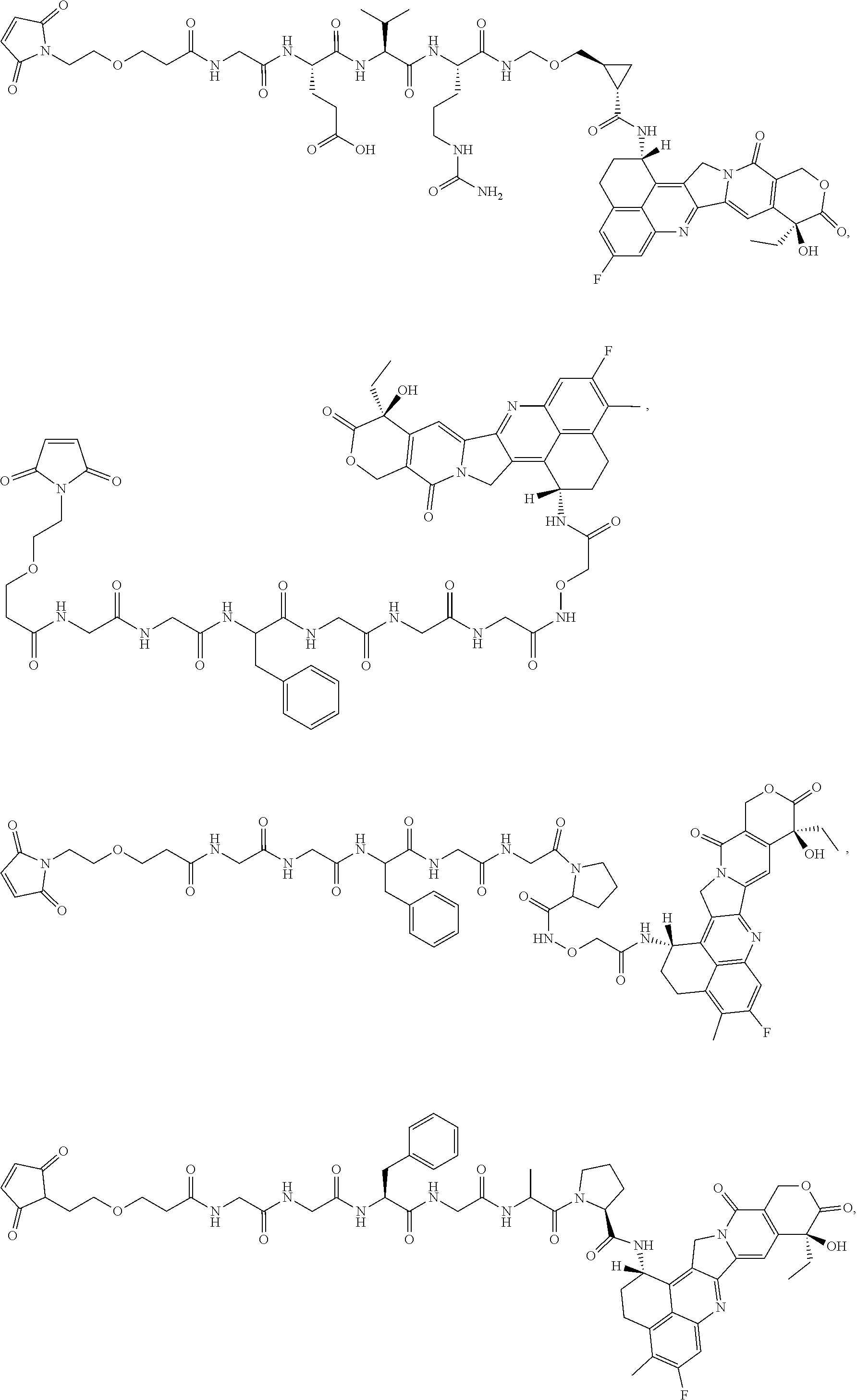 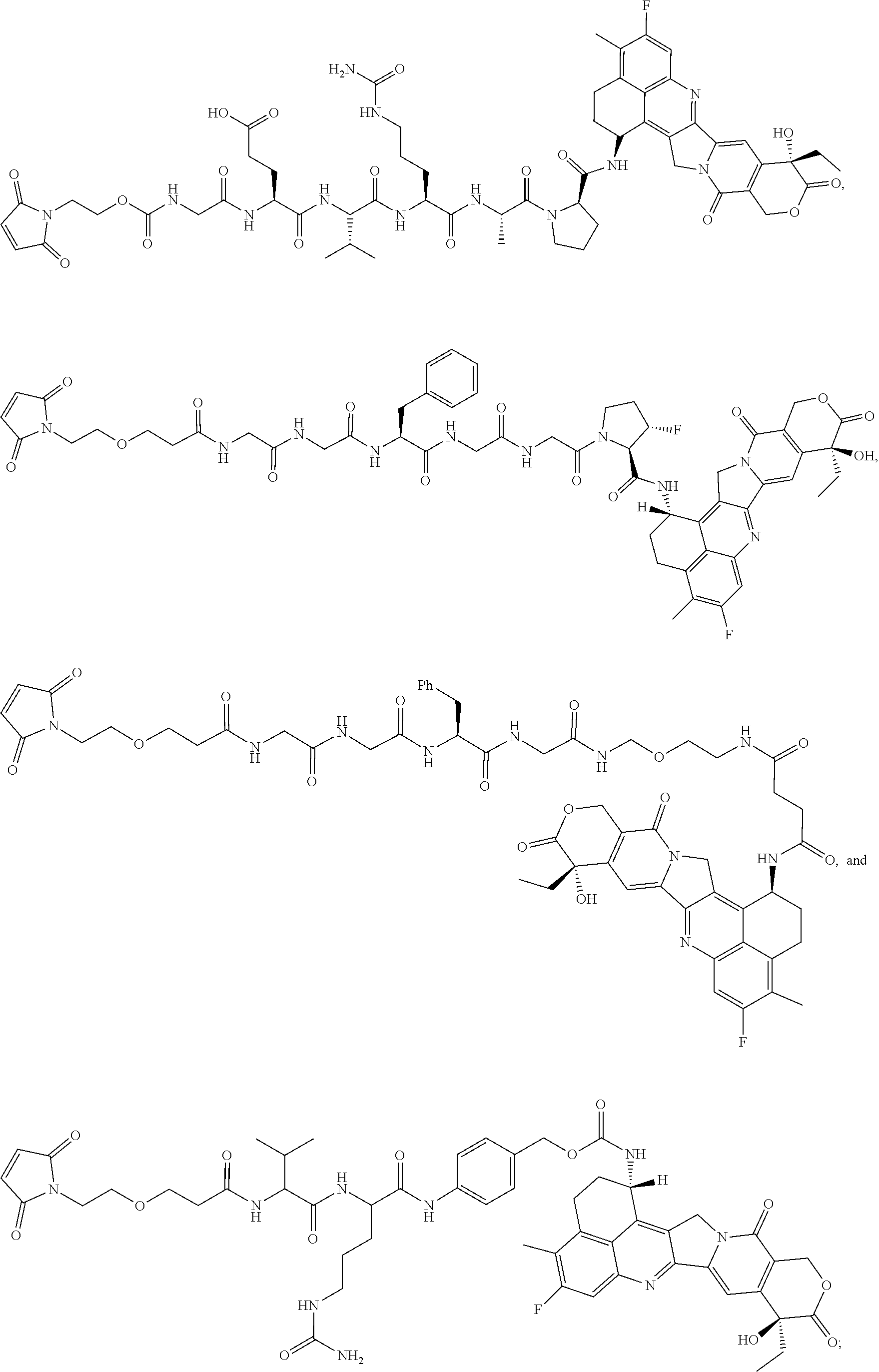
or a pharmaceutically acceptable salt or stereoisomer thereof.

65.-86. (canceled)

87. A method of treating a cancer in patient in need thereof, comprising administering to the patient an effective amount of a compound of claim 1, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

88. A method of treating a cancer in patient in need thereof, comprising administering to the patient an effective amount of a compound of claim 61, wherein the cancer is selected from the group consisting of lung cancer, kidney cancer, urothelial cancer, colorectal cancer, prostate cancer, glioblastoma multiforme, ovarian cancer, pancreatic cancer, breast cancer, melanoma, liver cancer, bladder cancer, stomach cancer, and esophageal cancer.

89. (canceled)

90. A pharmaceutical composition comprising a compound of claim 1, and a pharmaceutically acceptable excipient.

91. A pharmaceutical composition comprising a compound of claim 61, and a pharmaceutically acceptable excipient.

92. (canceled)
